(19) United States
(12) Patent Application Publication
(10) Pub. No.: US 2015/0245597 A1
(43) Pub. Date: Sep. 3, 2015

(54) HETEROZYGOUS MOUSE WITH AN INACTIVATED BRD1 ALLELE AND USES IN PSYCHIATRY

(71) Applicant: CAPNOVA A/S, Aarhus N (DK)

(72) Inventors: Anders Borglum, Aarhus C (DK); Ole Mors, Aarhus C (DK); Mette Nyegaard, Aarhus C (DK); Jane Hvarregaard Christensen, Aarhus C (DK); Per Qvist, Aarhus C (DK); Anto Preveen Rajkumar Rajamani, Aarhus C (DK); Gregers Wegener, Aarhus C (DK)

(21) Appl. No.: 14/430,093

(22) PCT Filed: Sep. 19, 2013
(86) PCT No.: PCT/EP2013/069524

§ 371 Date: Mar. 20, 2015

(30) Foreign Application Priority Data
Sep. 21, 2012 (GB) 1216939.7

(51) Int. Cl. A01K 67/027; A61K 38/02; A61K 39/395; A61K 49/00; A61K 31/7088

(52) U.S. Cl. CPC A01K 67/0275; A61K 49/0008; A61K 31/7088; A61K 39/395; A61K 38/02; A01K 2227/105; A01K 2267/0306

(57) ABSTRACT

The present invention encompasses genetically modified non-human mammals comprising a genetic modification that inhibits and/or reduces BRD1 activity in one or more tissue or cell, methods of producing the same, methods and uses for identifying compounds for treating a mental disorder and pharmaceutical formulations of said compounds.

The present invention encompasses genetically modified non-human mammals comprising a genetic modification that inhibits and/or reduces BRD1 activity in one or more tissue, methods of producing the same, methods and uses for identifying compounds for treating a mental disorder and pharmaceutical formulations of said compounds.

Scientists are increasingly being asked both to develop the use of animal models for studying psychiatric disorders, such as alcohol and other substance abuse, schizophrenia, depression, and anxiety. Using animal models in behavioural research allow researchers to test specific hypotheses under highly controlled conditions using methods that are either impossible or unethical to use in humans. For example, researchers can create genetically altered mice to examine the influence of specific gene products on behaviour.

The domain structure of BRD1 assigns the protein to a family of bromodomain-PHD finger containing proteins (BRPFs). The BRPFs have been identified in the MOZ/MORF complex that together with the ING5 tumor suppressor and EAF6 (homolog of yeast Esa1-associated factor 6) possesses acetyltransferase activity specific for histone H3. Detailed studies of BRD1 have shown that it is part of yet another histone acetyltransferase (HAT) complex (including HBO1 and its activator protein named ING4) and that this complex is responsible for the bulk of the acetylation of histone H3K14.

Mice homozygous for inactivated alleles of the Brd1 gene display a lethal maturation defect in embryonic hematopoiesis in the liver as well as impaired eye developmental and neural tube closure, emphasizing the importance of the gene in embryonic development.

In the genome of cell lines, BRD1 seems to bind promoter regions and at transcription start sites of a large number of genes strongly indicating its importance in regulating the expression of large gene sets. The BRD1 transcript is widely expressed. It has been observed by Northern blotting in human spleen, thymus, prostate, testis, ovary, small intestine, colon, and peripheral blood lymphocyte as well as in various human cell lines (HL60, HeLa, K-562, MOLT-4, SW 480, A549, and G361).

The BRD1 protein is found to be widely but differentially expressed in different human tissues. It is expressed in all parts of the adult CNS with a predominant subcellular localization in the nucleus, the perikaryal cytosol, and proximal dendrites. The long isoform of BRD1 predominantly localize in the nuclei of neurons in the hippocampus and cortex of humans and rats as well as in oligodendrocyte in the deep white matter in humans. A similar staining pattern has been observed in many other human tissues, such as the intestinal, prostate, uterus and breast epithelium together with the pituitary, tonsil, spleen, testis, adrenal gland and liver. Others human tissues show primarily nuclear staining, such as ovary, lung, stomach, thyroid gland, thymus and bone marrow, while nuclear and more pronounced cytoplasmatic staining is seen in parathyroid gland, salivary gland, pancreas, and kidney.

An attempt has been made to develop a BRD1 inactivated mouse (see Mishima et al., 2011 (supra.)) in order to investigate the role of BRD1 in disease and development. However, the attempt was unsuccessful; all double BRD1 knockout strains died during gestation (mostly by 15.5 days post coitus). The authors found that BRD1 has a pivotal role in embryonic development in multiple tissues and organs (although they focussed on the particular BRD1-associated phenotype of anaemia). For this reason, the role of BRD1 in adults remains elusive.

Accordingly, there is an ongoing need to provide a non-human mammal with altered BRD1 expression in the hope that it will be suitable model for one or more mental disorders. In view of the reported lethality of BRD1 knockout in mice, a BRD1 overexpression model appears to be the most viable model for investigating BRD1 activity.

However, the present inventors have surprisingly created BRD1 knockout strains of non-human mammal. Accordingly, the first aspect of the invention provides a genetically modified non-human mammal comprising a genetic modification that inhibits and/or reduces BRD1 activity in one or more tissue.

By “genetically modified” we include organisms having: exogenous genetic material, such as a gene, or a promoter or other regulatory element; modified host genetic material, such as amino acid deletion, insertion and/or substitutions in a gene or regulatory element, and epigenetic modification, such as methylation. The genetic modification may be made through a nucleic acid construct integrated (randomly or in a targeted manner) into the genome. Vectors for stable integration include plasmids, retroviruses and other animal viruses, mammalian artificial chromosomes (MACs) yeast artificial chromosomes (YACs), and the like. Preferably, the modification is stably transmitted in host cells. Preferably, the modification is a partial or whole gene knock-out.

By “non-human mammal” we include any mammal other than humans, for example, a cow, dog, cat, goat, sheep, pig, rabbit or rodent or rodent (for example, a mouse or rat).

Preferably, the non-human mammal is a rodent, preferably a mouse. Preferably, the genetically modified non-human mammal of the invention is substantially congenic, for example, at least 90% congenic, for example, at least 91%, 92%, 93%, 94%, 95%, 96%, 97%, 98% or at least 99% congenic. Most preferably the genetically modified non-human mammal of the invention is 100% congenic.

By “inhibits and/or reduces “BRD1 activity” we include that:
the amount of BRD1 mRNA and/or
the amount of BRD1 protein; and/or
the BRD1 acetyltransferase activity,
   of the genetically modified non-human mammal is lower than in negative controls (e.g., non-human mammals of the same or comparable genetic background having wildtype “BRD1” activity levels, for example, non-human mammals lacking genetic modification of “BRD1”). Methods of detecting and/or measuring the concentration of protein and/or nucleic acid are well known to those skilled in the art (see for example Sambrook and Russell, 2001, Cold Spring Harbor Laboratory Press) as are methods of determining BRD1 acetyltransferase activity (see below).

Preferred methods for detection and/or measurement of protein include Western blot as e.g. described (Christensen, et al., 2012, Eur. Neuropsychopharmacol. 22(9):651-6), immunosorbent assays (ELISA), antibody microarray, tissue microarray (TMA), immunoprecipitation, and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR) as e.g. described Christensen, et al., 2011 (supra.), nanoarray, microarray, macroarray, next-generation RNA sequencing (RNAseq) and in situ hybridisation.

Preferred methods for detection and/or measurement of acetyltransferase activity are described in Mishima et al., 2011 (supra.). BRD1 acetyltransferase activity was determined by measuring the amount HB01-BRD1 complex or H3K14 acetylation using specific antibodies (see page 2444, left column, fourth full paragraph to right column, first full paragraph and the Supplemental Methods, which are incorporated by reference herein). HB01-BRD1 complex or H3K14 acetylation may also be quantitatively determined using mass spectrometry. Additionally or alternatively, BRD1 activity may be extrapolated from mRNA and/or protein level or amount (for example, using Quantitative Real Time PCR).

The present inventors found BRD1 inactivation to be associated with aberrant behaviours (including psychosis-like behaviour, aberrant social behaviour, impaired cognitive behaviour and depressive-like behaviour—see Example 1, below) directly implicating BRD1 in various mental disorders that previously, it had at best, been circumstantially linked to.

As the BRD1 gene is highly expressed in the adult CNS (Bjarkam et al., 2009 Brain Struct Funct. 214(1):37-47; Severinsen, et al., 2006, Mol. Psychiatry 11, 1126-1138) and is implicated in epigenetic regulation of a large set of genes (Mishima et al., 2011 Blood, 118(9):2443-2453), it is thought that BRD1 serves important roles in the brain during adult life.

Linkage studies in various human populations performed by separate research groups implicate BRD1 in metal disorders including schizophrenia (SZ) and bipolar affective disorder (BPD) (for example Severinsen, et al., 2006, supra.; Nyegaard et al., 2010, Am J Med Genet B Neuropsychiatr Genet. 153B(2):582-91).

Severinsen, et al., 2006 supra., suggests that chromosome 22q12-13 may contain one or more shared susceptibility genes for schizophrenia (SZ) and bipolar affective disorder (BPD). The authors previously reported association between microsatellite markers located at 22q13.31-qtel and both disorders. Their 2006 paper reports an association analysis across five genes (including 14 single nucleotide and two microsatellite polymorphisms). BRD1 showed association with both disorders with minimal P-values of 0.0046 and 0.00001 for single marker and overall haplotype analysis, respectively. A specific BRD1 2-marker ‘risk’ haplotype showed a frequency of approximately 10% in the combined case group versus approximately 1% in controls (P-value 2.8×10(−7)). Expression analysis of BRD1 mRNA revealed widespread expression in mammalian brain tissue, which was substantiated by immunohistochemical detection of BRD1 protein in the nucleus, perikaryal cytosol and proximal dendrites of the neurons in the adult rat, rabbit and human CNS. Quantitative mRNA analysis in developing fetal pig brain revealed spatiotemporal differences with high expression at early embryonic stages, with intense nuclear and cytosolar immunohistochemical staining of the neuroepithelial layer and early neuroblasts, whilst more mature neurons at later embryonic stages had less nuclear staining.

The genetically modified non-human mammal of the first aspect of the invention may exhibit one or more phenotype associated with a mental disorder.

In rodents these mental disorders are associated with one or more of the following symptom areas that may be tested as indicated in Table 1.

BRD1 is particularly associated with the following mental disorders: Schizophrenia; Bipolar Affective Disorder; Major Depressive Disorder; Generalized Anxiety Disorder; ADHD; Childhood Autism; and Dementia. For more information on the symptoms and classification of these mental disorders (except ADHD) in humans see “The ICD-10 Classification of Mental and Behavioural Disorders (Diagnostic criteria for research)” World Health Organization, 1993 which is incorporate by reference herein—in particular, Sections F20 (Schizophrenia), F30 (Bipolar Affective Disorder), F32 (Major Depressive Disorder), F41.1 (Generalized Anxiety Disorder), F84 (Childhood Autism) and F00-F03 (Dementia). For more information on the symptoms and classification of ADHD in humans see “Diagnosis and management of ADHD in children, young people and adults (National Clinical Practice Guideline Number 72)” 2009, The British Psychological Society and The Royal College of Psychiatrists; pages 18-26 which is incorporated herein by reference—in particular pages 18-26.

Clinically diagnosed schizophrenia is associated with a much broader range of mental disorders in first-degree relatives than previously reported. Almost any other psychiatric disorder among first-degree relatives increased the individual's risk of schizophrenia. The population attributable risk associated with psychiatric family history in general was 27.1% whereas family histories including schizophrenia only accounted for 6.0% (Mortensen, P. B., Pedersen, M. G. & Pedersen, C. B. Psychiatric family history and schizophrenia risk in Denmark: which mental disorders are relevant? Psychol Med 40, 201-10 (2010)). This epidemiological data clearly demonstrates that schizophrenia share risk factors, including genetic risk factors, with most mental disorders.

In addition, symptom dimensions such as anxiety, depression, hyperactivity, cognitive impairment and psychotic symptoms are shared between schizophrenia, bipolar disorder and other mental disorders showing that some symptoms and genetic risk factors are in part unique and in part overlapping (Burmeister, M., McInnis, M. G. & Zollner, S. Psychiatric genetics: progress amid controversy. Nat Rev Genet 9, 527-40 (2008)).

Not only symptoms, but also the full syndromes are shared among some mental disorders, e.g. the full syndrome of depression as it occurs in bipolar disorder is identical to the syndrome defining unipolar depression (single episode or recurrent). Depressive episodes are common in schizophrenia either as preceding the psychotic illness (life-time comorbidity) or concurrent with schizophrenia (concurrent comorbidity) (WHO. The ICD-10 classification of mental and behavioural disorders. Diagnostic Criteria for Research. World Health Organization, Geneva, 1993. (1993); and American Psychiatric Association. Diagnostic and Statistical Manual of Mental Disorders, Fourth Edition—(DSMIV). APA (1994)).

Pharmacological treatment of psychotic symptoms is efficient in schizophrenia—but also in bipolar disorder and psychotic depression. Likewise, antidepressants are used for treating the depression syndrome in any mental disorder, e.g. bipolar, schizophrenia and mental retardation. In addition antidepressants are used for treating anxiety disorders and for eating disorders. Thus pharmacological evidence support shared disease mechanisms in mental disorders (Kaplan, B. J. & Sadock, V. A. Comprehensive Textbook of Psychiatry

Current evidence show that the same genetic variation, e.g deletions and duplications or common genetic variation, e.g SNP's, convey susceptibility to a range of mental disorders (Purcell, S. M. et al. Common polygenic variation contributes to risk of schizophrenia and bipolar disorder. Nature 460, 748-52 (2009); Williams, H. J. et al. Most genome-wide significant susceptibility loci for schizophrenia and bipolar disorder reported to date cross-traditional diagnostic boundaries. Hum Mol Genet 20, 387-91 (2011); Glessner, J. T. et al. Autism genome-wide copy number variation reveals ubiquitin and neuronal genes. Nature 459, 569-73 (2009); ISC. Rare chromosomal deletions and duplications increase risk of schizophrenia. Nature 455, 237-41 (2008); Merikangas, A. K., Corvin, A. P. & Gallagher, L. Copy-number variants in neurodevelopmental disorders: promises and challenges. Trends Genet 25, 536-44 (2009); Morrow, E. M. Genomic copy number variation in disorders of cognitive development. J Am Acad Child Adolesc Psychiatry 49, 1091-104 (2010); Stefansson, H. et al. Large recurrent microdeletions associated with schizophrenia. Nature 455, 232-6 (2008); and Williams, N. M. et al. Rare chromosomal deletions and duplications in attention-deficit hyperactivity disorder: a genome-wide analysis. Lancet 376, 1401-8 (2010)).

Specifically for the chromosomal region harbouring BRD1 (22q13.3), deletions have been found in patients with autism, autism like behaviour and mental retardation (Cusmano-Ozog, K., Manning, M. A. & Hoyme, H. E. 22q13.3 deletion syndrome: a recognizable malformation syndrome associated with marked speech and language delay. Am J Med Genet C Semin Med Genet 145C, 393-8 (2007); Goizet, C. et al. Case with autistic syndrome and chromosome 22q13.3 deletion detected by FISH. Am J Med Genet 96, 839-44 (2000)).

The importance of the BRD1 gene during neurodevelopment has previously been shown by us via analyses of its expression in embryonic neuroepithelial cells and neuroblasts, as well as its differential tempo-spatial expression at the mRNA level in the developing pig brain (Severinsen, J. E. et al. Evidence implicating BRD1 with brain development and susceptibility to both schizophrenia and bipolar affective disorder. Mol Psychiatry 11, 1126-38 (2006)). Recent findings, that mice homozygous for inactivated alleles of the Brd1 gene, in addition to a lethal maturation defect in embryonic haematopoiesis in the liver, display impaired eye developmental and neural tube closure, further emphasize the importance of the gene in embryonic neuronal cells (Mishima et al., 2011, supra.). Furthermore, as the BRD1 gene is highly expressed in the adult CNS^{16,18} and is implicated in epigenetic regulation of a large set of genes (Mishima et al., 2011, supra.), it is very likely that BRD1 also serves important roles in the brain during adult life.

Abnormal neurodevelopment is the key feature of a number of mental disorders such as mental retardation, autism, ADHD and schizophrenia, and due to the central role of BRD1 in neurodevelopment, it is possible that genetic variation in BRD1 is implicated in a range of mental disorders besides schizophrenia and bipolar disorder.

Methods for modeling human depression in rodents are well known in the art. For more information see Cryan & Mombereau, 2004, Molecular Psychiatry, 9, 326-357 (in particular, Tables 2 and 3), Cryan & Holmes, 2005, Nat Rev Drug Discov. 4(9):775-90 (in particular, Tables 1, 2 and 3), and Kas et al., 2011, Sci. Transl. Med., 3(102):1-6 (in particular, Table 1) which are incorporated herein by reference.

Hence, the one or more phenotype associated with a mental disorder is preferably selected from the group consisting of: basic neurological function (e.g., using Irwin battery, hidden food, hotplate, rotarod, and/or home cage locomotion tests); motor activity (e.g., using the open field test); positive symptoms (e.g., using the prepulse inhibition test); psychomotor agitation (e.g., using the hyperlocomotion in response to novelty or stress test), psychostimulant supersensitivity (e.g., using the hyperlocomotion in response drugs test); depression (e.g., using the tail suspension and/or forced swim tests); anxiety (e.g., using the bright open field, elevated plus maze, light/dark fear conditioning tests); anhedonia (e.g. using sucrose preference testing); cognition/memory (e.g., using the object recognition, 8 arm radial maze, T maze, continuous alternation, spontaneous alternation, morris water maze, fear conditioning, place recognition, and/or attentional set shifting tests); negative symptoms (e.g. using the social interaction test, and/or a three chamber test for sociability and preference for social novelty; cortical thinning (e.g., using anatomical examination); critical developmental stages (e.g., using age-matched developmental stages); disease progression (e.g., using longitudinal phenotypic assessment); environmental factors (e.g., using maternal infection, stressful events, cannabis use, social defeat tests); and genetic background/epistasis (e.g., using crossing mutant lines).

It is preferred that the host/background mammal from which the genetically modified mammal of the present invention is derived is diploid and, consequently, contains two copies of the BRD1 gene in each nucleated, non-reproductive cell (mature red blood cells lack a cell nucleus; spermatozoon and ova are haploid).

Nearly all mammals are diploid organisms, i.e., have two homologous copies of each chromosome, usually one from the mother and one from the father, although all individuals have some small fraction of cells that display polyploidy. The tetraploid (i.e., having four homologous copies of each chromosome) viscacha rats Pipanacoctomys aureus and Tympanoctomys barrerae are the only known exceptions. Human cells have 23 pairs of chromosomes (22 pairs of autosomes and one pair of sex chromosomes), giving a total of 46 per cell. The house mouse (Mus musculus) has a total of 40 chromosomes and the brown rat (Rattus norvegicus) has a total of 42.

Hence, in the genetically modified non-human mammal of the first aspect of the present invention, the genetic modification may be a mutation in one or both genomic copy of the BRD1 gene. The genetic modification may be a mutation in one genomic copy of the BRD1 gene. The genetic modification may be a mutation in both genomic copies of the BRD1 gene. Where the mammal is non-diploid, the genetic modification may be a mutation in any number of the genomic copy (or copies) of the BRD1 gene.

By “mutation” we include deletion, addition or substitution of one or more amino acid encoded by the BRD1 gene and/or deletion, addition or substitution of one or more nucleotides in its flanking regulatory sequence. Substitution or addition may be with any one of the 20 genetically encoded amino acids (other than the original amino acid). Substitution or addition may be with a hydrophobic or hydrophilic amino acid. Substitution or addition may be with an acidic, basic or neutral pH amino acid.

The genetically modified non-human mammal of the first aspect of the present invention may comprise a mutation in a coding or a non-coding region of the BRD1 gene.

Where the genetically modified non-human mammal of the first aspect of the present invention is a mouse, the mouse may comprise a mutation in exon 1B (amino acids 15 onwards), exon 2, exon 3, exon 4, exon 5, exon 6, exon 7A, exon 7B, exon 8, exon 9, exon, 10, exon 11 and/or exon 12 (amino acids 1-184), or any combination thereof. Alternatively or additionally, the mouse may comprise a mutation in exon 1A, the intron directly downstream of exon 1A, exon 1B (amino acids 1-14), the intron directly downstream of exon 1B, the intron directly downstream of exon 2, the intron directly downstream of exon 3, the intron directly downstream of exon 4, the intron directly downstream of exon 5, the intron directly downstream of exon 6, the intron directly downstream of exon 7A, the intron directly downstream of exon 7B, the intron directly downstream of exon 8, the intron directly downstream of exon 9, the intron directly downstream of exon 10, the intron directly downstream of exon 11 and/or the intron directly downstream of exon 12 (amino acids 1-184), or any combination thereof.

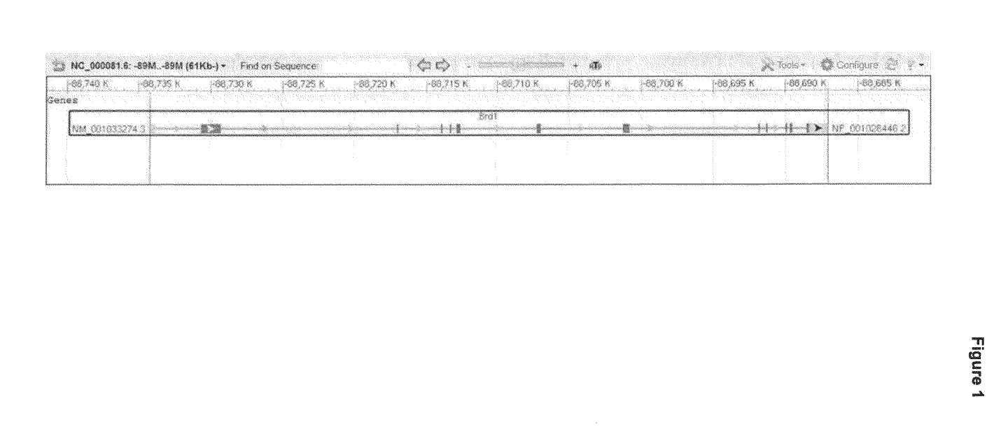

The mouse BRD1 gene is located on the complement (-) strand of chromosome 15, position 88687035-88734219, and spans 47185 bp. For full sequence see Table 3. The gene comprises 5137 bp (see FIG. 1) and consists of 12 exons of which all 12 are coding (see Table 4). However, two different variants of exon 1 exist as a result of alternative transcription start (1A and 1B; see Table 5). In addition, at least one alternative transcript has been found in which exon 7 is shorter by 393 bp (exon 7B). Both variants are protein coding (Brd1 (long) and Brd1 (short)). For a detailed overview of exons see Table 4.

The BRD1 gene encodes an 1189 aa protein, Brd1 (long). It contains 3 well described domains; a PHD-zinc-finger like domain, a bromodomain and a PWWP domain (Mishima et al., 2011). For predicted structure of the protein see Table 5, protein sequence Table 6. The 7B transcript variant (ENSMUST00000109380) encodes a slightly shorter protein (Brd1 short) of 1058 aa (Table 7). Brd1 (long) and Brd1 (short) share the first 786 aa and the last 272 aa, thus leaving all 3 domains intact in both variants.

Where the genetically modified non-human mammal of the first aspect of the present invention is a rat, the rat may comprise a mutation in exon 1B (amino acids 15 onwards), exon 2, exon 3, exon 4, exon 5, exon 6, exon 7, exon 7-long (where present), exon 8, exon 9, exon, 10, exon 11 and/or exon 12 (amino acids 1-184), or any combination thereof. Alternatively or additionally, the mouse may comprise a mutation in exon 1A, the intron directly downstream of exon 1A, exon 1B (amino acids 1-14), the intron directly downstream of exon 1B, the intron directly downstream of exon 2, the intron directly downstream of exon 3, the intron directly downstream of exon 4, the intron directly downstream of exon 5, the intron directly downstream of exon 6, the intron directly downstream of exon 7A, the intron directly downstream of exon 7B, the intron directly downstream of exon 8, the intron directly downstream of exon 9, the intron directly downstream of exon 10, the intron directly downstream of exon 11 and/or the intron directly downstream of exon 12 (amino acids 1-184), or any combination thereof.

In Rattus norvegicus the BRD1 gene is located on the complement (-) strand of chromosome 7, position 129366021-129413531, and spans 47511 bp. For full sequence see Table 8. The gene comprises 4500 bp and consists of 12 exons of which all 12 are coding. However, two different variants of exon 1 exist as a result of alternative transcription start (1A and 1B). Although not incorporated in the gene prediction of the UCSC Genome Browser, evidence exists for a long version of exon 7 as in mice and humans. For a detailed overview of BRD1 gene exons see Table 9.

The genetically modified non-human mammal of the first aspect of the present invention may comprise:

(i) One or more mutation substituting, deleting or inserting one or more nucleotide in the promoter or enhancer sequences of the BRD1 gene (resulting in reduced amounts of BRD1 mRNA);
(ii) One or more mutation introducing one or more premature stop codon in exon 1B to 11 (resulting in no expression of BRD1 mRNA or nonsense-mediated RNA decay and, thereby, reduced amounts of BRD1 mRNA);
(iii) One or more mutation affecting splice donors, splice acceptors or intronic branch sites (interfering with proper splicing of the BRD1 mRNA, resulting in either the production of aberrant non-functional BRD1 protein or reduced amounts of BRD1 mRNA due to nonsense-mediated RNA decay); and/or
(iv) A reduction in copy number of the BRD1 gene e.g., complete deletion of one or both copies of the BRD1 gene (resulting in reduced amounts of BRD1 mRNA).

The genetic modification of the non-human mammal of the first aspect of the invention may inhibit and/or reduce the expression of one or both genomic copy of the BRD1 gene (preferably both).

By “inhibits or reduces expression” we include that the amount of mRNA and/or protein of the genetically modified non-human mammal is lower than in negative controls (e.g., non-human mammals of the same or comparable genetic background having wildtype “BRD1” activity levels, for example, non-human mammals lacking genetic modification of “BRD1”).).

The expression of BRD1 in the genetically modified non-human mammal may be reduced by at least 10%, for example, at least 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or at least 100%.

Alternatively or additionally, the genetic modification of the non-human mammal may inhibit and/or reduce the normal function of one or both genomic copy of the BRD1 gene (preferably both).

By “normal function of one or both genomic copy of the BRD1 gene” we include acetyltransferase activity. Any suitable method for detection and/or measurement of acetyltransferase activity may be used. Preferred methods, as described in Mishima et al., 2011 (supra.), are discussed above. Additionally or alternatively, BRD1 activity may be extrapolated from mRNA and/or protein level or amount (for example, using Quantitative Real Time PCR) (i.e., mRNA levels taken to be indicative of BRD1 activity).

The genetic modification may be achieved using site-specific recombination.

Most site-specific recombinases are grouped into one of two families: the tyrosine recombinase family or the serine recombinase family. Serine recombinase family is also sometimes known as resolvase/invertase family, while tyrosine recombinases are known as the integrase family, which reflects the types of reaction that most known members in each family have evolved to catalyse. Typical examples of tyrosine recombinases are the well known enzymes such as Cre (from the P1 phage), FLP (from yeast S. cerevisiae) and λ integrase (from lambda phage) while famous serine recombinases include enzymes such as: gamma-delta resolvase (from the Tn1000 transposon), Tn3 resolvase (from the Tn3 transposon) and φC31 integrase (from the φC31 phage). Preferably, the genetic modification is achieved using the Cre-lox system

The genetically modified non-human mammal of the first aspect of the invention may comprise:
(i) One or more mutation introducing premature stop codons in exon 12 (resulting in the production of a truncated BRD1 protein and, thereby, in reduced activity either due to elimination of the BRD1 protein by protein quality control systems or reduced functional activity of the aberrant protein);
(ii) One or more mutation affecting splice donors, splice acceptors or intronic branch sites (interfering with proper splicing of the BRD1 mRNA and resulting in either the production of aberrant non-functional BRD1 protein or result in nonsense mediated RNA decay and, thereby, in reduced amounts of BRD1 mRNA);
(iii) One or more mutation substituting, deleting or inserting amino acid residues in the nuclear localization signals of BRD1 (resulting in faulty intracellular localization of BRD1 and, thereby, in reduced BRD1 activity);
(iv) One or more mutation substituting, deleting or inserting amino acid residues in the plant homeodomain finger, the bromodomain or the Pro-Trp-Trp-Pro domain (interfering with the three dimensional structure of the BRD1 protein and, thereby, in reduced activity either due to elimination of the BRD1 protein by protein quality control systems or reduced activity of the aberrant BRD1 protein); and/or
(v) One or more mutation substituting, deleting or inserting amino acid residues in the nuclear receptor binding signals (interfering with the three dimensional structure of the BRD1 protein and, thereby, in reduced activity either due to elimination of the BRD1 protein by protein quality control systems or reduced activity of the aberrant protein).

The activity of BRD1 in the genetically modified non-human mammal may be reduced by 100%, for example, 99% or less, 98% or less, 97% or less, 96% or less, 95% or less, 94% or less, 93% or less, 92% or less, 91% or less, 90% or less, 89% or less, 88% or less, 87% or less, 86% or less, 85% or less, 84% or less, 83% or less, 82% or less, 81% or less, 80% or less, 79% or less, 78% or less, 77% or less, 76% or less, 75% or less, 74% or less, 73% or less, 72% or less, 71% or less, 70% or less, 69% or less, 68% or less, 67% or less, 66% or less, 65% or less, 64% or less, 63% or less, 62% or less, 61% or less, 60% or less, 59% or less, 58% or less, 57% or less, 56% or less, 55% or less, 54% or less, 53% or less, 52% or less, 51% or less, 50% or less, 49% or less, 48% or less, 47% or less, 46% or less, 45% or less, 44% or less, 43% or less, 42% or less, 41% or less, 40% or less, 39% or less, 38% or less, 37% or less, 36% or less, 35% or less, 34% or less, 33% or less, 32% or less, 31% or less, 30% or less, 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less or at least 5% or less.

In the genetically modified non-human mammal of the invention, BRD1 activity may be inhibited and/or reduced in all, or substantially all, cells in the mammal.

By “substantially all cells in the mammal” we include that BRD1 activity is inhibited and/or reduced in at least 90% of the cells in which it is normally expressed in negative controls (e.g., non-human mammals of the same or comparable genetic background lacking the genetic modification). For example, BRD1 activity may be inhibited and/or reduced in at least 91%, 92%, 93%, 94%, 95%, 96%, 97%, 98% or at least 99% of the cells in the mammal.

Alternatively, in the genetically modified non-human mammal of the invention, BRD1 activity may be inhibited and/or reduced in a selection of cells, for example, cells of the CNS neurons; glia cells, forebrain, prefrontal cortex, hippocampus, amygdale, hypothalamus, gabaergic neurons, dopaminergic neurons, glutamitergic neurons and/or serotonergic neurons.

As noted above, BRD1 expression has been observed in human spleen, thymus, prostate, testis, ovary, small intestine, colon, peripheral blood lymphocyte, human whole brain, cerebellum, cerebral cortex, medulla, spinal cord, occipital pole, frontal lobe, caudate nucleus, corpus callosum, hippocampus and thalamus and in the spermatocytic population in the seminiferous tubules (ST) of mice.

The CNS (central nervous system) comprises the brain and the spinal cord. The PNS (peripheral nervous system) comprises nerves and ganglia outside of the brain and spinal cord. Both are composed primarily of two broad classes of cells: neurons and glial cells.

Selective BRD1 inhibition and/or reduction may be achieved by any suitable means for example:

1) Crossing F mice with mice expressing Cre under the control of a promoter which is specifically active in CNS neurons and glia cells e.g. the Nestin promoter (restricting phenotypes to those dependent of the CNS) (F mice (strain 1) are homozygous for conditional inactivation of BRD1 (e.g., a BRD1 knockout allele); R mice (strain 2) are heterozygous for constitutive inactivation of BRD1; W mice are wildtype);
2) Crossing F mice with mice expressing Cre under the control of a promoter which is specifically active in CNS neurons (restricting phenotypes to those dependent of this specific cell type in the CNS);
3) Crossing F mice with mice expressing Cre under the control of a promoter which is specifically active in forebrain e.g. the CamkII promoter (restricting phenotypes to those dependent of this specific brain region);
4) Crossing F mice with mice expressing Cre under the control of a promoter which is specifically active in prefrontal cortex (restricting phenotypes to those dependent of this specific brain region);
5) Crossing F mice with mice expressing Cre under the control of a promoter which is specifically active in hippocampus (restricting phenotypes to those dependent of this specific brain region);
6) Crossing F mice with mice expressing Cre under the control of a promoter which is specifically active in amygdale (restricting phenotypes to those dependent of this specific brain region);
7) Crossing F mice with mice expressing Cre under the control of a promoter which is specifically active in hypothalamus e.g., the Sim1 promoter (restricting phenotypes to those dependent of this specific brain region);
8) Crossing F mice with mice expressing Cre under the control of a promoter which is specifically active in gabaergic neurons (restricting phenotypes to those dependent of this specific cell type);
9) Crossing F mice with mice expressing Cre under the control of a promoter which is specifically active in dopaminergic neurons e.g. the tyrosin hydroxylase (TH) promoter (restricting phenotypes to those dependent of this specific cell type).
10) Crossing F mice with mice expressing Cre under the control of a promoter which is specifically active in glutamitergic neurons (restricting phenotypes to those dependent of this specific cell type);
11) Crossing F mice with mice expressing Cre under the control of a promoter which is specifically active in serotonergic neurons e.g. the PC12 ets factor 1 (PET1) enhancer region (restricting phenotypes to those dependent of this specific cell type); and/or
12) Infusions of Cre-expressing lentiviruses into specific brain areas of F mice (allow the reduction in BRD1 expression in any brain region accessible for infusion without confounding issues of brain development).

The activity of BRD1 may be reduced by approximately 50% in all, or substantially all, cells in the mammal.

However, it is preferred that BRD1 expression and/or activity in the liver (i.e., hepatic cells) is the same as, or substantially the same as, that of negative control (e.g., non-human mammals of the same or comparable genetic background having wildtype “BRD1” activity levels, for example, non-human mammals lacking genetic modification of “BRD1”). For example, the construct used to modify BRD1 expression and/or function may be under the control of tissue-specific promoter that is not active in hepatic cells such as the rat nestin promoter (see, for example, Dubois et al., 2006, Genesis, 44:355-360) which has little or no activity in tissues of the heart, liver, thymus and lung.

It is particularly preferred that that BRD1 expression and/or activity in cells other than neurons and/or glia is the same as, or substantially the same as, that of negative control (e.g., non-human mammals of the same or comparable genetic background having wildtype “BRD1” activity levels, for example, non-human mammals lacking genetic modification of “BRD1”). For example, BRD1 may only be differentially expressed in the CNS or PNS compared to negative control.

By “the same, or substantially the same as” we include at least within 50% of, for example, at least within 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or within 100%.

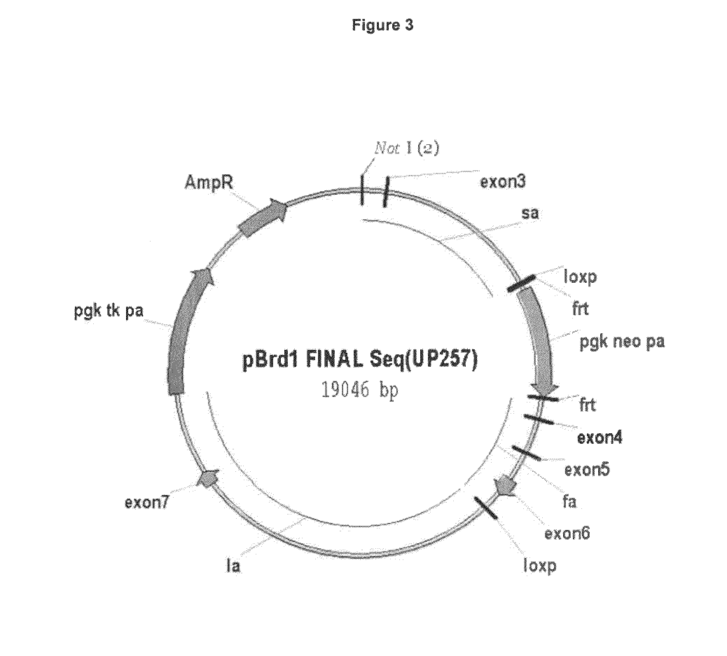

The genetic modification of the non-human mammal of the invention may comprise modification using the vector described in FIG. 3 and Table 12 (SEQ ID NO: 32). F mice (strain 1) are homozygous for the conditional KO allele, R mice (strain 2) are heterozygous for the constitutive KO allele.

BRD1 activity may be reduced by approximately 100% in all, or substantially all, cells and/or tissues in the genetically modified mammal of the invention. If so, it is preferred that the modification of BRD1 expression and/or activity is induced at a developmental stage wherein hematopoietic activity of the thymus and/or bone marrow is sufficient to sustain life, for example, postpartum. In mice, hematopoietic activity of the thymus and/or bone marrow may be sufficient to sustain life at 15.5 days post coitus (dpc), 16 dpc, 16.5 dpc, 17 dpc, 17.5 dpc, 18 dpc, 18.5 dpc, 19 dpc, 19.5 dpc, 20 dpc, 20.5 dpc or 21 dpc. The modification of BRD1 expression and/or activity may be induced using any suitable means known in the art, for example, an inducible promoter (e.g., the tamoxifen-inducible system described in Erdmann, Schutz & Berger, 2007, BMC Neuroscience, 8:63).

BRD1 activity may be reduced by approximately 50% in all, or substantially all, cell and/or tissues in the genetically modified mammal of the invention.

Alternatively, BRD1 activity may be reduced by approximately 50% in a selection of cells, for example, cells of the CNS neurons; glia cells, forebrain, prefrontal cortex, hippocampus, amygdale, hypothalamus, gabaergic neurons, dopaminergic neurons, glutamitergic neurons and/or serotonergic neurons.

Alternatively, BRD1 activity may be reduced by approximately 100% in a selection of cells, for example, cells of the CNS neurons; glia cells, forebrain, prefrontal cortex, hippocampus, amygdale, hypothalamus, gabaergic neurons, dopaminergic neurons, glutamitergic neurons and/or serotonergic neurons.

Hence, the first aspect of the invention may comprise or consist of a genetically modified non-human mammal comprising a genomic mutation which is capable of reducing and/or inhibiting BRD1 activity in one or more tissue or cell type.

The mammal of the first aspect of the invention may be selected from the group of mammals consisting of: cows, dogs, cats, goats, sheep, pigs, rabbits, mice and rats. Preferably the mammal is a rodent. More preferably the rodent is a rat (e.g., Rattus norvegicus or Rattus Rattus). Equally preferably, the rodent is a mouse (e.g., Mus musculus).

In one embodiment the genetically non-human mammal of the invention is a mouse that is at least 15.5 days post coitus old, postpartum or adult (at least 21 days postpartum old).

A second aspect of the invention provides a polynucleotide sequence comprising SEQ ID NO: 32.

A third aspect of the invention provides a method of generating a genetically modified, non-human mammal as defined in the first aspect of the invention, comprising the steps of:

A) Genetically modifying a host non-human mammal strain to be heterozygous for a inactivated BRD1 allele (constitutive or conditional inactivation);
B) Where the BRD1 allele in step (A) is conditionally inactivated, generating offspring heterozygous for a constitutively inactivated BRD1 allele.
C) Intercrossing of the heterozygously modified non-human mammal strain produced in step (A) or (B) to produce a non-human mammal strain homozygous for an inactivated BRD1 allele.

Preferably, the non-human mammal is a rodent (e.g., a rat or a mouse).

The conditionally inactivated BRD1 allele may be inactive, or substantially inactive, in liver cells. Preferably the conditionally inactivated BRD1 allele is inactive, in liver cells. Preferably the inactivated BRD1 allele is under the regulation of the rat Nestin promoter. Preferably the method uses Cre-Lox recombination. Preferably the method uses site-specific recombination between the loxP sites flanking exon 3-5 of BRD1, promoted by the Cre-recombinase encoded by the transgene of hemizygous B6.Cg-Tg(Nes-cre)1Kln/J mice (The Jackson Laboratory) which expresses the enzyme under the control of the rat Nestin promoter and enhancer (see, for example, R. Feil, 2007, “Conditional somatic mutagenesis in the mouse using site-specific recombinases” Handb. Exp. Pharmacol., (178):3), which is incorporated herein by reference.

A fourth aspect of the invention provides a cell isolated from a genetically modified non-human mammal as defined the first aspect of the invention.

Methods of isolating cells are well known to those skilled in the art (see for example Molecular Biology of the Cell. 4th edition. Alberts B, Johnson A, Lewis J, et al., New York: Garland Science; 2002).

The cell may be a cell of the PNS or CNS. The cell may be a neuron or glial cell. Preferably, the cell is a neuron from the CNS.

A fifth aspect of the invention provides a method for identifying a compound for treating a mental disorder comprising the steps of:

(a) providing a test compound;
(b) administering the test compound to a genetically modified non-human mammal defined in the first aspect of the invention;
(c) determining whether the test compound reduces and/or inhibits the one or more phenotype associated with a mental disorder exhibited by the genetically modified non-human mammal; and
(d) identifying the test compound as a compound for treating a mental disorder if it reduces and/or inhibits the one or more phenotype associated with a mental disorder exhibited by the genetically modified non-human mammal.

The mental disorder exhibited by the genetically modified non-human mammal may be selected from the group consisting of Schizophrenia; Bipolar Affective Disorder; Major Depressive Disorder; Generalized Anxiety Disorder; ADHD; Childhood Autism; and Dementia.

The phenotype associated with a mental disorder exhibited by the genetically modified non-human mammal may be selected from the group defined in Table 1. The test used to determine whether the test compound reduces and/or inhibits the one or more phenotype associated with a mental disorder may be selected from the group defined in Table 1, for example:

### Basic Neurological Function:

Full basic physiological characterization may be carried out in a functional observational battery (Irwin's test) supplemented with assessment of basic motor-coordination skills in accelerating rotarod settings and nociception levels as tested in a Hotplate setup. General locomotion may be assessed in an open field (OF).

### Positive Symptoms:

Gaiting and re-activity of the startle reflex may be investigated by Acoustic Startle Response (ASR) and Pre-Pulse Inhibition (PPI) tests. In addition to baseline scores mice may be tested during pharmacological challenge; PCP (2.5 and 5 mg/kg s.c.) and amphetamine (2.5 and 5 mg/kg s.c.).

### Psychostimulant Supersensitivity:

Psychotropic drug-induced locomotor hyperactivity may be established by injections with PCP (1.3, 2.5, and 5 mg/kg s.c.), amphetamine (1.3, 2.5, and 5 mg/kg s.c.) and cocaine (10, 20, and 30 mg/kg s.c.) as opposing saline vehicle s.c. and measured by recording both horizontal locomotor activity and rearing activity in an automated photo-cell equipped home-cage.

### Depression:

Depressive equivalent behaviors may be assessed by forced swim test (FST) and tail suspension test (TST).

### Anxiety Assessment:

Anxiety equivalent behaviors may be assessed by bright open field (BOF), light and dark box (LDB), elevated plus maze (EPM) and fear conditioning (FCS).

### Anhedonia Assessment:

Anhedonia is defined as the inability to experience pleasure from an activity usually found enjoyable, and includes the motivation or desire of an individual to engage in an activity (“motivational anhedonia”), and the level of enjoyment derived from the activity itself (“consummatory anhedonia”).

Anhedonia may be assessed by sucrose preference testing.

As an example, sucrose preference testing may be carried out in the following way. Mice in their home cage are presented with two dual-bearing sipper tubes—one tube containing plain drinking water, and the second tube containing a 2-4% sucrose solution. Prior to beginning testing, mice should be habituated to the presence of two drinking bottles (one containing 2% sucrose and the other water) for three days in their home cage. Following this acclimation, mice should have the free choice of either drinking the 2% sucrose solution or plain water, for a period of four days. Water and sucrose solution intake should be measured daily, and the positions of two bottles should be switched daily to reduce any confound produced by a side bias. Sucrose preference should be calculated as a percentage of the volume of sucrose intake over the total volume of fluid intake, and averaged over the four days of testing. A bias toward the sweetened drink is typical, and failure to do so is indicative of anhedonia/depression.

### Cognition/Memory:

Exploratory and working memory components may be addressed by various types of Y-maze alternation tasks including spontaneous alternation test with dark phase testing, continuous alternation, and delayed alternation. Both baseline and induced behaviour (PCP 1.3 mg/kg s.c. and 2.5 mg/kg s.c.) may be assessed (PCP 1.3 mg/kg s.c. and 2.5 mg/kg s.c.). In all Y-maze tasks, alternation will calculated as the percentage of right choices out of the total arm entries.

Spatial learning and spatial working memory may be tested in the Morris Water Maze (MWM). Learning may be scored based on latency to escape while memory may be scored based on frequency and time spend in each zone of the maze.

Context as well as cue dependent learning and extinction retrieval may be assessed by fear conditioning system experiments (FCS). Working and visuo-spatial memory may be assessed by the 8-arm radial maze.

Medial frontal cortex functions may be assessed by the attentional set shifting test following a modified version of the protocol stated in Colacicco et al. 2002 Behavioural Brain Research 132: 95-102. The test may be split into 4 test days (1. Simple discrimination (SD), 2. Compound discrimination (CD)+compound reversal (CDR), 3. CDR repetition (CDRrep)+Intra-dimensiona (ID) shift and 4. extra dimensional (ED) shift) in order to keep mice motivated. Test may be balanced with equal numbers of 1) mice shifting from odor to media and 2) mice shifting media to odor and exemplars within pairs may be selected so mice did not show any preference (or avoidance) toward one over the other.

### Negative Symptoms:

Social behavior may be assessed by social interaction tests and/or assessing the “preference for novelty”.

Social behavior may be assessed by a social interaction test including recording and scoring of active social interaction, passive social interaction and aggressive interaction to monitor how mice respond to an unknown partner in a 10 min trial. Social memory may be tested by repeating the test after 48 hours.

Sociability—and “preference for novelty” may be assessed in a three-chamber box using a test comprising the following three phases. Phase I: Both cylinders should be left empty and the target mouse introduced to centre chamber and behaviour recorded for 10 minutes. Phase II: An unfamiliar mouse should be placed in one of the cylinders and a similar-sized toy mouse placed in the other. Phase III: Familiar partner should remain in its cylinder, and the toy mouse replaced by an unfamiliar mouse. The target mouse should be removed at the end of each phase and reintroduced at the start of the next. Test for remote social memory should be conducted one week later, with the unfamiliar mouse from Phase III in one cylinder and new unfamiliar mouse in the second cylinder. Animals should be scored on time spend in each compartment and time spend within a 3 cm distance of cylinders.

### Data Collection and Analysis:

Social interaction, continuous- and delayed alternation, FST, TST, LDB and EPM may be scored manually whereas the remaining tests may be scored automatically. Ethovision XT 8.0 may be used to score the OF and BOF. TSE FCS 8.06 may be used to score the FCS. Appropriate tests of statistical significance may be used to assess the behavioral differences between model mice and their controls and the possible enhancement obtained by administration of the compound. Appropriate multivariate statistics with STATA12.0 may be used to adjust for the effects of potential confounders.

A statistically significant enhancement in one or more of the phenotypes of the indicated mouse strains by a screened compound would indicate that it exhibits beneficial properties in other animals and in humans with equivalent diseases.

Preferably, the compound for treating a mental disorder acts by one or more of the following mechanisms:

1) Up-regulation of BRD1 levels (mRNA or protein);
2) Up- or down-regulation of genes regulated by BRD1 (mRNA or protein);
3) Up-regulation of BRD1 activity;
4) Increase of BRD1 dependent histone modifications;
5) Inhibition of removal of BRD1 dependent histone modification;
6) Enhancement of BRD1 dependent signal transduction in neurons;
7) Enhancement of BRD1 dependent neurotransmission;
8) Enhancement of BRD1 dependent neuroplasticity;
9) Increase of BRD1 dependent neurogenesis;

A sixth aspect of the invention provides the use of a genetically modified non-human mammal comprising a genetic modification which inhibits and/or reduces BRD1 activity in one or more cell or tissue, for identifying a compound for treating a mental disorder.

The mental disorder exhibited by the genetically modified non-human mammal may be selected from the group consisting of Schizophrenia; Bipolar Affective Disorder; Major Depressive Disorder; Generalized Anxiety Disorder; ADHD; Childhood Autism; and Dementia.

The phenotype associated with a mental disorder exhibited by the genetically modified non-human mammal may be selected from the group defined in Table 1. The test used to determine whether the test compound reduces and/or inhibits the one or more phenotype associated with a mental disorder may be selected from the group defined in Table 1 or described in respect of the fifth aspect of the invention (above).

A seventh aspect of the invention provides a method according to the fifth aspect of the invention or a use according to the sixth aspect of the invention, wherein the genetically modified non-human mammal is as defined in the first aspect of the invention, or is generated according to the method defined in the third aspect of the invention.

An eighth aspect of the invention provides a compound obtained or obtainable by the method according to the fifth or seventh aspects of the invention.

A ninth aspect of the invention provides a pharmaceutical composition comprising a compound as defined the eighth aspect of the invention and a pharmaceutical carrier or excipient.

It will be appreciated by persons skilled in the art that the medicaments and agents (i.e. polypeptides) will generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA, which is incorporated herein by reference).

For example, the medicaments and agents can be administered orally, buccally or sublingually in the form of tablets, capsules, ovules, elixirs, solutions or suspensions, which may contain flavouring or colouring agents, for immediate-, delayed- or controlled-release applications. The medicaments and agents may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The medicaments and agents of the invention can also be administered parenterally, for example, intravenously, intra-articularly, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the medicaments and agents will usually be from 1 to 1000 mg per adult (i.e. from about 0.015 to 15 mg/kg), administered in single or divided doses.

The medicaments and agents can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the medicaments and agents can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route.

For application topically to the skin, the medicaments and agents can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Where the medicament or agent is a polypeptide, it may be preferable to use a sustained-release drug delivery system, such as a microsphere. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Sustained-release immunoglobulin compositions also include liposomally entrapped immunoglobulin. Liposomes containing the immunoglobulin are prepared by methods known per se. See, for example Epstein et al., Proc. Natl. Acad. Sci. USA 82: 3688-92 (1985); Hwang et al., Proc. Natl. Acad. Sci. USA 77: 4030-4 (1980); U.S. Pat. Nos. 4,485,045; 4,544,545; 6,139,869; and 6,027,726. Ordinarily, the liposomes are of the small (about 200 to about 800 Angstroms), unilamellar type in which the lipid content is greater than about 30 mole percent (mol. %) cholesterol; the selected proportion being adjusted for the optimal immunoglobulin therapy.

Alternatively, polypeptide medicaments and agents can be administered by a surgically implanted device that releases the drug directly to the required site.

Electroporation therapy (EPT) systems can also be employed for the administration of proteins and polypeptides. A device which delivers a pulsed electric field to cells increases the permeability of the cell membranes to the drug, resulting in a significant enhancement of intracellular drug delivery.

Proteins and polypeptides can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and polypeptide delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Protein and polypeptide pharmaceuticals can also be delivered orally. One such system employs a natural process for oral uptake of vitamin B12 in the body to co-deliver proteins and polypeptides. By riding the vitamin B12 uptake system, the protein or polypeptide can move through the intestinal wall. Complexes are produced between vitamin B12 analogues and the drug that retain both significant affinity for intrinsic factor (IF) in the vitamin B12 portion of the complex and significant bioactivity of the drug portion of the complex.

The skilled person will appreciate that the most appropriate formulation will depend on a number of factors including route of administration, patient type (e.g. patient age, weight/size).

Exemplary embodiments of the invention are described in the following non-limiting examples, with reference to the following figures:

FIG. 1: Genomic position and structure of the mouse Brd1 gene (modified from Entrez Gene)

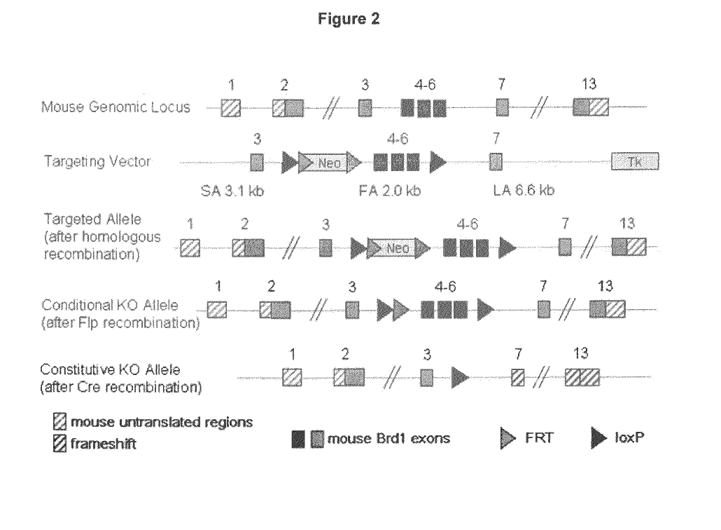

FIG. 2: Targeting strategy overview

Targeting strategy allows generation of conditional and constitutive knock-out (KO) alleles. Exons 4-6 has been flanked by loxP sites. Selection marker has been flanked by frt sites and introduced into intron 3. Conditional KO allele after in vivo Flp-mediated removal of selection marker. Constitutive KO allele after in vivo Cre-mediated recombination. Deletion of exons 4-6 should result in loss of function by removing the exons encoding the Bromo domain and generating a frameshift to downstream exons. Note: Exon numbering not in accordance with conventional numbering. Exon 1 should be 1a, exon 2 should be 1 b and the remaining exons should be as indicated minus 1.

FIG. 3: Targeting vector (pBrd1 FINAL Seq (UP257))

Note: Exon numbering not in accordance with conventional numbering. Exon 1 should be 1a, exon 2 should be 1b and the remaining exons should be as indicated minus 1.

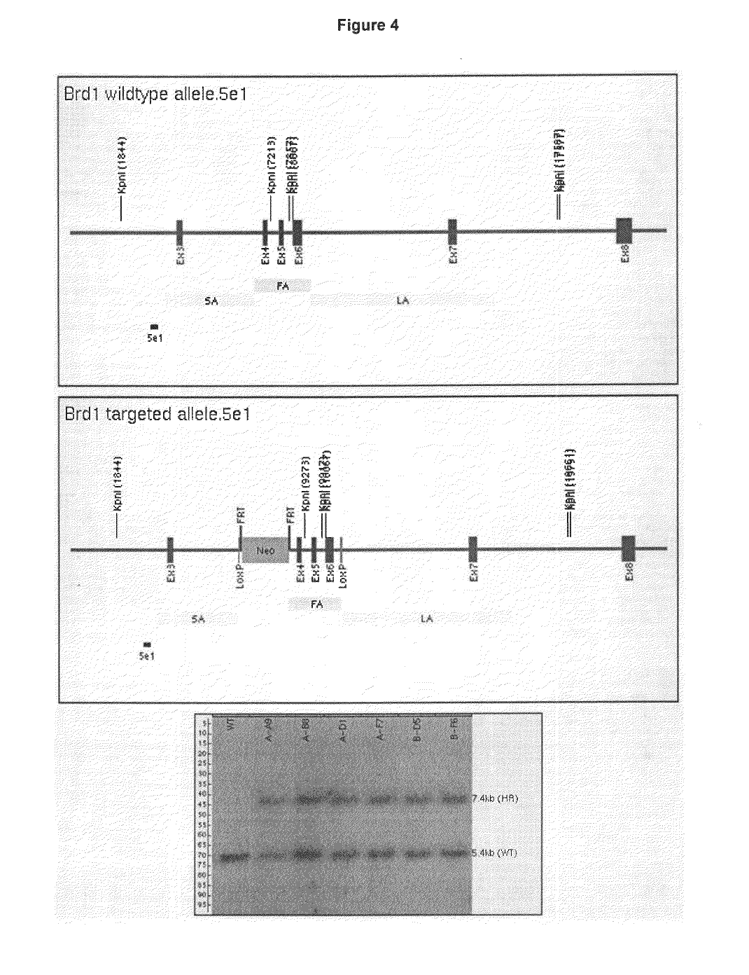

FIG. 4: Southern blot analysis of ES cell Transfection

Material: Genomic DNA from WT, A-A9, A-B8, A-D1, A-F7, B-D5, B-F6.

Method: Digestion with: Kpn1, Probe: 5e1

Results: Detects correct HR at 5′ side in all clones

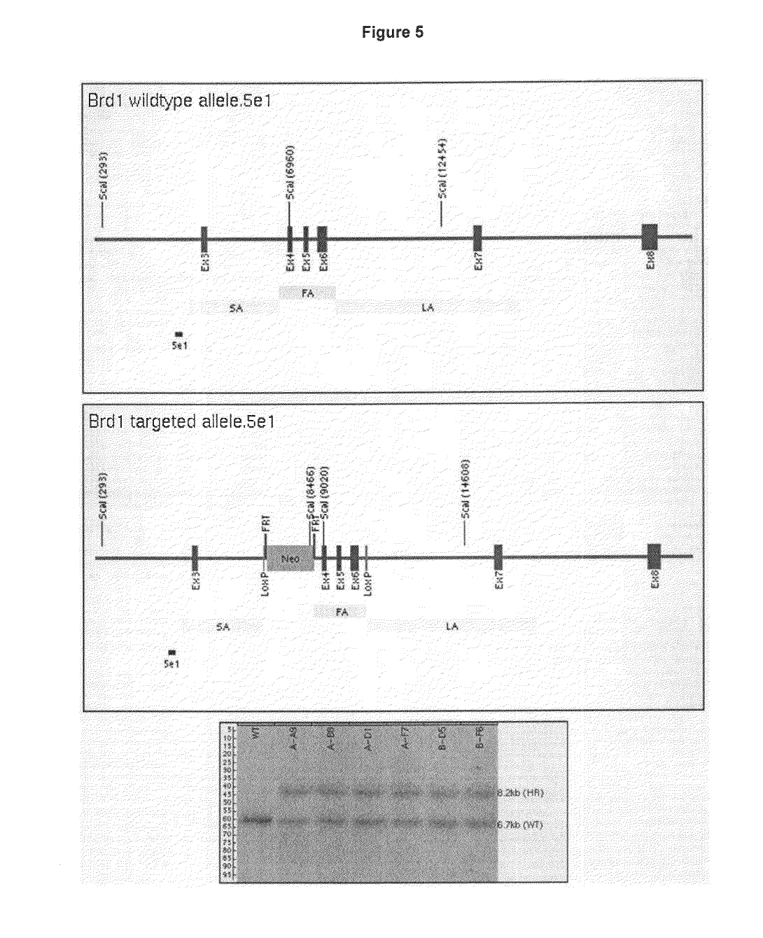

FIG. 5: Southern blot analysis of ES cell Transfection

Material: Genomic DNA from WT, A-A9, A-B8, A-D1, A-F7, B-D5, B-F6.

Method: Digestion with: ScaI, Probe: 5e1

Results: Detects correct HR at 5′ side in all clones

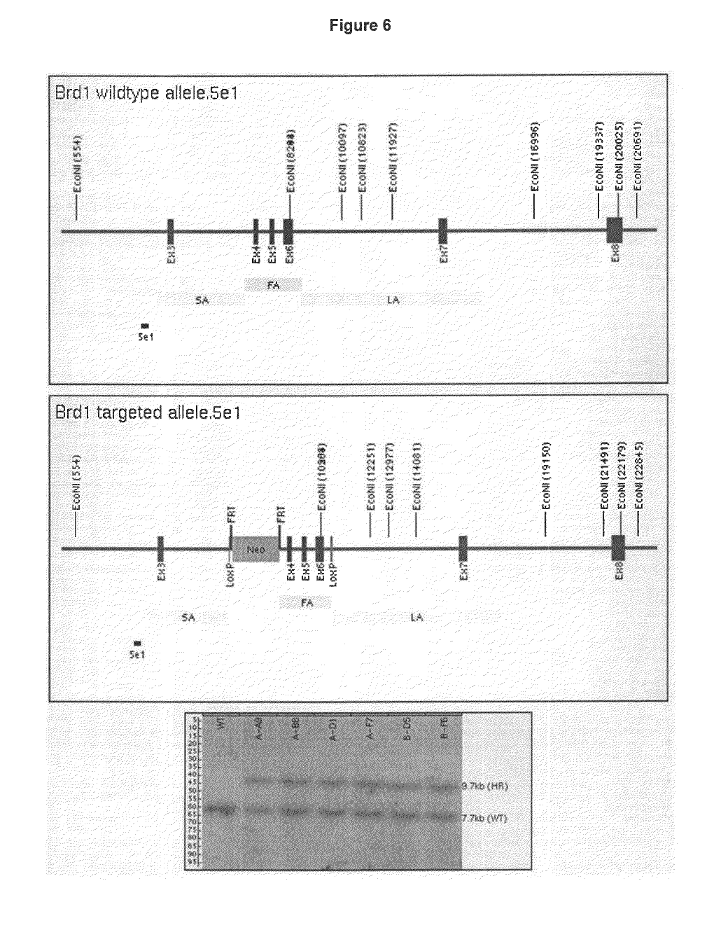

FIG. 6: Southern blot analysis of ES cell Transfection

Material: Genomic DNA from WT, A-A9, A-B8, A-D1, A-F7, B-D5, B-F6.

Method: Digestion with: EcoNI, Probe: 5e1

Results: Detects correct HR at 5′ side in all clones

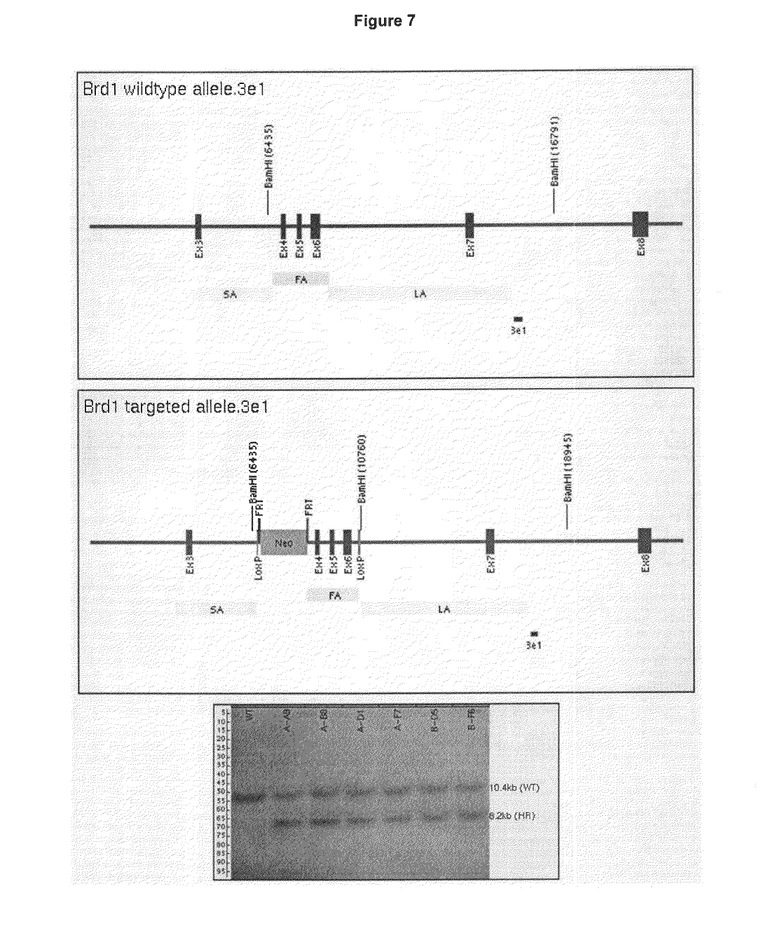

FIG. 7: Southern blot analysis of ES cell Transfection

Material: Genomic DNA from WT, A-A9, A-B8, A-D1, A-F7, B-D5, B-F6.

Method: Digestion with: BamHI, Probe: 3e1

Results: Detects correct HR at 3′ side and corecombination of distal loxP site in all clones

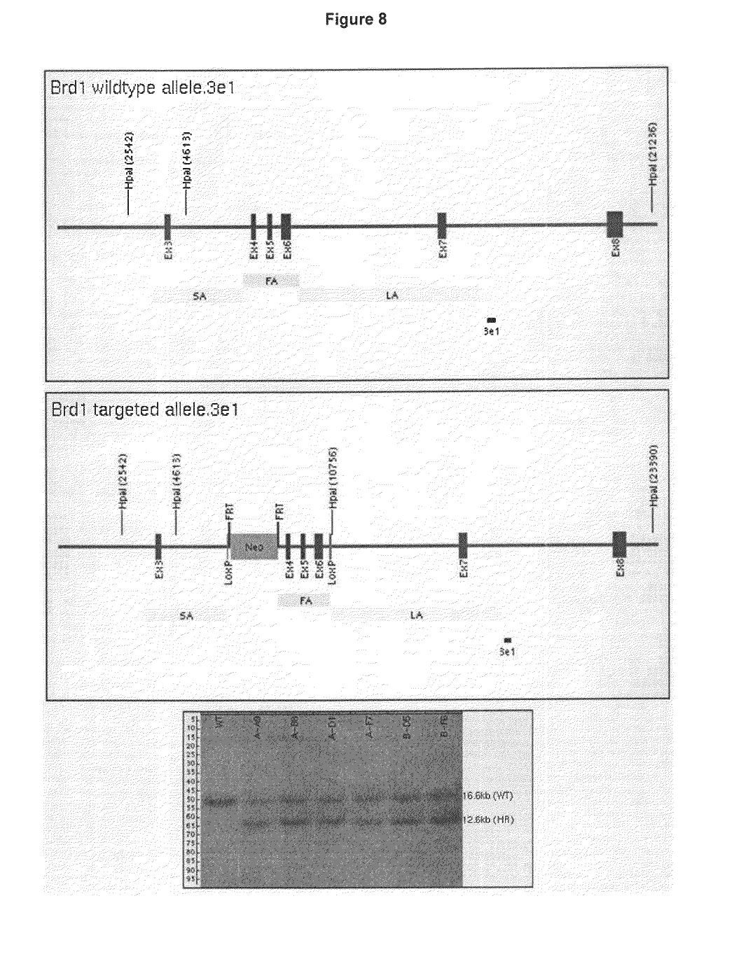

FIG. 8: Southern blot analysis of ES cell Transfection

Material: Genomic DNA from WT, A-A9, A-B8, A-D1, A-F7, B-D5, B-F6.

Method: Digestion with: HpaI, Probe: 3e1

Results: Detects correct HR at 3′ side and corecombination of distal loxP site in all clones

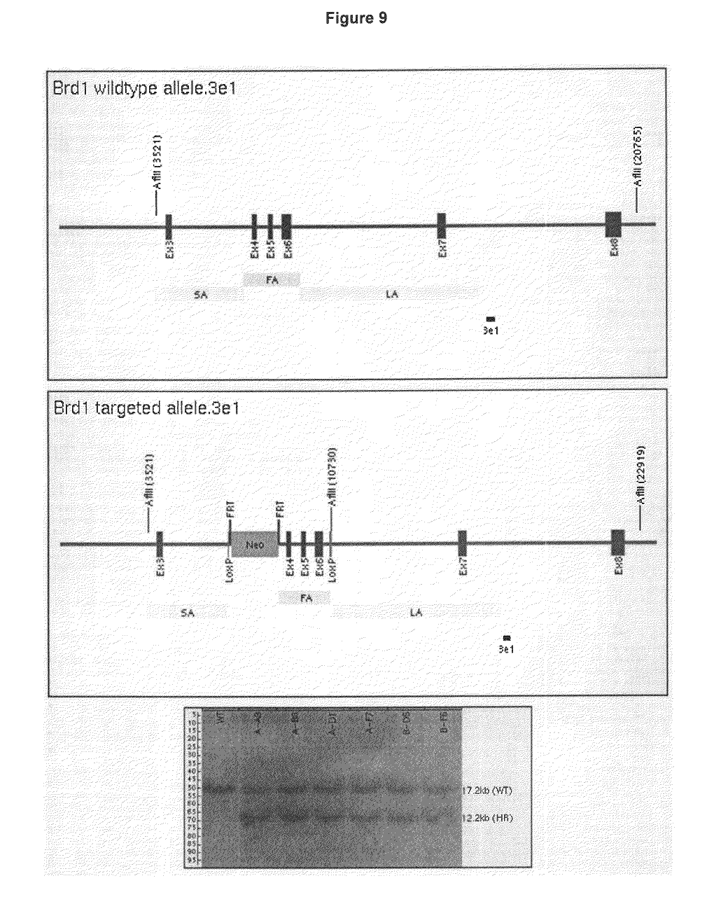

FIG. 9: Southern blot analysis of ES cell Transfection

Material: Genomic DNA from WT, A-A9, A-B8, A-D1, A-F7, B-D5, B-F6.

Method: Digestion with: Affil, Probe: 3e1

Results: Detects correct HR at 3′ side and corecombination of distal loxP site in all clones

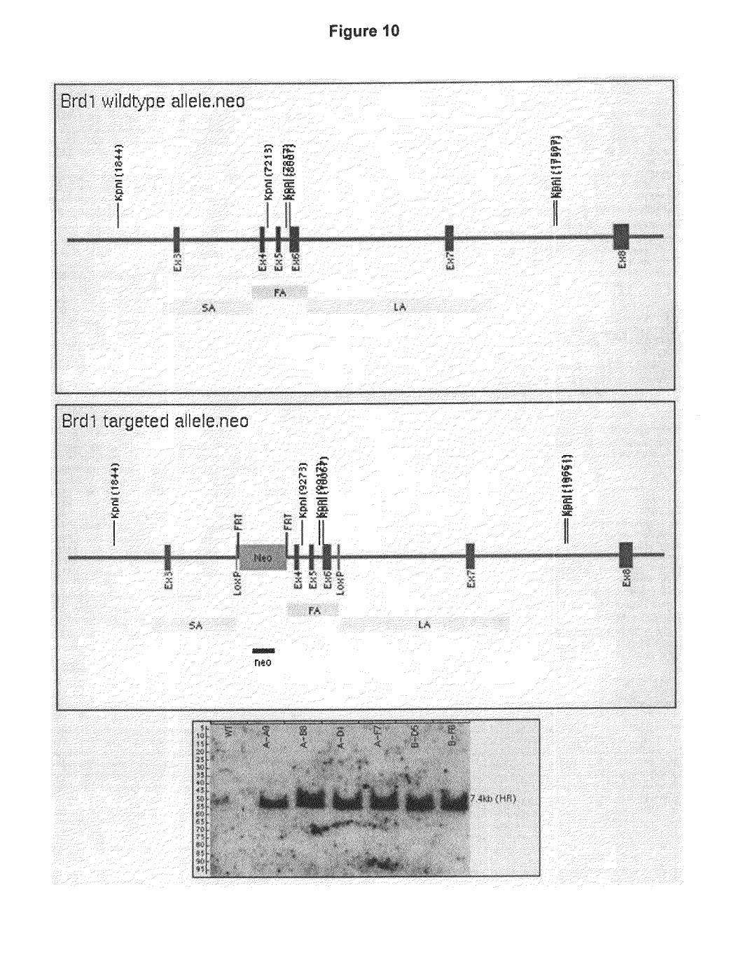

FIG. 10: Southern blot analysis of ES cell Transfection

Material: Genomic DNA from WT, A-A9, A-B8, A-D1, A-F7, B-D5, B-F6.

Method: Digestion with: KpnI, Probe: neo

Results: Detects correct HR at 5′ side and single integration in all clones

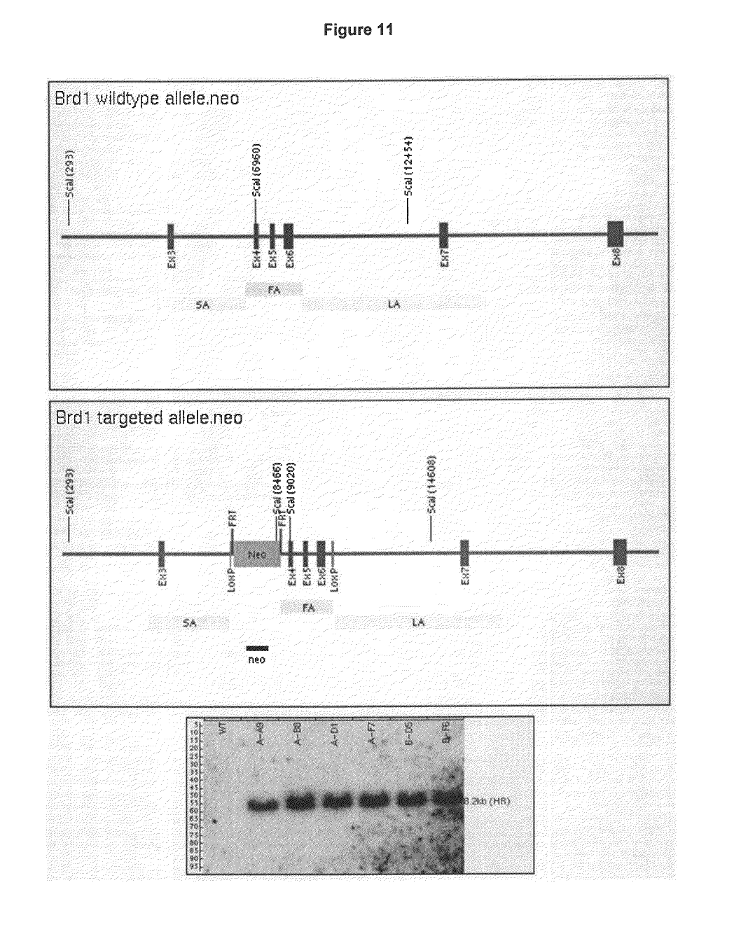

FIG. 11: Southern blot analysis of ES cell Transfection

Material: Genomic DNA from WT, A-A9, A-B8, A-D1, A-F7, B-D5, B-F6.

Method: Digestion with: ScaI, Probe: neo

Results: Detects correct HR at 5′ side and single integration in all clones

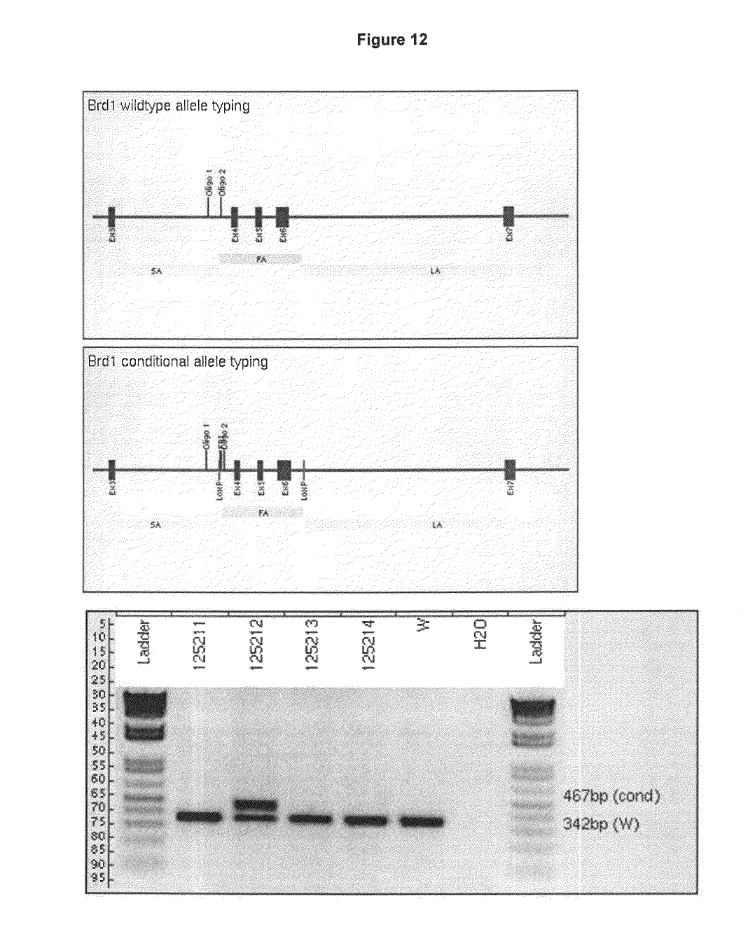

FIG. 12: Genotyping Analysis According to PCR SOP 1643

The fragment amplified with oligos 1 (1643_{—}27: GTAAGAGTACCGTGGTTAGC)+2 (1643_{—}28: GAGGTACAAACCTAAGCTACC) detects heterozygous/homozygous wildtype and conditional alleles. Due to highly palindromic repeats structures (FRT, multiple cloning site, loxP) in the conditional allele, an additional shorter artefact fragment might be visible in case of long electrophoretic separation.

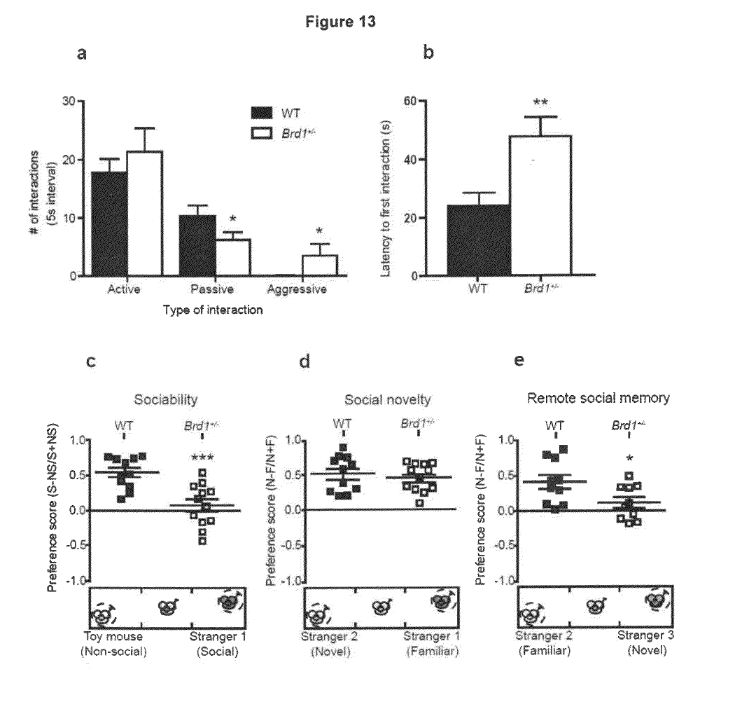

FIG. 13: Social interaction test and three chamber test for sociability and preference for social novelty

W mice are labelled Brd1^{+/+}. R mice are labelled Brd1^{+/-}.

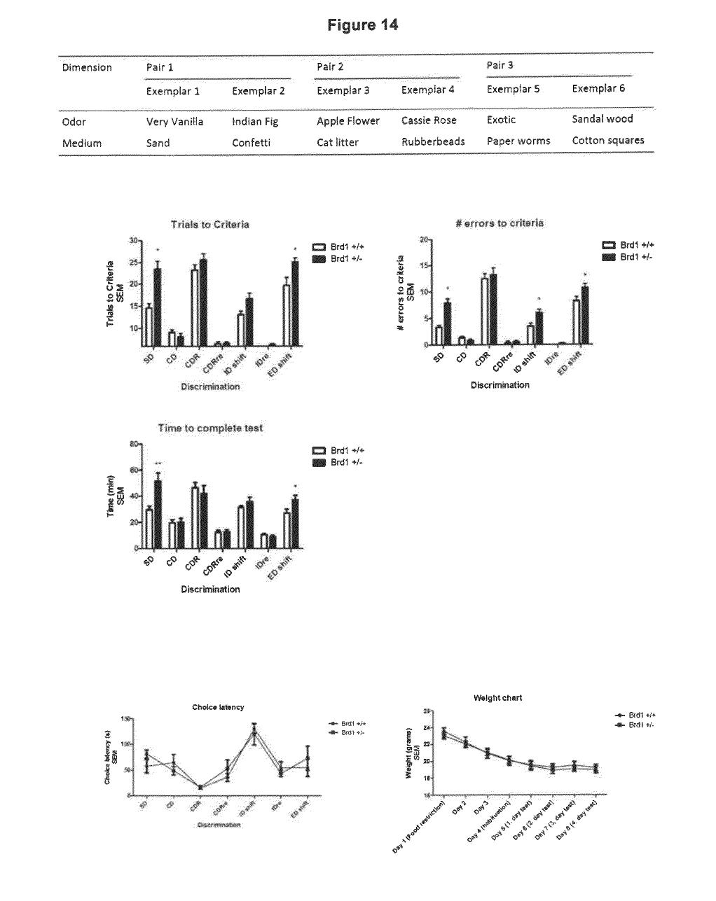

FIG. 14: Attentional set shifting test for cognitive impairment

W mice (n=9) are labelled Brd1+/+. R mice (n=9) are labelled Brd1+/−

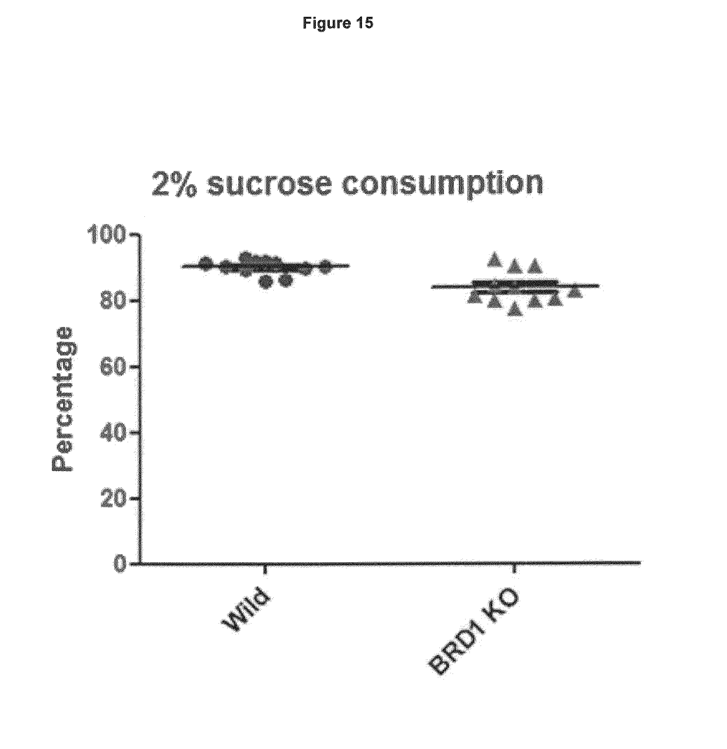

FIG. 15: Sucrose preference test for anhedonia

W mice (n=11) are labelled “wild”. R mice (n=11) are labelled BRD1 KO.

### EXAMPLES

### Example 1

### Data

We produced the targeted allele of the BRD1 gene with loxP sites flanking exon 3-5 as well as a frt site-flanked neomycin resistance gene by homologous recombination in C57BL/6 NTac embryonic stem (ES) cells. Correct homologous recombination and single integration was confirmed by Southern blotting analysis. Chimeric males (>50%) resulting from transfers of blastocysts injected with targeted ES clones into pseudopregnant mice were bred to Tg(ACTB-Flpe) tg/+ females (congenic C57BL/6 NTac genetic background, TaconicArtemis) to remove the neomycin resistance gene and generate offspring heterozygous for a conditional deleted allele, L mice. Mice heterozygous for the conditionally inactivated allele (L mice) were bred to homozygousity (F mice) by intercrossing.

Mice heterozygous for a constitutively inactivated allele (R mice), that is in which the function of one allele of the BRD1 gene is eliminated in all cells throughout development and adulthood, were produced by crossing L mice with ART12 rosa(Cre) KI mice (congenic C57BL/6 NTac genetic background, TaconicArtemis) to induce in vivo Cre-mediated recombination. Production of larger numbers of R mice and wildtype (W mice) litter mates for further investigations was achieved by continuously crossing of male R mice with the female C57BL/6 NTac mice (Taconic).

Efficient inactivation of the BRD1 gene was evaluated at the RNA level by quantitative RT PCR (Table 11).

Our strain 1 comprising F mice is fundamentally different from the mice produced by Mishima et al., as it is homozygous for the conditional deleted allele.

Our strain 2 comprising the R mice is different from the Mishima et al., mice by several means:

1) It is derived from strain 1 by in vivo Cre-mediated recombination;
2) We have ensured that the KO allele (conditional as well as constitutive) is contained in a congenic C57BL/6 NTac genetic background by using C57BL/6 NTac ES cells, FLP and CRE deleter mice on a congenic C57BL/6 NTac genetic background as well as applying continuously crossing to C57BL/6 NTac mice. Mishima et al. generated their mice by the use of “R1 embryonic stem cells according to the conventional protocol” and their “Brd1-deficient mice were backcrossed to the C57BL/6 background >5 times.” This is not sufficient to ensure a congenic C57BL/6 background—only by repeated breeding with C57BL/6 mice for 10 generations one will achieve an approximate 99.9% of the genomic background to be of C57BL/6 origin;
3) We have confirmed correct homologous recombination and single integration by Southern blotting analysis. This has not been reported by Mishima et al., thus the possibility for erroneous integration in their strain exists;
4) We have confirmed the efficiency of the inactivation of the BRD1 at the mRNA level by quantitative RT PCR in several organs systems whereas Mishima et al. do not provide data regarding this;
5) We have applied a strategy which is predicted to abolish the function of the BRD1 gene by several means. Firstly, the deletion of exon 3-5 results in frameshift and a premature stop codon in exon 6 which would lead to degradation of the BRD1 mRNA by nonsense mediated RNA decay. Secondly, if this system should appear to be inefficient, we have ensured that the function of the encoded protein should be compromised not only due to the framshift and stopcodon in exon 6 but also by the deletion of a functional important domain (the bromodomain) encoded by exon 3-5. The strategy of Mishima et al. relies on the removal of exon 1 b containing the ATG start codon as well as the region encoding the PhD finger domain of the protein. Since this deletion does not result in frameshift it leaves the possibility for production of an aberrant protein by usage of alternative downstream ATGs.

Vector Construction ET:

Mouse genomic fragments were ET subcloned using RP23 BAC library and recloned into the basic targeting vector harbouring the indicated features (see FIGS. 2 and 3). The confirmed sequence of the final targeting vector is shown (see Table 12).

### Transfection of ES Cells

Transfection date: 20 Dec. 2007
Transfection method: Electroporation
Vector: pBrd1 Final cl 1 (UP0257)
ES cell line: C57BL/6 NTac
Selection method: G418 resistance, Gancyclovir resistance
ES Clones analyzed: 182

### Analysis Method: Southern Analysis

Targeted clones identified: 11
IDs of expanded clones: A-A9, A-B8, A-D1, A-F7, B-D5, B-F6
IDs of validated clones: A-A9, A-B8, A-D1, A-F7, B-D5, B-F6
Quality control: Mycoplasma test
ES cell line: C57BL/6 NTac

ES Cell Culture (B6):

The C57BL/6N ES cell line was grown on a mitotically inactivated feeder layer comprised of mouse embryonic fibroblasts (MEF) in DMEM High Glucose medium containing 20% FBS (PAN) and 1200 u/mL Leukaemia Inhibitory Factor (Millipore ESG 1107). 1×107 cells and 30 ug of linearized DNA vector were electroporated (Biorad Gene Pulser) at 240 V and 500 uF. G418 selection (200 ug/mL) started on d2. Counterselection with Gancyclovir (2 uM) started on d5 after electroporation. ES clones were isolated on d8 and analyzed by Southern Blotting according to standard procedures after expansion and freezing of clones in liquid nitrogen (see FIGS. 4-11).

Production of Chimeric Mice:

after administration of hormones, superovulated Balb/c females were mated with Balb/c males. Blastocysts were isolated from the uterus at dpc 3.5. For microinjection, blastocysts were placed in a drop of DMEM with 15% FCS under mineral oil. A flat tip, piezo-actuated microinjection-pipette with an internal diameter of 12-15 micrometer was used to inject 10-15 targeted C57BL/6 N.tac ES cells into each blastocyst. After recovery, 8 injected blastocysts were transferred to each uterine horn of 2.5 days post coitum, pseudopregnant NMRI females. Chimerism was measured in chimeras (G0) by coat colour contribution of ES cells to the Balb/c host (black/white). Highly chimeric mice were bred to strain C57BL/6 females. The C57BL/6 mating partners were mutant for the presence of a recombinase gene (Flp-Deleter). Germline transmission was identified by the presence of black, strain C57BL/6 offspring (G1) (see Tables 13-20 and FIG. 12).

### Genotyping Analysis/PCR Standard Operation Procedure

### PCR SOP ID: 1643

Genotyping PCR performed according to SOP 1643 detects heterozygous/homozygous wildtype and conditional alleles.

Primers
(SEQ ID NO: 33)
1643_27: GTAAGAGTACCGTGGTTAGC
(SEQ ID NO: 34)
1643_28: GAGGTACAAACCTAAGCTACC

### Reaction

### 5 μl PCR Buffer 10× (Invitrogen)

### 2 μl MgCl2 (50 mM)

1 μl dNTPs (10 mM)

### 1 μl Primer 1643_{—}27 (5 μm)

### 1 μl Primer 1643_{—}28 (5 μm)

### 0.4 μl Taq (5 U/μl, Invitrogen)

### 37.6 μl H₂O

### 2 μl DNA

### Program

### Standard

### 95° C. 5′

### 95° C. 30″

### 60° C. 30″

### 72° C. 1′

35 cycles

### 72° C. 10′

Expected Fragments [bp]
342(W), 467(cond), 342(W)+467(cond)

### PCR SOP ID: 1307

(a.k.a. ART Generic GEN FLPe)

Genotyping PCR performed according to SOP 1307 detects the Flp transgene and the 1307+Control creates an additional

control fragment at 585 bp (PCR-ID 1260).

Primers
(SEQ ID NO: 35)
1307_1: Flpe_as_GGCAGAAGCACGCTTATCG
(SEQ ID NO: 36)
1307_2: Flpe_s_GACAAGCGTTAGTAGGCACAT

### Reaction

### 5 μl PCR Buffer 10× (Invitrogen)

### 2 μl MgCl2 (50 mM)

1 μl dNTPs (10 mM)

### 1 μl Primer 1307_{—}1 (5 μm)

### 1 μl Primer 1307_{—}2 (5 μm)

### 0.4 μl Tact (5 U/μl, Invitrogen)

### 37.6 μl H₂O

### 2 μl DNA

### Program

### Standard

### 95° C. 5′

### 95° C. 30″

### 60° C. 30″

### 72° C. 1′

35 cycles

### 72° C. 10

### PCR SOP ID: 1307+Control

(a.k.a. ART Generic GEN FLPe)

Genotyping PCR performed according to SOP 1307 detects the Flp transgene and the 1307+Control creates an additional

control fragment at 585 bp (PCR-ID 1260).

Primers
(SEQ ID NO: 37)
1307_1: Flpe_as_GGCAGAAGCACGCTTATCG
(SEQ ID NO: 38)
1307_2: Flpe_s_GACAAGCGTTAGTAGGCACAT
(SEQ ID NO: 39)
1260_1: GAGACTCTGGCTACTCATCC
(SEQ ID NO: 40)
1260_2: CCTTCAGCAAGAGCTGGGGAC

### Reaction

### 5 μl PCR Buffer 10× (Invitrogen)

### 2 μl MgCl2 (50 mM)

1 μl dNTPs (10 mM)

### 1 μl Primer 1307_{—}1 (5 μm)

### 1 μl Primer 1307_{—}2 (5 μm)

### 1 μl Primer 1260_{—}1 (5 μm)

### 1 μl Primer 1260_{—}2 (5 μm)

### 0.4 μl Taq (5 U/μl, Invitrogen)

### 35.6 μl H₂O

### 2 μl DNA

### Program

### Standard

### 95° C. 5′

### 95° C. 30″

### 60° C. 30″

### 72° C. 1′

35 cycles

### 72° C. 10′

Expected Fragments [bp]
343(targ)
Expected Control Band [bp]
585(c)

### REFERENCES

N. J. Armstrong, T. C. Brodnicki, and T. P. Speed, “Mind the gap: analysis of marker-assisted breeding strategies for inbred mouse strains,” Mamm. Genome 17(4), 273 (2006).
Y. Mishima, et al., “The Hbo1-Brd1/Brpf2 complex is responsible for global acetylation of H3K14 and required for fetal liver erythropoiesis,” Blood 118(9), 2443 (2011).

### Behavior of BRD1 Inactivated Mice

### General Neurological Assessments:

Full basic physiological characterization was carried out in a functional observational battery (Irwin's test) supplemented with assessment of basic motor-coordination skills in accelerating rotarod settings and nociception levels as tested in a Hotplate setup. General locomotion was assessed in an open field (OF).

No differences were observed between R and W male mice in the general neurological examination whereas R female mice came out with a lower score in both grip strength test and wire maneuver test. In the rotarod test, R and W female mice showed similar learning potential albeit R female mice stayed on the rotating rod for a significantly shorter time (p=0.017). R female mice displayed markedly reduced growth which became apparent around the 5th week of living. No such difference was noted between R and W males. R and W mice did not differ on general locomotion.

### Psychosis-Like Behaviour:

Gaiting and re-activity of the startle reflex was investigated by Acoustic Startle Response (ASR) and Pre-Pulse Inhibition (PPI) tests. In addition to baseline scores mice were also tested during pharmacological challenge; PCP (2.5 and 5 mg/kg s.c.) and amphetamine (2.5 and 5 mg/kg s.c.).

Both male and female R mice showed exaggerated baseline startle response—more pronounced in females than males. Both groups habituated to the startle during baseline test and displayed similar responses compared to W mice during PPI challenge tests. In the PPI test female R mice showed clearly reduced baseline inhibition of the startle at all prepulse intensities, whereas this only became apparent in R males at high prepulse intensities (15 db above background noise level) and challenged with PCP (5 m/kg s.c.). No differences were noticed between R and W mice when challenged with amphetamine at any dose.

Psychotropic drug-induced locomotor hyperactivity was established by injections with PCP (1.3, 2.5, and 5 mg/kg s.c.), amphetamine (1.3, 2.5, and 5 mg/kg s.c.) and cocaine (10, 20, and 30 mg/kg s.c.) as opposing saline vehicle s.c. and measured by recording both horizontal locomotor activity and rearing activity in an automated photo-cell equipped home-cage.

R males displayed clear sensitivity to both PCP and Cocaine in the drug-induced locomotor hyperactivity test (dose 5 mg/kg s.c. and 30 mg/kg s.c. respectively). For Amphetamine, on the contrary the response was the opposite with an obvious hypoactivity compared to W mice at the same dose (5 mg/kg s.c.). The tendencies were the same for both horizontal and rearing activity.

### Social Behaviour:

Social behaviour was assessed by a social interaction test and/or the three chamber test for sociability and “preference for social novelty”, and included recording and scoring of active social interaction, passive social interaction and aggressive interaction to monitor how mice respond to an unknown partner in a 10 min trial. Where the “social interaction test” was performed, social memory was tested by repeating the test after 48 hours.

When tested for direct social interactions using the “social interaction test”, R males did not differ from their WT littermates on total time spent investigating an unfamiliar mouse of same genotype (FIG. 13a), however, they spent less time engaged in passive interactions (FIG. 13a; t test, P<0.05) and, a tendency towards differences in social behaviour was noted with 3 out of 13 R pairs displaying aggressive behaviour whereas only one episode was observed among the 15 W pairs Subsequent application of a zero-inflated Poisson regression statistical analysis to these data revealed that this difference in occurrence of aggressive behaviour between R and W mice was statistically significant (FIG. 13a; IRR=12.67, P<0.05). R mice also showed a significant increase in latency to first social interaction (FIG. 13b; t test, P<0.01).

In a test for sociability and preference for social novelty, R male mice lacked the preference for social stimuli in the form of prioritized exploration of a real mouse over a toy mouse (FIG. 13c; t test, P<0.001)—however, they acknowledged formerly-introduced mice by displaying preferential exploration of novel mice over familiar mice to the same degree as did WT mice (FIG. 13d). In an extension of this test, we exposed target mice to the same novel mouse (now familiar) and a new novel mouse one week after the first test to assess long-term social recognition memory. In this setting, R mice displayed significantly less preference investigating the new novel mice compared to WT mice (FIG. 13e; t test, P<0.05).

### Cognitive Behavior:

Context as well as cue dependent learning and extinction retrieval was assessed by fear conditioning system experiments (FCS).

R mice learnt slower than W mice (p=0.002) and had context dependent learning deficits (p=0.0003). R male mice also had cue dependent learning deficits (p=0.02). They did not exhibit persistent anxiety behaviours during extinction retrieval phase.

Spatial learning and spatial working memory was tested in the Morris Water Maze (MWM). Learning was scored based on latency to escape while memory was scored based on frequency and time spent in each zone of the maze. Exploratory and working memory components was addressed by various types of y-maze alternation tasks including spontaneous alternation test with dark phase testing, continuous alternation, and delayed alternation. Both baseline and induced behaviour (PCP 1.3 mg/kg s.c. and 2.5 mg/kg s.c.) was assessed (PCP 1.3 mg/kg s.c. and 2.5 mg/kg s.c.). In all Y-maze tasks, alternation was calculated as the percentage of right choices out of the total arm entries.

No differences were observed between genotypes in the MWM. A significant reduction in alternation was observed in R mice in the spontaneous alternation and continuous alternation test when challenged with PCP (1.3 mg/kg s.c.) (p<0.01 and p<0.05, respectively). In the delayed alternation task a clear baseline difference was obvious between genotypes at 90 sec. delay (p=0.016).

Attentional set shifting was tested to evaluate medial frontal cortex function following a modified version of the protocol stated in Colacicco et al. 2002 Behavioural Brain Research 132: 95-102. The test was split into 4 test days (1. Simple discrimination (SD), 2. Compound discrimination (CD)+compound reversal (CDR), 3. CDR repetition (CDRrep)+Intra-dimensiona (ID) shift and 4. extra dimensional (ED) shift) in order to keep mice motivated. Test was balanced with 5 mice shifting from odor to media and 4 mice shifting media to odor and exemplars within pairs were selected so mice did not show any preference (or avoidance) toward one over the other.

R mice took much more trials to complete SD, likely reflecting some aspect of learning deficit, and R mice performed significantly worse in the ED shift and possibly ID as supported by the analysis of errors to criteria (FIG. 14). The latter reflects a selective cognitive impairment. Choice latency shows that R and W mice were equally motivated to locate the reward which was expected as food restriction resulted in similar reduction in body weight in both groups of animals (app. 15%). Results for ‘Time to complete test’ showed that groups of animals remained equally motivated to find reward throughout the tasks with differences at SD and ED mirroring the significantly more trials required by R mice to complete the tasks.

### Depressive-Like Behaviour

Depressive equivalent behaviours were assessed by forced swim test (FST) and tail suspension test (TST). Depressive equivalent behaviours (FST and TST) were assessed with anti-depressants (e.g. imipramine at two doses: 1 mg/kg and 10 mg/kg and Fluoxetine: 5 mg/kg, and with normal saline vehicle subcutaneous (SC) injections.

R mice had more depressive equivalent behaviours than W mice during TST (p=0.003) and FST (p=0.001). These phenotypes were more pronounced in female mice. Observed differences in the depressive equivalent behaviours were reversed by both Imipramine and Fluoxetine. Imipramine at the dose of 10 mg/kg had larger effect sizes than Fluoxetine.

### Anxiety Assessment:

Anxiety equivalent behaviours were assessed by bright open field (BOF), light and dark box (LDB) and elevated plus maze (EPM).

R and W mice did not differ on their anxiety equivalent behaviours during BOF, LDB and EPM.

### Anhedonia Assessment:

Anhedonia-equivalent behaviours were assessed in by the sucrose preference test.

Female R had more anhedonia-equivalent behaviours than W mice during the sucrose preference test, as they show less sucrose preference (p=0.003) than W mice (FIG. 15).

### Data Collection and Analysis:

Social interaction, continuous- and delayed alternation, FST, TST, LDB and EPM was scored manually whereas the remaining tests were scored automatically. Ethovision XT 8.0 was used to score the OF and BOF. TSE FCS 8.06 was used to score the FCS. Appropriate tests of statistical significance were used to assess the behavioural differences between model mice and their controls. Appropriate multivariate statistics with STATA12.0 were used to adjust for the effects of potential confounders.

### Example 2

### Preferred Pharmaceutical Formulations and Modes and Doses of Administration

The compounds of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The compounds of the present invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for administration. A device which delivers a pulsed electric field to cells increases the permeability of the cell membranes to the drug, resulting in a significant enhancement of intracellular drug delivery.

Compounds of the invention can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of administration is the ReGel injectable system that is thermosensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Compounds of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient (Derossi et al., 1998, Trends Cell Biol., 8, 84-87).

Preferably, the pharmaceutical formulation of the present invention is a unit dosage containing a daily dose or unit, daily sub-dose or an appropriate fraction thereof, of the active ingredient.

The compounds of the invention can be administered by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical exipient diluent or carrier selected with regard to the intended route of administration and standard pharmaceutical practice.

The compounds of the invention can also be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Generally, in humans, oral or parenteral administration of the compounds of the invention is the preferred route, being the most convenient.

For veterinary use, the compounds of the invention are administered as a suitably acceptable formulation in accordance with normal veterinary practice and the veterinary surgeon will determine the dosing regimen and route of administration which will be most appropriate for a particular animal.

The formulations of the pharmaceutical compositions of the invention may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Preferred unit dosage formulations are those containing a daily dose or unit, daily sub-dose or an appropriate fraction thereof, of an active ingredient.

A preferred delivery system of the invention may comprise a hydrogel impregnated with a compounds of the invention, which is preferably carried on a tampon which can be inserted into the cervix and withdrawn once an appropriate cervical ripening or other desirable affect on the female reproductive system has been produced.

It should be understood that in addition to the ingredients particularly mentioned above the formulations of this invention may include other agents conventional in the art having regard to the type of formulation in question.

### Example 3

### Exemplary Pharmaceutical Formulations

Whilst it is possible for a compounds of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen-free.

The following examples illustrate pharmaceutical formulations according to the invention in which the active ingredient is a polypeptides, polynucleotides and/or antibody of the invention.

### Example 3A

### Injectable Formulation

| | | |
|---|---|---|
| Active ingredient | 0.200 | g |
| Sterile, pyrogen free phosphate buffer (pH 7.0) to | 10 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### Example 3B

### Intramuscular Injection

| | | |
|---|---|---|
| Active ingredient | 0.20 | g |
| Benzyl Alcohol | 0.10 | g |
| Glucofurol 75 ® | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### Tables

TABLE 1

| | | | Implication of |
|---|---|---|---|
| | | | phenotype to |
| Symptom | Mouse test | Response* | psychiatric disorder |
| Basic neurological | Irwin battery | No change | Normal olfaction, |
| function | Hidden food | No change | nociception, and motor |
| | Hotplate | No change | performance are |
| | Rotarod | No change | regarded as |
| | | (male) | prerequisite for optimal |
| | Home cage locomotion | No change | performance in the |
| Motor activity | Open field | No change | tests described below. |
| Positive symptoms | Prepulse inhibition | Decreased | Regarded as impaired |
| | | (males only | sensorimotor gating, |
| | | with PCP) | as seen in e.g. |
| | | | schizophrenia. |
| | Acoustic startle response, | Increased | Regarded as a |
| | optionally, with and without | | biomarker for stress |
| | pharmacological challenge | | responsiveness |
| | (e.g., PCP, 2.5 and 5 | | |
| | mg/kg s.c.; amphetamine | | |
| | 2.5 and 5 mg/kg s.c. vs. | | |
| | vehicle) | | |
| Psychomotor agitation | Hyperlocomotion in | No change | |
| | response to novelty or | | |
| | stress | | |
| Psychostimulant | Hyperlocomotion in | Increased with | Regarded as drug- |
| supersensitivity | response to drugs (e.g., | PCP and | sensitive psychosis- |
| | PCP, 1.3, 2.5 and 5 mg/kg | cocaine | like behavior |
| | s.c.; amphetamine 1.3, 2.5 | | |
| | and 5 mg/kg s.c.; cocaine | | |
| | 10, 20, 30 mg/kg s.c. vs. | | |
| | vehicle) | | |
| Depression | Tail suspension test | Increased | Immobility is |
| | | (females) and | recognized as a |
| | | reversed by | phenotype of |
| | | Imipramine and | depression. |
| | | Fluoxetine | |
| | Forced swim | Increased | |
| | | (females) and | |
| | | reversed by | |
| | | Imipramine and | |
| | | Fluoxetine | |
| Anxiety | Bright open field | No change | |
| | Elevated plus maze | No change | |
| | Light/dark | No change | |
| | | (females) | |
| | Fear conditioning: | | Recognized as |
| | Conditioning | Decreased | impaired conditional |
| | Context dependent | Decreased | learning and |
| | learning | | associative memory |
| | Cue dependent learning | Decreased | with no persistent |
| | | (males) | anxiety (normal |
| | Extinction retrieval | No change | extinction retrieval) |
| Anhedonia | Sucrose preference test | Decreased | Decreased preference |
| | | | for sucrose is |
| | | | recognized as a |
| | | | phenotype of |
| | | | anhedonia and/or |
| | | | depression |
| Cognition/memory | Object recognition | Not determined | |
| | 8 arm radial maze: | | Recognized as |
| | Re-entries to baited arm | Increased | impaired working and |
| | | (males) | visuo-spatial memory |
| | Entries to non-baited arm | Increased | |
| | T maze | Not determined | |
| | Spontaneous alternation | Decreased with | Recognized as |
| | | PCP | impaired working |
| | Continuous alternation | Decreased with | memory |
| | | PCP | |
| | Delayed alternation | Decreased | |
| | Morris water maze | No change | |
| | Fear conditioning: | | Recognized as |
| | Conditioning | Decreased | impaired conditional |
| | Contextual memory Day 3 | Decresaed | learning and |
| | Contextual memory Day 7 | Decreased | associative long term |
| | | | memory |
| | Place recognition | Not determined | |
| | Attentional set shifting: | | Recognized as |
| | No. trials to complete | Increased | impaired executive |
| | Errors during set shifting | Increased | functioning (comprising |
| | (ED and ID) | | working memory, |
| | | | reversal learning, |
| | | | attentional set-shifting |
| | | | and sustained |
| | | | attention) |
| Negative symptoms | Social interaction test: | | Change in the profile is |
| | Active interaction | No change | recognized as aberrant |
| | Passive interaction | Decreased | social behavior |
| | Aggression | Increased | |
| | Latency | Increased | |
| | Three chamber test for | | Recognized as a |
| | sociability and preference | | phenotype of social |
| | for social novelty: | | withdrawal and |
| | Sociability | Decreased | impaired long term |
| | Social recognition | No change | recognition memory |
| | Remote social memory | Decreased | |
| Cortical thinning | Anatomical examination | Not determined | |
| Critical developmental | Age-matched | Not determined | |
| stages | developmental stages | | |
| Disease progression | Longitudinal phenotypic | Not determined | |
| | assessment | | |
| Environmental factors | Maternal infection/stressful | Not determined | |
| | events/cannabis use/social | | |
| | defeat | | |
| Genetic | Crossing mutant lines | Not determined | |
| background/epistasis | | | |

*= response of R mice as compared to W littermates

TABLE 2
Genetically encoded amino acids

| Amino acid | Short | Abbr | Side Chain | Hydrophob | pH | Polar |
|---|---|---|---|---|---|---|
| Alanine | A | Ala | —CH₃ | X | — | — |
| Cysteine | C | Cys | —CH₂SH | — | acidic | — |
| Aspartic acid | D | Asp | —CH₂COOH | — | acidic | X |
| Glutamic acid | E | Glu | —CH₂CH₂COOH | — | acidic | X |
| Phenylalanine | F | Phe | —CH₂C₆H₅ | X | — | — |
| Glycine | G | Gly | —H | X | — | — |
| Histidine | H | His | —CH₂—C₃H₃N₂ | — | basic | X |
| Isoleucine | I | Ile | —CH(CH₃)CH₂CH₃ | X | — | — |
| Lysine | K | Lys | —(CH₂)₄NH₂ | — | basic | X |
| Leucine | L | Leu | —CH₂CH(CH₃)₂ | X | — | — |
| Methionine | M | Met | —CH₂CH₂SCH₃ | X | — | — |
| Asparagine | N | Asn | —CH₂CONH₂ | — | basic | X |
| Proline | P | Pro | —CH₂CH₂CH₂— | X | — | — |
| Glutamine | Q | Gln | —CH₂CH₂CONH₂ | — | basic | X |
| Arginine | R | Arg | —(CH₂)₃NH—C(NH)NH₂ | — | basic | X |
| Serine | S | Ser | —CH₂OH | — | acidic | X |
| Threonine | T | Thr | —CH(OH)CH₃ | — | acidic | — |
| Valine | V | Val | —CH(CH₃)₂ | X | — | — |
| Tryptophan | W | Trp | —CH₂C₈H₆N | — | basic | — |
| Tyrosine | Y | Tyr | —CH₂—C₆H₄OH | — | acidic | X |

TABLE 3
Sequence of mouse BRD1 gene (UCSC Genome Browser on Mouse December 2011
(GRCm38/mm10) Assembly); genomic position Chr. 15: 88687035-88734219

GCTGGGGAGCGAGCAGCGCCTCGGCAGGCGTCCGAGCAGCTCCGCGTCCGCGTCCTCCGCCCGGCCGGGCCCCGAGCCGGCCTCAG
CCGGCCGTGCCGGCGCCGCCGACCCCGCCCGAGCCGCGGCGCCCTGCGGGCCCGGAGCCGCTGGCCGAGCGCGCCCCGGAGCCCGG
CGGGGCACGGCTGCGCGGCCGTTGGCGGAGGAGCCGCGGCGCCATTAGCGCCGCCTCGGCCGCGCCGGCCTCCGCGCCCGCCCGCC
CGCCGGGCTCCCGCGGCCGCGGCGCCCCCGAAGGTGAGTGTCTGACGGTCGCCGTTCGCCGCCCGCCTCGCCGGCCGGGGCGGAGG
TGCAGGCGCCATGTTTGGAGGCGGCAGCGGCGGCTCCGCATTGTCCGCGGGCGGGGAGGCCGGAGAGTCGGGGCGGCGAGGCCCCG
AGGCCGTGAGGCCTGGCGGGCGCGGGAGCCGGAGGGACCGAGAAGGCCGGGCGGACGTGCGCCGCCGTGAGCCGGCGCGGCCGGGG
ACGCCGGAGATCGGTGCCGGCGGCTCGCCCAAGAGGCCGGGTTCGGGAGGCGAGGCCGCGGCGAGATCGCGGAGGCGGAGGCCGCA
GCCGGGTGGGGGCGGAGAGGGACACGGAGGCCGCGGCGGGGTCGGGGAGACAGAGGAGTAGAAGGAGGCCGCCGCGGCGCGGGAGG
CGCGGCCAAGAGAATGGAGCGATCGGCAGGGCTCAGTAGGCGGGGAGGCCGCCGGGCCGGGCGGGCGGGCTCTGGGCAGCTCGGCT
GTCTGGGCGGCTGGGGCGGCCGAGGGGCCGGGCGTCGGACAGCGGAGGAGGCGGAAGGCCTGGGGTCTCGTGGCGTCTGCCCACGT
CCTCGCCCGTAGCCTTGGCGGTGCGGAGCGGGTCGCATTATGTAACAGATCGGTCCGATCTATTTTGCCAAGACAGGAAACTCCCT
TGAAGAGGGACGGGCTCGGAAGATTTCCTAAGTGGAGCGGGGCCTGGTATCTCCGGAGCAAGCCCGCAGCTCCGCCACAACTCCGT
GGATGAGTGCAGGAAACGCCGAGAAACGAGCGCGCGTGCGCGGCTTTCTTGGGCCTTTAGGAGAGAAGCAACTTTCCTGTGCGCTT
AATTTGCAGAAAACGCAGCTCCTCATGGTGCCCTGCAGTTGTGACACACTTACACACACCTAGGAAACGGCCCCCCTTCATGGAGG
ACATTCACTTCACCCAGCTGCGACTGTTTTAGAGTATCTGTCATCTGGTAACAAGTAGTTACAGAATTTCCCTATTACTTAGTTAC
TGTTTTATCACTTGTTGGGTCGCGTGCACTGTCCTGAGTCTGTGTTTTTCTCTCCGGATGGTCACCTTAGAGTAAGGTGTGTCTCT
TTCCTGTGTGCTTTTACGGTGAGGGGTGGAAGCTAGGAAGAGTTTAAATGGCTTGTCCGCAAACCGGGCCGGAAATGAACGGAGCT
GATTTTGAGCATGGAGTCTTTCCCCTCGTTTTGCCGGCAAAGCTTTTTAGGATGCGTTTAGCCCAGTGATTTCTGGAGAAGCATGC
TTGTTGCCTTTGCTGATTCCTCCGTGGAGAGATGCTTGTTCCTGCATAGAGCCAGAGGGGTAAAGTGCTGGGTATATGAAAATGAG
GAAGTAGATGAGATTGTTGGTCACTGTGCCGGGCAGTACTGTTACATGTCCGCTTTCCCCTGGTCACAACTACCTTTTCAAATTAC
AGAGTAGCTGTGGCCATTAAGTATTAGGTTCAGTTCTTGTAGAAAAGTGGTTTAAAGACAGTCCTTCAGTGCTCACTAGAAGAATG
TGGGATTTGACAGGCTGGCTACAGTACTTTACTGGAGAGGAGAAAATTACATGTTTGTCTTTAATCTGGGAGCTGTTGCTTCTGCC
CGTGGTTCTTTTTGGGAAGGATATGGTGCTGACACCTGGATTTGCACCTATCTCGACTTAGGGATGCCACTAGAGGCCTAGGGCAG
GCTAGGGTTGCTTTGACAGTTTCCTGAGAATCCAGTGTTGAGTAGGCACCTGGAAGTGCCTCAGAAGCAGGTGCATTGGGGTCTGG
CTGACTACAGTGTCTTCATATTCTTCTTGTTCATAGAGAGATAGTATAGAATGTGGCTTTCTGCAGCTTGTAAAGTCTGTCTTTAA
AAATGCATTGTAGAGATTTCCTTTTGGGACTTAAAACATGAAGTCTGCTCTTTGAGGGCTTTTCCCAAAGACTAGTAAGATAACTA
TGAGTTGTGAGTTCAGGCTCTGGTGCGCGCGTTCTCTCTCTCTCTCTCTCTCTCTCTCTCTCTCTCTCTCTCGCCCTTCCTCTGGA
CTATCCTGATATTTCAACTTGGTATATTTGGGAGTCAGTCTAACTCTACTTCTTGTCAGTAAAATAGGTTTGTTGAGCTGGAGGGG
CGCGAGCGAGTGCTCCTGGCACTTGATGCTCCATGTGCTCATTCTGCTTGCCCAGTGGTTCTGAGTGGGCTTGTCTGCTCATAAGG
TCCATAGATACCACGGCATGTCAGAGTCCACTACAAGGAATGCGAATATAGGCTCTTGGCGCCCTGGTTTTGTCCATCCTGGAAAT
GAGCAAATCTCTGCATTGAAGTTTTCAGGCGTGTGAGCCAGAGATAAAGGGTGGCGGGGAGGCCACTGCAGGCTGTGGTTTGAGGG
AACCTGTCCTTTCTTGGGAGCAAGAACTGAGCATTTTCAGGTGTGTCAGGAAGAGAGCAGAGATGGCCCTTGATTATCTTGCCCAC
TGCTAGGTTTGCTTGAAGAGTATGTGGCTTAGCATACCCAGGTCCTGGCCTAATGAGAGGGAAAGGCTGGTGGTGCCCACGGCAGT
TTCCAAGGTGGTCACTGCTGAGGTGTCCTGAAAGCTACACTGTGCTCTTGGGGCAAAAATATCCCACAGATCAGCTCAGCGTTCCC
TTTAGTCCTGTGTAGGATGTGTTTGTGGAAAGAATGGACTACTCTATGCTGTTGACTTATGGAAGCTTCTGGGCCCCTGCAGGAAA
GTTCCCAGGAGCGCTCTGCTGGGCAGTAGTGAGAAAGAAAGGAGGTTGCTTAGGAATTGCTAAGAGTAGGTGGCCACAGCCCAGTA
GGCGGCTGCTTTGTGGCCACAGGTCTCTGCTGTGAAGTCTGGCAGAAAAACAATCTATACTTGTAGGAGAGAGGCCTCGCTCTTAA
CTCTGGAGACTGTGTTGCTGTTTGGGGCTTACTTTTGGCTTGGTCTAAAGAGGTGTCTTGTGGGTGGAATGCACCTGTGCCCTAGC
TATTCAGCAGGAACCCTGAGGGCTGCAGCTTCCTGCTGTCTCCGGCCTTATCTGTACCTTTACCTGGGTGTGGTGAGGGAGAGGCT
TGCTGAAATGTGAGACATTGTTTGGAAGTCTTCTTCAGAGCCTTTAAACTCTGAGCTTTGTTTGCGGGAGATTTGTTAGTGCTACC
CAAGCACATTTTGTAGTTCTCTGAAGGCTTCTGTCATCCTGCATAGAGGTAACTTTTCCTTTGACTTTATTTTAGGTAATCATTGC
CAAATGAGGAGGAAAGGACGATGCCATCGAGGTTCTGCAGCGAGGCATCCTTCTTCCCCGTGCAGTATTAAACACTCCCCCACTCG
AGAAACACTGACCTACGCACAAGCTCAAAGGATGGTGGAGATAGAAATCGAAGGGCGCTTGCATCGGATCAGTATTTTTGATCCCT
TGGAGATCATACTAGAAGATGACCTCACTGCTCAGGAAATGAGTGAATGTAACAGTAATAAGGAGAACAGCGAGAGGCCGCCTGTT
TGCTTAAGAACTAAGCGTCACAAAAACAACAGAGTCAAAAAGAAAAATGAAGTCCTGCCCAGCACCCACGGCACACCGGCGTCAGC
CAGTGCCCTTCCCGAGCCCAAGGTGCGGATTGTGGAGTACAGTCCTCCCTCTGCACCCAGGAGGCCCCCTGTGTACTACAAGTTCA
TCGAGAAGTCAGCCGAGGAGCTGGACAACGAGGTAGAGTACGACATGGATGAGGAAGACTACGCCTGGCTAGAGATCATCAATGAG
AAGCGGAAGGGTGACTGCGTCTCTGCCGTGTCACAGAATATGTTTGAGTTCCTGATGGACCGCTTCGAGAAGGAGTCTTACTGTGA
GAACCAGAAGCAGGGTGAGCAGCAGTCCTTGATAGATGAGGACGCTGTTTGCTGCATCTGCATGGACGGGGAGTGCCAGAACAGCA
ACGTTATACTCTTCTGTGACATGTGCAACCTGGCTGTGCACCAGGAGTGCTATGGGGTACCCTACATCCCCGAGGGCCAGTGGCTT
TGCCGCCACTGCCTGCAGTCTCGGGCCCGCCCTGCGGATTGCGTGCTGTGCCCGAATAAGGGCGGTGCCTTCAAAAAGACAGACGA
TGACCGCTGGGGCCACGTGGTATGTGCCCTGTGGATCCCAGAGGTTGGCTTTGCCAACACGGTATTCATTGAGCCCATTGACGGTG
TGAGGAACATCCCTCCTGCCCGGTGGAAACTGACATGCTACCTCTGTAAGCAGAAAGGCGTGGGTGCCTGCATTCAGTGCCACAAA
GCAAATTGCTACACAGCATTCCATGTGACATGTGCCCAGAAGGCTGGCCTATACATGAAGATGGAGCCTGTGAAGGAGCTGACTGG
AGGCAGCGCCACGTTCTCTGTCAGAAAGACTGCTTACTGTGATGTCCACACGCCTCCAGGCTGTACCCGGAGGCCGTTGAACATTT
ATGGAGATGTTGAAATGAAAAATGGTGTGTGTCGAAAAGAAAGCTCAGTCAAAACGGTCAGGTCTACGTCCAAGGTCAGGAAAAAA
GCAAAAAAGGCTAAGAAAACACTGGCTGAGCCCTGTGCGGTCCTGCCGACCGTGTGCGCTCCGTATATCCCCCCTCAGAGGTAAGT
GCATCTGAGCTTCCGGCTCCGATGGGCCTGAAGGGAAAGACTTGATGGTGGACACAAATCCGGGCCAGCAGGAGTTCTGCCACACC
TCTGTCCCACTTCCTGATAGTCTTCGTCCTAAGTTGTAGCCTTTAATTGACTGGCTACTGTGGAGTGGGGTGTAAAGTGTAAGGCA
CGGATTGGGATAGTTTACAGTTGTCACCTGTTGGCCTGGAATATAAGGTAGGTACACTCACGGGAGCCACAGCCACACTAGTATTC
ATTCAACCCTGGGTTTCTGGACTTCATAGCATCCTAAGTTTTGTTTCTAGCTATAATGCCGTTAAACTCCCTTATTACCAGATTTG
AGGACCTTGTGTGAAAGCATCTGGTTGGGAAAGTGAACTACCATCCTCAGTAAGGTAACCTTTGAGGTGAGGTTAGAACAGGAGCT
GCTGTCAGCAGGCAGATGGTGGTCTGTCTTCTACTGGCCTTGAACTCACAGGGATCCTCTGCCTGCCTCCCAAGTGCTCCCACCAT
ACTTGGCACATTGTATGTTCCTGGTGGGAGGACTTGTCCTCTGCAGTTTAGGGACTGCTTCAGCTTCTTCAGTCTGCATTGGGCTG
CCCTCTCTCCTGTATCTTCTCCACTACTCTCTGGTTTGCTGTTTTTGTTCCATTATTTCAAAAAATGTTCCTTTTCACATCATAGC
CTGAGGATGCCAAATAAATCCACTCTTTTTGTATCTGTTTGAACCCTTTTTTGAGCCTTAAGGAAGTAATTTTCTGTGAAGGGGGT
GTGGGCTTTTAGTTGGGTCAGGTCTGTAAAGCCCCAAGGAGATAAAGTTCATGTGAAGCAGACAGCAACCCACATGGGTTTTACTG
TAAACTGCTCCATAAAAACGTTCATTCTGTAGCGAACTGGTAGACAGTAGATTTCAGAGGTTTTTTTTTGGGGGGGGGGGGAGATC
TGGTCTCTGTATCTTTGGCTGTTTTAGAAAGCCTATAGACCAGGCTGTCCTGGAACTCCATCCGCCTGCCTCTGCCTCCTGAGTGT
GCTAGGATTGAAGACATGAGCCACCAGCATTGGCTCAGAACCTGTCTTTAACATAGTGAACATTAGGCTTTTTGTGTTACTTTCTT
ATGAATGTCTGGTTTGAAGAAATTAATCTTTTTTTGTTTTTGTTTTTGTTTTTTTGAGACAGGGTTTCTCTGTATAGCCCTGACTG
TCCTGGAACTCACTTTGTAGACCAGGCTGGCCTCGAACTCAGAAATCCGCCTGCCTCTGCCTCCCGAGTGCTGGGATTAAAGGCGT
GCGCCACCACCACCACCGGGGGAAATTAATCATTCTTGCTAGCATGCGGTGATTGATTCCACTATGGAGTTGGGTAGCAACTGCCT
TTGTATTAGAGTTTAAAACGGGTAAATAAATGCTTTTTTTTATAGACCTATTCCTACTACTTAGAGTCAGTGAGTCAGAAACAGAG
ATCTCGTAACCCCTTGTTCAGAGAAGAGTCCTGGTAGAACCAGCATGCCTGACTTCTGTGCCTATAGAGGCGGAAAGGATAGGGTT
CTATGAGAGCTCAGGAAAGTTTAGCTTTACCGAAATTGAAGTAAGTGAAGCAGCAGTCTGCTTGCTCTCGCTGGAGTGCCAAATAT
TCCGTGTTCCAGGTGATGGGTGCGATCCTGCACCCCGGCCTGTGGTTCCTGATGTTCAGGTTTTGGAACATGAAAGCTGCCAGGTG
GGTGGGACTTGCAAGGAGGATCTGCAGTGAGAACAAAGACCATCGAAGAAGCTTGAAGCTTTAAAAAAATCTTCCAGGGTCTGTTG
TAGAATTCAGCAGATTCTATTTGTGCATTGTGGCCCGTGTTTCCTTCCCCAGACAAGGTCTTATCTGTAGCCCAAGACTGCCTGAG
GCTTATGGAACACAAGTCAGGTTGGCCTCAGACTTGTGAGTCTCTTGCTTCAACCCGTCACATGCTCACTGTCCTGTCCTAGCTTG
TCTTACTTTGTTTTGTCATGTTGTGTTTTGTGACAGAATCTCACTCTATATCCCAGGCAGGGTTGAAACTTTTTTTAAAGATTTAT
TTTTTATTTATTTTTATTGTATATAAGTACACTGAGCTGTCTTCAGACACTCCAGAAGAGGGAGTCAGATCTCATTACGGATGGTT
GTGAGCCACCATGTGGTTGCTGGGATTTGAACTTCAGACCTTCGGAAGAGCAGTCGGATGCTCTTACCCACTGAGCCATCTCACCA
GCCCGAGCCTTGGCCTCTTGAATGATGGATTTAAAAGCATAAGCCACTGTGCATAGCTGCTTGCTACTACTGCTGCTGTTGCTTTT
TTAATTAATTAATTAATTATATGTAAGTACAATCTAGCTGTCTTCAGACACTCCAGAAGAAGGCATCAGATTTCATTACGGATGGT
TGTGAGCCACCATGTAGTTGCTGGGATTTGAACTCAGGACCTTTGGAAGAGCAGTCGTGTTCTTAACCGCTGAGCCATCTCACCAG
CCCCCTGCTGTTGCTTTTTACAGATTTATTATTCATTTTGTATGTGTGAGTGTTTTGCCTGTATGTATATATGTGCGCCATGTGTA
TGGCTGGTTCCCTGCAGTCAGAAGAGGACTTCAGATGCCCTGGGAGTAGAGTTGCCGATGATTTTTTGTGGGTCAGCAGTGGGGTG
CAATGGAATACAGTTGGACAGCTTTAACCAGTAGACTTCGGACAGGCAGTGCTGGTCAACTTGGCTTACACTTTTAATCCCAGCCA
TTGGGAAGCAGAGGCAGGAGGATTTCTGTTTAGAGTTCAAGGCCATCCTGGTCTATGTGGTGAGCTCCAGGACAACCAGGGCTATG
GAGAGAGACTGTGTCCAAAAGAAAAAAAAAGTTTGGGGAAGGTTGAAGAAGGAAGGTCAAAAGAGTACAGATTTTGTGGGTTTTTT
TGTTTTTGTTTTTGTTTTTGTTTTTTTGTTTTTTTTGTTTTTTTTTTCCGAGAAGCCTGTTTTGAGCCTTAAGGAAGTAATTTTCT
GTGAAGGGGGTGTGGGCTTTTAGTTGGGTCAGGTCTGTAAAGCCCCAAGGAGATAAAGTTCATGTGAAGCAGACAGCAACCCACAT
GGGTTTTACTGTAAACTGCTCCATAAAAACGTTCATTCTGTAGCGAACTGGTAGATAGTAGATTTCAGAGGTTTTTTTTTGGGGGG
GGGGAGATCTGGTCTCTGTATCTTTGGCTGTTTTAGAAAGCCTATAGACCAGGCTGTCCTGGAACTCCATCCGCCTGCCTCTGCCT
CCTGAGTGTGCTAGGATTGAAGACATGAGCCACCAGCATTGGCTCAGAACCTGTCTTTAACATAGTGAACATTAGGCTTTTTGTGT
TACTTTCTTATGAATGTCTGGTTTGAAGAAATTAATCTTTTTTTGTTTTTGTTTTTGTTTTTTTGAGACAGGGTTTCTCTGTATAG
CCCTGACTGTCCTGGAACTCACTTTGTAGACCAGGCTGGCCTCGAACTCAGAAATCCGCCTGCCTCTGCCTCCCGAGTGCTGAGAT
TAAAGGTGTGCGCCACCACTGCCTGGCTTTTTTTTTTGGGTTTGTGTGTGTGTGTATTTTGTTTTTTTGTTTTTTGTGGCAGGGTT
TTCCTGTATGGTATGGTCCTGGTTGTCCAGGCTGGCCTTGAAGTTGACATCTGCCTGTTCCTGCCTCCCAAAGGTGTGTACCACCA
ATACCCTACCTATTTTTTTTTTTTCCTAAGAAAAATATTTTGATGCCTGTTTTTCTGTGCTCTTCTGTGACCCTGCTCATCCACCC
GATTCTGTGTAGCAGGAGGAACGAGCAAGACCAGGTAAAGGGCAACGCTTCGTAGTTGTCCCCCCCTTACCCCCCCCCCCAAACGA
AGTACCAGTCTCGGTAACTTCCCTGCCCTGGCCATATGAGGCCGTAATTTATCTCCAGAACAGAAGCTGCTGGTGAGTAGCTGTGC
CTGCCCAGATCTGGACTTGACTCACTCAGATCGCCTCTGTGCCTTGGAGAATGGGTGTGCAGTTTATTCAGTGCCGAGGTGTACGT
TGTGACTTGGTGCTGGGTCAGCAGTGAGACTGAGGCACCTTCTGTTTGCTGTTTACACTGCCAGTCCTTGATCTGGCTTTGGGAAA
AGACCAGGTGGTGTGTGAACACCCGATGCACTTCATCAGGTAGACTAGGGTTTGCTTTTACATATACTGTTCTGGCTTGGATTTTG
TGCACACCCCCTCCTCCATGCTTCTGCTAGTTAACTTGTCAGCTTCTCTCTCTCTCTCTCTCTTTCTTTCTCTCTCTGTTAATGGC
ATAGCTGTTTGTTTGTTTGTTTGTTTGTCTATTTCGAGTTTTAGAGAAACGTCTTTTTCTCTTGTGTGGTCCTGACTCTAAATTTT
TGAGACAGGGTCTCACTGCGTGACCTTGGCTGACCTGGAGCTTGCTATGTAGCCTCAGACTCCCCCGTGCCTCTGCCTCCTGAGCT
GGGACTAAAGGCGTGTCAACAGCATGCCTGATTTAGTTACCAGTTTTGAAAACAGTACATGTAAAATATTGTATATAATTTGAATT
TTGCTCTTTCTTTGCTAGTGGTATGTGTCACACTCTCTGGGGATGCGACATTGCACTGCTGTGAGCCACAGCCTCAGTGAGCGGCA
CAAAGGATGGCTGAGCACTTGGTGGGAGCTGTGCTGTTTAACTGGGCTGTTGGGTAGCATGGCTGCTTTGACTTGTGTGAGGTGAT
CAGCGTGTAGCCTCCTGTCAAAGAGCGTCTGTATTTGATAAACATTTCCATCTGCCACGGTTGGCGCCATCCCTTCCAAGTGGAAG
CCCTGCCCTGTATGTCCTGGGAGCAGTGTAGGGAGGGCTTGCTGCTGTGCCAGGGCCTTGGAAAGCAAGCAGATGCATCTACTGTA
GAGATGCTGGGGAAGAAGCATTTGAACGACCGAGAAGTACAAAATGACACACTGATGTGGAAGGCAGAGCCCATCTGACAGCCAGT
CTGAGATGAGTGGGTCTACCTGCTCATCTCGTGCCCTTAGGAAGCTGGGTCAATCATACCGAGCTGAAATCACTGTATACTGACTC
TTCCCACCGTCTGGACACCTTCCCCTAGTGGACTGTTGTCCCTGGGCACTCAGCAGAGAGGGCATCTCCAGTATGACTGATTTCCT
CTTTTTGTTTTTAAAGATTTATTTTTATTTTATGTATATGAGTATACTGTGCTTGTACAGATGGTTGTGAGCCTTCATGTGGTTGT
TGGTTGTTTGGAATTGAATTTAGGACTTCCGCTTGTTCCAGTCAACCCCTCTTGATCCAGTCAACCCTGCTTGCTCCGGCCCAAAG
ATTTATTATTATAAATAAGTACACTGTAGCTGTCTTCAGATGTACCAGAAGAGGGCATTAGATCTTATTATGCATGGTTGTGAGCC
ACCATGTGGATGCTGGGATTTGAACTCAGGATCTTCTGAAGAGCAGTCAGTGCTTACCCACCGAGTTACTTTGGAATAGGTAGAAG
TAGATACTTACTTCATTGCTGGGGGCAGGCTGTTCTTTGGTCTCTCTACTGCTGCTGTGAGTCAGTCCACTTGAAGCTAACAGTGG
GCCTTCGTGGGACCCTGAGGTCAGCAGGACTCTCAAGTTTGGTCCACATTAGAAAAAAAGATTGCATTACATGGTCATGTGCCCAC
GGGGCATGGGTTCTAAGTTATCCTTTGCAGTGGGGAGGGGCACTTGCATGCCCTGTCCTGTCCATGCCCACCTTCTAGAGGTAATC
TTGGTGCCTGGTTGTTGCTCCATACCGTGACTCCAGCTCCATGCCCCTAACCCAGCCTGCCTCACACAATACTCGGGCCCTCTGAG
TATTAGGAAGACCATTCTGATTATTGCTTTGTTCTGAGGGGCCAGAGCATTGGGCAGATATTACCAAATGGAAGGTCAGGGGCCAG
AGGGCCGGGAGGTGGGCAGACCTGCCACTGCCAGGACATGGGTTGGGTGTTGTCTCTGCTGACACCACGTGAGCCGCTGCTCTGAC
TGCTCTTCAGCTTTCCTGGCTTTGGATGCTTTGTCTTTGTCTGGTGTGTTTCCCTCTGGTTCACTCAAGTTAACCGTCCTTATGTT
ATGGTGACTGTCAACCATAAATTATTTTTGTTAGGAATCTTGGAGGTTTGACAAAGGGGTCACGACCTACAGGTTGGGAACCACTG
GTCTACAGTATTGCTGGTCTTTTTACTTGTTTGAGGCATGTCTGTGTTGACCAGGCTGACCTAGAGCTACCTGCCTTTGCCTCTGA
ACTGTTGGGATTAAAGGTGTGTGCCGCCATTCCTCCATGTTTCTGAGGGTGATGTTTCCTGGCAGCTAGTTTCACATCTTTGTCAA
GACTTGAAAACAAGTGCAGATTGAGGGTTGTTTGGCCTGGCCAGTCTTTCCTATGATTATTAGCATCAGTGATAGTCCTCGGTCCC
TGGGCTTTTGTCCTCCCGAGTTTGTGCTGGTTTGTCAGTTGCTTGAAGAGGCTGGGAAGTTACCCAGTACATAGGACCTGGGCATT
GTGTGGAGAGGAGGCCCGGAGTGTCAAGAGAGGAGCCATTTCTCACTACCTCAGGGGAGATGAATAGTCAACCATATGATAGCATT
TATAATACAGTTGGCCTCTGCCACAGTTGGCCTGTCACCTCTGAGATCTTGGCCCTGCTTATTTTCTGTGGCAAATGCCTTCTTAT
AAGCAGCCGAAGAAGGTGCGACTTGCCAGCTCTCTTTTCGACTAACTTGTGTTTTTTGGCAATTCCAGGTTTCACATGGCCATCTA
TTGACTTGGGTGTATAGTCTGTGTCTAGAGGTAAATTGTAGACTTTTGAGTCCTTGGAGGCAAAAAAACCTAGGCTTTAAAAATGA
TGCTTTATTTTTTTATTTTTTATTTTCATGATGTAATGATGCTATTTGTTGATACTGTAAGGTTAGAGACACTTGTCGGCCTGACC
ATGAGCTGTCCTGAACATGAGTGGAGTTCATTATAAAGATGTAGGATGTGTAGGAAATGTTGCATCAAGAAAGGAGGCTGGTTTGT
AAAATTCACTCTCCAGAGGTGACTGTGTGGAGCATCTGGAGAGATTGTGGGTCTATGCACATGTATGGGTAGAAGTCATGTTCTTT
TCTTACTTTTCATGATTTTTGTCTAGGGAACTCTAAGGAAAATCAGAGACTAATGTAACCTGAGTTATCAAGTATAGCAGCAAGCC
ACAGTTACCGTGGAGGCCTGCAATCTCTGGGTTCATTCTCCTGCTTAGAACAGCATTCATAGCCGGCAGTGGTGGCGCACGCCTTT
AATCCCAGCACTTGGGAGGCAGAGACAGGCGGATTCCTGAGTTCGAGGCCAGCCTGGTCTACAAAGTGAGTTCCAGGACAGCCAGG
GCTACATAGAGAAACCTTGTCTCAAAACAACAACAACAAAAAACCAACAACAACAAAAAACCAACAACAACAAAAAAAGAGAACAG
CATTCAGGTGACTCTGGGACTTGCGTGCACTTGACATCCTTGGGCACGGCTTGTTTTCTCATTTCTAGTGATAGCTGTGATTGACA
AAGGGGAAAATAAGCTTTAAGAAGTACAGAGAAAACCTATTGGTTCAGCAACTTAACTTCAAAAGTTCCTGTGACTGGTTCATCCT
CCTTGCCTGTACCTCCCTCTGTCCCAGCCATGTGACTCCATGACTGCAGCTGTAAAGAACCTTCTCAGAGCTGTAGATTGATGCTA
ATGAAGTGAGTGCTGGTCGGTCCTTTTCTGTGAAAAGTGTCCCCAGAGGTCAGGGAGGCTTTGGGGTTCTGGAATTGTTTGTTGCG
GATGGTATGTGGGAGCCTAAGAGCCTGTTCCTCCATACTGCTGTGGTTCCTGCTGTGTAGACCTTCCTGCTGGCTCCCAGCCCCAC
AGTTTCTCACCTCCTGTGTTCTTGGTCTGGTTCCACAGTATTGCTGAGCATAGGGGTAGCTCATAGCACTACGGGCTTTTTACTGA
CTGTCCCATGACTGCATGGTTGTCCCCATGACATCAGTGTTCTGTGGGAATTCTGGTAGGGACGACCTTGCCACTCACATAGGTTT
ATTTATTTATTTATTTTTTCTTTTCTTTACTTGAGATAGGGTTTCTTCATAGGTTCGGCTGGAAATTACTATGTAAACCAGACTGC
CTTGCCTCTGCCTCCCTAGGACTGGGATTAAGGTTTCTACCACCACACCTGCTAATATGAGAGTTAACTGGTGAGGCCCTGTCTCA
ACAATAGCCACAACTCCACCCACCCCACTTTCCAAAATGTCCCTCCCCCGATTAAATTAGCCGTTTGTGACTTTGTTAGGATACAG
GATTTTTGTTTTTATATATTATATATAAAATTTATAAATTTTAGGCTATCTTTGTAGACAGTATCTGTGCAAATGGCAGTTTTGTT
GGGCTTTTCTGCTTTTTTAGCTTTTTACTCAAAGTCAGACGAGGCCTGCCTTTTGAGCTGCCCAGAACGGGATTGACTCTGATGCA
TGCATGTACTGTATGTATGTTCCTACATAGTGTATGTAGGGATTTGTTTGTTTTGTTTTTTAAGCAGTCTTACTGTGAAGCCCTAG
CTGCCCTGAAACTTATGTGTAGACCACAGAGATCCATCTGCCTCTGTCTGAGTGCTGGGGAGAATTCTTAACAATCAGTACTATTT
AATTCATAATGGAGTCACTGGTTTTGTTAAAAGCCGGTTACTGGGCTGGTGAGATGGCTCAGTGGGTAAGAGCACCCGACTGCTCT
TCTGAAGGTCCAGAGTTCAAATCCCAGCAACCACATGGTGGCTCACAACCATCCTTAATGAGATCTGACTCCCTCTTCTGGAGTGG
CTGAAGACAGCTACAGTGTACTTACATATAATAAATAAATAAATCTTTTTAAAAAAAAAAAAGCCGGTTACTACTTCCCTGGGGGA
GGGGTGTTAGTGTTGGGCGGAGGTGGAGCTGGCCCTGTTTCTCTTGCCGTTCTTTTACTTTTTTTTTTTTTTTCAAGACAGGGTTT
CTCTGTGTAGCCCTAGCTGTCCTGGAACTCACTTTGTAGACCAGCTGGCCTAGAACTCAGAAATCCGCCTGTCTCTGCCTCCCAAG
TGCTGGGATTAAAGGCGTGCCACCACGCCCGACTACTTTTACTTTTTAAGACAGTCTCTCTGTGCAGCTTAGGTGGCCTCCGCGAG
ACTCTGAAGTGCTGGGTGACAGGTATGCCGCCATGCCCAGCTTTCTTCCTGGTTTGTTCTTACTGTCGGAGGAGTTCGAAATCCTG
GCCATGTGTATAAGTAGACTATAAAAGTGGCTTGTTGTGGTACTGTATGTGCAAAGCTACAAGTTGGCTGTAAGCAGTGCATTCCA
CTTTAGACCTAGGGTCTTTCTCACTAAAAGTGGATACAACCTAGGCCGAGAAAGCTTAGAAGGACCCGACAGTGTGAGTCACTTGC
CACCTTCACTTTGTAAACATAACTTCACACTTTTCAGCAAAATGGTCCAGTTAATTTTTCTCTTGTATTTTTGTTTTTATTAGCTG
TATTTAGGAAGGCTTGAGAAACTTGTGAGTGTATTCTTGCTAACATTTAAAATTTTTAAATAGACTATAATATTAAGAAATTCATA
GCTGGGCGTGGTGGCGCACGCCTTTAATCCCAGCACTTGGGAAGCAGAGGCAGGCGGATTTCTGAGTTCGAGGCCAGCCTGGTCTA
CAAAGTGAGTTCCAGGACAGCCAGGGCCATACAGAGAAACCCTTTCTCGAGAAACAAAAAAAAAAAAAAAACAACAACAACAAAAA
AAGAAATTCATAAGACAGATGTGTGGTTATTAAGTTACAATGGAACAATTGTAGCGATTGTTTGTGTCGTGGAGCCCTTCTTGTTG
CATGGCTAGGGCTGAAAGTGGTTTGGCTCCTGTAGGGCTTGCTTCATGGGCTTTTCCTCTCTGTAATCTTGGTTTATTTGTGCTTT
TGACATAACACTCATCAGATTTTAGTTGCAATAACTATGCAGATAAGATTGGGGAGTTTATAAAGGATTTTTTTTTTTAGCTCACA
ATTTGAGGAGCTGAGAGCACAAGATTGGACATCACATCAACTTATTCTGTGGCTGAATCAGTGGGGCAGTGGCATCGTGGCGAGAG
CGTGTGTGGGACGTAGAAATGCTGCAGTGAGATAGGACTCCAGAGCACAGGGAGTGGCCAGCCTGGTCTTCCTGCTGGGTACCTAT
CTCCAGGGATCTGGGACAGAGTATCCAGACTAGAGTAGCGCCTCTGTTTCCTTCTAGAGATCCATTTTGGTCATGTCTACTTCCAG
GTTCCTGTGTGCGTGGGTCTCAGGTCTGTCTGTGTTGGTTGTCTGTCAGTGGTAGTTTGGCCTGTTCTTCCTGTGGTTTCTGAGTT
GGTAGTTGGCCTGATCATTGATGAGTGTGGGATGAACTTGTTGGACATGCTTGCTTTTGGCTGGTCTGCTTCTGAGGACCTACAGT
ATTAGTGCCTGTTGTCTACCTTTCTCCACAGTGTGCAATTGCTCACCAGGGGGGAGTCAGACTCTGCTTATGTAGTGTTTGGATAC
ATACCTGTAGAGGACATATTTTAAATTTGTTTGTTTGTTTTTGTTGTTGTTGTTGTTTGAGACAGGGTTTCTCTGTGTAGCCTTGG
CTGTCCTGGGACTTACTCTGTAGACCAGGCTGCCCTCGAACTCAGAAATCCGCCTGCCTCTGCCTCCCAGGTGCTGGGATTAAAGG
CATATGCCACCACTGCCTGGCATTAAATGTATTTTCTATAAATCTTGTTTACAACTTGCAAGCTATTTACAGTTTCCCAAGTTCTT
GCACTGGGGAAGGTGTGGGTCTAGTATGAAGTTGGAAGCTTTATTAAAGCAAATTGCTAATTATTACTATTTTTTTTGACTTTTAA
ATTGTTAACAAATCTTGTATCTGGCTGGGAGCGGTGGTGCACACTGTTAATCTTAGCACTGGGAGGTAGAGGCAGAACTCTGAGCT
CATGACTATAAAGCTAGTTCTGGGACAGCCATGGCTCCATTACACAGAGAAACTGTCTTGAAAAAAACAACAAGAAGCAAGCAAAA
GTCTTCTATCTCTGCGCTGCTTCTGAAGGTTAAAGTAACCATCAGTGTAGTGTTGAACATCTGTTTGCTGTACAGATGTTACACCT
CAGTCAGAAGTGAAAACACAAGCTGTTACCAACACTGCAGCTGTGGCGTGGCCGGGCCTCCTGCCCGCTCCATGGAGACTTTGGTC
CATCCTCAGGTGTCGTGGTTGCCTTCTGGTGCAGCCTGGTGTCCTGCCTCTTGATGGGTTTGTCATTGGAGATAATGCTTCGTGGT
CTTGGTGTTTGACCCACCACATTGAGCATGCAGAGCCGCAGAGGGCACTGCATCATCCAGCGGAGCTCAGCCAGGAGGCTCGACCA
CCTCGAGGTTTGAAGCATTCTCAAGAGCAAGCAAACCTTGGCAGAGCCGGGCCTTGGCAGAGCCGGGCCTTCCAGCTGATGCTGGT
GTTCTTGATTGCGTTCTTTAAAAAAAAAGTGAACTTAGAAAATTTTAAAGCCTGTTGTGTAATTTTGATGTGTGGTACAGTGAAGG
AACACCTTCTTGTAGCCTTTTGTAGTGGGATTTGCTGGAGTTTGTCTTTCAGTGTCTTTGTGAGGCGGCATACCAAGCCCCATCTT
CTTCAGAGGGAGGGAAGCAGGCTGTGGTATAAGCAGCCGCGCAGAAGCTCTCTGGCCGGCATTCACAGCACTCACACACAGCCTGA
GGGCTTTGAGCCTCCCTTCTGCAGAGGTTTTTACAGCTTGGCACGAGGATGGTTGTCATTTACTAGGAGCAGACCATGTTCCCAGC
CTGAACTCAGTGGGTGGGCTGCTCTGCTTGGAGAGTTTCTTAAGGTTGAGTGTGCCCAGCGCTGGTGGCGCCAGCTGTGAGCGCAG
GCTTTGACCTCCAGTCCATCCAGTCGGCAGCATCTCAGCTGGCAGTGGTCAGTAGCCGTCACTGTGTGTGTAGACAGGAGCACAGG
GGCAAAGTGGTTAAAGTTTTGTTCACCTGTGTCTGCTTTAGACGTTGAACCTGGTGACTCTTGTGGAGGATGAAATCTGTAGTTAG
TTGAAGGTTATGAACTGTTTTCAGGGACAGGCTCAGGGAGAGAACTGCAGTGTCCTGTCTAGTTTTCTAAATGCAAACACGTTTAA
ATATCCCTTTCGAAGCTAAACTCTCAGTTTTTTCATGTTTTAGATTAAATAGGATTGCGAATCAGGTGGCCATTCAGCGGAAGAAG
CAGTTTGTGGAGCGAGCCCACAGCTACTGGTTGCTCAAAAGGCTGTCTAGGAATGGTGCTCCCCTGTTGCGGCGGCTCCAGTCCAG
CCTGCAGTCCCAGAGAAACACGCAGCAGGTATGTGTGCTCTTCTGCTTTTCAGTTACATGGGCTGCCCCCCCCCCCCCCCCCCAGG
CTGGATGTGCTGCTGACCCTAAGCCCCGGGCCTTAAACTCTACTAAACTGCAGGTTATTCGGGTGGCTCCTGTATCCTCAAGGTTT
GCTGTGACTTTGGGGTTGAGTTGTTCTTTACTCTGACAAGTGTCTGCTCTGTGCCCAGTCCTCTGTCAGTTCCAGGGAAGGAAGGG
ACTGCTCAGAGAACCTGGCTCAACTTCAGCTGCATGCATAGTCAAGACAGAGAGGGAGGCCTGATGAAGTCTATGCAGTTCCTCTA
CACATTGCCCAAAAACTAGGTGTCTGGTAATACCTGCTGGTTCCACTGGGAGGAGCTAGTCATTTCATCTGTAAAATAGCAACCAA
CTTTAATGGAAGTTTAAGTCTGTAGAATCCTGTGACTCCCCATGGCTGTCACAGGCATGGCTGTGAATGAGCTTAGGGTTCTCATC
CTGTATCCTGGCTGTCAGATGAGCAGTGGTACTGGAGCCCTGTTGTATGGATCAGACCCTTGTGTCTGCAGGTTACCAAGTATTGC
TCTTCTGGGAGTTAACAACTTGCTGGACTCTGTCTGGGTCTGATCTGAATGGAAGGGGCCTCCCCAGTGTTAGATCTTCTGTTGCC
TTCTACAAGCCAACGTTGTCTATTATTCACTGAGGACACATACCTCCTTGGAGGCTACTGGAATGTCCTAGTTAGGGGTTTCCATT
GCTGAGAAGAGACACAGTGAAGGCAACTCTTACAAGGGACAACATTTAACTGGGCTGACTTCACAGGTTCAGAGGTTCAGTCCATT
ATCATCAGGCCGGAAGCATGGCAGTGTCCAGGCAAGAGGGTCTTAGAGCTATTGGTCATGAAGTGGGGAAGTGTTTGGTAACCCTG
GGCACTGGGAGGAATGATTGCCTATGTGACGGTAGGTAGCAGTGTTGGAAAGAGAAGTCCGGGAGTGGGTGGCTACTTCTGAGCTT
CCCCTTCTCAGAAGTCTCTTCCTGGGAAGAATTCCAGCATTGATTTCTATGTAGCAAAGCAGACTGCTTCGGAATCGTACCGGGAC
AGCGGGTTTACAGATGGGATGATCTGTGTAGATTTGTGTACAGGGTCCTGTCTTCGTGAGCCTATAGCATGGTGGAGTGCAGACAG
TGGCTCAATTACCCATGACCTTTTAAAGATGAAAACCAGGCCAGGAGCAAACCACTTGAGTTTTGCCTATCCCTAAATATACAAGC
TCAGGCCTGTTGGAAACCTATCCAAAATGCTCTTATGTTACTCAGAAGTCTGTTTCTAAGGAGCAGGAAGCTGTCCAGATGATGCT
AGGATATTTGGTTCCTTTTTTCTTTGTTTATTTGGAGATAGGGTCAACCTGAATCTTGCTATATATGCTGGCCTTGAACTCGCAGA
ACTCAGTCTCTGCCTCCTAAGAGTTGAAATTAGAGGTGCACATGGCCACAGCTGGCAATGTTTGTGAACTCCCCTTTCCATGTATT
TGCTCCCTTTGCCTATATGTGATGAGTGAGGTACACTGTGCATTACTGTGGGCGCTAAAGTGTGCATCAGGACAGACCATGCCATT
CCCATCCTGTGCTGCCATTTTCATACCATGAAGAGTGGCTGTTTATACAGTTGGGTTGGTGACACTTTGCTCCGAGACCCTCCATC
TTTGACCGTTGTGCTGGTAGCTTGAGTTGCAGTCTCTGCTGTGGTGTCACTGGGCCATGAGAGGCAAAGCTGTCCAGAGAGAAGGG
GCTCCTGTGTGTTCTACAGCTGCAAGGCAGCACTTTGCTTGTGGCTGGCAGATGTAGATATTTATTTAGGTTACTGTCTAGCAGTA
GTGCAGAAGGACAAACTTTTGGGTAGGTCATTTTCCATCCCTTTATAATAGGGACAGGCAGGACATATGGCTTACTGTGAGGAGGT
AATCCCATACATTTTCCACAGAGTAGAGAGTAGGGGATAGCTTTGGATAATGACTTGTGTTGGATGAGAAACCAAGTCTTGGACAG
GTTCACTCTGGGGAGGCAGAAAGAGAAGTATGGGGTGGCAGGAAAGGAGATCTGGGTTGGGGGAGCAGAGCTCTGGGGAACGTGGT
TGGATAAGATGCATGGAATTCTGAGAGGATGAGGCATGTTGAATTTCTTGGCAAGTGACTGGAAAACCTGGTGCTTTGTAGATAGG
GCTCTGGTCTTGTTTGGTGTTCCTTGGTTGCTATCAAGGGATGTGTGCTATCCCTGTGGCAGTAGGTCTTGTCCCCGTACATTTGT
GAAGTAGTAAGAGTACCGTGGTTAGCCTTGAGGGGCTTACTAGGCTTCTGGCTGCTTCTCCTGCTTAGAACTCTGAGCTGCTTCTC
CTGCTTAGAACTCTGAGCAGCAGCTCAAGGATCCACCTCCCTCTGGTGCTGCAGAGCTAGGCTGCTTCCCTGCTACTGTCTGTCTC
TTGGTGCTTCCACTTTGTTGGCTAGGATAGAGAAGTGCTGGTGCAGGATGCTGACCAAGTGCTATTTGGTGTACTGCCTGAGAAGG
CAGCTGTGACTGGCAACTACAGTGCCCACGCCTAGAACTGAACCTGCATAATATTCCGCCGCCAGTAAGGGTAGCTTAGGTTTGTA
CCTCTTGTGTATCTCCTTTCTCGTACTCCCTCCATTCCTGCCTCCTGGAGTCAAGCCAAGACCCCGTTGTGTCGACTAGACCTTCC
TGTCCCATTGTCACAGCACATTTATAGGGACTGGGTACATTTATAGAGACTAGATCCCAGGTCCTGCTACCCTTTTAGTCTTACCT
GTTGGATGAGCTTGTTAGATCCCTGGCAGGAAGAACTTTGGGGTGTGACTGATGGAAAGTTTCCTCTAATTTTCTCAGAGAGAAAA
TGATGAAGAGATGAAAGCTGCCAAAGAGAAGCTAAAGTACTGGCAGCGGCTGCGACATGACCTAGAGCGTGCACGCCTGCTAATTG
AGCTGCTGCGCAAGCGGGAGAAACTCAAGAGAGAGCAGGTGAGGAGGGAGGCCCTTGGGTTCTGCCACCCTCTGGGCTGTCCCTGG
ATAGACGTCTTGCTGCCGTCATGGAGTGCTCTGGAGTGGCCCCTGTGTACCTGCTGAGTTAGTGCTGTCCCCACCCTGTAGCATAT
CATATCCCTACCCTATAGTTGGTCCTGTGGTACCTCTGTGTTGTCCTTTTCGATTAGCCACCTCTGGAGTATACGGGGTCTTAAAG
GAGACCCCTGCCGTGGAAGAAGTACATGTCCTTGCACAGAGAAGGCAGCTTTGTGGTGGGATGGTAGCTGGCACGTAGGCTGCTCT
GTGCTGCTGGTTCAAGTGGCGCTTCTGTGATTGTGCAGTACGTGGAGGTGCGGTGATCTCCAGGAGAGGTGTCCCTACACTCCTCT
GGAGACAGTGTATGCAGAGGTGTCCCTGCATCTTCTAGAGACAGTGTATGCATGCTGTTGTTGCCAGGTGAAGGTGGAGCAGATGG
CTATGGAGCTCCGGCTGACGCCGCTAACTGTGCTGCTACGCTCAGTCCTGGAGCAGCTACAGGAGAAGGACCCTGCAAAGATCTTT
GCCCAGCCCGTGAGTCTCAAGGAGGTGCGTGTCCCTGCGACTGAGCTCTTCGGCTGCTTGCTTAGGAAGCATGCAACTGGGGAGAG
GTTACCTGCATTCTTAATTCTCATTAGTTAGTAGTTAATGAATTTTTGGTGAATAGTATTTTAATTATAAAAGATTGTACCTCGTT
GTAAAGCACTGAAAGTGCATAGGTGAAAATTTCTACTTAGAACTTAACAATTGGTGATGATAGCCCCCCTGGTACCCCATCTGTTT
GTACTTTTAGTTGAAGTAGGTTGGGAGGGTCTCTGCAGTGATTGGGCTTAGTTTGTATTGGCTTAGTGTTGTTATGTGAAATTAGT
TTCAGGTGTGGTTGATTTTGTAAATGTTTATTTTCCCTCCTAAAATTAGGTACCAGATTATTTGGATCACATTAAACACCCCATGG
ACTTTGCTACAATGAGGAAACGGCTAGAAGCTCAAGGGTATAAAAACCTCCATGCCTTTGAGGAGGATTTTAATCTCATTGTAGAT
AACTGCATGAAGTACAATGCCAAGGACACCGTGTTTTATAGAGCTGCAGTGAGGCTGCGCGACCAGGGAGGGGTTGTCCTGAGGCA
GGCCCGGCGAGAGGTGGAGAGCATTGGCCTGGAAGAGGCCTCGGGAATGCACCTGCCTGAGCGACCCATCGCAGCCCCTCGGCGGC
CCTTCTCCTGGGAAGAGGGTAAGAACTGTATCCAGGAGGACAGCGGATGCTTTTTCTCTCAGACTGCACTCACTAAGACTCCAGCA
TGCCGGCCGAGTGAGTGCTCCTGAGGTGCATGCGCCTTGTATGGGCACCACGTGGGCCTCGCCATGTTTTCACATACCCACTGCGA
GAAACACATATCTAGGTGCTGAAGGCCCCGAAGACACTATAGTTGAGGATGCATCCCCAAAGGGTCTGACCTTGCTTCTGAGGTCA
TGCATTGAGAAGGCAGCTATTCATTAGTTGTCATATTTCAGCTGAGAAGCAAAAGCAGGAGCTAATGTTGGCTGTGCCTCTGATCC
TCTCTCTGGGATGCTTGCAGGTGTTTATTGAGGGCCCAGCCTTAGCCTGCTTCTAGGACATGGCCTAACCCTTCTAACTCTCCAGG
GCAAGCTTGTACTCTGGGCCCCACCGTGCACATGCTGTTGTGCTCTTCATTAATTTCTTCCAAGTAAGGAGCTGTTTTTAAAGATA
AGGTCTCAGTGGGTAGTCTTGACTGGCCTGGAACTCAAAATGTGGATCAGGCTGGCTTGGACTTGACAGAAGTCCACCTGCTTCTG
CCTCCTGAGTGCTGTGGTTAAAGATGTGCATTACCATACCACATCTGGCCTCCAATCATTTCTTGTAAGCTTCTTGCCCCTGGATT
GTTTATTCTGTAGGTAAATGTCTACAGTAGGTGAATGGGGTTTGGTGGTCAACCTTGGAACTTTTATTCACAAAACCCAAGATCCT
ATGTTCCTGATTTGACCTACCTTTTCTCCTGCTATTGACTGTTCAGGAAAATGGTGGAATCGTACGGACTTAGGTTTTATCCGGTA
CGTTTCCTTCTCCTGGATGACCAGCTGCCTGGTCACTGTGGCCTGACTCGTGAGGTCAGAGCCCTTGGAGACTCCTCACTTCTGGC
TTCCTGTGTATCTGACCCAGAGAAACTGTCTGTCTCAGGCATCTCTAGGGCATACAGGATAGGGTTGAATTCTTTTTTTCTCAAGA
TAGGATGTAGTGCCACACTCAGGAAGCTAAGACAGGAGGTTCACCACAAATTTAAGGTCAGTCTAAACTATAGTGATTTCTAGGCT
AGTGAGTTACACCCTGAGACCCTGCCTAAAAACCAAAACTGATCCTAACAGTATAATTAGAAAGAAAAGCAGCCAGGCCAGAGTGT
GGCTTAGTAGTGTTTCTTTGCATGCACAACATTTGGGTTCAATGTCAACACAGCATAAACTGGGTTGATACAAAGATTAGAATTTA
AAGGTCATATTGGCTATAGAGTGAATTAAGGCTAGCCTGGGTTACATGAGACCTTGCTTTGAAAAATAGATATGCATGCACCCACA
CAGGTGACAAGATTTCTGAAACCCTAGATAGGTCCAGCAGGAACTGAGCCTGATAGCCACCAGGATTACAGAGCGACTCTCAGATC
TTCACCTGCATCCATGTTCTTTTCTCCAGATTGTGTGGGAGGCAAGGGTGGGCTCCAGCCTCATCTGTTGTGGCCGTGACTGTGCT
TTGGGTGGTATCGGCTGCCCTGAGAAGCAGAGGAGCCCAGTGACATCTGGGAGTCTTTGACCCCACAGCTTCTGATTCTCGTGCTC
TGTAGATGGGCAGGGCTCAGAGGCCTCACAGTTGAGATTCCAGGAAACTGGCTTTGTCATTGCTAAATAAATTTCTGTGCCAGACT
TTTTGCCAAAAAGGAAAGTAATAATGAAAAGTACAAATTTATTTCTTACTCAGTGATTGCAGTAGAAAGCATGACCTGTGGCAGGG
TGAGCTCTGGGTACTCTGCCGCTGTCTTGAGCCTGCAGTAAGGAAGATACTTGTCTTAGTTAGGGTTTTTCTGTTGTGAGCAGACA
TCATGACCAAGGCAAGTCTTACAAGGACAACATTTAGTTGGGGCTGGCTTACAGGTTCTGAAGTTCAGTCCATTATCATCAAGGTG
AAAACATGGCAGCATCCAGACAGGCATGGTGCAGGAGGAGCTGAGAGTTCTACATCTTCATCTGAAGGCTGCTAGCAGAATATTGG
CTCCCAAGCAGCTAGGAGCCCACACCCACAAGGCCATACCTCCCAAAAGTGCCACTCCCTGAGCTGAACATATAATATACAACCAT
TACATTCCACCCCCTGGCCCTCATAGGCTTGTCCAAACATAAGCCTATGGGAGCCATACCTACACATAGCATAATGCAAAATACAT
TTAGTCCGACTTCAAAAGCCCCCATAGTCTATGGCAGTCTCAACAATAATCGTCCAATAACTTAACTGTAATCCCCAAAGCAAGAC
AGGAAGCCAGCTGGGCTCTGCATCTCCATGTCTGATGTCTTCAGATCTTCTATTCCTTTTTCATCTTTGTTGACTGCAACAAACTT
CTTTCTCCTGGGCTGGTTCTACTCCCTGGTAGCATAGCAGCTTTCCTTAGCAGATAGTCCAACTACCACTCTGGTATCTCCAAGGC
AGCTTCTTGTTTTAATGTCTGGGCCTCCTCTCCAAGGTGACGTCACTTCCCCAGCTCTGCCCTCGGTAGCTCTAAGCTCAGGTTGA
TCCCTCCACTGCCGCTGCTGCTCTTGGTGGCCATCATCTCCAATACACTGGGGGCTTCCGCTGCAACTAGAGCCTCTCTAGGCTCT
CTTCATGGTGCCAAGCCTCAACTCCTTTGCATGGCCCCTTCAGTCCTGGGCCATCATCTGCAACCGAGGCTGCACTTTGATCAGTG
ATCTTCCGCCTCAGCTGCTCTTCATGGCCCCTTCATGCCTCAAGGCCAGTGCCACCTGGGGGACCATTGCAGTCACCCAGCATAGC
TGCAGCATGAGGTGCAACCTTGGCTGTCTCTGGAACACAGCTTCTTGGTGCTCAGAAAACACTTCCAGTGATGCTGGTTGTCGTCA
TGATTTATTTATTATATGAGTACACAGTTCTCTTCAGACACACCAGAAAGAGGGTATTGGGCCCCTGTTACAGATGGTCGTGAGCC
ACCATGTGGTTGCTGGGAATTGAACTCAGGACCTCTGGAAGAGCAGTCAGTGCTCTTAACCACAGAGCCATCTCTCCAGCCCTGCC
GGTCTCTTAATCACTGCTAATGCCTTAGCTCCCGCTAACCAGCATCAGCTGTCCCAGGAGTCTTTCTCCTCGTGATTATAAAGCCA
GAGACACATGGCCGAAGCTGCTTGCTGGAGCTGGAACATGGCCCCTAGTTCTATTGCGTCATCACTAGCTTCCAGCTTTCGCGCTC
CTTCAAGGCCTAAGTTTGTCACGTGGGGATCTTGCTCAGAACTCTGAGATATGCAAGCCTGACTCCTGGGATTAGAGGTGTGTACC
AGCACGCCCGGAATTAAGCTTTTCTTCACCTACAACTTGATCTGTCCTTGAAAGTAGAGATCTGCCTGCCTTTGCCTCCAGGAATT
AAAAAGCTTGTTCTGCCCAGTATAGACCAAAACTTAACTGGGTGGGATCTTGCCCCAAGGTCACTAGTCCCTTAATTCAAACTAAT
GTCCTTGAACACATTCAGCTCCATTCACTTCCAGTATTCCTTTCTAACCTTGCAATGCTTATTCACATGCTCTTCCTGAGAACAAA
GTCTACGATGGGCCTTTCTAAGGCTTCCTTTGTCATTGTAATTAACCTGAGCCTCCTTAGCCTCAGGCAGACTCTTCAGCCAAGGG
CAAAAATAGCTACTTCTTCACCAAACTACAAAAACAAGGCTCTAGACCACATAACTGAAATTCCTCACTGAAACCTCTTGTGCTGG
GTCTACACAGTTCCGATTACTCACAGCAACAAAGTGTTCCATAGTCCAGCTAGGATAGACCATGAAGCCCCACTTGAAACATTCTG
TGGCCTTCCAAATCCCAAGTTCCCCAACCTACATTCTTATAAGCAAAAACACGGTCAGGCCTATTACCGCAATATCTCAGTCCCTG
GTGCCACCTGTCTTAGAGTTTTTCTGCTGTGAGCAGACACCATGACCAAGGCAAGTCTTCTAAGGACAACATTTAATTGGGGCTGG
CTTACAGGTTCCGAAGTTCAGTCCATTATCAAGGTGGAAACATGGCAGCATCCAGACAGGCATGGTACAGGAGGAGCTAAGAGTTC
TACGTCTTCTGAAGGCTGCTAGCAGAGTACTGACTCCCAGGCAGCTAGGAGCCCACCCATGAGGCCACACCTACTCCAACAGGGCT
ACACCTCCTAGCATTGCCGCTCCCTAAGCAGAGCATATACAAACCGCAATACTGGCCCTGTTGAAAGAGAAGCCAACCAGCAGAGC
CTGCAGGTCTAGCACTCAGGTTGAGGAGGGAGGATTACAAGTTTGAGGCCAGCCTGGACTCAGCAAGCACAAAACAGAAGAAAGGA
GGCTTGAGAAGTTGAGTGGTGGTTTTTGTTGCGGTGACTGTAAGCCAGTTGGACAGTGTTTGTCGTGTCCCACTGCTAAGTTAGTG
CTGTTTAGACAGGGCGCTAATGAGTCTCCTAGGCCAGCTACCAGGTCTGGGCAGGGCTCATTTATGGTAGGTGTCTCTGTTGGCCC
TGCTGTTCCTTTGGTTTTATCTTCGCATAGATTAAATAATTTTTTGGCTATTTCACTAATTTAAGTCCTGCAGTCAATGTTCCTAG
AGTCTGGGGAGACCTGCGGACTCTGCAGCCTAGTTTCCTTTTGGTCATGATGTATGTGCAAGAACTTGAGCTAGGATGATGTTCAC
AATGTATAAACAGTCCATGTGAACATATTTACACACACGCAGCGTCTGTCAGTAGTCCATCTTGCGTCTATGTTGGTGCACTCAGA
CATGTCTGGTGGTCTTTGTGCCTCTCACTTTTTACAGAGCAGGACTGAGTTGGGTCTTAGTCCAGGAAAAGCCATGTGTGTTACCC
ACATCTCCTCTGCTACGGCCACACTAGTCCTTTGTGTACTACTGACTGAAGGAGTGTCTTGTCTCTTTTTTTCCCTCTTTGTGACA
ACAGCCTTGTCATAGGTTCAGAATCAGGGTAGAGAGGAGTATGTATGGCACCAAATGGTGAAATTGGAACACTTGGGAGGCAGGGG
CAGGCAGATCTCTGAGTTCAAGGTCAGCCTGTTACAGAATGAGTTGCAGGACAGCCTGGGTTACCCAGAGAAACACTGTCTCAAAA
ACAAACAAATAAAACAAAACAAACCCAAGAAGCTAAATAAACAAACAAAGATTAAATGAATTTGAAGCCTGCGCTTTGGCCGTGGG
CAGGCCCAGGCACATAGTTAAGACAGATGTGTTGTTATCAGAGGCGGCCATGAATCCGAATCCTGTGGCTAATGATACGTGTTTTT
GGTTCAGTGGACAGGTTGCTGGACCCAGCCAACAGGGCCCACATGAGCTTGGAGGAGCAGCTGAGAGAACTTCTGGACAAGTTGGA
CCTGACCTGCTCCATGAAGTCCAGCGGCTCACGGAGTAAACGGGCAAAGCTGCTTAAAAAAGAGATTGCTCTTCTCCGAAACAAGC
TGAGCCAGCAGCACAGCCAGGCTCCGCCCACAGGGGCAGGCACGGGAGGCTTTGAAGATGAGGCTGCTCCACTGGCCCCGGACACA
GCGGAGGAAGGTAAGCATGGGGTAGGAGGGCCATACCTCACGGGCTCGGGGCTCTCTTGACAGGCTTAAATGATGCTCTGTAGTAA
TGATGAGCTTGTACATTTTGAAGGTCACGGAACTCTTGGTTACTGGATATTCCTGCTAGGCTTTTTTTGATGCTCTTTGAAAGGAT
GTTTTGGTGTGTTCTGTCTGCTGTATTTTGGCACTTAGTTTACAAGCTTAAAGGAACAGAATGAGATTTTCTTTTAACTCGAGCTT
GAAAGACTTAGAAGGAATAGTTTAGATCCAATACAGTGTTGAAGGTGGCTTCTATGGTGGGAATGGCAATAACTTAGTTGTATTTT
GTTAATTGAGGCAGAGTATTATGTGAGTAGACACCCTAGAATTGTTTTTACCTTGTCTACGTAGGTCAGAGGACAGCTAGTTGGAG
TTGGTTTTCCTGGCATCTTAGCACGCTTGGGGATCAAGCGCAGGTGGTTAGGCCTTGTAAGCACCTCTGCCCTTAGCTAAGCCCTC
CTGCGGCTGGAGTTAGGAAAGGAGGACTGGCTAGAGAACAGCCCAGCCTTGGGCTGGGCATGGTGGGAGGAGTCTGACGTGCACAG
ACCTGTTCCCAGACTCTCCCTCCACCTCAGGCCTTTCCTGTGGCTCACCTTCAGTGGACACTGTCTTATTCTGGCAGCGTGAGTGA
CTTCTGGGGAAAGAGCTGGATAGCTGAGATGTTAGGGTGGAGAGGAAGGAAGGGAGGAAGTACAGAAGAGGCTGTCTGCCCCGTGC
GATCCACGAGATGAGCAGGTCATTGTGTGGAGGGAGGGAGGCTTCTGTGTGTGGTGCATCTAACTGGCATGTTTGATGGTACAAGC
ACCCTTTAGTCCACTTGTCTTGACATCACCACATTTCAACTCCATGAAATGGAAAGAAAAATAAGACCTACTTCTTCTGCCACTGC
TATTAGCAGCTTGACTTAGGATCTCCCTGTGCATTTTTTTTTTCTGCCCCATCCAAATAAGAAAAACATTAACACAAGACCATTGT
CACCATAGTTTGCATTTTTTTGATCTGTATGGCTGCCTGTCTTAGTAGATGTGACTTTGCCCTATTCCTCAGAGTGACATGGTTTC
AGTATGTTTATGCCATGTTAAATTTAGTCTTATAATTTTAACAGTTGGTGACAATCTTCTAACCCACTTTCCCCTTCTCTGGTTGC
TTCTTTTATATGGTTATGCTAGGCAACCAGCAGAAGCTAGGGCCAACACCAGAGTTCTCCTGGCCTTACATCCTTCTAGTGTGTTC
ACTTGTAAACTCACAAACACCCTTGGCCTTGCCATTAGGTAACAAGTTTGATTGGTCCACACAGTAAAGGTTTTATTCCTCAGTGT
GTGACACGTTTTCTCCTCATTTTCTAAAAGCCTAATGACCTGCACATGGCAATTTTCTGCCTCTGTTGGGGCCTCTATGCTTTCTT
TAAGGAACATTGCTCATGGGACCTTTGACAAACAGATGCATCCAGGATACAGTTATTGTTTGCATTCTGTGGTGAGGCCCATATAG
TGCCATTGCCTGGTTCTCATGGCAGCCCTTCTCAGGCTCCTCTTGTCACTGCTTGAACTGGCCCAGTAGGACCCTTGGTCCAGCCA
CTTAGTGAGTGACCTGTACACTTTGTCCTAAAGAGTCAGCTGGGGAGAAGGGTTAGGCAGGACCGCTCACTGACATTGCAGTAGCT
TTACAGGATTGAGGGTCTGTCCACCTTTTGTATCTAAATTGGAGGAAGAGCAGTGCTATTGGAAGACTGGATCTGGTGCGTTGCAC
TGCTGCGGCCACTTCACAGGAAGCACATTGGTGCTACCCGAGACCCGGGCCTAACATTGCTGCTGGCCAGTGTTTAAGATGCAGGA
AAGGGGCACTTTGCTTTTAGCTGAGAAGAAAGATGAGTGGAGAAGGAAAGAGCCTGACAGTTTGTTCTGAGGCAGAGCTGTGAGGG
TGGAATTTAGGGCCTCTTAAAGAGACTGAGTTCCAGACAGGCAACAGGGGAGCACTTCAGTTCTGGTGAACAGCAGACACAGAACT
GTGAAATTGCTATATGCATGTTGGGACAGAACCCTGAACTCAAGACATTACGTAGTAATTCAGCATATTCTTCCCAAAGAGGATGT
TTTGGTTGGATGCAGTCATACATCCTAGAGGCAGAGGCAGGTAGAGCTCTGAGTTCAGAGGCCAGCCTGATGTATAGAGTAAGTTC
TAGACCAGCTAGGGCCCTGAGACATCATGACCAATTAAAAAAAAAAAAATCTGTGATTATTATTTTTTTTTTGAGAAGGCTCATAG
ATTTTTCACACCTAGGAAGGCATATCTTAATATAAAATAAGCAATTTCACTTAAATTGTAATTAAACAACATTTTTGTGTATTATA
CCATGTAGGGTGTTTGCATTAGAGGAAACATCCCCTGAAGGCTAACATCTGAGGAACAAAACAGGCCCTAGCTGTCCTGGACAGTG
GACATGCCTGGCTTGCTTGTACAAAGGGCAAGCTGTTTGTCAGGAGGCCTCCCATGCTGACCTTAGGGTTGAAGAGTTCAGTCAGT
TGAAGTCTGAGGGACACATGGAATGGGGCCATGATAAACCTGGGGACAAGCTTGAGCTCTTAGACGTTTTTACTCATTCATTTCTA
ACTAGGAGCTTTGGTGAGCTCAGAGTCTATGTGCTGGTGATTACTCTTGGCCAGATCAGCACTTCCAGGGGGACATCACTGTTGCT
GCAGCAGCCATGTGCTGTCCCTACTGTATGTACCCCATATTGAATACAGTACACACTGTTCTTTCAGGGCTGGCAGAAGGGAGCAG
GATAGATCGCTGGTGTGGATGGTGCAGTCTCTAGTTATGGAAAGTCTCTGCACACTTTGCTGTGGGATCCAGAGTTATCTGTGGCT
TTGGTGGAAGCATTCGGTTGGCTTGGTGGCCTTGTGTATAGAGAATCATGGTCAAAGGGACTAGCTGGTCCTGAGTAGATGTCTGT
CGAATCCGGATGTGATAGTTGCTAGCAGACAGTGAGGTTTTTAAAAGGACAATGTTTAACGTTTGTATATTAACTGCCAGTAAGGT
TTTTCTTCCTGCCTGAGGGACCTGATGGGAGTGTTAGCTATGGCACTGGTGCTGCCCTGTGTTCTGGCGTGAGAGTTCACTCATCA
AGGAGCCTGACGCCTTGGGTGTTGCTAAATCTATCTCAGTGTGAGTTTTAGTGCTTTGTGTAGCCTAGCCCTATGGCTGCTGGAGA
TGGTGCTTCACTTGGGCCTGGGCAACGCCTTTTGTATCCAGTGTGATTGTTTTTGTAACACCCAGGAGTATGCCAGTGAACTATAG
GGCAGTAGTTGGGAACCTGGGCTCCTCCACCTCATTGGTTGTCACAGAGCAGGGAGAATGCAGGACTGGAGTGTAGAGGGGACCAT
AGATGGGTGTGACTAGCTATGCAGTCCCTGTGGGCAAGCAGCTTTTGATAGACAGTGGTTGGGGGGGATGAAATGTGGTGGAGACC
TTGTGGGAAGGGACAGCATGTTCACTTGTTGTCTTAGCAGCAGTGACCGAATCTGAAAAGTTAAGCAGGAGGCAGAAAATAGGTCT
TTGGTACCTCTTAGCCATGGAGAGAACGGATGGAAGATCTACAGTGCCTGGAGCCCTGGGCAGGAGGCCTCTGGTACCATTCTCTG
GTAGTCTTGTATGTAGGGATTGGATTGGACATCCTGGAAGCCTCAGGATAAGCTGCCTGGAGTGAGGGAAGAGGTACAGAGCTGTG
GAGGAGGAGGAGGAGGAGGAGGAGGAGGAGGAGGAGGAGGAGGAGGAGGAGGCGGCGGCGGAGGCAGGCTACAGGCGGCAAACTGA
AGTACGCAGTGTGAGCCCAATGGTGGGCATCCAACACAGCCATGTGCTGTTCTACCAGCCTAACACACCATCCAGCGACTTCTGTT
TTCATATTTGTGTGCACCAAGAGTGCCATGCACGGCTCTGCACTGTTCCTAATCCCATCTTCTCAGTGTCCCTGTATGCTTTGCAA
ATAATACTGTCCTAAAGCTGTTACTGAACCTAACCCGAGTGGGCAGGAAGAATGTCGTCATATCCAACATGTCAGTGATTCAAGAT
TTGGCATTTCCACTTCATTGGAAGTTTAACCTCCCAAGTACAGAGGCTTTGGTTCTGTGACATGAGTTTGTGCACTCCTTATCTTG
TGGGTAAAGCTTTTCAACCAAAGGCCTCTTAAAAGGCTGTTTGAGGCTGTTTCCACTGCTTGGTGGTGGAGTTGTCTCCAACAGGC
TTCTCATACACACAATTAGCCCAAGCCACCTGACTTGCCAGAGTTGCTGTTTCCAGTCTGGTGTTGCTGAGTCCCGAGGAGATCTT
GGCGCAGAGCCTTTGTTCTGAGTTAATCAAGTGACGCAGGGACATCCTCAAGGCTTTTAAGTTGTGTAGCCTTTTACTTTCAGAGT
GACTTTTAAATGTAAAGTATTGTGACATAGTGTAAATGTTTTGTGGGAAACTTGTAGTTTGAATAAAATAGAAACCATACCTAGGG
ATAACATATGCATGCTCATGCTACTTTGGAATGTTGAAACTGATGCTGTTGAATTTTCTGTCATATGTCCTGTAGTGAGAGTCTCA
GAATCTTGCCCAGAAAGAACCTTTAGCAGTTGAGCCCAGCGGGCTGAGCCTTCCACGATGAGCTCTGCTGCTGTTTGTTGCTTGAG
CTTTGTGTGTGGAGACAGGAAGGTGCTTCTCCCTGGTCAGGTCCATATTGGTTTTGTTTTTATTTTATTTTATTGTATGACTGTGT
GCCAAGTGCACAGGCCTTGTGCCACAGAGGCCAGGTGAGGGCGTTGGATTTCTTGGAGCTGGAGTTCTAGAGGGTTGTTGGCTGCC
CACGTGGATGCTGGGAACCGAACCTATGCCCCTTACAGGAGCAGCAAGTGCTCTTAGCCACTGAGCCAGCTCTTCAGCTCCCATGT
TGGTAATTTGTAAATACCTTACTAAAGAGGTTGAAATAATTTTGGGGGTCTTTTTTATTTTTAGAGATATGAAGGTAGAGTGGGGG
AACTAAGGGAGTATTGTCTGTGTATCCTAAGGGAGTGGAAGACCATGCTGCCTCGTTAGAGTCTCCCAGCCACTCACCTCCCCAAA
GTTGGGCTTTGGCTTAGAAAGTCCTGGCTAGCCGGGCGTGGTGGCGCACGCCTTTAATCCCAGCACTTGGGAGGCAGAGGCAGAGG
CAGGTGGATTTCTGAGTTCCAGGACAGCCAGGGCTACACAGAGAAACCCTGTCTCGAAAAAACAAAAACAAAAACAACAACAAAAA
AGAAAGTCCCGGTTAGACAGTTGAGTATTTGTTTCTTCTCTTTATCACTCTGTGGCGCAGCATAAACTGCTGTGGGACCTTTAAGG
CTCCCTGAGTTCTGTCACTGTCTCACCCTCCTCAAGGTAAGTAAATACTGATGCAGAGATTGTCCTGAACTCAGATGAGGTTTTTA
ATATTGCTGTGTGTTAAATGCTCTTTCACAGTTTTTTCCAGAAAGTAACTTGTGCACCTGGGCTTAGGACACCAGGCCCGAAATCT
TTTGTTAGGGAAACACACAGTGTTACCTGACGGCCGGGGCTACACACTGCTCCGCAGACCAATGTGTGTGATGCTGTTTCCTTAAT
TGAATTAGTGTTTTGGTGGACTTCACATTTATATAAGTTTTATAATAGATTTTATAATCTTCAGTTTTCAAAATCACTTTATTTAT
AATTTTTTCAGGAGCTAACTCTCCCCCTAAACTTGAACCATCAGATGCATTACCTCTTCCTTCAAACTCGGAGACTAACTCAGAAC
CACCAACCCTCAACCCAGTAGAACTCCACCCCGAGCAGAGTAAACTATTCAAAAGAGTCACATTTGATAATGAATCACATAGCACT
TGCACTCAGAGCGCACTGGTAAGCGGACACCCTCCAGAGCCCACCCTCGCCAGTAGTGGCGATGTGCCGGCGGCGGCGGCCTCCGC
AGTGGCGGAGCCATCAAGCGATGTAAACAGACGCACTTCTGTTCTCTTCTGCAAATCGAAAAGTGTAAGCCCCCCAAAGTCTGCCA
AGAACACTGAAACCCAGCCAACTTCTCCTCAGCTAGGGACCAAAACCTTTTTGTCTGTAGTCCTTCCGAGGTTGGAGACTCTACTG
CAGCCAAGGAAAAGGTCGAGGAGCACATGTGGAGACTCCGAAGTGGAGGAGGAGTCCCCGGGAAAGCGCCTGGACACAGGTAAATG
GCAGGGGCAGCTCTCCCCCAGGGCTCATAATAGAAAACCATGGTGCTCAGCTTTTGTTTTTGCGGCATCCTCTCACTCATGTCAAC
ATGTCAGTGATTCAAGATTTGGCATTTCCACTTCATTGGAAGTTTAACCTCCCAAGTACAGAGGCTTTGGTTCTGTGACATGAGTT
TGTGCACTCCTTATCTTGTGGGTAAAGCTTTTCAACCAAAAGACCTCTTAAAAGGCTGTTTGAGGCTGTTTCCACTGCTAAAGCTG
TAAGCTGTCCTCTGGCAGTGGCCATTCAGCCTCTTGGCAGCCCAGCAGCTGGCTATGCAGTGGGCATGGCTGGCTCCGCCCCTCCC
TTGTTCCTTTCTTGTTGACTTGTATGTAGTTTGATTGCATACCTTGACTATTGTGTGCATGTATATGTGTAAACTGGGACCTGGGA
ATGGCCACATCTGGCACTAGGTGCCCGGGGGGTGGGGTCTTCTAAGAGCAGTTCCCACAGCTCAGAACCATCAATTTAAACCTGAA
CCTTCCTTACTGAGGGCTGCTTCTTCCCTGAGCTCTTTGAAAATATGCTGTCATCTCATTACTTGTAACACTTCATACTTGGCTTA
AGGAGTACCGTGATGTTCCCTCAGTGGTTTTGTATGTTGTTTTAGTACATGTGCGCCTGACTAGGAGGGAAGCACATATCAGGGTG
CACATACTACACATGCCTGGTAAAATCCCACTCAAGCCTTTCTCCTTTACTGCCAGGCTTTTTTCTTTCTAATGGAGGTAGTCTGT
CTGTCTGTCTGTCTGTCTGTCTGTCTGTCTCTCTTTCTTTTTTTTAAATGCTAAACTTGCTGAGTAGTCATGGCTCACCCCTTAGA
GCAAGTTCCAGGAAAGCCAGGGCTACACAGAGAAACCCCGTTTTTTAAAAGACAAACAAAAAAAAGCTAAGTGAGTTTGGTTGAGT
GCTAAAGTGTGGTGTGGGTTGGGGAATAAATCTTGAGAGAACCGGAAGTTGATTGTCTCCTTTCACTCTGGGGTTGAACTCAGGTC
ACTGAATTCCCACTGAGCTATCACCTAGTCCTATTGTTAACCTGAGATGTGTTATTTTCTAAGATGTTGCCTGTGTGCCTATACCT
TTAGCATCCTGTGCTGTCCTTGACTTGACGGCTCTTACTTGGCTTCCTCAAATTCCCTGTGTTGTGCTTGACAGCACTGGCTTGGC
TTCCTCACAGCCTCCTCCTGTCTAATCCCTAGATTAAACACTGCGGAAGCGGGTGCTTGTGTTCTTAAACGATGATTGCCAGCATC
AGATGTGATGTTCAACGTCTGCTGTCTGTGTAAGGCAGGTTTGCACTTTGCTTTTGGTCCTAGGTGCCTAGGATTAGTGTCTTAGT
TATTTTTCTATTGCTGTGAAGAGATACCACGAGACAAAGGCAACTTATAAGAGAAATGATGGAATTTGTGGCTCATGGTTCTTGAG
GTTTAGTCCATCCCCATCATGGCAGGGAGCATGGCAGGCATTCTGGCAGGCATGGTGCTAGAAAGTTAGCCGAGCGCCTACATCTG
ATCCATAAGCTTGTTGGGATGGTAGAGAGAGGAGAGAGATTGTGCAAGCAGGAGAGAACGCTAACTGGAAATGGCTTGTGTTTTGA
AACCTTAAAACTGGTGACACACCTTTTCCAATAAGGTCATATCTCCTAATCTTTCCCAAACAGTTGCACTAACTGGGGATAAACAT
TCAGATATATGATCCTGTGGGGACAGTTTCATTGAAAGCACCACATTCCGTTTCTTGGGCCCCTGTAGGCTTGTGGCTATATCACA
ATGCAAAGTGTATCTAGTCCAACTTCAGAAGTCCCCATTGTTTCATAATTTCACTGGTTTGAAAGTTCAGAGTCTTCTGAGACTCC
TAATTTTAACCTCTTGTAAAAGCAAAATAAAAAATCACATACTTCCAACATAAAATACATTAGCATTCCAATAGTTAGGAAATACC
AGACCATAGCAGGCCTTTAACCCAGCCAGGCAAACTCTGAATCCTCTGGCTCTGTGTCCAGTGTCAGGACTGACAGAGATGGCTCT
CCCCTTCCAGCTTTGCTGACTGCAGAACATCTCTGAGGAACTGCTTCCATGTTGTGTTTGTAGCTCTCCTTGGTAGACATCTCATG
ACTTTGGCAACTTCAACATCGGGACATCTAGCACAATTCAGGCAGCTTCACACAGCAGCCTTTCCGACTTCCCCATGCAGGGATTG
ACCTGCCAGGAGCCTGGTTTCAGTGGCTTTCCCTAACAGGAGGAAGAGTCCACAACTCCTTATTCCTGTATCCTTCCAGACTGTGA
AGTCAGAGCCACCAGGCTGGAGAGCTGTGTTAGGCGTCAGCTTGCCCTGCTTGAGTGACGTTGGCATTGGCTTTGGTTTGTTATTT
ATTGCTTTTTAGGAACAGATCATTCCTTAGCCCCGTTCTTCTTGCCTGAGTAGTCTCGTCGTAAGGACGCCACTCCCGTTACTTCA
TTTCTCCTGACCTCTGTCAGCACAAGCCTTGTCTTTTTTTTTTTTTTTAAGATTTATTTATTATTATATATAAGTACACTGTAGCT
GTCTTCAGACACTCCAGAAGAGGGAGTCAGATCTTACGGATGGTTGTAAGCCACCATGTGGTTGCTGGGATTTGAACTCAGGACCT
TCAGAAGAGCAGTTGGGTGCCCTTACCTACTGAGCCATCTCACCAGCCCCAAGCTTTGTCTTGAACATTGATGGGGGCCATTCTCA
GACCACCACAGTAAGGAAAGTTGCATTCTCATCCAGGCCTCTGGTGTTGGCTGGTCTTGTCTACATGTCAGCAGTGAGCAACACAT
AGGTCTTGGCTGATGAGGGAATTCTGTTTCCTGGAACCCTGTCAGGTGGTCCAGTCAGAGGGTTGGGAGGGCAAGGCTGGCCTGGT
AGTTAGAGTGGACTATTGACACCTCTTCATCTTTGTTTCTCCATCTGTTTGAGTTCCCTGGCCTGAAGCACCACACCTTGAAGAGC
ACTTCTTGCCAAAGTCAATACATTGTGGTTTCTTCCCCCCATCTCTCTCTCTCGTGTGTGTGTGTGTGTGTGTGTGTGTGTGTGTG
TGTGTGTGTGTGTGTGTGCGCGTGTGTGTGCGCGTGTGTGACTGGCACCTTGTGGCAAGTTGCTGTGTACTATGGAGTGCCTTTTA
ATGTGTGTGTGTGTTGGGGGTGGGGTTGGGGTTAAAAAATTGTCTCTCCCAGCGTGCATTAGACATGTACAGCAAGTACCTTTACA
CTGACACATCTTGGTGAACCTTTCAAAAAATGTTGAGACAAGTTGGTCTTGAACCTTTTCTATAATAAAGGCCTTTAGCTCATTAG
CCTCTTACCTCAGCCTCTGAAGTAGCTGAAATCACAGACCTGTACTAGTAGGCCCAGTTAATTTAATTTTTATTTTGCAGTCAGTA
ATTTTATAGAAATTTCTCTCAATGCCCTCTTGTTTTGATGAGTTGATAATGGCACTTTAAAATCCAATAACCCTTGGTTTTAATGA
AGCAATTTTAATATGCCACAGGAAGTAGATTGAAACTGAAGTTATCTCAGTTCTTGTTGGAAGTTTCTGAAACTATATAGCTTTAG
CTTTTCTTAGCCACATTTAGTGAAAGACTCTGGTGTCTAAATCCTTTGTCACTGAACTGATCATGGCATGCTGTGCTCTGCTGCTA
GGAGATTGCTCAGTGTCTCAATACCAGGTGCCTTGCACGCACAGGATGGCTTTCTGCCTCCTCAGGGTTCATATCTACCAGCAGCA
GAAAGTTTGTTAACCCTGTAAGACACTGTAGAAAAAGCTTTCACCATGTAGCTGCTGTCCAGAGCTCCTGCTTTGTACCTGGCAGC
TTCTGCCAGGAAGCCTAGTTAGCCATGTGGGCTCTGCTGGGCCATTTGTCGCCGGGGGCTACTAATCTCGAGGTCATGAAATGTTA
TGTGTTTGCACATCTCAGTTCTTTTCTGGGTTCTCAGTGACAACGGTGAGTCCCAAGGAGTCTTAACTTTAGGATGTGTGATCCCC
AGTGCCTTTGATCCTGACTGAAATGGAGATTTCTGCTTTCTTATTCCAGAATGGCAGTAGCTTTCAGTGGATGCATGATGAATTCC
TAATCGCACTCCTGAGCAGCCGGGAGCCTTGTTAGCACTAAGATCTGACCCTCAGGAACAGGAGGCGTCCACTGCTGCATCTGCCT
GGCCCATGGTGGGCCAGGCCTGGGCTGAACGGGCcCATCCTACCATCGGTGcTGGcTGTGCCTCCACTTGAACCTTGTGGTGCTCT
CTGCGCACCTGGATTTCTTGTTTCAAGTTGCAGTTCTTCGCTGTTTGAGGACTTGGAGTATTCAGAACCTTCTGGTCTTTTCCAGG
TTCATCGGGCACTGAACTTGTAGGGAATTCTCTGGTGCTCTCCAGTGCACTGCAAGATTCCAAGTTAGATTAAGCGTGGACTTACC
TATTTTAAAACTGCCCACCCACAGGCCTCAGCTTGCTCATGCCTGCAGGACAGGCAGGCCATGTGGGCAGTGCCGAGCATGGTGTG
ACTGCTCTTATGGTTTTCATATTTTTTGGAGCTGGCTCTGTTTCGTAGGTTTTTTTTACTCTGCCTGTTTATTTCCATCAATGGAC
CGTCAGGCCAGGACCTGTGTCACCTCTTACTCGTACTCTGTGGTGTGGAGATTCTCAATGAAATGTGTGGTGTGGTAGTGGTGAGC
AGGTGAAATGTCTTCCCTGGCCATGGACTGTTGAGGGAGGAGATGATCCCTGCCCTTGCAGTCAGACTAAATGGCTTCTCACTGTT
TTCCAGGTTTTCAGTTAACCACTAATGTGCCTGGGTAGCTCACTCTTTGGATCCTAATCCTTTTCTCTTAACCTCGACTTGGACTG
GAGTTCTGCTAAATGGCCTCTTGGAATGCAAAGCCTTCGCTGCCTCCTTACCTTCTCCTAGTTCTTGAGGACCACATTGGAATCAC
CTGCTGAGCCATCCTCTAAACAGACACCTACCTAACCTTGAAGGAGATCTGTCCTGGGCCAGCGTACCCCTCTCTCAGCCCAGCAG
TTGAGAGGAGCCTGGTGCCTGAGCAGATGTCTCTCGGTGCCTCCGTCTTGCTGGTGCTATAGCAGAGCCTGCTGTAGCTTGGACAA
CACAATCCAACAGTTTGAGCCTCATCCTGAGCACACTCAGAACTGACCTGGGATGGCCGGGGGCTCCGGTTAGGCCGTCGTGGTCT
AGGTGTCTGAAGGGACTGACTGCACATACACTGCCCGAATGGGCCTAAATAGAGCTCCTTACTTGGTTGTTAGCATCTTTTATTTC
TTTGTTCTGTTCTCCCTCCTTTCTTCTCTCTCAATGTTTCTTGGATTCGTAGGCATTGCTAATCTAGTTGGAACCTGTGCAGATAT
CTGCAGAGCCAGCTGAGAAGTCCTCCTGCAGTGCCCTTGAGTTGGGAGGGCCTTTGGCATGGCCTCTGGCTTGTGTTGGCCATGTG
CAGCTGTCTTCATAGACTGTACTTATGAAAAGCAGAGTGGTGGGTGGGGTGGTCTTGCTCACTGTGCTTTATTGAAAGGTGGAGCG
ATGCACCCAACATAAAGTTCTTTTGGAGACAACAAAGTCAAGTGTGATCAGAGGACAGTAATAGATOCTTTTOCTGCCACCCTTAG
ATGGTTGCATTCCTAGCCTAGGGCACGGTCCAGCCTGGGACACAAGCTTGTCGATGTGCACTAGGTGGGAACAAGCTGGAGCTTTG
GGCAGCATGATTCTGTGCTGTCTCAGAGGAACCTGTGCTCCGGAGGCGTCTGTGGTGGCAGGTGTATCAGCAAGGATGCACTCAGT
GACTACAGTCTCAGGCACAGCTCATGGCTTCTGTAGAGGGTGGGAAGCCTGTCAGTGCCCTGATGCTCTCACGTGCTGCAGCCTGT
GAGCTCTTGGCACTGTGCTGGTTGGGAGCCCAGGGTAGAAGGGATCACGTCCCTACTTCCCCACTGTGTGCTTCTGAGCTTTCCAG
GTAGACCACCTGGGACCTCTCCCTGGCCTGTGGGTGAGAGCTGGAGACCTGAGCACCTCAGAGGACCCTTTATTGGCCACTCCGTG
TAACCCAGTTGTGCTGTAGAAGACCTGGGCTAAGGGGGAGCTTTTAGGGAAGATGTGGTAGTTAAAAGGCTGGGGCTGGGCCTGTC
TTGGGACACAGTTGCAGTGGTATTTTGGTGGGTTCCTCTGGGCCTGAGTAAGGTATTTGAACACTCTCTTTGGAGGCCTCAGGACC
CTCTGTGACTGACATGTGCTTGGGGTCCTTGGTCTTGTGTAGGAGACATCTCACCTTTGTTTTGGTTGTCAAGAGCAGTAGGCATG
TGTGTGATCTTCTACAGTAAGCTTTGGGGGGGGAGGTGAGGGGGTGGGAGGAGCATCATTTGGCTCTCCTTCCTGCAGCTAATTGG
AGAGTCATTGTAGGAAGTGGATCACAAAGAGCGAAGCAGAGACACAAAGCTCTTTACTTGGCTCTGTGTCAAAGCCATGTCATCTT
CAGACAGTGTCTGCAAGAGCTTTTCAAGCTTGTGTCTCCGGCCCCTGTTGCTTCTCAGGGCATGTCCAGGGCCTTGGAAGCCGACA
AGAATCTGGCTGGGCCATGCAAGCACCGTAGTTGTGTTTGGGCTGTAGCACCCTATGAAAAGCAGGCCTGGAGATGGCTCTGCTCA
GCGGGCCCCAGAGGTCTTTCCTGAAAGCTGCTCAGGTTTCATAGTGGCTCTGCCAGCTTCTCAAGGTGTTAGGTATTTTTATGTAA
TGTGTGAAAACTTTCTGTAAACTTAGGAGCCCAGATGCAGTGTGCCCTAATAATTAGACACTTGGGAAAATGAGAGAGAGATTTAA
TCATATTTTTTCCTTTCTCAAAGTTATAAATGTTCTCTTAGTTTTTCTAAACCCCTTCCCCCACAAGGGACTGTTTAGGCCCTGAC
AAAGTACCTTGCTATGGGTAAAGCTGTTGCCATCTTTGTTGGGAACACCAAGTGTGTGTGTGTGAACTGTACTTCTGGGTTCTTCT
AGGTTTCTTTCTAGCACAGAGATGCCAGTGTTGGGGACCTGCTTGTCCAAGACCTTTAGACTCTGCAGACCTGCCTGCAGGTGCCT
GCTCTGCCTAGCCATGGCAGAGATTCTGGGCTTGTAGTCTTCTAAGATCTTGAGTCCTGGAGCAAGGGCTTTGCCTCCGTCCGTCC
ATCCAGGTGACAGGGCCATCTCTGTGTTGACTTTGCTAACCTAAGTCAGCAGGTGTCCATGTCCGAGTTGTGTGTTGCACCGGCCA
AGGCAGCACCACGCTCTGTTGCCTCCTAAGAGTTGCTGGGCCTTGAGGCCCTTTAGGAGAGGGGTGTGGCTTCTTCCCTGTCTTGT
GTCTTCTGCTTTGCCAGTGAGCAAGCAAACGAGAACTTCTTGAAGCGTTTTTGACCTTTTTTAGCACAAGCAGGTCCTTTCCCAGG
CGTGATATGGGAGAACGCAACGAAGACCTTGTAATCTAGACAGTCACCTCATACTTTTAAGAAAATGTTTTTCAAAAATAAGTTTA
CATGTTTTACTTTGGAAAATAGTTTAAAAAATTTTTTTAAGGTTATATGGGGAAGATGGGTATATGTGAACAAAAAGAGTGTTGTC
TGTTTGCTGTTCCCGTCCCCTTCTCTTTCTTAAGCTGGTGTAGCCAGCAGGAGCCATGCAAGCGCACAGCCTGGGGACAGGATCCT
TCTGATTGAGGGAGGTCTGCGAGGACCATGGGTGGCCGGGCCTTTCCTGCTTTACCGACATAGAGTCAGGGTCAGACTTGCCTGCC
AGGAATGTGGTGTGGCCTTGACTCAGATTGGTCTTTATTAAAGCACTTCACAAATCTCCAGATGCTGTGCTTGCCTTTGTGCAGAC
ATTGTACCTCAGGGAGACCGTGGCCGCATGGCTCAGCCTCCTGCCAACTTTACATTCTTCCTGCTTGGGGACCTGACATCGCTCGG
ATGACTTGGGCCCACACTTGCAGGTTTAAAGTGTTTTATCCATATTTTTAAAAAGTTCTTGTTAACGTTGATTTTTTTTTAAAAAA
ATTTAATTTAATTTTACTTTGGCTGGTGGTCAGACCTAGGGCCTTGCATGCCTGCTAGGCACGTGCTCTCCCACTGGGATAGACTG
CCCTGGTTTTCCTTCTTTTTCCTTCCTTTCTTTCTCTCTTTTCTTACTCCCTCCCTTTTTGTTTGTTTTTGTTTTTAAAGCCTAAT
TTTCTATACTAGTCCTTAAAGTTCTACATGGTCAGTGTGTTTTGTGGAATCTTGTAGTGTTCCTCCACTCTAGGGCACTGGAGTGT
TTGTGTCTCGACACCGCTTATTCCTTGTCTAGTGGACAGTCTAGTTTTGATGTAAGCTCCCAGCCCTTACTCCATGTGATCTGTTA
ACTTGGGAAGTGGTAACAGTTTCTTTTTGCTTTGCTCCCCTGGGACTAGTTGAGCATGTGCAGAGCTGCTCTGACTTTCTGTGGTC
TGTTGTGTTCATTTTACTCAGCAGTGCCTCTGCATTTGTCCACGGAGGTCACAGGAGACATGAGATACTGGCTTTTGTGTGGACAG
TGTTCTTTGTGGGGCCAGTGATGTAGCTCAGGTGGTAGAGTGCTTAACTAGCATGCACAAAACTCTGGGCTTGAACCCCAGCACCA
CACAGACCAGGTCTGGAGGCAAGATCAGAAATTCAAGAGCAACCTGAACTCTATGAGACCCTGTCTTACAAAAGCAAAGTTATTTG
GAAAACACTGTAGTTTTTAAGGAAGAGAGAGAGACGTAGGACTAAGTTGGGCTAAAGCTACTGCTCTGGTGTGTTGTGACTGAGCA
TCCGTCTGCTTCTTGCCTTCCAGGTCTCACCAATGGCTTTGGGGGTGCTAGAAGCGAACAGGAGCCAGGAGGGGGCCCAGGGAGGA
AAGCTGCGCCCCGGCGGCGCTGTGCATCTGAATCCAGTATTTGTTCCAGCAACAGCCCACTCTGCGACTCAAGGTAGGCCCGGTCC
TCTGGAGATGGAGATGGACTGCCCTGGAGCTAGTTCATGGGTGTGCTTTGCCATCAGGAACAGCTTCTCGGGATAAATTGATTTGT
TTAGTCGGATTTAACTGAAGTCAGAAGTTGATTTAAGTTAGTTTATAATTAAACTAACACTTTATACCTCCCACGCCCCAAATCTT
TTCTCTGATTAAGATTGTGATGTGCAGTGCCCTGCCTATGTGTACTGTAGTGGCCACTGTCGAGTGGGTAAGGGTGACCCAAGTGG
CCTCCTGGGGACAGGCTTACTTTTCTTGGGTCTCCACACCACATTGTCCGTTGGCAGCCTGGCACCTGGACTAGGATAAAGACACA
GGCGGGGGACGCTAAACTGTGCTCTCAGTTTGATTCATCTCTGCTTTCCTCCGAAGCTTTAGCACACCCAAGTGTGGGCGAGGGAA
ACCTGCCCTTGTGCGAAGGCACACGCTAGAAGACCGAAGCGAGCTGATATCTTGTATTGAAAATGGAAACTACGCTAAGGCGGCCA
GGATTGCAGCTGGTAAGTTGGGATACAGATAATGGATGGAAAGGCAGTGTCTGGTCTTGGTGGCTTGGGGCTGTGAGTCAGGCACA
CTCCCCCCCACCCCCTGCGCGCGCGCGCGCGCGCGCGCGCACACACACACACACACACACACACACACTTGAGGAAAGGAGTAAAC
TCATGCTTACTTAACTCACTGAGGTGAGAACTGCTGCCTGCCTGGGGTTTCAGGTCTGTCCTGGGCTGCGTGGCACTTGGCTCTAG
GAGTTCCTCATCGTTAGGTCTATTCAGGAGGAGATCTGCTTTCTGACTGAATGTGTCCCAGCAGGGTGGTCATCCCTAGCTTCAGG
CCACAGTGTAAGGGAGTGTGTGTGTCTGCCGGGCTGATGCTGTTTGTTTAGATTTCCCTGTCAGTGGACCGGGCCAACTCCAGGGA
TAGAAATCCTCGCTGTTTGAGGCTTGTGGGGCAGAGACCTGGGATTGAGAAGGGCTGGAGACTGCAGGGAATCTCTGTGGCTCTAG
AGGCTGCAGTGCATTCAGTGTGATAGGAGTACTGGAGGCCCTGAGTTACAGCGCCACTAATAGATTGTGCTGCTGTGAGGTGGGAC
ACACCATTTACCAACAGTAGTCAGTGAGGGCCTGTACACACACAGTACATACACAGTGGACTCCTTTTTTTTTTTTTTTTTTGGTT
TTTTGAGACAGGGTTTCTCTGTGTAGCCCTGAATGTCCTGGAACTCACTCTGTAGACCAGGCTGGCCTCAAACTTAGAAATCCGCC
TGTCTCTACCTCCCAAGCTGGGCTCAAAGGTGTGTGCCACCACTGCCCGGCCACAGTGGACTCTTGAGGTGTGTCCTGGGCTGCTG
GACGTGCTCAGCGAGGCTCAGAAGAGCGATGTCGTGGTAGTTTGGAGCAAGCCAGGACTTGTATTTGGCTGTTTGGTTGTGTGATA
GGCATCTGGTACATGCTTAAGGATCCCATCTTTAGAATGGAGGTTCAAGTATGGTGAGGTACAGGGGACACGAAGTCATAGGCCTT
AGAACTGGGGGTGGTGGGAAGCAGGGAGGCCTTGGACAGGCTTCTAGGCCTCCCTTCCCCTGGAGGAACAGTGAGGTAGAACTGTC
CTGCTCCAGCAGCTGGGAAGCGGGGCCTGACAGGAGAGTGGGGCTTTTTCTAGCCCCAGGCTAGGAGACTGTGCTGAGTGTGTTAG
CGGTTCTCCTGCTTGCTCTGACTCTGCTGGACCTTTTCTCCTAGAGGTGGGCCAGAGCAACATGTGGATTTCCACTGACGCTGCTG
CCTCCGTCCTGGAGCCCCTGAAGGTGGTATGGGCCAAGTGCAGCGGCTACCCCTCCTACCCAGCACTGGTGAGTCTGCAGGCAGGG
AGGAGGGTGTTGTGGTGGGACCTGGGGAGGGGCCCCAGTGCATGCTCTGTACCTTGCAGTTCCTCTGCTGCCAAAGGTGTATGATT
GTTGTCGCTCTGGAGGCAGGTGTGTGGATGGCTGTGAGCTTAGAAGGCTCTGAGTTTGAGAGTACTGTGAGTCACTGGAGCATGTT
TTTGGCAGATTATTGACCCCAAGATGCCACGAGTGCCTGGCCACCACAATGGCGTCACCATCCCTGCGCCGCCGCTGGATGTGCTG
AAGATCGGTGAACACATGCAGACCAAGTCCGAGGAGAAGCTCTTCCTTGTTCTGTTTTTCGACAATAAGAGGAGCTGGTGAGTGTG
CTGTCTGCAGCAGGCAGAGCTGGGGTTCTATCCGACCTGGGGCTTAGCTTGACCCATGCTGAGTAAGGGTGTCTCCAAGTAGTTTT
TTTTCCTGGTCCTTGTCTGCTGCTGCCTAATGACACCTGGGGATTGTGAGGTGCTGGTCTCTTCTGACAGCTCCTATCACTTCGTC
GATCCCTGAGTGGCTCAGACTGTCTTCAGTTCTTGATCCAGGCTCACTTGCAGTGGGCTTTCTGAACCCACTGCTCTGCCCCTTTC
CATCCTGTCCATCTTCCTGTCCTCTCTCCACCCAGAGCTATTGGAACTCCTCTCATGGTTGAGATCCTAATACTCCCTGAGGGTGA
TATCTGCTGACATCTTGACCATATTTAGTTGAATCCAGCCCTTTCCTATGCAGACCATTGTAACTGGGTCCTTTCAGCTGGCCATG
CTTAGGACTGAAAGGTGCTTCAATACCATGGAGGGGCCCCTCTTGGGGTGCTACCAGGTTCCCTGTGGTCCTCTTTTTCTCTCTGC
TGACTCTGGCTCCTGGCAGTTCATCATTGAGGCTTACACTGGCTTTGCCCACTTGATGGTTCTGTTTGTAGTCTTTTCACCCACCC
TAGGATGCCTCTTCACTCTGCTCCAGGGTTCACTGACCTATATTATGTGCACACACACATTCTAGTTTGTCCCTTGTGTGCATATG
TGTATGTACCTGGGCTCACCTAGTGGGTCACTCACTTGCGTTATCTTGGGCCTGTTCTGTGTGCATAGCTGTATGTTCCAGGTTTA
TCTCTGTAGCCACTTGGCATAAGCTTGAGAAAGAGAGTTGTATTGTGGTTTTCGTGCCTTAGCTGAGTCCAGAGAGGACTGAGTAG
GTGGGTCCCCTACGCACCCAATCCATCCTGCATGAGGCCCAGCGTGTGGGAGCTTGGGTTGGGTACCACCAGGTTCCTTCCTGTGT
GCATGGGCTGATGGCTGGTGAGCCACACCGAGTATGAGCTGGTGGTTCTTATGCTCTGACTTCTCTTTAAAAGGCAGTGGCTTCCC
AAGTCCAAGATGGTCCCTCTTGGTGTCGACGAGACCATCGACAAGTTGAAAATGATGGAAGGGAGGAACTCTAGCATCCGGAAGGC
TGTGCGGATTGCATTTGATCGAGCCATGAATCATCTGAGCCGGGTCCATGGGGAGCCAGCCAGTGACCTCAGTGACATTGACTGAG
GTGGTTTCCAGCAAAGGCGGTGGCCAAAGCCTCAGCCAGCCGGGAGCTCTGTCCATAGTGTTGATAAGCTGTACATGTTTGTATAT
TGTTCAGAACTTAACTTATTCTGGTTTTCTAGGCGTAGTTCTTTAATTCTTTTTCCCCTGGGGAGGGGAGGTTTCACTTCCAAGTT
TTCTATGAAACCATCTGGTCTTGGCTTTGCAAGTGAGGAGGGTCTGTTGCGAGCAGTGTGGTGTTGGGGTCCCACTGCAGGTGCCG
AGGGCCGAGGCCTCACTTATTCTAATCTGTAGGGTTTTTTTTTTTTTTTAAAGACTTTTGAATGTTTAATAATTTTGTAGATCATG
CTCTTTACACAGAGTACCGCTTATTTAATAAGACGGGATGTAAATTTACAATGACAAATGTGTATTTTAAGAAAGAAAATGACATT
ATTTTGAATGGTACTTTGTGCAAAGAGGGAATAAATTTATGCTGTGTGCATCACTTGCAAATCACCAAAAAATGTCCCGCCAGCTG
CTGCCGGACAGGGCCCGTTCTCCTCGTTGATCTGACTGCCCTGAGTCTCCTGCTCTGCCCTGGCTCCTGCAGGCGTGCCTCCCAGC
GGGTTATTTATTGTAGAAAGTGTACTCATTTGCTTTATAATGAAAAATAAATTTGCAAAGGTATATTGATATGCATTTTTATACAG
GCACATAAAAATTCAACTTGGTGTGGGAGCAGAATGTGTTGCGAGGTTATATACACGACTGGCCTGTGTGTACTTTGATTTTGTAA
CTTGTAATCTTTTGTTTACAATGAGGAGCTTTCTGTAACTTGTTTTCATTTAGAACACTTTGGTAGCAATAGACTTTGGATACATT
TTGTATGGTACATGTGATGTATATAGAATTAGTCCTTTATTTTTATTTCTAAGAGGTAAAGCATTATGTTAGGGGAAAGGCAGGGT
GGGTTTCCAAATTTGCATTTTTATATTAAAAATAAAGTGAAGATTTGGACAGTGTGGCCCTCTCATTCCTGCATCACTAGGAGGCT
GGGTGAGCTGTAGCCTGAGGGACGTGAGGGACTCGGAGCACCGGGCCTGGAGTGGGTGGTGTGACACACTTGATCTAACAGCTGAC
TCGGGATGGCATTATTTATTATTTTGCCTAATCATATTTTTATTTTAAAGCTAAATAGTTACTAAAAATTTTAAATGTTCTTTTAA
ATCTACATGTTTGTAATATCTCCATAGAAACTTGAAAATAAAAAGTCTTCCTTTGGT
SEQ ID NO: 1

TABLE 4
Size, position and sequence of BRD1 exons in mouse. Red marks start- and stop
codons. Highlighted area marks coding part of the gene (UCSC Genome Browser on Mouse
Dec. 2011 (GRCm38/mm10) Assembly)

| Functional | | Genomic | |
|---|---|---|---|
| structure | Size | position | Sequence |
| Exon 1A / | 291 | 88733929- | GCTGGGGAGCGAGCAGCGCCTCGGCAGGCGTCCGAGCAGCTCCGCGTCCGCGT |
| Promotor | | 88734219 | CCTCCGCCCGGCCGGGCCCCGAGCCGGCCTCAGCCGGCCGTGCCGGCGCCGCC |
| | | | GACCCCGCCCGAGCCGCGGCGCCCTGCGGGCCCGGAGCCGCTGGCCGAGCGCG |
| | | | CCCCGGAGCCCGGCGGGGCACGGCTGCGCGGCCGTTGGCGGAGGAGCCGCGGC |
| | | | GCCATTAGCGCCGCCTCGGCCGCGCCGGCCTCCGCGCCCGCCCGCCCGCCGGG |
| | | | CTCCCGCGGCCGCGGCGCCCCCGAAG |
| | | | SEQ ID NO: 2 |
| Exon 1B | 1381 | 88729324- | GTAATCATTGCCAAATGAGGAGGAAAGGACGATGCCATCGAGGTTCTGCAGCG |
| | | 88730704 | AGGCATCCTTCTTCCCCGTGCAGTATTAAACACTCCCCCACTCGAGAAACACT |
| | | | GACCTACGCACAAGCTCAAAGGATGGTGGAGATAGAAATCGAAGGGCGCTTGC |
| | | | ATCGGATCAGTATTTTTGATCCCTTGGAGATCATACTAGAAGATGACCTCACT |
| | | | GCTCAGGAAATGAGTGAATGTAACAGTAATAAGGAGAACAGCGAGAGGCCGCC |
| | | | TGTTTGCTTAAGAACTAAGCGTCACAAAAACAACAGAGTCAAAAAGAAAAATG |
| | | | AAGTCCTGCCCAGCACCCACGGCACACCGGCGTCAGCCAGTGCCCTTCCCGAG |
| | | | CCCAAGGTGCGGATTGTGGAGTACAGTCCTCCCTCTGCACCCAGGAGGCCCCC |
| | | | TGTGTACTACAAGTTCATCGAGAAGTCAGCCGAGGAGCTGGACAACGAGGTAG |
| | | | AGTACGACATGGATGAGGAAGACTACGCCTGGCTAGAGATCATCAATGAGAAG |
| | | | CGGAAGGGTGACTGCGTCTCTGCCGTGTCACAGAATATGTTTGAGTTCCTGAT |
| | | | GGACCGCTTCGAGAAGGAGTCTTACTGTGAGAACCAGAAGCAGGGTGAGCAGC |
| | | | AGTCCTTGATAGATGAGGACGCTGTTTGCTGCATCTGCATGGACGGGGAGTGC |
| | | | CAGAACAGCAACGTTATACTCTTCTGTGACATGTGCAACCTGGCTGTGCACCA |
| | | | GGAGTGCTATGGGGTACCCTACATCCCCGAGGGCCAGTGGCTTTGCCGCCACT |
| | | | GCCTGCAGTCTCGGGCCCGCCCTGCGGATTGCGTGCTGTGCCCGAATAAGGGC |
| | | | GGTGCCTTCAAAAAGACAGACGATGACCGCTGGGGCCACGTGGTATGTGCCCT |
| | | | GTGGATCCCAGAGGTTGGCTTTGCCAACACGGTATTCATTGAGCCCATTGACG |
| | | | GTGTGAGGAACATCCCTCCTGCCCGGTGGAAACTGACATGCTACCTCTGTAAG |
| | | | CAGAAAGGCGTGGGTGCCTGCATTCAGTGCCACAAAGCAAATTGCTACACAGC |
| | | | ATTCCATGTGACATGTGCCCAGAAGGCTGGCCTATACATGAAGATGGAGCCTG |
| | | | TGAAGGAGCTGACTGGAGGCAGCGCCACGTTCTCTGTCAGAAAGACTGCTTAC |
| | | | TGTGATGTCCACACGCCTCCAGGCTGTACCCGGAGGCCGTTGAACATTTATGG |
| | | | AGATGTTGAAATGAAAAATGGTGTGTGTCGAAAAGAAAGCTCAGTCAAAACGG |
| | | | TCAGGTCTACGTCCAAGGTCAGGAAAAAAGCAAAAAAGGCTAAGAAAACACTG |
| | | | GCTGAGCCCTGTGCGGTCCTGCCGACCGTGTGCGCTCCGTATATCCCCCCTCA |
| | | | GAG |
| | | | SEQ ID NO: 3 |
| Exon 2 | 157 | 88716906- | ATTAAATAGGATTGCGAATCAGGTGGCCATTCAGCGGAAGAAGCAGTTTGTGG |
| | | 88717062 | AGCGAGCCCACAGCTACTGGTTGCTCAAAAGGCTGTCTAGGAATGGTGCTCCC |
| | | | CTGTTGCGGCGGCTCCAGTCCAGCCTGCAGTCCCAGAGAAACACGCAGCAG |
| | | | SEQ ID NO: 4 |
| Exon 3 | 132 | 88713886- | AGAGAAAATGATGAAGAGATGAAAGCTGCCAAAGAGAAGCTAAAGTACTGGCA |
| | | 88714017 | GCGGCTGCGACATGACCTAGAGCGTGCACGCCTGCTAATTGAGCTGCTGCGCA |
| | | | AGCGGGAGAAACTCAAGAGAGAGCAG |
| | | | SEQ ID NO: 5 |
| Exon 4 | 129 | 88713298- | GTGAAGGTGGAGCAGATGGCTATGGAGCTCCGGCTGACGCCGCTAACTGTGCT |
| | | 88713426 | GCTACGCTCAGTCCTGGAGCAGCTACAGGAGAAGGACCCTGCAAAGATCTTTG |
| | | | CCCAGCCCGTGAGTCTCAAGGAG |
| | | | SEQ ID NO: 6 |
| Exon 5 | 313 | 88712616- | GTACCAGATTATTTGGATCACATTAAACACCCCATGGACTTTGCTACAATGAG |
| | | 88712928 | GAAACGGCTAGAAGCTCAAGGGTATAAAAACCTCCATGCCTTTGAGGAGGATT |
| | | | TTAATCTCATTGTAGATAACTGCATGAAGTACAATGCCAAGGACACCGTGTTT |
| | | | TATAGAGCTGCAGTGAGGCTGCGCGACCAGGGAGGGGTTGTCCTGAGGCAGGC |
| | | | CCGGCGAGAGGTGGAGAGCATTGGCCTGGAAGAGGCCTCGGGAATGCACCTGC |
| | | | CTGAGCGACCCATCGCAGCCCCTCGGCGGCCCTTCTCCTGGGAAGAGG |
| | | | SEQ ID NO: 7 |
| Exon 6 | 261 | 88707034- | TGGACAGGTTGCTGGACCCAGCCAACAGGGCCCACATGAGCTTGGAGGAGCAG |
| | | 88707294 | CTGAGAGAACTTCTGGACAAGTTGGACCTGACCTGCTCCATGAAGTCCAGCGG |
| | | | CTCACGGAGTAAACGGGCAAAGCTGCTTAAAAAAGAGATTGCTCTTCTCCGAA |
| | | | ACAAGCTGAGCCAGCAGCACAGCCAGGCTCCGCCCACAGGGGCAGGCACGGGA |
| | | | GGCTTTGAAGATGAGGCTGCTCCACTGGCCCCGGACACAGCGGAGGAAG |
| | | | SEQ ID NO: 8 |
| Exon 7A | 498 | 88700773- | GAGCTAACTCTCCCCCTAAACTTGAACCATCAGATGCATTACCTCTTCCTTCA |
| | | 88701270 | AACTCGGAGACTAACTCAGAACCACCAACCCTCAACCCAGTAGAACTCCACCC |
| | | | CGAGCAGAGTAAACTATTCAAAAGAGTCACATTTGATAATGAATCACATAGCA |
| | | | CTTGCACTCAGAGCGCACTGGTAAGCGGACACCCTCCAGAGCCCACCCTCGCC |
| | | | AGTAGTGGCGATGTGCCGGCGGCGGCGGCCTCCGCAGTGGCGGAGCCATCAAG |
| | | | CGATGTAAACAGACGCACTTCTGTTCTCTTCTGCAAATCGAAAAGTGTAAGCC |
| | | | CCCCAAAGTCTGCCAAGAACACTGAAACCCAGCCAACTTCTCCTCAGCTAGGG |
| | | | ACCAAAACCTTTTTGTCTGTAGTCCTTCCGAGGTTGGAGACTCTACTGCAGCC |
| | | | AAGGAAAAGGTCGAGGAGCACATGTGGAGACTCCGAAGTGGAGGAGGAGTCCC |
| | | | CGGGAAAGCGCCTGGACACAG |
| | | | SEQ ID NO: 9 |
| Exon 7B | 105 | 88700773- | TCCTTCCGAGGTTGGAGACTCTACTGCAGCCAAGGAAAAGGTCGAGGAGCACA |
| | | 88700877 | TGTGGAGACTCCGAAGTGGAGGAGGAGTCCCCGGGAAAGCGCCTGGACACAG |
| | | | SEQ ID NO: 10 |
| Exon 8 | 136 | 88691749- | GTCTCACCAATGGCTTTGGGGGTGCTAGAAGCGAACAGGAGCCAGGAGGGGGC |
| | | 88691884 | CCAGGGAGGAAAGCTGCGCCCCGGCGGCGCTGTGCATCTGAATCCAGTATTTG |
| | | | TTCCAGCAACAGCCCACTCTGCGACTCAAG |
| | | | SEQ ID NO: 11 |
| Exon 9 | 128 | 88691208- | CTTTAGCACACCCAAGTGTGGGCGAGGGAAACCTGCCCTTGTGCGAAGGCACA |
| | | 88691335 | CGCTAGAAGACCGAAGCGAGCTGATATCTTGTATTGAAAATGGAAACTACGCT |
| | | | AAGGCGGCCAGGATTGCAGCTG |
| | | | SEQ ID NO: 12 |
| Exon 10 | 110 | 88689862- | AGGTGGGCCAGAGCAACATGTGGATTTCCACTGACGCTGCTGCCTCCGTCCTG |
| | | 88689971 | GAGCCCCTGAAGGTGGTATGGGCCAAGTGCAGCGGCTACCCCTCCTACCCAGC |
| | | | ACTG |
| | | | SEQ ID NO: 13 |
| Exon 11 | 155 | 88689509- | ATTATTGACCCCAAGATGCCACGAGTGCCTGGCCACCACAATGGCGTCACCAT |
| | | 88689663 | CCCTGCGCCGCCGCTGGATGTGCTGAAGATCGGTGAACACATGCAGACCAAGT |
| | | | CCGAGGAGAAGCTCTTCCTTGTTCTGTTTTTCGACAATAAGAGGAGCTG |
| | | | SEQ ID NO: 14 |
| Exon 12/ | 1446 | 88687035- | GCAGTGGCTTCCCAAGTCCAAGATGGTCCCTCTTGGTGTCGACGAGACCATCG |
| Terminator | | 88688480 | ACAAGTTGAAAATGATGGAAGGGAGGAACTCTAGCATCCGGAAGGCTGTGCGG |
| region | | | ATTGCATTTGATCGAGCCATGAATCATCTGAGCCGGGTCCATGGGGAGCCAGC |
| | | | CAGTGACCTCAGTGACATTGACTGAGGTGGTTTCCAGCAAAGGCGGTGGCCAA |
| | | | AGCCTCAGCCAGCCGGGAGCTCTGTCCATAGTGTTGATAAGCTGTACATGTTT |
| | | | GTATATTGTTCAGAACTTAACTTATTCTGGTTTTCTAGGCGTAGTTCTTTAAT |
| | | | TCTTTTTCCCCTGGGGAGGGGAGGTTTCACTTCCAAGTTTTCTATGAAACCAT |
| | | | CTGGTCTTGGCTTTGCAAGTGAGGAGGGTCTGTTGCGAGCAGTGTGGTGTTGG |
| | | | GGTCCCACTGCAGGTGCCGAGGGCCGAGGCCTCACTTATTCTAATCTGTAGGG |
| | | | TTTTTTTTTTTTTTTAAAGACTTTTGAATGTTTAATAATTTTGTAGATCATGC |
| | | | TCTTTACACAGAGTACCGCTTATTTAATAAGACGGGATGTAAATTTACAATGA |
| | | | CAAATGTGTATTTTAAGAAAGAAAATGACATTATTTTGAATGGTACTTTGTGC |
| | | | AAAGAGGGAATAAATTTATGCTGTGTGCATCACTTGCAAATCACCAAAAAATG |
| | | | TCCCGCCAGCTGCTGCCGGACAGGGCCCGTTCTCCTCGTTGATCTGACTGCCC |
| | | | TGAGTCTCCTGCTCTGCCCTGGCTCCTGCAGGCGTGCCTCCCAGCGGGTTATT |
| | | | TATTGTAGAAAGTGTACTCATTTGCTTTATAATGAAAAATAAATTTGCAAAGG |
| | | | TATATTGATATGCATTTTTATACAGGCACATAAAAATTCAACTTGGTGTGGGA |
| | | | GCAGAATGTGTTGCGAGGTTATATACACGACTGGCCTGTGTGTACTTTGATTT |
| | | | TGTAACTTGTAATCTTTTGTTTACAATGAGGAGCTTTCTGTAACTTGTTTTCA |
| | | | TTTAGAACACTTTGGTAGCAATAGACTTTGGATACATTTTGTATGGTACATGT |
| | | | GATGTATATAGAATTAGTCCTTTATTTTTATTTCTAAGAGGTAAAGCATTATG |
| | | | TTAGGGGAAAGGCAGGGTGGGTTTCCAAATTTGCATTTTTATATTAAAAATAA |
| | | | AGTGAAGATTTGGACAGTGTGGCCCTCTCATTCCTGCATCACTAGGAGGCTGG |
| | | | GTGAGCTGTAGCCTGAGGGACGTGAGGGACTCGGAGCACCGGGCCTGGAGTGG |
| | | | GTGGTGTGACACACTTGATCTAACAGCTGACTCGGGATGGCATTATTTATTAT |
| | | | TTTGCCTAATCATATTTTTATTTTAAAGCTAAATAGTTACTAAAAATTTTAAA |
| | | | TGTTCTTTTAAATCTACATGTTTGTAATATCTCCATAGAAACTTGAAAATAAA |
| | | | AAGTCTTCCTTTGGT |
| | | | SEQ ID NO: 15 |

TABLE 5
Predicted domains of mouse Brd1 protein (Pfam)

| Source | Domain | Start | end |
|---|---|---|---|
| Pfam | Zf-HC5HC2H 2 | 11 | 130 |
| Low complexity | n/a | 157 | 178 |
| Low complexity | n/a | 234 | 246 |
| Coiled coil | n/a | 235 | 255 |
| Coiled coil | n/a | 257 | 277 |
| Low complexity | n/a | 274 | 293 |
| Coiled coil | n/a | 280 | 307 |
| Pfam | Bromodomain | 313 | 396 |
| Coiled coil | n/a | 446 | 466 |
| Coiled coil | n/a | 483 | 503 |
| Low complexity | n/a | 599 | 618 |
| Low complexity | n/a | 629 | 640 |
| Low complexity | n/a | 709 | 742 |
| Pfam | PWWP | 800 | 897 |

TABLE 6
Amino acid sequence of mouse Brd1 (long) (Ensembl); Sequence ID
ENSMUSP00000105007 (Brd1 (long))

MARKGRCHRGSAARHPSSPCSIKHSPTRETLTYAQAQRMVEIEIEGRLHRISIFDPLEIILEDDLTAQEMSECNSNKENSERPPVCLRTK
RHKNNRVKKKNEVLPSTHGTPASASALPEPKVRIVEYSPPSAPRRPPVYYKFIEKSAEELDNEVEYDMDEEDYAWLEIINEKRKGDCVSA
VSQNMFEFLMDRFEKESYCENQKQGEQQSLIDEDAVCCICMDGECQNSNVILFCDMCNLAVHQECYGVPYIPEGQWLCRHCLQSRARPAD
CVLCPNKGGAFKKTDDDRWGHVVCALWIPEVGFANTVFIEPIDGVRNIPPARWKLTCYLCKQKGVGACIQCHKANCYTAFHVTCAQKAGL
YMKMEPVKELTGGSATFSVRKTAYCDVHTPPGCTRAPLNIYGDVEMKNGVCRKESSVKTVRSTSKVAKKAKKAKKTLAEPCAVLPTVCAP
YIPPQRLNRIANQVAIQRKKQFVERAHSYWLLKRLSRNGAPLLRRLQSSLQSQRNTQQRENDEEMKAAKEKLKYWQRLRHDLERARLLIE
LLRKREKLKREQVKVEQMAMELRLTPLTVLLRSVLEQLQEKDPAKIFAQPVSLKEVPDYLDHIKHPMDFATMRKRLEAQGYKNLHAFEED
FNLIVDNCMKYNAKDTVFYRAAVRLRDQGGVVLRQARREVESIGLEEASGMHLPERPIAAPRRPFSWEEVDRLLDPANRAHMSLEEQLRE
LLDKLDLTCSMKSSGSRSKRAKLLKKEIALLRNKLSQQHSQAPPTGAGTGGFEDEAAPLAPDTAEEGANSPPKLEPSDALPLPSNSETNS
EPPTLNPVELHPEQSKLFKRVTFDNESHSTCTQSALVSGHPPEPTLASSGDVPAAAASAVAEPSSDVNARTSVLFCKSKSVSPPKSAKNT
ETQPTSPQLGTKTFLSVVLPRLETLLQPRKRSRSTCGDSEVEEESPGKRLDTGLTNGFGGARSEQEPGGGPGRKAAPRRRCASESSICSS
NSPLCDSSFSTPKCGRGKPALVRRHTLEDRSELISCIENGNYAKAARIAAEVGQSNMWISTDAAASVLEPLKVVWAKCSGYPSYPALIID
PKMPRVPGHHNGVTIPAPPLDVLKIGEHMQTKSEEKLFLVLFFDNKRSWQWLPKSKMVPLGVDETIDKLKMMEGRNSSIRKAVRIAFDRA
MNHLSRVHGEPASDLSDID
SEQ ID NO: 16

TABLE 7
Amino acid sequence of mouse Brd1 (short) (Ensembl; Sequence ID
ENSMUSP00000105006

MRRKGRCHRGSAARHPSSPCSIKHSPTRETLTYAQAQRMVEIEIEGRLHRISIFDPLEIILEDDLTAQEMSECNSNKENSERPPVCLATK
RHKNNRVKKKNEVLPSTHGTPASASALPEPKVRIVEYSPPSAPRRPPVYYKFIEKSAEELDNEVEYDMDEEDYAWLEIINEKRKGDCVSA
VSQNMFEFLMDRFEKESYCENQKQGEQQSLIDEDAVCCICMDGECQNSNVILFCDMCNLAVHQECYGVPYIPEGQWLCRHCLQSRARPAD
CVLCPNKGGAFKKTDDDRWGHVVCALWIPEVGFANTVFIEPIDGVRNIPPARWKLTCYLCKQKGVGACIQCHKANCYTAFHVTCAQKAGL
YMKMEPVKELTGGSATFSVRKTAYCDVHTPPGCTRRPLNIYGDVEMKNGVCRKESSVKTVRSTSKVRKKAKKAKKTLAEPCAVLPTVCAP
YIPPQRLNRIANQVAIQRKKQFVERAHSYWLLKRLSANGAPLLARLQSSLQSQRNTQQRENDEEMKAAKEKLKYWQRLRHDLERARLLIE
LLRKREKLKREQVKVEQMAMELRLTPLTVLLRSVLEQLQEKDPAKIFAQPVSLKEVPDYLDHIKHPMDFATMRKRLEAQGYKNLHAFEED
FNLIVDNCMKYNAKDTVFYRAAVRLRDQGGVVLRQARREVESIGLEEASGMHLPERPIAAPRRPFSWEEVDRLLDPANRAHMSLEEQLRE
LLDKLDLTCSMKSSGSRSKRAKLLKKEIALLANKLSQQHSQAPPTGAGTGGFEDEAAPLAPDTAEEVLPRLETLLQPRKRSRSTCGDSEV
EEESPGKRLDTGLTNGFGGARSEQEPGGGPGRKAAPRRRCASESSICSSNSPLCDSSFSTPKCGRGKPALVRRHTLEDRSELISCIENGN
YAKAARIAAEVGQSNMWISTDAAASVLEPLKVVWAKCSGYPSYPALIIDPKMPRVPGHHNGVTIPAPPLEVLKIGEHMQTKSEEKLELVL
FFDNKRSWQWLPKSKMVPLGVDETIDKLKMMEGRNSSIRKAVRIAFDRAMNHLSRVHGEPASDLSDID
SEQ ID NO: 17

TABLE 8
Sequence of rat BRD1 gene (UCSC Genome Browser on Rat Mar. 2012
(RGSC 5.0/rn5) Assembly)

CATTGTTTGCTTCGCTGGGGAGCGAGCAGCGCCTCGGCAGGCGTCCGAGCAGCTCCGCGTTCGCGTCCTCCGCCCGGCCGGGCCCC
GAGCCGGCCTTAGCCGGCTGTGCCGGCGCCGCCGACCCCGCCCGAGCCGTGGCGCCTGCGGGTCCGGAGCCGCTGGCCGAGCGCGC
CCCGGAGCCCGGCGGGGCACGGCTGCGCGGCCGTTGGCGGAGGAGCCGCGGCGCCATTAGCGCCGCTCGGCCGCGCCATCTATATC
CGCCGCTCGCGCCACACACTCGCCCTCCCGCTCCATCCACACCCCCGACCCCCGCACCGCCCCACGCCCTCCCTCACAGCAGCGGC
CCCCGCCGCGATTCCGCCCCACCTATCCCCGGTTCGCCCACACCTATAACCTTCTCCCCCCCTCCTGAGCACATCAGCCGGTCCCC
CCCCCCCCCAAGATTCTAGGTACACTTACGCCAAGCGCCGCCACTCCCCATCTTGCACAAAAAACAAAAGAAGAGGATCACACGCC
TTCTGCCATACATCCCCGCCCCGACTGCCACGGCCTCCGAATCCGCCCGCCCGCCGGGCTCCCGCGGCCGCGGCGCCCCGAAGGTG
AGTGTCTGACGGTCGCCGTTCGCCGCCCGCCTCGCCGGCCGGGGCGGAGGTGCAGGCGCCATGTTTAGAGGCGGCAGCGGCGGCTC
CGCATTGTCCGCGGGCGGGGAGGCCGGAGAGTCGGGGCGGCGAGGCCCGGAGGCCGTGAGGCCTGGTGGGCGCGGGAGCCGGAGGA
ACTGAGAAGGCCGAGCGGGCGAGTGCCGCCGTGAGCCGGCGCGGCCGGGGACGCCGAGATGGGTGCCGGCGGCTTGCCCGAGAGGC
CGGGTCTGGGAGGCGAGGCCGCGGCGAAATCGCGGAGGCGGAGGCCGCAGCCGGGTGGGGGCGGAGAGGGACACGGAGGCCGCGGC
GGGGTCGGGGAGACAGAGGAGTAGAAGGAGGCCGCCGCGGCGCGGGAGGGGCGGCCAAGAGAATGGAGCGGGCGGCAGGTTTCAGG
AGGCGGGGAAGCCGCCGGGCCGGGCGGGCTCTGGGCGGCCCGGCTGTCTGTGCAGCTGGGGCAACTGCGGGGACGGGCGTCGGACA
GCGGAGGAGGCGGAAGGCCTGGGGTCTCGTGGCGTCTGCCCACGTCCTCGCCTGTAGCCTTGGCGGTGCGGAGCCGGTCGCATTAT
GTAACAGATAGGTCCGATCTATTTTGCCAAGACAGGAAACTCCCTTGAAGAGGGACGGGCTCGGAAGATTTCCTAAGTCGAGCGGG
GCCTGGTATCTCCGGAGTAAGCCCGCAGCTCCGCCAAACTCCGTGGATGTGTGCAGGAAACGCCGAGAAACGAACGCGCGTGCGCG
GCTTTCTTGGGCCTTTAGGAGAGAAGCAACTTTCCTATGCTTAATTTGCAGAAAACACTGCTCCTCATCGTGCACTGCAGTTGTGA
CACACTTACACACACCTAGGAAACCGCCCCCTTAATGGAGGACATTCACTTCACCCAGCCGCGACTGTTTTAGAGTATCTGTCATC
TGGTAACACATAGTTACAGAATTTTGATATTATTTAGTTACTGTTTTATCACTTGTTGGATCTAGCACTGTTCTGAGTCTGTGTTT
ACTCCTCAGATTGTCACTTTAGAGTAAGTGTCTTTCCTGTGTGCTTTCACAGTGAGGGGTAGAAGCTGGAAGAGTTTAAATGGCTT
GTCTACAAACCAGGCAGGAAATGAACTGAGCTGATTTTGAGCAGAGTCTTTCCCTCTTTCTGCTAACAAAGCTTTTTAGGATGCGT
TTAGCACAGTTATTTCTGGAGAACCATGCTTATTGCCTTTGCTGATTCTTTCATGGAAATGCTCATTCCTGCATAGAGCCAGAGGG
TCAAAGTGCTGGGTGTATGAAAATGAGGAAGCAGATGAGATTGTTGGTCACTGCTGGGCAGTGCCTCTAAATGCCCTCTTTCCCCC
GGTCACAATTACATTTTCAAATTACAGAGTAGCTGTGGCCATTAAGTATTAGGTTCAGTTCTTGTAGAAAAGTGGTTTAAAGACCT
TCAGTGCTCACTAGGAGAATGTGGGGTTTGACAGGCTGGTTACAGTACTTTACTGTAGAGGAGAAAATTACATGTTTGTTTTTAAT
CTGGGAGCTGTTGCTTCTGCCTGCCTCAGTAGTAAATTGTGAAGCATCCGAGGTGAACTGTGGTTCTTTCTGTGCAGAATATGGTG
CTGACACCTGGATTTGCACCTATCTCATCTCAGGGATGTTGCTAGAGGCCTAGGGCTGGCTAGGGCTGCTTTGATGACAGCTCCCT
TAGAATCCTTTGCTGAGCAGGCACCTGGAAGCTCCTCAGATGCAGGTGCATTGGGGTCTGCTGTTCTTGTTCATAGAGCGATAGTA
TCTACAGAATGTGGGTTTCTGCAATCTGCAAGGTCTGTCTTTAAAAATGCGTATAAGATTTGCAGAGATTTCCTTTTGGGATTTAA
AACATGAAGTCTGCTCTTGGAGGGCTTTTCTCAGAGACTAGTAAGATAAGTATGAGCTGAGAATTCGGGGTTCCTGGAGAGCCCTG
CTTGTGGGCTATTCTGACATTTCAACTTGGTATATTTTGGGAGTCAGTCTTTATCTACTTGTCAGTTGAGTGGGCTTGTTCAGTGG
GAGGCATGAGTNNNNNNNNNNNNNNNNNNNNNNNNNNNNNNNNNNNNNNNNNNNNNNNNNNCCTCAGCTAGGCGTACTTGGAGAGT
ATGTGGCCCAGCCCACAGCCAGGTCCTGGCCTGAGTGAAAGGGAAAGGCTGGTGCTGCCACGGCAGCTTCCAGGGTGGTCACTGCT
GAGGCGTCCTAAAAGCTCCATTGGGCTCTTGGGGCAAAGATCTCCCACAGACCAGCACAGCGTTCCCTTCAAGTCCTGTATGGGAT
GTTTGTGGAAAGAATGGACTACTTTATGCTGTTGAGTTATGGATGCTTCTGGCCCCCAGCACAAAGTTCCCAGGAGCACTCTGCTG
GGCAGTAGTGAGAAAGAAAGACCTAAGGGATTGCTAAGAGTAGGTGGCCACAGGCCATAGTCTCTGTTGTGAAGTCTGTCAGTAAA
ACAGTCTGACTTGTGGGCAAGAGGCCAAGCTCTCAGCTCTGGAGACCTATGTTGCTGTTTGTAGGTAACTTTTGGCTTGGTCTAAA
AAGGTGACTTGTGGGTGGAATGCACCTGTGCCCTAGCTATTCAGCAGGAACCCCGAGGGCTGCAGCTTCCTGCTGTCTTCCCTGAC
TGTCAGTACCTTTACCTGGGTGTGGTGAGGGAGGTTACTGTTGGAGGCTTGTTGTAATGTGTTTGGAAGTCTTCAACTCTGAGCTT
TGTGGGGTGATTTGTTAGTGCTGCCCAAGCATATTTTGTAGTTTTCTGAAGTCTTCTGTCACCCTGCATGGAGTTAACTTTTCTTT
GACTTTATTCTAGGTAATCATTGCCAAATGAGGAGGAAAGGACGATGTCATCGAGGTTCTGCAGCGAGGCATCCTTCTTCCCCGTG
CAGTATTAAACACTCCCCCACTCGTGAAACATTGACATACGCACAAGCTCAAAGGATGGTGGAGATAGAAATCGAAGGGCGTTTGC
ATCGGATCAGTATTTTCGATCCCTTGGAGATCATTCTAGAAGATGACCTCACTGCTCAAGAAATGAGTGAATGCAACAGTAATAAA
GAAAACAGTGAGAGGCCACCTGTTTGCTTAAGAACTAAGCGTCACAAAAACAACAGAGTCAAAAAGAAAAATGAAGTCTTGCCCAG
CACCCATGGCACACCGGCTTCAGCCAGTGCCCTTCCTGAGCCCAAGGTGCGGATTGTGGAGTATAGTCCTCCATCTGCACCCAGGA
GGCCCCCTGTGTACTACAAGTTCATCGAGAAGTCAGCCGAGGAGCTGGACAACGAGGTAGAGTACGACATGGATGAGGAAGATTAC
GCCTGGTTAGAGATCATCAATGAGAAGCGGAAGGGCGACTGTGTCTCTGCCGTGTCACAGAACATGTTTGAGTTCCTGATGGACCG
CTTTGAGAAGGAGTCCTACTGTGAGAACCAGAAGCAGGGTGAACACCAGTCCTTGATAGACGAGGACGCTGTGTGCTGCATCTGCA
TGGATGGCGAATGCCAGAACAGCAACGTTATACTCTTCTGTGACATGTGCAACCTGGCTGTGCACCAGGAGTGCTACGGGGTGCCC
TACATCCCTGAGGGCCAGTGGCTTTGCCGCCACTGCCTGCAGTCTCGGGCCCGCCCTGCGGATTGCGTGCTGTGCCCGAATAAGGG
TGGTGCCTTCAAAAAGACAGACGATGACCGCTGGGGCCATGTGGTATGTGCACTGTGGATCCCAGAGGTTGGCTTTGCCAACACGG
TATTCATTGAGCCCATCGATGGTGTGAGGAACATACCTCCTGCCCGGTGGAAACTGACGTGCTACCTCTGTAAGCAGAAAGGCGTG
GGTGCCTGCATTCAGTGCCACAAAGCAAATTGCTACACAGCATTCCATGTGACGTGTGCCCAGAAGGCTGGTCTGTACATGAAGAT
GGAGCCTGTGAAGGAGCTGACTGGAGGCAGCACCACCTTCTCTGTCAGAAAGACTGCTTACTGTGATGTCCACACACCTCCAGGCT
GTACCCGGAGGCCTCTGAACATTTATGGAGATGTTGAAATGAAAAATGGTGTGTGTCGAAAAGAAAGCTCAGTCAAAACGGTCAGG
TCTACATCCAAGGTCAGGAAAAAAGCAAAAAAGGCTAAGAAAGCACTGGCTGAGCCCTGCGCGGTCCTGCCGACCGTGTGTGCTCC
ATATATCCCCCCTCAGAGGTAAGTGCATCTGAGCTTCAGCTCAGATGGGCCTGGAGGGAAGGACTTGATGCAGGACACAAGTCAGG
GCCTGCAGGAGTCCTGGCACATCTCCACCGCACCTCCTGATAGTCTGTGTCCTAAGCTGTAGCCATTCATTCACTACTGCCCAGTG
GGGCGTAAGTGCAAGAGAAATTACAGATTGGGATAATGTATGGTTCTTAGTCACCTGTTGACCGTGAATATAAGGTAGGTATGCTC
AATGGGAGCCACAGCCACACCAGTGTTCAACCCTGGGCTTCTGGATCTCAGCATCCTGAGTTTTGTTTCTATCTACAATGCCATTA
AACTGCCTTCTTACCAGATTTTAGGACCTTGTAGAAAAGCATCTGGAAAAGTGAACCACCATCCTCAGTAAGGTGACCATTGAGAT
GAGGTTAGAACCAGGGCTGCTGTCAGCAGGGAGATGGTGTTCTGTCTTCCTGCCTGCCTTGAACTCCCAGGGATCCTCTCCCTGTC
TCCCTGGTGCTGGGATTCAGGATGCTCCAACCATACTTGACTTCTTCTACTACTTACGTCTGCAGTAGTGCACATGTCGCTTGATC
TGCAGGAGGGCTGCTGTGCCAAGCCCTGCATCTGTGTTCCCTGAGGGAGGACTTGTCTCCTTGTGTCTTCTGGACTTGCTCTGTGG
CATGCTGTTTTTGTTCCATTATTTCAAGAAATGTACTGTATATCACATCATAGCCTGTGAATGCCAAGTGAATCCACTCCTTTTGC
ATCCATTTGAATCCATTTTAGAGCCTTGAGGAAGTGATTTTTTTGTGAAGGGGGCGTGGACTTTTAGTCTGGTCAGGTTTGTAGAG
CCCCAAGATGACGAAGTTCATGTGAGGCAACTGCCCTAAAGCAACATACATGGTGGACAGTAGATTTCAGGGGTGTGTGTGTGTGT
GTGTGTGTGTGTTTGTTTGTGTTTTGAGATCAAGTCTCTTGTGTTCTTGGCTGTCCTGGAACTCCCATCCACTGCCTGCCCCTGCC
TTATGAGTGCTATGATCAAAGGCGTGCACCACCACCACTGCTCAGAAGCTGTCTTTAACACAGCGAGCGAACATTAGTTTTGTGTG
TGTGTGTTATTATGAATGTCTGGTTTGAAGAAGTTAATCATTCTTACTAGCATGCTGTGGTTATACCGTGGAGTTGGGCATTGTGC
CACAGTGGGCTTACTGTTTGAGCTAGGAGCACAAATGGAAAAAGAGCTAGCACTGCCTTTATGCTAGAGTTTGAAATGGGTAAATG
CTGTTTGTTTTTGTAGACCTATACTTCTAGTCAGTGAATCAAACACAGAGGTCTCATAACCAACCCCTTGTTCAGAGAAGAGTCCC
ATTAGGACCAGCATGCCTGAAAAGTTTTTCAGCCTGACTTAGAAGATGGTCTCTCTGGGATTTCTGTGTCTGTAGAGGCAGAAAGC
ATAGGGTTATGTGAAGAGCCTCTGCTCAGGGAGGTTCTGCTTTACCAAAAGTGAAATAGCTGAGCCATCAGTCGTCTTGTTGCTTT
CTCTGGCCAGTGCCAGATGCTCTGTTGCAGGTGGTGTGATCCTGCACCCTGTCCTGTGGTTCCTGATGTTCAGGTTTTGGGACATG
AAAGCTGCCAGGTGGGCGGGACTGTTGCAAGGAGGATCTGCAGGTGACAACAAAGACTCTGTCCTTCAGAGCCATTGAGGAAAGAA
CTCGAAGCTTTAAACTTAAATCTTCCAGGGTCTGTTGTGGAATTCAGCAGATAAGAGGTGCATTGTGGCTCGTGTTTTTCTCCTCC
AGACAAGGTCTTATTTATAGGCCAAGACTACCTGGGGCTTATGGAGCACACGCCCGGTCGGCCTCAGAACTGTGAGTCTCTTGCTT
CAGCTTGTCAAGTGCTCACTGTCCTGTCCTGGCTTGCCTTCCTTTGTTTTGTCCTGTTTTGTTTTGTGACAGGATTTCACTATATA
ACCCAGGCAGGTTTCAGACTGCCAGCCTCAGCCTCGTGAATAATGGAATTACAGGTGTAAGTCATCATGCATAGCTGCTTGCTGCT
GCTGCTGCTGCTTTTTAAAGATTTATTCATTTTGTATGTGTGTTTTGCCTGTACCTATATATGCGTACCATGTGTATGACTGGTTC
CCTCTACTCAGATCCCCTGGAACTAGAGTTGGGGTGGGTTTTGAGTCACCAGTGAGGTGGAATTGAATATGGTTGAACAGCTCCTT
GGCACCAGCTGTAACTAGTAGACTGAGGGCAGGCAGTGCTGGGGACCATACTGTACTGTACCGTTCCGTGCCCAGCCCTTGGGAAG
CAGAGGCAGGAGGATTTCTGTACAGAGTTCAAGGCCACCCTAGTCTATGTGGTGAGCTCCAGGAGAGCCAGGGTTACATAGAGAGA
CTGAGGAAGGTCTAAAGAAGACAATTTTTGTGTGGGTTTTTGTCCTTTTGTGTGTGTGTGTGTGTGTGTGTGTGTGTGTGTGTGTA
TGTTTTCTGAGGCAGGGTTTTCCTGTATGGTCCTGGCTGTCCTGGAACTCACCCTGTAGACCAGGCTGGCCTTGAACTTGACATCT
CCCTGTCCCTGCGTCCCAAAGGCGTGCACCACCACTACTCTGCCAATTTCTTTTAAGTATGAGTACATACTATAGTATGAATTTTC
TGGGAATTGAACTCAGGACCTCTGGAAGAGCAGTCAGTGCTCTTAACCGCTGAGCCATCTCTCCAGACCTTGCCAATTTTTTTTTT
TTTTTTTAAAGAAAAACATTTTGATGTGTTTTTCTATGCTCTTCTGATAGAATTTGCTGTTACCCTGCTCATCCATCTGATTCTGT
CTCACAGAGGAGGAAAGAGAAGGAACAGGTAAAGGGCAAAGCTTATTTGTTCCCTCCCCTGAAGTATGAAGCCTGGTTCTTCCCTG
CCTTGGCCATATGGGGCCATATTCTTTATCCCTGGAACAGGCTGCTGGTGAGCAGCAGCTGTGTGTGCCCAGATCTGGGACTTGAC
TCAGATAGCCTCTGTCGCCAGATGCTTGGCGTGCAGTTTACTCAGATGCCCAAGTAGTGGTTGGTGCTGGGCTAGTGAGACTGCTC
TCCTCCTCTTTGCTGTTCACACTGCCAGTCCTGATGTCTCTGGGAAAAGACCAAGTGACGTGTGAGCACTTGATGCATTCAGTAGG
TAGACCGGGTCTTGCCTTTTCGTGTGGTCTCTGGTTTGAGTTCTGTACACTCCCCTCCCCATGCTTCTGGTGGCTTACTGTATGGC
CAGTCCCTGACTTAAGATTTGACTTACCAGCTTTTTTCTCTTTTTGTTAATGGCACAGCTGTTTGTTTGTTTATTTAGCATTTGAC
AGAAAGGGTTTTTCCTCTTGCATGGTACTGACTGTACGAGAACTCGCTCTGTAGGTCCTGCTGGCCACCAACTCAGAGATCTGTCT
GTTTTTGCTTCCCAAGTGTTGTGATTAAAGACGTGTACCAACACACCTGACTTTTTTCTCTCTACCCTCTCTTCCCTCCTCCCCTA
GTCAGTCAGTCAGTCTGTCTGTCTACGTAAGTTTTTGAGGTAAGATCCGACTCCCTCCATGTGCCTCTGCCCCCTGAAGGCTGGGG
TTGAAGGCGTGTCCACAGCATGCCTGGCTTAGTTACCAGTTTTGAAAAACGGTACATGCAGAAATATTATGCATTCAGTAAAAATA
TAATGTGAATTTTGCTCTTTGTGGGCTGGTATGTATCATACTCCTTGGGGATGCTGGACATTGCGCTGGTGTGAGCCAGGGCTTCA
GTGAGCTGCACGAAGAGGGGGCCACGTCGTTTAACTGGGCTGTTGGGTAGCATCGTTGTAAGAATGAACCTTTGACTTGTTATGTG
AGGTTGTCCGCATGTAGCCTCCTCCTGGCAAAGAGCATCTGCATTTGATAAACATTTCCGTCTGCCACGGTTGGCTCCTTCCCTGC
CAGGTGGAAGCCATGCCCTGCATCTCCTGAGAGCAGTGTAAGGAGGGCTTGCTGCTGTGCAGGGCCTTGGAAAGCAGGCAGATGCA
GCTACAGAAGTGATGTTGGGGAAGCATTTAAACAACCAACAGAAAAGTACAAATGACACACACTGTCATGTGTGGAAGGCAGAGCC
CACCTGAGAGCTGGCCACTGGAAAGAGAGGAACTCTCTGTCTGAGAGATGAGTGGGCCCGCCTACTGAGCTGCAGCAGCGTGCCCC
TAGAAAGCTGGATCACTGTCGAGCTGAAATCACTGTGTACCGACTCTGTCCCCAACCTGTACACCTTCCCTTAGTGGATCTGGGTC
TCCTGTGCATCTCCAGTGTGGCTGATGTCCTCTTAATTCTTACATAGTTACTTTGGCATAGGTAGAAGTGGACTTCCTTTAGACGT
TTATTGGGGGTAGGGTTGTTCCTTTGCCTCACTACTGCTGCTGTGAGTCCACTTGAAGCTGACATTGGGATGGACTTAGTGCCAGA
GCTGCCCAGAGTCTCTTCTTGTGGGATGGAGCAGGACTTCCAAGTCTGGCTGTTAGAATTTTTTTAGTGATAACATTTCAAAACAT
AAAAATAAAGGGAACAAAAAGGACTGCATAAGAAAGAAGGACTGCATTACATCACCCAGTTATTTGCTGCTAGTGTTCTGCTGTGA
ATTCTGACAATCCTTATGTATGGGTTGTAGGAGAGGCCTTACATGGTCATGTGCTCTCAGGGCGCAGGTTTAAGTTACCCTTTGCA
GTGGGGAAGGACACGCAGGTGTCCTGTCCTGTACATGGTCACCTTCTAGAGGTGATCTTGATACCTGGTCGTTGCTCCATATCTGT
GCCTCCAGCTCCATGCCCCAACCCAGCCTGCCTCACACAACACTCAGGCCCTGTGAGTCATAGGAAGACCATTCTGATACCTGCTT
TGTTCTAAGGTGCTAGAGCATTGGGTCAAATGGAAGGTAAGGGGCCAGAGGGCCTAGAGGTAGGCAGGCTCTACCTAGAGGCAGGG
GACACCATTTTGGCTGTTTAGTCTTTTCAGCTTTCCTGGGTTTGGATGGTTTGTCTTTGTOTTCTGTGATACCTGGAAGAATGTTC
TTCCTTTTGTTCTTATTTTATAACTGTAATTTTGCTACTGTTACAATTAAATATTTTTGGAGATAGAGTTTTGCCAAAAGGGTCAC
AGCCTACAGATTGAGAACCACTGGTCTACAGTATTGATGGTcTTTTTTGTTTGTTTGAGACAGGTCTATGTTGACCAGGCTGGATT
TGAACTCAAGAGATCCACCTGCCTCTGCCTTCCAACTGCTGAGAATAAAGGTGTGTGCCACCGTGCCTCAGTGTTTCAGAGTGTGT
TGTTTCTTAGATCTAGTTTGACATCTTTGGTGGGACTTCAGAACAAGTCCAGACTGAGGGTGGTTTCTGCCTGACCAGTCTTCCTA
TGATGATTAGAATAGGTGTTCATCCTCAGGGCCTGGGGTCTTCCTTCTCTGTGGGGCTTTTGTCTTCATACTTTTGTGCTGGTTTG
TCAGTTACCTGAGTAGGCTGGGAAGTTACTTACCACATAGGCATGGGCATGGTGAGAGTGTCTGAGAGGAGCCTTCCTTTTCTTAT
TTCATGGGAGGAGAACAGTCAACCATATGATAGCACTTATAATACACTTGGCCCCTGCCTAGAGGTAAATTGTAGACTTTTGGGTC
CTGGGAGGCAGAAAACCTAGGCTTTAAAATGATGCTTGGGTTTTTTTTTTTTTTTTTTTTTTATTTCCATGACGTAATGATTCTAT
TCATTGATACTAAGGTTAGAGACTCCTGCCAGCTTGACCATGGAGCTGTCCTGAATATGAATGGAGTTCATTATAAAGATGTAGGA
TGTGGGGTTGGGGATTTAGCTCAGTGGTAGAGTGCTTGCCTAGCAAACGCTAGGCCCTGGGTTCAGTCCCCAGCTCCGAAAAAAAG
AAAAAGAAAAAAAAAAAGATGTAGGATGTATAGGAAATGTTGCATTAAGAAAGGAGGCTGGGGTGGAGAGATGGTTCAGTGTTAAG
AGCACTGCTCTTCCAGAGGTCCTGAGTTCAATTCCTGGCAACCACATGGTGGCTCACAACTATCTGTAATATTAGAGATCTGGTGC
CCTCTTCTGGTGTGTCTGAAGACATTGATGGTGTACTCAAATACATAAAATAAGTAAATAAATCTTTAAAAAAGTGAAAAAGAAAG
GAGGCTGGTTTCTAAAATTCACTGTCCAGAAGTGACTGGGGCATCTTGAGGTTGTGGATTTGTCTGTGCACACGGATGGGCAGAAG
TTCTTTTCCTACTTTTCATGATTTTTGCCTAGGACAAACCAGAGACTAACATAATCTGAATCATCAAGTGTCACAGAGAGCCTCAG
TTCCCTTTGGAGGCCTGCAGTCCTGGATCCATTCTCGTTCTTAGGGCGGCATTCCTGTGCATTCCCGTGCATTCCTGTGCATTCCC
GTGCATCTGGTTGGTTTGTACCAGCTTCTTTCTGCAAGGCTTGCCTTCTACTTTCTAGTGATTGCTGGAATTTATAAAGGAAAAAA
AAAGCTGTAAGAAGTACAGAGAGGGGTTGGGGATTTAGCTCAGTGGTAGCAAGCGCAAGGCCCTGAGTTCGGTCCCCAGCTCCGAA
AAAAAGAAAAAAGAAAAAAGAAAAGGAAAAAAAAAAAAGAAGTACAGAGAACCATTTGTTGAGCAACTTAAGCTGTGACTGCTTAG
TCCTCCCTTGCCTGTACCTCCCTTTGCCTTGTTTAGAAGCCAGTCCCAGCCATGTGACTGTGTCCATGACTCTACAGTATATAAAG
ATCCTTCTCAGAACTGAAGATTGATGCTGACTGATGAAGTGAGTGTTGATCCGTCCTTTTCTGTGTAGAGTAAGCCCAGTGGTCAG
GAGCCTTTGGGGTCCTAGAATTGTTTGTTGAGGATGGTGTGAGGGAGCCTAAGTGTTCCAGCCCCACAGTTACTTACCTCATGTGT
TCTTAGTCTGGTTCCACAGCATTGCTGAGCCTGGAGGTAGATCATAACACCTGGGGCTTTTTACTGACTGTCCCATGACTGCATGA
CTGTCCCCGTGACATCAGTGCTCTACAGGAATACTGACTGGTAGGGACTACCCTGCCACTCACATAGGGTTTTTTTTTTTTCTCTC
CCCTTCTTTTTTTAGAGATAGGGTCTCTTCATAGGTCTGGCTGTCCTGGAAATCACTATGTAATCCAGACTGCCTTGCTTCTGCCT
CTCTAGTACTGGGATTAGGGTTTATACCACCACACCTGGCTAACGTGAAAGGATTACCTGGTGAGGCCGTCTCAAAACAATAACCA
CAACTCTACCCACCTTCTTTTCCAAAATACCCCCAAAATTACCCTTTTGTGACTTTGCTAGGTTTTTTGTTTTTATATCAATATAT
AATACATCTTAGGTTATTTTTGTAGACAGCATCTGTGAAAATGGCAGTTTAGGTGGGCTTTTTTGGTCTGTCAGCTTTTTTACTCA
ACCTCAGACGAGGCCCTGCCTATTGAGCTGCCCAGAAGGAGATTGACTCTGCTATGCAATGTACTGTGCTGTGTTTTTTGTTTTTT
TGGTTTTTTTTGTTGTTTGTTTGTTTTTTAAAGCAGTCTTACTGTGTAGCTCCAGCTGGCCTGAAACTTGTATGTAGACCAGGCTG
GCCTCCAACTCACAGAGATCCATCTGCCTCTGTGTATTGAGTGCTGGGGAGAATTCTTAATAATAAGTAATATTTAAATCACAACC
GAGTCACTTGTTTTTAAAAAAGACTAGTTAGGATTTCCATGGATGGATACTAGTATTAGAAGGGGACGAGGTAGCCCAATGTTTCT
TGCTGTTCTTTCCTTTCCTTTTGAGATGGTCTCTCTGTGCAGCTCAAGTGGCCTCAACCTCACCATCTTTCTGCCTCAGACTCGGA
AGTGCTGTGTGTGCGGCCTGCCCCGCTTTCTTCCTGGTTTGTTCTTACTGACGGAGCTGCTGGAAATCCTGCCCTATGTAGACGGT
ATAAAAGTGACTTTGTGTGGCAGTGTATGTATCAAGTTACCAAGTTAGCTGTAAGCAGTGACTTCCACCTTAGACCTAGGCCTGTA
AAGACTGGAATGGCGGGCAGTGTACGTCACTTGCCACCCTCACTCTGTACGCATCTCTTCACACCTCTCAGCATAACGGTCCAGTT
AGTTTTCCTCCTGTGTTTTTGTTTTGTTTAGTAGCTGTGTTTAGGAATGCTTGAGGTTTTTGAGTGTACTCTCGCCAGCATTTAAA
ATTTTTAAATAGACTATGATATTAAAAGATTCACAAGACAGACGTGTGGTTAATAAGGTACAATGGAGTGATTGTAATTAGTCTGT
GTCCAGTGAGCCCTTGCTTGTGGCAGCCCCTGCACAGTCCCTTCCTATGAGTGTTCTGGCTTGTGTTAGGTTTGCTTCATGGACTT
TTCCTCTAGTAATCTTGGTGTATTTGTGCCTTTGATATAATGCCTGTACCAGATTTTAGTTATAAAAGCTAAATAGACAAGGTTGG
ATAGTTTATAAAGAAGTTTTTTTGAGCTCACAATTTGGGGAGCAGAGAGCACAAGATTGGGCTTCACATCAACTTACTGTGTGGCT
GAATCCTATCCGGGCAGTGGCATGCATGGTGGTGGGAGCATGTGTGGGAAGTGGAAATGATGTGGTGAAATAGGAAGCCAGAGCAC
AGGGAGCTGCCGGCTTTTGGTCTTCCTGCTGGCACCTGTCACTCCAGGGATCTGGGGCAGGCTAGGGTAAGTGCCTGCTTCCTTCT
AGAGGTTCATTTTGGTCTTGTCCACTTCCAGGTTCCTGTATGCTGTGGGTGGGTCTGCCTTGTGTTGGTTGCCTGTCAGTGGTATG
GTTTGGCCTGTTTTCTCTGAGTTAGTTGGTAGTTGGCTTGAGTGTTGATGAGTGTGGGGTGCACTGGTTGGACATGCTTGCTCTTG
ACTGGTCTGTTTTTGGGGAGCTACAGTATTGTAGTGCCTGTTGTCCACCTTTCTCCGAGGTGTGAGGTTGCTCACGGGGTGAGTCA
GGGTCTGCATGTAACGTTTGGGTACATCCTGTAGAATACATGGAAATTATTTTTATATAAGTCTTGTTTACAACTTGCAAGCTATT
CACAACTTCCCAAGTTCTTGCACTGGAGAAGGGGGTGGGGCTAATATGAAATTGGTATCTTAATTAAAGCAAATTGCTAACCATTA
TTTCTTTGGATTTTTAAATTGTTAAAAAATTCTTGTATCTGGCTGGGTATGGTGGTGCACATGGTTTATCTCAGCACCTGGGAGGC
AGAGGCAGAACTCTGAGCTCATGGCTAGCCTGGCCTATAAAGCTAGTTCCGGGACAGCCATGGCTCCATTATTACACAGAGAACCC
TGTCTTGAAAATCAACCAAAACCAGCCAAAATTCTTGTATCTCTGTACTGCTTCCTGCAAACATTAAAATAACCACGAGTGTAGTG
TTAAACATTTGTTCGCATGTTGTTGGGATGTGTGTAGTTTACTGCCCAGATGTCACACCTCTGAAAACACAAGCAGTACTAGTGAA
GTAGCCAGGCCTCCTGACCTGCTGATGTAGCTTCCTGTGGGTCTTGATCACTGTCTGCTGTAGCCTGGTTTCCTGTCTCCATCTGG
GTGTCCTTTGGTGGGTTTGTCATTAGAGATGATGCTTCGTGGACTTGGTGTCTGACCCACCCACACTGAACAGGCAGAGCCACCTA
GAGCAGTGCACCATTTAGTGGAGCTCAGCCAGGAGGCTTGACAGCCTCACTGTGTGAAGCATTCTCACGGGCAAGCCGGCCTTGGC
AGAGCTGGGCCTTCTACCTGTGCTGGTGTGTTTTGATTGTTCTGTGGAATTTAGGTTTGCATTCTTCTTCTTCTTCTTTTTTTTTT
TTTTAAAGCAAGAAAAAACGAAAAAACTGAACTTCGAAAATTTTAGAGCCTGTTCTGAAATTTTGATGTGTGGTACAATGAAGGAA
CACCTTCTTGTAGCCTTTTGGAGTTTCATTCTTTTGAAATTGTGGGGTTTGGTGGAGTTTGTCTTTCAGTATCTTTGTGAGGCACA
CTGAGCTCTTTTTCTGCGGCTGTGGTGTAAAGCAGCCCAGAATTTCTCAGAGGTTTTTACAGCTTGGTGCTGCTAGTCCACAAAGG
ACGAAGTTTCTCAGATGGTTGTCATTTACTAAGAGCAGACTGTTCCCAACCTAAGTGAGTGGGTGAGCCACTCTGTTTCTGGAGTT
TCTTCAAGGTTCAGTGTGACCAGGGCTGGTGGTGCCACCTGGTGAGAGCAGGCTGTGACCTCAGAGTCCAGCCATCAGCATCTCAG
CTGACAGTGATCAATAGTGGTTGCTGTGTGTGTAGATAGGACGTCACACAGGAGCAGTTTGTTAAGCTGTTTCTTTTAGATGTTTG
ACCTGATGACTGTTTTGGTGGATGAAATCTTTAGTTAGTTGAAGGTTATGAACTGTTTCTATAGTACCAGGGACAGGCTCAGGAGA
GAACTGCAGTGTTATTGAAGGTAACATTGTCCTGTCTAGTTTTCTAAATGCAAACACTTTTTAATGTGCTTTTCAAAGCTAAACTC
TCAGTTTTTCCATGTTTTAGATTAAATAGGATTGCGAATCAGGTGGCCATTCAGCGGAAGAAGCAGTTTGTGGAGCGAGCCCACAG
CTACTGGTTACTCAAAAGGCTGTCTAGGAATGGTGCTCCCCTGCTGCGGCGGCTCCAGTCCAGCCTGCAGTCCCAGAGAAACACGC
AGCAGGTATGTGTACATGCTCATCTGCCTTTCGGTGTCACGTGCCTCCAAACACAGGCTGCCCCTTCAGGCTGGATGTGCTGACCC
CGACCCCTGACCCCTGAGCCTTGAACTACTAAGCTGCAGATTATTCAGGTGGCTCCTATTTGCCCAAGGTTTGCTGTGGCTCCAGG
GTTGAGTTGTGCCTCCTTCAGCCCAGGGAAAAGGGAGTGCGAAGAGAACCTAGTTCAGCTTGAGCCACATGCATAGTCAAGACAGG
AGACCTGATGAGGCCTATGCAGTTCTCTACACATTGCCCGAGAAACACATCTGGTGGTTTCTGCTGGTTCCAGTGGGAGGAGCTGA
TCATTTCATTTGTAACATATCAACCAAGGTTATCGGAAGTTTACAACTGTAGAATCCTGTTGCGTCCCTGTGGCTGTCACAGGTAT
GGCTGTCACAGGCACAGTTGTGGGATGAGCTCAGGGCTCTCATCCTGTATCCTGTCTGTCAGATGTGTAGTGGTTCTGGAGCCCTC
TTGTGTGGATTAGACACTTATATCTGGAAGTTACCAAGTATTGCTCAATGGACGAGGTTCAGTCCATTATCATCCTGGCGGGAAGC
ATGGCAGCATCCAGGCAGACAGAGTGCTGTTGAAGGAACCAAGAGTTCTACGTCTTGATCCAAAGGCAGTTAGGAGAAGACTGCTG
TCTTCTAGGCAGCTAAGAGGAGGATCTCAAAGCTCACCCCCACAGTGACATACTCCTTCTAAAGAAAGGACACACCTCCTAACAGT
GCCACTCCCTGGGCCAAGCATGTACAAACCACCAGATAGGGTATCTGAAGCAGCCCATCTGAGGAGTGTCATGGTATAACCAGGCT
TTGAAGTGCCTAGAACAGTAGAGGCTGTTTTAGTTGTTGAGTGGATCAACCATGTTCTTTACGTCAACATACTGTGAGAGGCTGCT
GTTCTCAATACCGCCCTTAACTCTTGGCGCCCAGCCCTACCATGAGATGGCTTGTCCTGGGAAAGAGGGACTCTCCTTTGTACCAG
AGGAGAAAAAGGTTTTAGGGCAAGAGAGTCTTAGGGATATTGGTCATTAAGTGGGGGAGGTGTTTGGTAACCCTGGGCACTGGTAG
GAACGATGCTTATGTGATGGTGGCAGTGTTGGGAAGAGAAGATGTGGAGAGAAAAGTGGAGAATCCAGACCACAGGTATTGTCTTG
GGCTAGAGGAGAGTCCTAGTGAACCGCTGAGGAAGAGTGTTTTGGTAGATTATCAGAATGGAATGCCGTTAGTATAGTGGTGGGGC
CTTCAGCTGTTGGCTTCTTCTCCTCTGCATACATAGGAGCATAGAACAAAGGGCATGGGATGCCAGTGTCCCTGTGGGTAGCTTCC
CTAGAGAGGTGGATCTGGGAGGAAGAAGAGGCTACAGGAAGGAAGATAGGAAGGAGGTGATAAGGGAAGGAAACAAGGCTGGCCCC
CTGATGGTCATCCCTGTCAAAGGGCAGGCCTTTGCACAGGACATTGAGGCCGTCAGAGAGAGGGAGCACCGAATTGTGAGGATGCA
AGCTCTGTGGTGTTCTGGGCTGGGTCATTCTAGAATTACCAGAAGGGAAGTAGAAGGCTTTGTCCATGGCAGAGAGGTCTGCTCTG
CTGTGTTGGACCAGGCAGGACATGAAATTGGAAGTTGTAAACTATACCCACATTGTCTTAGTAAGGCTTGATGTAATGGCCCTGAG
CCTTGCCTTTGACATCGTGTATGCTCCGTCTCAGCCTGGATCTATAGACTAGAAATACTGAATGTTAGAATTTGACTTACCTTTGA
CTTTAGCTGTCTCTGCTCAGCCCAGCTTTGGAAAAAGGCTGTGCGATTTCTCTACTGTGACTAACCTTGTGGAGGATGGATGAGGC
ATAGGGATGGTGGGACAGGATGAGCTGCTATGAGAGGACATCACTGACGTTGGTGTTGTGGGGAGTCTTTCACTGTGGTGGCTAGA
AGCTTCCCAGCTGTGCGGTGACTCCGTAGGCCCTACTCTGGTAGGAAAGCAGGCATGTTGCTTGTGCCCTCTGCTGAGGTATAGTA
GGAGGTGGGTTGGTGTGGCTCCTTAGATTTGGTCCAACAGTTTGTCAGGTGCAAGCCCCCATTCATCCTGTTTTGGTTTTTTTTTT
TTTTTTTTTTTAATTTCCTGTTTTCCTTTCCCTCTAGCTCTGGGCCTCTACTTGTACCCATTTATTCATAGAATTCTGGAAGTCTT
GGGTCTGACATGGCTGAGCCTAGCTGCCCTTAGGGTCATGGTTGAAGTGTATGGGAGCCACTGCTGCCGATCTGCTGTGTGCTTCA
CAGATACGCTGACCAGTTCTCCCAGTACAGGGGCCCTTGGCCTCACTGCTGGACTGGTCCTTGTCACAGGGCTGGGTTTCTGCCGT
CCTCCTTTATCCCAGCACTAGATCGTGACCTGTGTTAGGAGTGGAAACACTGAATGCTTGTGCTCTTCTTGGGCGTGAGCTTCCTC
TTCTCAGAAGTCTCTCCTGGAAGACTCCCAGCATTGGTTGCTATGTACCAAAGTAGACTGCTTCAGGATCGTACTGGGAAAGCTGG
TTCATAGATGGGATGGTTGGTGTAGATTGGTGTACAGGGTCCTGTCTTCATGAGCCTGAGGCATGTTGGAGTACAGACAGTGGCCC
AGTTACCCCATGACCTTATAAAGATTAAAACCAGGCCACGAGCAAACCACCGAGTTTTGCCTATCCCTAAATACTCAAGCTCAGAT
CTATTGGCAATCGGGAGATTTCTTTTGCTTCATGGGGGTTCTCTGTGAGTAACCAAGTCTGTTTCTAAGTAGCAGATAGGAAGTTG
TCCAGATGTTAGGGTATTAGTTTCTTTTTTCTTTGTTTATTTTTGAGACCGGGTTGACCTGGAGCTTGCTATATATGCTGGCCTTG
AACTCATAGAACTCAGTCTCTGCCTCCTAAGAGTTGAAATTAAAGGTGTACATGGACACACCTGGTGGTGGTTGGTTTCTGAACCT
CCCTTTCCTTGTATTTACTTACTTGGCCTATATGAGATGATACTGTCATCAACCCCAACTAAATGCTTAAGAATTGTCGGTAATAT
CAGGTACAGCGTACATTACTGTGGGTGCTGAAGTATGTGCATTGAGACAGATCATGCCATACCCATTCTGTGCTGTCATTTTCAAC
CATGAAGAGTGGCTGTCGACAGAGTTTTTGGTCGGTGACACTTTTCCCTGAGATCCTCCATCCTTGACCAGTGTGCTGGTAGCTTG
GGTTGCAGAATCTCTGCTGTGGTGTCATTGGGCTGTGAGAGGCAAAACTGTCCAGAGAGAGAAGGGTCTCATGTCTGTGTTCTACA
GCTGGCTGTCAGCACTTTGCTCGTGGTTGACAGATGTGGCTATTACTGTCCAGTAGTGCAGAAACTTTTGGGTAGGCTATTCTCCA
TCCCTTTACCATAGGGACAGGACACTGTGTTACTGCAAGGAGGTCATCCCATGTCTTTAACACAGAATAGAGAGTGGGGATATAGC
TTTGGATGATGACTATTGTGTTGGATGAGGACCCGGGTCTTGGACAGGCTCACTATGGGGTGGCAGGAAAGAGTGATATCTGGGTT
GGGAGAGCAGAGCTCTGGGGAACTTGGTTTAAATAAGATGCATGGATTACTGAGAGGATGTGGCATGTTGAATTTCTTAGGAAGTG
GCTGGAAAACCTGGTCCTTTGTAGATAGGGCTCTGGTCTTGTTTGGTGTCCTTGGTTGCTATCGAGGGACATGTGCTATCCCTGTG
GCATTGGCTCTTGTCCCCTGTACATTTGTGAGGTAGTAAGAGTACCCTTTGGACATTTCAGCCTTGAGTGGCTCCATCAGGAGTCT
GTCGTCGTCTTCTTTTTTTTTTAAATTTATTCATTTATTATATATAAGTACACTGTAGCTGTCTTCAGATACACCAGAAGAGGGCA
TTGGATCTCTTTTTTTTTTTTTTTTTTTTTTGGTTCTTTTTTTCGGAGCTGGGGACCGAACCCAGGGCcTTGCGCTTCCTAGGCAA
GCGCTCTACCACTGAGCTAAATCCCCAACCCCGGATCTCTTTACAGATGTTGCAAGCCACCATGTGGGGTTGCTGGGAATTGAACT
TAGGACCTTTGGAAGAGCAGTCGTTGCTCTTAGCCGCTGAGCCATCTCTCCAGCCCGGGGTTTCTGTCTTCTTAATCCTGCTTAGA
ACTCTGAGCTTCTCAAGGATTCACATCCCATGTGACCAGGCAGAGCCCCACTGCTTTTCTGCTACTGTCTGTGTCGCTTGACTTCC
CAGTGCTGTACTTTTTGCACATTTTGATGGTTAGGGTAGAGAGGGGCTGGTGCAAGATGCTGACCAAGTTAGGAGAGGTGCTATCT
GGTGTACTGCTCTGTCACCTGAGAAGGCAGCTGTGACTGGCAACTACAGTGCCCATGCTAGTCTATGGGGTTAGTTAGAAGTGATC
CCTACACTTACCTGCCGAGCCCCGAACTGAGCCTGTGTAATATTCCGCTGCCAGTAAGGATTGCTTAGGTTTGTACCTTTTGTACA
TCTCCTTTCTAATACTCCCTCCATTCCTACCTCCTGGAGTCAAACCAAGACCCCTTGTGCCGTGGTCCCATTAGACCTTCCTGTTC
CTTGTCACTGGGTCCCAGGTCCTGTTACCCTTTTAGTCTCACTTGTTGTATGAGCTTGTTAGACCCCTGGCAGGAACTTCTGGCTT
TGACTGATGGAAAGTTTCATTTAATTTTCTCAGAGAGAAAATGATGAAGAGATGAAAGCTGCCAAAGAGAAGCTGAAGTACTGGCA
GCGGCTACGGCATGACCTAGAGCGTGCCCGCCTGCTGATCGAGCTGCTGCGCAAGCGGGAGAAACTCAAACGGGAGCAGGTGAGTG
TGTGGGGCCCTCGGGAGCTGCCACCTTCAGGGCTGGCTCTCTCTAGATGGACATCTTGCTGCTGGCCCCTGTGTACCTGCTGATTC
TGTGTGCTGTCCCCTCCCTACAGCATATCCCTACCTTATAGTTGGTCCTGTGGTACCTCTGTGTTCTTTTTGGGTAGCCACTGCCT
CAATGTCTTAAAGGAGAATACTTGTCCTTGCAGAGAGAAGGCTGCCTTGTGGTAGGGTGGTAGCGTTCACGTAGGCTGCTCTGTGC
TGATGGTTGGAGTGTCGCTTCTGTGATTGTGCAGTATGTGGAGGTGCACGATCTGTCTCTAAGAGAGCTGTCCCTACACTCCTCTA
GAGATAGTCTATGCTGTTGTTGCCAGGTGAAGGTGGAGCAGATGGCTATGGAGCTCCGGTTGACAcCTCTGAcTGTGCTGCTACGC
TCAGTCCTGGAGCAGCTACAGGAGAAGGACCCTGCAAAGATCTTTGCCCAGCCCGTGAGTCTCAAGGAGGTGCGTGCTGCTGTGAC
TCTGTTCTTTTTCATGTGGTTGGATCCATACTGCTGCTTGGTTAGGAAGCACGGGACTAGGGAGAGCAGGTTACCTGCTTCCTTAA
TTCTCATTATTATTTAATATTTAATGAATTTTAGTGGATAGTAGTTTAATTATAAAAGATTGTGCCTCTTTGTAAGGCACTGAGAA
TTTCTACTCAAAAATTAGCTATTGGTAAAGAGAACCCTGCTGGTTCCCCATCTGTTGTACTTTTAGTTCAAGGAAGTAGGTTGGGA
GGGTCCCTGCAGTGACTGGGCTTAGTTTGTATTGCCTAGAGTTGATGGGAGGGCGGGGCGGAGTTGTATGTCTCAGGTGTGATTGA
CTATAGAAAGCATGAAATAAGTTTTGATTTTTTTTCTTTGGTTTGTAAATGTTTATTTTCCTTCCTAAAATTAGGTACCAGATTAT
TTGGATCACATTAAACATCCCATGGACTTTGCTACAATGAGGAAACGGCTAGAAGCTCAAGGGTATAAAAACCTCCATGCGTTTGA
GGAGGATTTTAATCTCATTGTAGATAACTGCATGAAGTACAATGCCAAGGACACCGTGTTTTATAGAGCTGCAGTGAGGCTGCGAG
ATCAGGGAGGTGTTGTCTTGAGGCAGGCCCGGCGTGAGGTGGATAGCATCGGCCTGGAAGAGGCCTCGGGAATGCACCTGCCTGAG
CGACCCATCGCAGCCCCTCGGCGGCCCTTCTCCTGGGAAGAGGGTAAGAACCCGGCACTGCATCCAGGAGGACAGCGGATGCTTTT
TCTCTCAGACTGTACTTATTAAGACTCCAGCATGCAGGCAGCATGCGTGCTCCTGAGGTGCATGTGCACCGTATATGCAGCACATC
TCACATGGGCCTTGCCACATTTTCACACACTTACTGCAAGAAGCAGGGGTCTAGGTGGTGAAGGCCGTGAAGACACCATAGTTGAG
CATTCATCCCCAAAGGGACTAGCCTTGCTTCTGAGGAGGTCTTGCAGTGAGAAGGCAGCCATTAGTCATCATATTTCAGCTGAGAA
ATAAAAGCAGGAACTAAAATTGGCTGTGCCTCTGATCCTCTCTCTGGGATGCTTTCAGGTCCTCAGAGGGCCCAGCCTTAGCCTGT
TTTTAGGACATGGCCTAACCCTCCTAGCCCTTCAGGGTGAGCTTGTACTCTGGACCCCACCAGGCACATGCTGTTGTGCTGTTCAT
TAATTTCCTCCAAGTACGGTGCTGATTTTGGAGATAAGGTCTTGATGGGCAGCCTTGGCTGCACGTGGGTCAGGCTGGCTTGGGAT
TGACAGAAGTCACCTGCTCCTGCCTCCTGAGCGCTGTGGTTACATGTGCACCGCCATGTCTGGCCTTCAAGCAGTTCTTGTGAAGG
TTTTGCCCCTTAATCTTTATTTTGTAGGTGCATGAAGTTTGCTTGTATTTACCTAAGATCCTGTGTTCCTGTTTTGACTGCCCAGG
ACATGGTGGAACTGTACTGACTTAGGTTTATCCAGTGCTTTTCCTTCTCCTGGATGGTCAGCCAGCTCTGACTCTGCCTTTGCTTT
CCCATTGGTTATATTTGTGACTTAGTGACGTCAGGGCCCTCAAAGGCTCCCTCACTCCCCAGAGAAACTGTCTCTTTAGTACTCGC
GCTTCTGCAGGGCATACAGGATAGATAGAATTCTTTTTTTCAAGATAGGATGTAGTGCCACACTCAGGAAGCTAAAACAGGACATT
TGCCGCAAATTCAAGGCCAGTCTAGACTGTAGTGATTTCCAGGCTACTGTGAGTTACACTCCGAGACCCTGCCTAAAAACCAAAAT
TGATCCAAAAAGTATAATTAGAAAGAAAACACAAGCAGGCCAGAGTGTGGTTTAGTAGTTTCTTTTCATGCACAAAGGGTTCAGTG
TCAGCACAGCATAAACTGGGTATGTTGATACAAAGATTAGGATTTAAAGGTCATATTGGCTACATAGTGAATTAAGGCTAGCCTGT
GTTACATGAGACCTTGTTTGGAAAAATAGATGCATTGCACACAGACAGGTGAGAGACAGGTGAGAGATTTGTGCAACCCTAGATAC
AGGTCCAGTGCAACTGGTTAGTGGGAGCCATCTTGTGCTGAGATGTCCCCCGAGCAGGAGACGAGCCTGATTGCGCCCAGGATTAG
AGTGACTCTCAGTCCTTCATGTACATCCTGTTCTTTCTTCAGCCTGTGTGGGAGGCAAGGGTAGTGCTCCAGTCTTAGCTGATGTG
GCTATGACTGCTCTGAATGGTATTGGGTGCCTTAGAAGCAGAGGAGTAATGGCGTCTGGGAGTCTCCGACCCCATAGCTTCTGATT
CTCATGCTCTGTGGATGGACAGGGCCTGGAGGCCTCAAAGTTGATACTTCCAGGAAACTAGCTTTGCCAAAGGGGAAAGTAGTAAT
GAAAAGCACAAACTGATTTCTCCCTCAGTGATTGAGTAGGATGAGCTCTGGGTACCTCTGCCACTGTTTTGAGCCTGCTCTAATGA
AGATGCTTGTCTTAGGGTTTACTGCTGTGAACAGAAACCCCTCCAGAAACCCCCTATCCCATCCCCCTTCATCCTCCTACTTCTAT
GAGGGTGCTCCCTCACCCACCGACCCACTCCTTCCCACCTCCCCCTGACATTCCCCTACAAGCAACATTTAATTGGGGCTGGCTTA
CAGGTTCAAGGTTCAGTCCATTATCATCAAGAAGGGAACATGGCAGTATCCAGACAGGCACACGGTGCAGAAGAAGCTAAGAATTC
TACATCTTCCTCTGTAGGCTGCTAGTAGAATACTGGCTCCCAGGCAGCTAGGAGCCACGCCTACTCCAACAAGACCACACCTCCTA
GCAGTGCCACTCCTGAGCCTTGCCTATACAAACCATCACATTCCACTCCCTGGCCCCCAGAGGCTTGTTCAGACAAGTCTGTGAGA
GGCCATACCTAAACATAACATAATGCAAATTACATTTAGTCCAATTTCAAAGTCGTGGTCTCAACAATGTTCTAAGTTCAAAGTCT
CTTCAGAGATTCATTCAGTTGTTTAGCTAATCTCCAAAGCAGGACAGGAACCAGCGGGGCAAAGTTTGCATCTCCATGTCTGTCAA
AGTGATCTTCAGATCACCCACCCCCTTTGCCATCCTTGTTGACTGCAGCAACGTCTTTCTTCTGGGCTGGCCCCATTCCCTGTTAG
CAGCTTTCCCCAGCAGAGTCTCCAAGGCCACCTCTGTTTTATAGCTTCTTGATTTAGCTTCTGGGATCCACTTACGATCCTCTGGG
CTCCTTCAAAGGGCTGGTGTCATGTCTCCAGCTCTGCCCTCTGTAGCCCTCTGAACTCAGAGGACCTGCCACTACTGTACTTGGTG
ATCATCCCATGGTACTGGCATCTTCAATACACTGGGGACTTCTGCTGCAGCTAGGCCTTACCAATAACCTCTCACAGGCTCTCTTC
ATGGTGCCAAGCCTCCTTTGCATGACCTTTTCAGTCCTGGGCCATCAACTACACCTGAGGCTGTACCTTCACCATGGCCACAGTGC
CCAGCCTCAGCTGCTTTTCATGACCCTTCCTACCTTCAAAACCAGTGCCACCCGGGTGACTCTTACACATTAATAAGTATGGAATA
CAGCTTCTTTGTGTTCTCAGAAAAAACTCCCAGAAGATTTCATCTCAGTGATGGTCTAATTTTTTTAATGAGTACAGTATAGCTCT
CTTCAGACACACAAGAACAGAGTATTGGTCCCTGTTATAGATGGTTGCGAGCCACCACGTGGTTTCCGGAATTGAACTCAGGACCT
CTGACCCCTGAGCCAACTCTTCAGCCCTGCTGGTCTCTTCTTAATCACCACTAATTTTTTAGCTCCAGTTAACTAGCATCAATTGT
CCCAGTAGTCTGTTTTCTCTTGACCAAAAAGCCAGAGACACATGACTAAAGCTGCCAAATTCTGCTGCTTGCAGGAGCTGGAATAT
GGTCCCCTTCTATAACACTGTCACCAGCTTCCTGTTTTCCACCCTAGCTCGGCTGTCTCGGTTCTTGCTCAGTAGATTGACCTTGA
ACTCAGAGATCGGCATGCCTGGCTCCTGGGATTAAAGGTGTGTAACACCAGGCCTGGATTTACGCTTTTCTTCACCTACAACTTGC
TCCTAGGCTGGCCTTGAATTTAGAGATCTGCTTGCCTTTGCCTGGGGAGGGGGGTCAAAGGCTTGTTCTACCTTGTCTGGACCTAA
ATTTAGCTGAGTGGGATCTTGCCCCAAGGTTCTGCCACTCCCTTAATTCAATTTATTATCTTTGAATATAGGTTTTAGCTCACTTC
CTGATTTCCTTTCTAACCTTGGTATGCTTATTCAAAACACTCTTGAATTTTAACCGGAGAAGAAAGTCTGTGATGGGTGTTTCCGA
GACGTCCTTTGTAAATGCAATTATTCTGAGTCTCTTCACCTTAGCCTCAGGCAGACTCTTCAGGCAAGGGCAAAAAGCAGCCATAT
TCTTCACCAAACTACAAAACCAGTCTCTAGGCCACAACTGAAATTCTTCTCCACTGAAACCTCTTGGGCCAGGTCTACACAGTTCA
AATCACTCACAGCAACAAAGTCTTCCATATTCCTACTAGAATATCCCTTAAGCCCTACTTAAAACATTATGGCTTTCCAAATTCAA
AGTCCCCCAAATGTACATTCTTCCACATGAAAACATGGTCACTCCTGTCACAGCAGTGCCCCAGTCCCTGGACCAATGTCTTACGG
TTCACTGCTGTGAACAGACACCATGACCAAGACCGCTCTTATATAATTGGGGCTGGCTTACGGGTTTCGAGGTTCAGTCCATTATC
ATCAAGGTGGGAGCATGGCAACAGGCAGACATGGTGCAAGAGGAGCTGAGGGTTCTCCATCTTCCTCTGGAGGCTGCTGACGGAAT
ACTGGCTCCCAGGCAGCGAGCCTACACTCACAAGGCCACACCTACTCCGACAAGGCTGTACCTCCCAACAGGGCCACTCCCTAAGC
CAAGCACATACAAATCCAAAAGAAGCGGACAAGCAGGGTGTGCAGGCCTAGCACTCAGTGGTTGAGGAAGGAGAGTCACTAGTGAG
GCCAGCCTGTGAGATCCTATCTCAGCAAGCGAAGAACAGAGCAAAAGGAAACCAGCATTGGAAAGTTTTGAGGGGAGGGGTGTTAA
GATTATTTTTTATTTTCGGTACTTCAGATTAAAGGAATTTTGTTTACCGGAACTCATTTGAGGTGTTAACTTTTAGATTTTGTTAG
AAATAGTGTGACTATGAGCCCTGAGGTAGCCAGCCGGGCAGGGTTTGCTCGTGTCTAGTGCTGGTCAGTGCTGTTCTTCAGACAGG
GCAGTTCGGGTTCTCACTGGTCAGCTGCCAGGTCTGGGCAGGTCTCCTTTATGCTGTGTATGTCTCTCTGTTGCCCCTGCTGGTCT
TTGGTTTTATCTTTGCAAGATTAAAGAATTTCTTTGGCTGTTTTACTAAGTTCTGTAGTCAGTGTTCTTAGAATTTGGGGAAACCC
GCGGACTGGGCGCCTGCTGTTGATGTGGGCGTAGTACCCTGCAGCTCCTGTTGGCTGTCTCACACATTTCTGGTGGTCTTCGCGCC
CCTCACGTTTTACACAGCAGGACTGTGTGGGAGCCTCTTCCAGGAGAGGCCACACACGCTTTCTGCATGTCCTCTGCTGTGGCCAC
GTTAGTCCTTTGTGTCACACTAACTGAAGGAGTGCCTTTTTTCTAGCGCCAGCCTTGTCATGTGTTCAGAATCAGGGTAGAGGGGA
CTATATATGGCATCAAATGGTGAAATGAAACAAAACAAAAACCAACCAACCAAACAAAAAAGAAATGGTGAAGCTTGTGCTATGGC
CATGGGCAGGCTTTAAAGAATACTTGGGATCAGTGTGTTATTCTTAGAGGAGCCCGAGAGTCGGGTGGCTGATGATGTCTGTTCTT
TGGTTCAGTGGACAGGTTGCTGGACCCAGCCAACAGGGCCCACATGAGCTTGGAGGAGCAGCTGAGAGAACTACTGGACAAGTTGG
ACCTGACCTGCTCCATGAAGTCCAGCGGCTCACGGAGTAAACGGGCAAAGCTGCTCAAAAAAGAGATTGCTCTTCTCCGAAACAAG
CTGAGCCAGCAGCACAGCCAGACCCCATCCATAGGGGCAGGCACAGGAGGCTTTGAAGACGATGCTGCTCCACTGGCGCCAGACAC
AGGGGAGGAAGGTAAGCATGATGGGGTGGGAGGGCCGTACCTCATGGACATGGGTGTCTCCTGACAGGCTTAGATGATGCTCTGTA
GTAATCAATCGTGAACTTGTAAGTTTTGAAGGTCACAGAACTCTTGGTCACTGGATAGTCCTCCTAGGTTTTCTTTTTAACTTGAG
CCTGAAAGACTTTACAAGGGATAGTTTATAGAGCTGATGCTGGATTGAAGGTGGCTTCTATGGAGGGAATAAGAAATTCTTAGTTG
TATTTTCTAAATTGAGGCAGAGTATTAGATGGTTAGATCCCCTGAAATTGTTTTTACTTTGTGTGTGTAGGTCAGAGGACTAGTTG
GAGTTGGTTTTCCTGGCATCTTACAACATCTGGGTATCAAGCCAAGGCGATGAGGCCTCGTGAGCACCTCTACCCCTTTGCCCCTG
CTGCCTTATGCCAGCTTTTTTTTTAAAGATTTATTTATTTATTATATATAAGTACACTGTAGCTGTCTTCAGATACACCAGAAGAG
GGCATCGGATCTCTTTACAGATGGTTGTGAGCCACCATGTGGTTGCTGGGAATTGAACTCATGACCTCTGGAAGAGCAGTCGGGTG
CTCTTAACCACTGAGCCATCTCTCCAGCCCTATGCCAGCTTTTTGAAAGGAATGATTCTTGCTAGAGTGGAGCCTGGCCCTGGCTG
GAGGGGACTGACGTATGCCAGCTTTTTGAAAGGAATGATTCTTGCTAGAGTGGAGCCTGGCCCTGGCTGGAGGGGACTGACGTGCT
TCTGAGCACAGGCCTCTCCCGACTCTCCGCTTCAGGCCCTTCCTGTGGGTCACCACAGCAGTGGACATGGTCTTACTCTGGCAGCA
GCAAGTGGCATCTGGGAAGAGCTGGATAGCTGAGATGTTAGGGTGGAGAGGAAGGGAGGAGTACAGAAGAGGCTGTCTGCCCAGTG
GGCTCTACACCTGATAAGCAGGTCATTGTGTGGTGGCACGTTTAGAGAAGCATAGCACCCTATAATCCACTTGCCTTACCGTCACC
ACATTCCAGTTCCATGAAATGGAAAGGAAAATAAAACTGCTTCTGCCACTGCTGTTAGCAGTTTGACTTAGTATCTTCCTGGGTAT
TTTTTCTGCCCCATCCAAATAAGAATATGAAAACATTAGCACAAGGCAGATGTAGCTGTGGTTTGCATTTGGTCTGTATGCTGACT
GTTAGTAGATATCCTCAGAATGACATGGTCTCAGTCATGCTTGTGCCATGTTAAATTTAGTCTTATTTTAATAGCTGGTGACAATC
TTCTAGCCCACTTCATCCTTCTCTGGTTGCTTCTTTCATGTGGTTATGCTAGGCAACCAGCAGAAGCTAGGGCTAACACTACTGAG
TTCTCCGGGCCTTACACCCTTCCAGTGTGTCCACTTGTAAATCCACAAACACCCTTTGCCTTGCCATTAGGGAACAGGTTTGTGTG
GTCCACACAGTAGAGGTTTTATTCTTCAGTGTGTGACACATTTTCCCCTCATTTTCTAGAAGCCAAATGATGTGCACATGGCTATT
TTCTGCCTCTGTTGGGGGCTCTATGCTTTCTTTAAGGAACTTTTATTGATGGGACCTTTGACAAACATGCATCCAGGGTACTGTTA
TTGTTTGCATTCTGTGGTGATTCCCTGTAGTGCCATTGCCTGCTTCCCATGGAGCCCTTGCAGGCTCCTCTTCCCACTGCTAGAGT
CGGACCCTTGGTCCAGCCACCCAGTGAGTGAGTCTGTGCGCTGTTTCTTGTGAAGAGTCAGCTGGGGAGAAGGTTTAGGCAGGACA
GCTCATGGATATTGCAGTTTGATATTATTGCTCTTGATAGAGAAACCTCTTTTCTCACAGCTGTGTGCAGGTGTGCAGAATCCCCT
CCCCACCTCCCCAACCCCCCAAGTTCCCTCACCAGTCTGGTTTTACAGGGCTGAGGAAGAGCAGTGCTATTGGAAGACCAGATCTG
GTGTTGTGTACTGCTGTGGCCCCTTTAAAGGAAGCAATAGGTGTTTCCTGAAGCAGAATTGCTATTGGCCAGTGTTTAAAATGCAG
GAAAGGAGCATTTTOCTTTTAGCTGAGAGGAAAGATAAATGGAGAAGGAAATAGCCTGATGGTTTGTTCTGAGGCAGAGCTGTGGG
GTGGAATTTAGGGCCTCTTAAAGAGATTGAATTCCAGACAGGCAGTGGGGGAGAACTTAAATTCTGCTGTAAACAACAGAAGCAGA
ACTGTGAAAATTGCTATATGCATGTTGGGACAGAACCCCGAACTCAAGACATTACGTAATTCAGCATATTCTTCCCCAAGAGGGTG
TTTTGGTTGGGTGCAGTCATACATCTCAGAGGCAGAGGCAGAGGCAGAGGCAGGGGCAGGGGCAGAGGCAGAGGCAGAGGCAGAGG
CAGAGGCAGAGGCAGAGGCAGGTAGAGCTCTATGAACTTACAGGCCAGCCTGATCTATAGAGCAAGTGCCAGACCAGCTAGGGCCC
TGAGACCTTATGACCAATTAAAAATAATTGTTTTTTGTTTGTTTGTTTGTTTGTTTTGTTTTGTTTTGTTTTTGAGAATGATCATA
GATTTTTTTTTCACACTAGGAAGGCTTATCAATATAAAATAAGCAATTTCACTAAAAACTGTAATTAAATAACATTTTTGTATTGT
AACATTTAGGGTGTTTGCATTAGAAGGAACATCCCAAAGGCTAATGTCTGAGGAACAAAATAGGTCTTATTCTCTTGGACAGTGGA
CATGCCCTGGCTTTCTTGTGCAACGGGAAGGCTGTTAGGAGGCCTTCCATGCTGAACTTAAGGTTGAAGAATTCAGTCAGTTGAAG
TCTAAGGGACACATGAAATAGGGCCATGATAAACCTGTGGGACAAACTTGAGCTCTTAGACCTTTTTATTCATTCATTTTTAACTA
GGAGCTTTGGGGAGCCCAGAGTCTATGTAGGTTGTGGGGTGTGGAAGACTCTGGGCCAGATCCGCACTGCCAGTTACTGTTCCTGC
TOTGTGCACCCCATGTTAAACTCCACTGAATGAGTGGCGACTGCTCCTTCAGGGCTGGCTGGAGAGGGAAGCAGGAGTAGATTGCT
GGCAGGGGTTGGTGCCGTTCCTAGCTGTTAAATGTGTCTACACATTCTGCTGTGGTATCCAGAGTTGTCAGTGGCTTTGGTGGAAG
CATTCAATTGGCTTTGTGTAGAGCGTCATGGTCAAACAGCATAGCTGGTCTGAGTGAAGTCTGTGGTCCGCATGTGAAAGAGGATG
GGCAGCTTTCCCTCTCTGCTCTGGGTTGTAAGTTGAGTTGGGGAGTTTTGAGTACTGCTATTCTTACTCATTTTCAAGTTGTGTGG
CACTGGTTCTGGAAGTTAACAGAACACGGTTTACAAGTAATGTTCAGTTGTTAGCAGACAGTGAGGTTTTGAAAATCAAAATGTTT
TTTTTTCTATTCTTTTTTTCCCGGAGCTGGGGACTGAACCCAGGGCCTTGCGCTTGCTAGGCAAGCGCTCTACCACTGAGCCAAAT
CCCCAACCCCTGAAAATAAAAATGTTTTACGTTTGTATTTTAACTGCCAGTAAGAGTTCTTTCTGCCTGAGGGAGGGACCTGATGG
AGTGTTAGCTGCAGCCCTGGCACTGCCCAGTGTGCTAGAGTGAGAGTTCACTCATAAGGAGCCTGACTGCCTCAGGGGTTGCTAGG
GCTCACTGTGGTGAGGAGACTAAGGAACACCCCCAGTTGTGGTCCATGTAACCATAAGGTTACTGGAGGCGATGCTTCACTTGACC
TGTATAGCCTTATGTATCCAGTGTGCTTGTTTCTGTAACACCTAGGAGTATGACAGTGAACTCTGGTGGTGGTTGAGACCCAGGGC
TTCTCCTCAGGTTGCTACAAAGCAGGGAGGATACATGGCTTGAGTGTAGAGGGGACCATAGATGAGTGGCCTGGCTATGCAGTCCC
TCGTGGATAAGCAGCTTTGGATTAGACAGTGGGTGCAGGGAATGGAGTGTGGTGGAGGCCTTGTGGGGAGGGACAGGCATGTTCAC
TTGTCTTAGCAGTAGTGACTGAATCTGGAAGTTAAGCAGGAGGCACAAAATGGGTCTTTGGTACCTCTAGGCTGTGGAAAGATGGG
AGAGCTACAGTGTCTGGAGCCCTGGGTAGGAGGCTTCTGGTGCTGTTCTCTGGTGGTCTTGTACTGCTTGGGGCTGCCCATTAATT
AGCCTTGGCCTTGAAGAGGCCAGAGGGACTGGATTGGACATTTTGGAAGCCTCAGTCAGGATAAGCTGCGTGGACTCAGTTAAAAG
GTACAGACCCATGAAGGAAGAGGAGGTAGGAGGCAGACTGGAGACTTCAGTGTAAGTGAGCCAGAAAGTGGCCACTCACCCACCCC
AGCTTATCTACCAGCCTGACACAGCAGCCAGTGGCTTCTGTTTTCATGTTTATGTACCAAGAATGCCATGCATGGCTCAGCACTGC
TCCTAATCCCATCTTCTCAGTGTCCCTGTGTGCCTTGCAAATCATACTGTCCTTCTGAAGCTGTTAATGAACCTAACCCAAGCGGG
CAGGAAGAGTTTCATATTGAACATGTAAGTGATTCAAGATTGAGCATTTCCACTTCATTGGAAGTTTAATCTTCAAGTACAGAGTT
TTGGTTCCTGTAGCAGGAGTTTGTGCAGTCCTTAACTCTTGGGTAAAGCTTTTCAACCACAGCCCTCTTAAACAGGCTGTTTGTTG
AGGCTGTGTCACCACTGTGGTGGGGTTGTTTCTTACAGGCTCCATAGGCACACAGTTAGCCCCGAGCCACTGACGTGCTGGAGTGG
CTGTCTCCAGTCTGGTGTCCCTCAGCTTTGTGTTGCTGGTAGGGGGAGGACAAGGAGACCAGTCTTGGCATAGAGCCTTTGTTGTG
AGTTAATCAAGTGACCCTGAGTAGCCTTTTATTTTCACAGTGACTTTTGAATGTAAAGTATTGTGACACAGTGTAAATGTTTTGTG
GGAGATTTGTACTTTGAATAAAGTAGAAACTATACCTAGTGGTAACACGTGCATGCTACTTTGGAATGTTGAAATGGATCTCTTAA
GTTTCCTACCACATGTCCTGTAGTGAGAATTTCTGAAAGAATCCTTAGCAGTTTAACCGGGGGGCCTAACCTTACACAGTGGGTTT
CACTGCTCTTCTGTTGTGAGCCCTTTGTGTGTGGAGACAGGAAGATATTTCTCCCTGGGCTTGCGTTTAGTGAGTAAGATGTCAGG
TCATATTGGTTTTATTTTTATTTTTATTTTATTTTACTGTATGAGTGTTTTGCCTCTGCAAGTGTGCCCAGTGCACATGCCTTGTG
CCACAGAGACCAGAAGAGGGTGTTGGATTGGTTAGAGCTGGAGTTAGAGAGAGTTGTTGACTGCTACGTGGGTGCTGGGAACCGAA
CCTCTGTCCCTTGCAGGAGCAGCGCGTGCTCTTAACCACTGAGCCAGCTCTTCAGCTCCCGTGTTGGTGATTTGTAAATACCTAAA
CTTCCTGAAGAGGTTGAAATAAGTTTGGGGGTCTTTTTTATTTTTAAAGATATGAGGGTAGAGTGGGCAACTCGCTGGTCTGTGTA
TTCTAAGGGAGCGAAGATGAGCCTACCTCCGTTAGAGTCCTCTCCAGCCACTTACCACCCCCAGACTTTGGCTTTGACTTTGGCTT
AGAAGCCCTGGTTCGGCAGTTCAGTGTTTGTTTTCTTTCTCTTCGTCACTTTGTGCTGCAGCATAAGCTACTGTGGAACCTTTATG
GCTCCCTGAGTTCTGTGACTGTTTCCTCAAGGTAAGTACATACTGATGCAGAGATTGTCCTGAACTTAGATAAGAGTTTTAATATT
GCTGTGTGTTAAATGCTCTTTCACAGTTTTTTCCAGAAAGTAACTTGTGCACCTGGGCGTAGGACACCAGGCCCGAAATCTCTTGT
TAGGGAAACACACAGTGTTACCTGAGGCCCCGGGCTGCACACGAGAGCAGACCATTGTGTGTGATGCTGTTTCCTTAATTGAATTA
GTGTTTTGGTGGACTTCACATTTATATAAGTTTTATAATAGATTTTATAATCTTCAGTTTTCAAAATCACTTTATTTATAATTTTT
TCAGGAGATAAATCTCCCCCTAAACTTGAACCATCAGATGCATTACCTCTTCCTTCAGACCCGGAGACTAATTCAGAACCACCAAC
CCTCAAACCAGTAGAACTCAACCCCGAGCAGAGTAAGCTATTCAAAAGAGTCACATTTGATAATGAATCACATAGCACTTGCACTC
AGAGCGCACTGGTAAGCGGACACCCTCCAGAGCCCACCCTCGCCAGTAGTGGCGATGTGCCGGCGGCGGCGGCCTCCGCAGTGGCG
GAGCCATCAAGCGATGTAAACAGACGCACTTCTGTTCTCTTCTGCAAATCGAAAAGTGTAAGCCCCCCAAAGTCTGCCAAGAACAC
TGAAACCCAGCCAACTTCTCCTCAGCTAGGGACCAAAACCTTTTTGTCTGTAGTCCTTCCGAGGTTGGAGACTCTACTGCAGCCAA
GGAAAAGGTCGAGGAGCACATGTGGAGACTCCGAAGTGGAGGAGGAGTCCCCGGGAAAGCGCCTGGATACAGGTAAATGTCAGGGG
CAGCCCTCCGGGGAACTCTTAATGTAAAACTGTGGTGCTGAGCATCCTCTCAGTCCTAAAGCTGCAGAATTGTTTCAACCAGCGGC
CATTCAGCCTCTTGGCAACCCAGCAGCTGGCCATACAGCAGTGGCATGTCTGGCCCCGCCCTCCTTTGTTCCTCCTCTTTCTCTGT
GGCTTTTCACCTATTGACTTTGAATGTGATTTGCGTACCTTGACTATTGTGTGCATGTGTGTGTAAACTGGTACCTGTGAATGGCC
ACACCTGGCACTAGGTGTCCTGGGGTGGTGGGTGTCGCCTAAGAGCAGTGCCCACAAACTCAGCCATAGATTTGAACCTGAACCTC
TCTTTACTGAAGACTGCATCTTCCCTGAGCTTTCTGAAAATATTCTGTCATCTCATTACTTGTAACACTTCATAATTGGCTTAAAG
AAAATTGTGATGTTCCCTCGATGTGTTTTGTATCTTGTTTTAGTACACGTGCACTTGACTGGTAAGTACATGTCAAGGTACATGTA
CTACTAATGCTTGGTAAAATCATACTCAAAACGTTTCTCCTTTTTTGGTAAGCTTTTTTCTTTTTTTTTCCCTTAAAGGTTTGAAG
GCNNNNNNNNNNNNNNNNNNNNNNNNNNNNNNNNNNNNNNNNNNNNNNNNNNAGACTTTTATGAGTTTGAGGCCAGCATGGTCTGT
AGAGTGAGTTCCAGGATAGCCAGGGCTACACAGAGAAACCCTGTCTCAAAAAAACAAAAACAAACAAAAAAAGCTAAATTAGTTTG
GTTGGATGCCACAGTGTACATACATATGGGGTGGGGTTGGGAATAAATCATCAGAGAACTGGGAAGTTCATTATCTCTGGGATTGA
ACTGGGGCCATCAGCTTGGTGGCACATGAATTCCCACTGAACCATCACCAAGGCCCACGGTTAACCTGAGAAGTGTTTTTATTTTC
TAAGACGTTGCCATGTGTGCCTGTACCTTTAGAACCTCCATGGAAATTCCCTGTGCTGTGCTTGGACAGTACTGGCCTGGCTTCCT
CACAGCCTCCTTCCTGTCTAATCCCAGATAAGACACAGTGGGGAATGGGTGTTTGTGCTCTGAGGCAGTGGTTGCCAGCATCAGAT
GTGATGTTCAATGTCTGCTCTGTTGGAGGCAGGCTTACTTACACCTTTGTTTTTTACCTGGATTAGCGTCTTAGTTACTTCTCTGT
TGCTATAAAGAGACACATGACAAAGGCGACTTCCTTGGAAGAGAAATGAGGTTCATGTTTCTTGAGGGTTAAGAGTCTGTCCCCAT
CATGGCAGGGAGATGTCAGGCATCCTGGCAGGCATGGCATTAGAGAGAGGAGAGATAGTGATCACAGAAGCAGGAGAGATCGCTGA
CTGGAAATGGCTTGGGTTTTGAAACCTTAAAACTGGCGACACACCTCTTCCAATAAGGTCATTCCTCCCTAATCTTTCCCAAACAG
TTACACTAACTGGGGACAAATATTCAAATACGTGATGCTGTAGGGATCATTCAAAACATCACATTCCATTTCTTGGACCCCTGTAG
GCTTGTGGCTATATCACAGTGAAAAGTGTATTTATTTAGTCCAACTTCAAAAGTCCCCATAGTCTCACAGTTTCAACAGTTTAAAA
GTTCAAAGTCTTCTGAGACTCCTGATTTTAACCCCTTGTAAAATCAAAATTAAAAAAAAAAACAAATCACATACTTGCAGCATACA
ATGGCACAGAAAATACATGAACATTCCAAAAGGCAGGAGAGGGAGCACAGTGAGGAAATACTAGACCACAGCAAGGCCTTAATCCA
GCAGGGCAAACTCCCAGTCCTTTAGCTCTGTGTCCAATGTCAAAGACTGAGGTGGCTTTCCTTCCAGCTCTGCGGATTGCAAACCA
TCTCCCTGATGAACTGGTTCCATGCTGTTTGTAGCTCTCCTTGGTAGACGTCCCGTAACGTTGGGAGCTTTAACATCTTGGCATCT
CCAACACAGTTCAGCCACACTCAGTAGCCTTTCGGACTTCCCCATGCAGAGACTGACCTTCAACGAGTCTGGTTTCAGTGACTTTC
CTTAAGGGAGGAGGAAGATTCCATACCTCCTTCATTCCTGTATTCTTCAACAAGACTCTGAAGTCAGAACGACTGGGCTGAAGAGC
TGTATTAGGCTGCCAGCTGGGATGGAACTTGGCCTGACTTGAATTACATTGGCATAAGCCTTGACTTGTTGCTTTTTAGGAACAGA
TCATTCTTTAGCCCTGTTCTTCTCACAAGGGTCAGCTGAGTAGAATCTCATCCTAAGGACACCACTCCTTTTATTCCATTTCTCCT
CCTCTCTGTTAGAACAAGCCTGGGCTCCATTATTAAATTTGGTTCTATTTCTTTTCTCCTTAAACTCTGTATTTTGTGCTTTCTTT
TTTCCACACTTGTTCTTTTTCATTGTAGATAACACATAAGAGTGATTACTAACAACTATACAACAGAGTTTATTAGATTAAATCCC
CCTCCCCCATTCTAATTTAGTTTCGGGCTGATTGTTGTTGTTGTTGTTTTTCCAAGACAGAAAGCCTCTCTTTGTAATCCTGGCTG
CCCTGGGTCTGTAGACCAGGCTGACCTGAAGCCTGGAGATCTGCCTGCTTCTGCTCCCATAGGCTGGGATAAAAGGCACACACCAC
CACCTCCTGCTATCTCCGGCTGATTCACATTGTTTGCAAAAACATATCACAAGAATGGTCTTTAGCCCAGTCGCTAATGTTGTTTC
CCTCTTAAAGCTCTTGAACGGGCCCTTCCTAGTCTACGTTGCTTTCAGGATGGTCTTCCAGGCTTCCTATTACTATGGCTCATTAA
CCCCACTTACAGTGTTCAACCAGTCCAAAGTCCCAAGGTTTTCTAAAAAGTACCATGGTCAGGCCGGTCACCCTAACTCCCTGGTA
CCAGCTTCTGTCTTAGTTACCTTTCTGTTGCTGTGAAGAGTAACTTGTAAGAGCAGGCATTTGATTTATGGCTCATGGTTCCATAG
GGTTAGAGTCTGTCAGTCCGACACCATTTTGGTGCTGAGAGCTCTTATCTGATCTACAAGCATGACTCAGAGAAAGGGAATGCTAG
CCCAACGGCCTGGGCTTTGGAACCTGGAAACCCCTCCCCAGCAACACAGTTCCTACAAGGCCACACCTCTAAATGTTACTAAACAC
TTCACCAACTGGGGACCAGGCATTCAAATGTGATCAGATGGGGGGCCATTCTCACTCAGACCACCACACTAAGGAAATTGCATTTC
CTTCCAGGCACTCTAGTGTTGGCTGTTCTTGTCTACACGTCAGCAATGAGCAATACATAAGTTGCTGATGAGTGAAATCTGTTTCC
TGGAACCTTGCCAGGTGGTCCAGGTCAGAGATTTGGAAGGGCAAGGCTGGCTTGGTAGTGACAGTGGACTGTTGGCACCTCTCCAT
CTCTCTCCATCTCTCTTGAGNNNNNNNNNNNNNNNNNNNNNNNNNNNNNNNNNNNNNNNNNNNNNNNNNNGNTTTATATCTGTCTG
TCTATCTTGGAGGTTGACTCTGGTGCTTCATGGCAAGTTGTGTACTATGGAACCCCTTTCAACTGTGTGTGTGTGTGTGTGTGTGT
GTGTGTGTGTGTGTGTGTGTGTGTGTGTGTGTGTGTATGCCACAGTGTGTGGGGGATCAAAAGATTGTCTCTACCATGTGGGTCCC
AGCATACATTAGACTTGTGTAGCAAGTACCTTTACCTTTGCACTGACCCATCTTGGTGACCCTTTCAAAAAAGTTTGAAGCAAGCT
GGTTTTGGACCTTTTCTATAACAAAGGCCTTAACTCATTAGCCTGTTACCTCAGCCTCTGAAGTAGCTGAAATCACAGGCCTGTAC
TAGTAGGCCCAGCTAATTTCATTTTTATTTTGCACTTAGTAATTTTATAGAAACTGTTCTCAATGCCCTCTTATTTAAATCCAGTA
ACACTTGGTTTTGATGAAGCAGTTCTAACATGCCAGAGGAAGCAGCTTGAAACGGAAGTTGTTTCACTTCTGGGTGTTTCTGAAGC
CATGTAACTTTAGCTTTTCTTAGCCGCATTTAGTGCAAGACTGGTGTCTTTTGAGCTCTCTGAAAGTCCTTTGTCACTGAACTGGC
CGTACTATGCTGTGTTCTACTGCTAGGAGATTACTCAGTGTCTCAGTACCAAGTGACTTGCGTGCACAGGATGGCTTTCTACCTCC
TCAGGGTTCATATCTTTCAGCAACAGAAAGTTTATTGACCCTGCAAGACACTGTAGAAAAAGCTTTCACCGTGGAGCTGCTGTTTT
GAGCCCCTGCTCTGTACCTGGCAGCTTCTGCCAAGTACTGTGGCTAACTAGGCCATGTGGGCTCTGCGAGACTGTTTGTCACCTCT
GGGTTACTAATCAGGAGTTCATGAAATGTTATGTGTGTGCACATCTCAGTTCTTTTCTGGGTTCTTAGTAATAACGATGAGTCCCA
CGGAATCTTAATAATAGACCTTTAGTTTGTGTGATTCCCCAGTGCCTTTGATCCTGACTGAAATGGAGATTTCTGCTTTCTTATTC
CAGAATGGCAGTAGATTTCAGTGGATGCATGATGAATTCCTAATCGCACTCCTGAGCAGCCGGGAGCCTTGTTAGCACTAAGATCT
GACCCTCAGGAACAGGAGGCGTCTACTGCTGCATCTGCTTGCCCGTGGTGGGCCAGGCATGGGCTGAATGGGCCCATCCTACCATC
GGTGCTGGCTGTGCCTCCACTTGAACCTTCTGGTGCTTTCTGCGCACCTGGATTTCTTGTTTCAAGTTGCAGTTCTTCGCTGTTTG
AGGACTTGGAATATTCAGAACCTTCTGATCTTTTCCAGGTTCATCTGGCACTGAACTTTTAGGGGAATTCTCTGGTGCTCTCCAGT
GCACTGCAAGATTCCAAGTTAGATTAAGTATGGACTTACTTATTTTTAAACTGCCCATCCACAGGCCTCCGCTTGCTCATGCCTGC
AGGACAGGCGGGGATGTGGGCAGTGCCGAGCATGGTGTGACTGCTGTTATGGTTATCATAATTTTTGGAGCTGGCTCTGTTTCGTA
GATTTTTTTACTCTGCCTGTTTTATTTCCGTCAATGGACCATCAGGCCAGGACCCGTGTCACTCCTTACTCATACTGTGGTGTGGA
GATTCTCCATGAAATGTGTGGTGTGGTGATAAGCAAGTGAATGTCTTCCATGGCCACAGGCTGTTGAGGGAGGAGACATTCCTGCC
CTTGCAGTCAGACTAAATGGCTTCTCACTGTTTTCCAGGTTCTCAGTTAACCACTAATGTGCCTGGGTAGCTCACTCTTTGGATCC
TAATCCTTTTCTCTTAACCTCGACTTGGATTGGAGTTCTGCTAAATGGCCTCTTGGATTGCAAAGCCTTCGCTGCCTTCTTACCTT
GCTCCTAGTTCTTGAGGATCACATTGGAGTCATCTGCTGAGCCGTCCTCTAAACAGACACTCAGACACACCTACCCCGGAGGAGAT
CTGTCCCGGGCCAGCAGTTGGAGGAGCCTGGTGCCTGAGCTGATGTCTCGGTGCCTCAGGTCTTCCTGGTGCTATAGCAGAACCTG
CTGTAGCTTGGACAACAAATCCAGCAGTTTTGCCTCATCCTGAGCACATCCAAAACTGACCTGTGATGACTGGGGGCTCTGGTTAG
GGCGTCTTGGTCTAGATGTCTGAAGGGACTGACTGTACACACATGTTGCCCTAATGGCCCTAAATAGAGCTCCTTACTTGGTTGTT
AGCATCTTTTGTTCTCTGTCTGGTGTCCCTTTCCTCTCCTTCCATGTGTCTTGGCTTACTAAGCACTGCTCGTCTAGTTATTAGCT
GTGCAGATATCTGCTGAGCCAACCGGGAAGTCCTGCATGGCCCTCAAGAGGGCATTTCGGCTTGGCTTCTGGCCTGTCGTGGCCAT
ATGCAGCCGTGTCTTCACTCATGAAAAGCAGAGTGGTGGGTGGGGTGGTATTGTTTGCCGTGCTTTTATCAGAAGGTGGGAACATG
CACCCACTGTGACATTCTTTTGGTGCCATCGGAAGACCATAGATGCCTCTGCTGCCACCCTCAAGTGGTCGTGTTCCGAGCCTAGG
ACGCAGGCTTAACAAGCTGGAGCTTTGGGCACACGATCCTGTGCTGTCTGAAATGAGTCCGTGGACTCTGGAGAGCTGTCTCTTGG
TAGTGGGTGTTACCAGCAAGGATGCACTCAGTGACTATAATATCTCATAGGCCTTGTGTGTTCTGTAGGTGAGAGCCTGGTCAGTG
TAGCTTACTAGGCCCCAACATTCTCACTTGCTGCAGCCCGTGAGCTCTTGCATTGTGCAGGTTAGGAGCCCATGGTAGAAAGGATC
ATGTCCCTACTATCCCACCTTGTGCCTCTCAGCTTTGCAAATAGACAACCTGGGACTTCTCCCTGGCCTGTGGGTGAGAGCTGAAG
ACCTGAGCACCTCAGGGTACCCTTTATTGGCCACTCTGTGTACCCCAGTTGTGCTGTAGATACCTGGGCCGGGGGGAGCTTTTAGG
GATGCTGTGGTAGTTACAAGGCTGGGGCTGGCCCACCTAGGGACATGTTGCAGTGGTACTTTGTGGCCTCCTTTGGGTCTGAGTAA
GGCAGGCGTTCGAACTCTGCCTTAGTCTTTGGAGACATCAGGACCCTCTGCACCATATGCATGGGGCCATTGGCTTTGTGTAGAAG
CCATCTCATCTTTTGCTCTGGTTGTCAGGAGTAGTTGGCATGTATGTGATCTTCTACAGTAAACTTCAGTGCTAGGAGGGCACTTT
CCTTTGCTCTCCTTTCCGCAGCTAATGGGAGAATCATTGTAGGAAGTGGATCACAAAGAGGGAGGCAGAGACTGCTCATTACCTGG
CTTTGGGTCACAGCCATGCATTCTTCAGACAGTGGCTGCAAGAGCTTTTCAAGCTCGTGTCTCTGGCCTGTGTTGCCTCTTGGGGC
ATGTCCAGGGCCTTGGAATAAGAGTCTGATTGGGCCATGCAAGCACTGTAGTAGTTTGGGCTGTAGCACCCTCTGAAAAGCAGGCC
CAGAGAACTGCTTGTCTCTGCAGGCCCCAGGGGTCTCTCCTGGAAGCTTCTCAGGTTTCACAGTGGCTCTGCCAGCTTTTCAAGGT
GTTACGTGTCTTTATGAAACGTGTGAAAACTTTCTGTAAACTTAGGAGCCCAGATGCAGTGTACCCTGGTAATTAAACACTTGGGA
AAATGGCAGAGACATTTAATCATATTTTTTCCCTTCTCAAAGTTATAAACTTTCTCTTAGTTTTTCCAACCTCCTCCAGACTCCCC
AAGGGGCTGTTTAGGCCCTGACAAGGCCCCTTGTTACAGGTAAAAGCTATTGCCATCCTTGTCGGGAATACCAAGTGTTTTTGGGA
ACTGTACTTCTGGGTTCTTTCCTGGGGTGTCTTCTAGCACAGAGAGGCTTGACCTGCCAGTTCTGCCTAGCCATGGCAGATGATTT
GGGGCTTGTAGTTTTCTAAGATCTTGGGTCCTGGAGCAAGGGCTCTGCGTTTCTCTGTCCATCCAGGTAACAGGGCTGTCTCTGTG
TTGACTTTGCTGACCTAAGTCAGCAGGTGTCCATTCTACGTTGTGTGTTGCACCTACCCAAGGCAGCACCATGTTCTCCTACCTCC
TAAGAATTCTTGGGCCTTGAGGCTTTTAGGAGAGGAATGCGGCTTCTTCCCTGTCTTGTGTCTTCTGCTTTGCCAGTGAGCAAACA
AGAAGCTTCTCAGAAGTCTTTTTAGCACAAGCAGGTCCTTTTCACAGGTGGGAGAATGCAATGAAGACCTTAGTCACCTCATACGT
CCAAGAAAATGTTCTTTAAAAATAAGTTTACATGCTTTACTTTGGAAAATAGAGCTTACATTTTTAAGGTTATATGGGGAAGATGG
GCATATGTGAACAAAAAGTGTTGTCTGTTTGCTGTTCCCGTCCCCTTCCCTTTCCCAAACTGGTGCAGCCAGAAGAAGCCAGACAA
GCACACAGCCTGGGGACATGATCCTTCTGATTCAGGGAGGTCTGCAAGGACCATGGGTGGATGTGCCTTTTTCTACTTACTGACTT
AAATTGAGGGTCACGCTTGCTTGCAAGGAATATGGTGGTTGCCTTGACTCAGATTTGCCTTTATTAAAATACTTTACAAATATCCA
GATGCTGTGGTTGCGTTTGTGCAGACATTATACCTGATGTATATCTTGGCTCAGCTTCCTGCCAAGTTCCACATTTTTTGGTGCTT
GGGGACCTGGCATTGCTCAGGTGAATTGGGCCCACACTTGCTAGTTTAAAATGTTTTATCTATATGTTTAAAAAGTCCTTGTTAAA
ACATTGATGTTTCTATTTTTTTTTTTTTTTTTTTTTTGCTGGTGGTCAACCCAGGGCCTGCATGCATGCTAGGCAGGTGCTCTGTT
ACTGGACTGTATTGCCCTGATCTCTCTTTNNNNNNNNNNNNNNNNNNNNNNNNNNNNNNNNNNNNNNNNNNNNNNNNNNTCCCCTC
CCTTCCTACAGTTGTGCATGACCAGTGTATTTTGTGGAGGACTGCAGTGTTCCACCTCCCTCGGGTACTGCAGGGCTCTCATCTAG
ACACTGCTACCTTGTCTAGTGAACAGTCTAGTTTTGTTGTAAGCTCACAGACCTTACAGCATGGGATACGTTAACTTGGGAAGTGG
TGACAGTTTCTTTTTGCTTTGCACTCATGGGACTCATTGAGCAAGTGCAGAACTGCTCTGACTTTTTCCTTACACAGCAGTGCCTC
TGCGTCTGTCCACTGAGGTCATGGGAGTGAGAGATGAGGTGCTGGCTTTTTTGTGTATAGAATGTTCTTTGTGGGACCAGTGATGT
AGCTCAACTAGCTGGACTAGCAGCCGGGAACTCTGGGCTTGAGCCCCAGCACCTCAGGAACCAGGGATGGAGGCAAGAAGATCAGA
GATTCAGGAGCAACCTGGACCTGAAACCCTGCCTCACAGAAGCACAGTTATTTGGAAAACTGATTGTAATTTTTGAGGAGGAGAAA
GGTATTAAGTTGAGCTAGTGCTCTGGTGTATTGTGACCATGTATCTGTCTGCTTCTTGTGTTCCAGGTCTCACCAATGGCTTTGGG
GGTACTAGAAGCGAGCAGGAGCCAGGCAGCGGCCCAGGGAGGAAAGCTGCGCCCCGACGGCGCTGTGCATCTGAGTCCAGCATTTG
TTCCAGCAACAGCCCACTGTGTGACTCAAGGTAGCCCTCTGCCTTCTGCAGCAGGGGCCTTGGCCGCCTTCTGAATGAAGATCTAG
AAAGATTGCCCTGGAGTTTTATGTGTGTGCTCTGCCATTCAGGGACAGTTTCTCATGGGATAAATTGAACCTGCTCAGTAGAATTT
AACAGAAGTCAGAAGTTACTCTAAATTATACTGTATAATTAAATCAGCATAATTAAACTCTGCACCTCCCACTCCCCAAACCTTTT
CTCTTACTAAGATTGTGGTGTGCACTGAGTGCTCTGCCTGCCGGTACTGTAGTGACCACTCTGTCAAGTGGCCTCATGGGGACAGG
CTTACTTTCCTTGGGTCTCCACACCACATTGTCCTTTGGCAGCCTGGCACCTGGTATAGGATGAAGCCCCAGGAAGGGCACTATAA
ACCGTAGTCTGAGTTTGACTCATCTCTGCTTTCCTCCCGAAGCTTTAGTACACCCAAGTGTGGCCGAGGGAAGCCTGCGCTTGTAC
GAAGGCACACACTGGAAGACCGCAGTGAGCTGATATCTTGTATTGAAAATGGAAACTACGCCAAGGCGGCCAGGATTGCAGCTGGT
GAGTTGGGATACAGACTTGGATGGAAAGGCAAGGTCTGGTCTTGGTGGCTTGGGGCTGTGAGTCAAGCCCCCCACATTAGAGGAAA
GGAATAAGCATATGCTTAACTCACTGATGCTGGGGTTTCAGGCCTGTCCGGAGCTGCATTGTATTTGGCTTGGGTAGTTCCTGATG
ATGGAATTTGTCCAGGACAGGGTCTGTTGAGGATATCTGCCTTGTAACTGAAGGTGTCACAGCAGGCTGATCATTCCTAGCTTCAG
GCTCCGTGTAAGGGAGGAGTGCCTGCTAGGCTGATGCTGTTTAGATTTTCCTGTTAGTGGACCAAGCCCACTCCGGGGATAGAAGT
ACTTGGCTGTTGTTTGAGGCTTGTCAGGGCAAAGACCTGAAATTGAGAAGGGCTGGAGACTGCAGGGAATGGGCTGTGGCCATAGA
GGCTGCAGTGCAGTGCGACAGCAGTGCTGGAGGTCCTGGGTTACGGAGAAGCTAGGACATGGTGCTGCTGAGAGTGGCATCACTTA
CCAACAGTAGTCGGTGAGGCCTGTACACAGGCGTGTCCTGGGCGGCTGGATAGACTCAGCGAGGCTCACAAGAGCCAGGTGGCAGT
AGTTTGGAATAAGCCAGAACTTGTATTTGGCTGTTTGGTTGTGTAATAGGCGTCTGGTACATGCTAAGGATCCTGTCTTTAGAATC
GAGGTTTAAGTATGGCGAGGTACAGGGGACCTGAAGTCCTAGGCCTTAGAACTGGGGGTGGTGGGAAGCAGGGAGCCCTTTGGCAG
GCTTCTAGGCCTCACTTCCACGGGAGGAACAGTGAGGTTCCTGCTTCTCCTGGCTGAGGTGTTGTAGAACTATCTGCTCTAGCAGC
TAGGGGAGCTGGGGCCTGAGAGGAGTGGGACTTTTTCTAGCCCCAGGCTTAGTGGCTGTGTTGGGTATTAGTGGTTCTCCTGCTTG
GTCTGACTCAGGTGTTCCTTTTTTCCTAGAGGTGGGACAGAACAGCATGTGGATTTCCACCGATGCCGCTGCCTCCGTCCTGGAGC
CCCTGAAGGTGGTGTGGGCCAAGTGTAGCGGCTATCCCTCCTACCCAGCACTGGTGAGTCTGGAGGCAGTGAGTAGGGTGTTCTGG
TGGGACCTGGGTTGGGGCGCCAGTGCATGCTCTGCACCTTGCAGTCTGTGCTGCCAAAGGTGTGATTGTCAGAGCTTGGGGCAGGT
GTATGAATGGCTGTGGGTTTAGGCATGTGGCTCTGAGTCACTGGAACATGTCCTTGGCAGATTATTGACCCCAAGATGCCACGAGT
GCCTGGCCACCACAATGGTGTCACCATCCCCGCCCCGCCCCTGGATGTGCTGAAGATCGGTGAACACATGCAGACCAAGTCTGAGG
AGAAGCTGTTCCTTGTTTTGTTCTTTGACAATAAGAGAAGCTGGTGAGTGTGGTGTTTAGAACTCTACAGCAGGCGGAGCTGGGGT
TCTGTTACACCTGGGGCTTCGCTTACCCATGCTGAGTCAGGGTGCCTCCGAGCAGTCTTTCCTGGTCCTTGTCTGCTGCCGCCTGG
GCACGGTGGAGTGCTGGTCTCTCCTGACAGCTCCTCTCACTTCCTAGATCCCTGAGTGGCTCAGGCTGTCTTCAGTTCTTGCTCTA
GACTCTCTTGCAATGGGCTTTTTGATCCTACAGCTCTGCCAGGACACAAGCTCCACCCCTTTCCACCCTCTCTATCTTCCTGTCCT
CTCTCTACCCAGAGCTACCGGAACTCCCTTATGCCACGGATCAGACCCTAATATTCCCTGAGGGTATTATCTAGTGACACCTTGAC
CATATTTAGTTGAATCCAGCCCTTTCCTGTGTAACTAGGTCCTTTCAGCTGGCCTTGCTTAGGACTAAGAGGTGCGTCAGTAGTTA
GTGTCCTGTTGAGTGGCCACTCTTGGGGAGATAGCAGGTCCCCTGTGGTCCTCTTCTCTCTGCCTGGCTCTGGCTCCTGGCAGTTG
GTGTTCATTGAGGCTTAGACTGGCCTTGCCCACTCGATGGTTCTGTGTATAGTCTTCTCACCCATCCTAGGAGGCCTCCTTACTCT
GCTCCAGAGTTCACTGGCCTATGTTATGTGTATACATATCCTGCTTGGTCCCTTACATACACGTGTACCTGGGCTCACCTAGTGGG
CCACACACTTGTGTTATCTTGGGCCTGTTCTGTGTGCATAGCTGTATGTTCCAGATTTATCTCCAAGGCCACTTTCATGTGTATGG
AAAAGAGGATTGTATTATGGTTTACATGCCTTACCTGAGTCCAGACAGGGCTGGCTAGGTGGATGCCCTATGCAGCTGAACTTCCT
GCATGAGGTGTCCAGCATGTGGGGAGCTTGGGTTGGGTACCGCCAGGTTCCTTGTTGTGCACATGGGCTGATGGTTAGTGGGCCAT
ACCAAGTATGGGCTGGCGGTTCTTATGCTCTGTCTTCTCTTTAAAAGGCAGTGGCTTCCCAAGTCCAAGATGGTTCCTCTTGGTGT
GGATGAGACCATTGACAAACTGAAAATGATGGAAGGGAGGAACTCTAGCATCCGGAAGGCTGTGCGGATTGCATTTGATCGAGCCA
TGAATCATCTGAGCCGAGTCCATGGGGAGCCAGCCAGTGACCTCAGTGACATTGACTGAGGTGGCTTCCAGCAAAAGGCAGTGGCT
AAAGCCACAGCCAACCAGGAGCCCTGTCAATAGTGTTGATAAGCTGTACATGTTTGTATATTGTTCAGAACTTAACTTATTCTGAT
TTTCTAGGTGTAGTTCTTTAATTCTTTTTCCCCCCCCCGGGAGGGGAGGTTTCACTTCCAAGTTTTCTATGAAACCATCTGGTCTT
GGCTTTGCAGGTGAGGAGGGTCTGTTCCGAGCAGTGTGGTGTGGGGTCCCACTGCAGGTGCCGAGTGCCGAGGCCTCACTTACTTC
TAATCTGTAGGGTTTTTTTTTTTAAAGACTTTTGAATGTTTAATAATTTTGTAGATCATGCTCTTTACACAGAGTACCGCTTATTT
AATAAGACGGGGTGTAAATTTACAATGACAAATGTGTATTTTAAGAAAGAAAATGACATTATTTTGAATGGTACTTTGTGCAAAGA
GGGAATAAATTTATGCTGTGTGCATCACTTGCAAATCACCAAAAAATGTCCCGCCAGCTGCTGCCGGCCAGGGCCCGTTCTCACCG
TTCTGACTGCCCTGAGTCTCCTGTTCTGCcCTGGCTCCTGCAGGCGTGCCTCCCAGCGGGTTATTTATTGTAGAAAGTGTACTCAT
TTGCTTTATAATGAAAAAATAAATTTGCAAAGGTATATTGATATGCATTTTTATACAGGCACATAAAAACTCAACTTGGTGTGGGA
GCAGAATGTGTTGCGAGGTTATATACATGATGGGCCTGTGTGTACTTTGATTTTTGTAACTTGTAATCTTTTGTTTACAATGAGGA
GCTTTCTGTAACTTGTTTTAATTTAGAACACTTTGGTAGCAATAGACCTTTGGATACATTTTTGTATGGTACATGTGATGTATATA
GAATTAGTACTTTATTTTTATTTCTAAGAGGTAAAGCATTATGTTAGGGGAAAAGGCAGGGTGGGTTTCCAAATTTGCATTTTTAT
ATTAAAAATAAAGTGAAGATTTGGACAGTGTGGCCCTCTCATTCCTGCATCACTAGGAGGCTGGGTGAGCTGTAGCCTGAGGTACA
TGTGGGAGCACTGAGGCCTTGAGTGGGTGGTGTGACCAGGTGTGACACACTTGATCTAACAGCTGACCTGGGGTGGCATTATTTAT
TATTTTGCCTAATCATATTTTTATTTTAAAGCTAAATAGTTACTAAAAATTTTAAATGTTCTTTTAAGTCTACATGTTTGTAATAT
CTCCATAGAAACTTGAAAAATAAAAAGTCTTCCTTTGGT
SEQ ID NO: 18

TABLE 9
Size, position and sequence of BRD1 exons in rat. Red marks start- and stop
codons. Highlighted area marks coding part of the gene (UCSC Genome Browser on Rat
Mar. 2012 (RGSC 5.0/rn5) Assembly)

| Functional | | Genomic | |
|---|---|---|---|
| structure | Size | position | Sequence |
| Exon 1A/ | 39 | 129413493- | CATTGTTTGCTTCGCTGGGGAGCGAGCAGCGCCTCGGCA |
| Promoter | | 129413531 | SEQ ID NO: 19 |
| Exon 1B | 1381 | 129408698- | GTAATCATTGCCAAATGAGGAGGAAAGGACGATGTCATCGAGGTTCTGCAGCG |
| | | 129410078 | AGGCATCCTTCTTCCCCGTGCAGTATTAAACACTCCCCCACTCGTGAAACATT |
| | | | GACATACGCACAAGCTCAAAGGATGGTGGAGATAGAAATCGAAGGGCGTTTGC |
| | | | ATCGGATCAGTATTTTCGATCCCTTGGAGATCATTCTAGAAGATGACCTCACT |
| | | | GCTCAAGAAATGAGTGAATGCAACAGTAATAAAGAAAACAGTGAGAGGCCACC |
| | | | TGTTTGCTTAAGAACTAAGCGTCACAAAAACAACAGAGTCAAAAAGAAAAATG |
| | | | AAGTCTTGCCCAGCACCCATGGCACACCGGCTTCAGCCAGTGCCCTTCCTGAG |
| | | | CCCAAGGTGCGGATTGTGGAGTATAGTCCTCCATCTGCACCCAGGAGGCCCCC |
| | | | TGTGTACTACAAGTTCATCGAGAAGTCAGCCGAGGAGCTGGACAACGAGGTAG |
| | | | AGTACGACATGGATGAGGAAGATTACGCCTGGTTAGAGATCATCAATGAGAAG |
| | | | CGGAAGGGCGACTGTGTCTCTGCCGTGTCACAGAACATGTTTGAGTTCCTGAT |
| | | | GGACCGCTTTGAGAAGGAGTCCTACTGTGAGAACCAGAAGCAGGGTGAACACC |
| | | | AGTCCTTGATAGACGAGGACGCTGTGTGCTGCATCTGCATGGATGGCGAATGC |
| | | | CAGAACAGCAACGTTATACTCTTCTGTGACATGTGCAACCTGGCTGTGCACCA |
| | | | GGAGTGCTACGGGGTGCCCTACATCCCTGAGGGCCAGTGGCTTTGCCGCCACT |
| | | | GCCTGCAGTCTCGGGCCCGCCCTGCGGATTGCGTGCTGTGCCCGAATAAGGGT |
| | | | GGTGCCTTCAAAAAGACAGACGATGACCGCTGGGGCCATGTGGTATGTGCACT |
| | | | GTGGATCCCAGAGGTTGGCTTTGCCAACACGGTATTCATTGAGCCCATCGATG |
| | | | GTGTGAGGAACATACCTCCTGCCCGGTGGAAACTGACGTGCTACCTCTGTAAG |
| | | | CAGAAAGGCGTGGGTGCCTGCATTCAGTGCCACAAAGCAAATTGCTACACAGC |
| | | | ATTCCATGTGACGTGTGCCCAGAAGGCTGGTCTGTACATGAAGATGGAGCCTG |
| | | | TGAAGGAGCTGACTGGAGGCAGCACCACCTTCTCTGTCAGAAAGACTGCTTAC |
| | | | TGTGATGTCCACACACCTCCAGGCTGTACCCGGAGGCCTCTGAACATTTATGG |
| | | | AGATGTTGAAATGAAAAATGGTGTGTGTCGAAAAGAAAGCTCAGTCAAAACGG |
| | | | TCAGGTCTACATCCAAGGTCAGGAAAAAAGCAAAAAAGGCTAAGAAAGCACTG |
| | | | GCTGAGCCCTGCGCGGTCCTGCCGACCGTGTGTGCTCCATATATCCCCCCTCA |
| | | | GAG |
| | | | SEQ ID NO: 20 |
| Exon 2 | 157 | 129397961- | ATTAAATAGGATTGCGAATCAGGTGGCCATTCAGCGGAAGAAGCAGTTTGTGG |
| | | 129398117 | AGCGAGCCCACAGCTACTGGTTACTCAAAAGGCTGTCTAGGAATGGTGCTCCC |
| | | | CTGCTGCGGCGGCTCCAGTCCAGCCTGCAGTCCCAGAGAAACACGCAGCAG |
| | | | SEQ ID NO: 20 |
| Exon 3 | 132 | 129393071- | AGAGAAAATGATGAAGAGATGAAAGCTGCCAAAGAGAAGCTGAAGTACTGGCA |
| | | 129393202 | GCGGCTACGGCATGACCTAGAGCGTGCCCGCCTGCTGATCGAGCTGCTGCGCA |
| | | | AGCGGGAGAAACTCAAACGGGAGCAG |
| | | | SEQ ID NO: 21 |
| Exon 4 | 129 | 129392565- | GTGAAGGTGGAGCAGATGGCTATGGAGCTCCGGTTGACACCTCTGACTGTGCT |
| | | 129392693 | GCTACGCTCAGTCCTGGAGCAGCTACAGGAGAAGGACCCTGCAAAGATCTTTG |
| | | | CCCAGCCCGTGAGTCTCAAGGAG |
| | | | SEQ ID NO: 22 |
| Exon 5 | 313 | 129391817- | GTACCAGATTATTTGGATCACATTAAACATCCCATGGACTTTGCTACAATGAG |
| | | 129392129 | GAAACGGCTAGAAGCTCAAGGGTATAAAAACCTCCATGCGTTTGAGGAGGATT |
| | | | TTAATCTCATTGTAGATAACTGCATGAAGTACAATGCCAAGGACACCGTGTTT |
| | | | TATAGAGCTGCAGTGAGGCTGCGAGATCAGGGAGGTGTTGTCTTGAGGCAGGC |
| | | | CCGGCGTGAGGTGGATAGCATCGGCCTGGAAGAGGCCTCGGGAATGCACCTGC |
| | | | CTGAGCGACCCATCGCAGCCCCTCGGCGGCCCTTCTCCTGGGAAGAGG |
| | | | SEQ ID NO: 23 |
| Exon 6 | 261 | 129386345- | TGGACAGGTTGCTGGACCCAGCCAACAGGGCCCACATGAGCTTGGAGGAGCAG |
| | | 129386605 | CTGAGAGAACTACTGGACAAGTTGGACCTGACCTGCTCCATGAAGTCCAGCGG |
| | | | CTCACGGAGTAAACGGGCAAAGCTGCTCAAAAAAGAGATTGCTCTTCTCCGAA |
| | | | ACAAGCTGAGCCAGCAGCACAGCCAGACCCCATCCATAGGGGCAGGCACAGGA |
| | | | GGCTTTGAAGACGATGCTGCTCCACTGGCGCCAGACACAGGGGAGGAAG |
| | | | SEQ ID NO: 24 |
| Exon 7 | 105 | 129379748- | TCCTTCCGAGGTTGGAGACTCTACTGCAGCCAAGGAAAAGGTCGAGGAGCACA |
| | | 129379852 | TGTGGAGACTCCGAAGTGGAGGAGGAGTCCCCGGGAAAGCGCCTGGATACAG |
| | | | SEQ ID NO: 25 |
| Exon 8 | 136 | 129370502 | -GTCTCACCAATGGCTTTGGGGGTACTAGAAGCGAGCAGGAGCCAGGCAGCGGC |
| | | 129370637 | CCAGGGAGGAAAGCTGCGCCCCGACGGCGCTGTGCATCTGAGTCCAGCATTTG |
| | | | TTCCAGCAACAGCCCACTGTGTGACTCAAG |
| | | | SEQ ID NO: 26 |
| Exon 9 | 128 | 129369932- | CTTTAGTACACCCAAGTGTGGCCGAGGGAAGCCTGCGCTTGTACGAAGGCACA |
| | | 129370059 | CACTGGAAGACCGCAGTGAGCTGATATCTTGTATTGAAAATGGAAACTACGCC |
| | | | AAGGCGGCCAGGATTGCAGCTG |
| | | | SEQ ID NO: 27 |
| Exon 10 | 110 | 129368845- | AGGTGGGACAGAACAGCATGTGGATTTCCACCGATGCCGCTGCCTCCGTCCTG |
| | | 129368954 | GAGCCCCTGAAGGTGGTGTGGGCCAAGTGTAGCGGCTATCCCTCCTACCCAGC |
| | | | ACTG |
| | | | SEQ ID NO: 28 |
| Exon 11 | 155 | 129368511- | ATTATTGACCCCAAGATGCCACGAGTGCCTGGCCACCACAATGGTGTCACCAT |
| | | 129368665 | CCCCGCCCCGCCCCTGGATGTGCTGAAGATCGGTGAACACATGCAGACCAAGT |
| | | | CTGAGGAGAAGCTGTTCCTTGTTTTGTTCTTTGACAATAAGAGAAGCTG |
| | | | SEQ ID NO: 29 |
| Exon 12 | 1454 | 129366021- | GCAGTGGCTTCCCAAGTCCAAGATGGTTCCTCTTGGTGTGGATGAGACCATTG |
| | | 129367474 | ACAAACTGAAAATGATGGAAGGGAGGAACTCTAGCATCCGGAAGGCTGTGCGG |
| | | | ATTGCATTTGATCGAGCCATGAATCATCTGAGCCGAGTCCATGGGGAGCCAGC |
| | | | CAGTGACCTCAGTGACATTGACTGAGGTGGCTTCCAGCAAAAGGCAGTGGCTA |
| | | | AAGCCACAGCCAACCAGGAGCCCTGTCAATAGTGTTGATAAGCTGTACATGTT |
| | | | TGTATATTGTTCAGAACTTAACTTATTCTGATTTTCTAGGTGTAGTTCTTTAA |
| | | | TTCTTTTTCCCCCCCCCGGGAGGGGAGGTTTCACTTCCAAGTTTTCTATGAAA |
| | | | CCATCTGGTCTTGGCTTTGCAGGTGAGGAGGGTCTGTTCCGAGCAGTGTGGTG |
| | | | TGGGGTCCCACTGCAGGTGCCGAGTGCCGAGGCCTCACTTACTTCTAATCTGT |
| | | | AGGGTTTTTTTTTTTAAAGACTTTTGAATGTTTAATAATTTTGTAGATCATGC |
| | | | TCTTTACACAGAGTACCGCTTATTTAATAAGACGGGGTGTAAATTTACAATGA |
| | | | CAAATGTGTATTTTAAGAAAGAAAATGACATTATTTTGAATGGTACTTTGTGC |
| | | | AAAGAGGGAATAAATTTATGCTGTGTGCATCACTTGCAAATCACCAAAAAATG |
| | | | TCCCGCCAGCTGCTGCCGGCCAGGGCCCGTTCTCACCGTTCTGACTGCCCTGA |
| | | | GTCTCCTGTTCTGCCCTGGCTCCTGCAGGCGTGCCTCCCAGCGGGTTATTTAT |
| | | | TGTAGAAAGTGTACTCATTTGCTTTATAATGAAAAAATAAATTTGCAAAGGTA |
| | | | TATTGATATGCATTTTTATACAGGCACATAAAAACTCAACTTGGTGTGGGAGC |
| | | | AGAATGTGTTGCGAGGTTATATACATGATGGGCCTGTGTGTACTTTGATTTTT |
| | | | GTAACTTGTAATCTTTTGTTTACAATGAGGAGCTTTCTGTAACTTGTTTTAAT |
| | | | TTAGAACACTTTGGTAGCAATAGACCTTTGGATACATTTTTGTATGGTACATG |
| | | | TGATGTATATAGAATTAGTACTTTATTTTTATTTCTAAGAGGTAAAGCATTAT |
| | | | GTTAGGGGAAAAGGCAGGGTGGGTTTCCAAATTTGCATTTTTATATTAAAAAT |
| | | | AAAGTGAAGATTTGGACAGTGTGGCCCTCTCATTCCTGCATCACTAGGAGGCT |
| | | | GGGTGAGCTGTAGCCTGAGGTACATGTGGGAGCACTGAGGCCTTGAGTGGGTG |
| | | | GTGTGACCAGGTGTGACACACTTGATCTAACAGCTGACCTGGGGTGGCATTAT |
| | | | TTATTATTTTGCCTAATCATATTTTTATTTTAAAGCTAAATAGTTACTAAAAA |
| | | | TTTTAAATGTTCTTTTAAGTCTACATGTTTGTAATATCTCCATAGAAACTTGA |
| | | | AAAATAAAAAGTCTTCCTTTGGT |
| | | | SEQ ID NO: 30 |

TABLE 10
Amino acid sequence of rat Brd1 (UCSC Genome
Browser on Rat March 2012 (RGSC 5.0/rn5) Assembly);
Sequence ID NP_001101573
SEQ ID NO: 31
MRRKGRCHRGSAARHPSSPCSIKHSPTRETLTYAQAQRMVEIEIEGRLHR
ISIFDPLEIILEDDLTAQEMSECNSNKENSERPPVCLRTKRHKNNRVKKK
NEVLPSTHGTPASASALPEPKVRIVEYSPPSAPRRPPVYYKFIEKSAEEL
DNEVEYDMDEEDYAWLEIINEKRKGDCVSAVSQNMFEFLMDRFEKESYCE
NQKQGEHQSLIDEDAVCCICMDGECQNSNVILFCDMCNLAVHQECYGVPY
IPEGQWLCRHCLQSRARPADCVLCPNKGGAFKKTDDDRWGHVVCALWIPE
VGFANTVFIEPIDGVRNIPPARWKLTCYLCKQKGVGACIQCHKANCYTAF
HVTCAQKAGLYMKMEPVKELTGGSTTFSVRKTAYCDVHTPPGCTRRPLNI
YGDVEMKNGVCRKESSVKIVRSTSKVRKKAKKAKKALAEPCAVLPTVCAP
YIPPQRLNRIANQVAIQRKKQFVERAHSYWLLKRLSRNGAPLLRRLQSSL
QSQRNTQQRENDEEMKAAKEKLKYWQRLRHDLERARLLIELLRKREKLKR
EQVKVEQMAMELRLTPLTVLLRSVLEQLQEKDPAKIFAQPVSLKEVPDYL
DHIKHPMDFATMRKRLEAQGYKNLHAFEEDFNLIVDNCMKYNAKDTVFYR
AAVRLRDQGGVVLRQARREVDSIGLEEASGMHLPERPIAAPRRPFSWEEV
DRLLDPANRAHMSLEEQLRELLDKLDLICSMKSSGSRSKRAKLLKKEIAL
LRNKLSQQHSQTPSIGAGIGGFEDDAAPLAPDTGEEVLPRLETLLQPRKR
SRSTCGDSEVEEESPGKRLDTGLTNGFGGIRSEQEPGSGPGRKAAPRRAC
ASESSICSSNSPLCDSSFSTPKCGRGKPALVRRHTLEDRSELISCIENGN
YAKAARIAAEVGQNSMWISTDAAASVLEPLKVVWAKCSGYPSYPALIIDP
KMPRVPGHHNGVTIPAPPLDVLKIGEHMQTKSEEKLFLVLFFDNKRSWQW
LPKSKMVPLGVDETIDKLKMMEGRNSSIRKAVRIAFDRAMNHLSRVHGEP
ASDLSDID

TABLE 11
levels of WT BRD1 mRNA as determined by quantitative RT PCR

| | Brain | Liver | Kidney | Heart | Muscle | Testis | Ovary |
|---|---|---|---|---|---|---|---|
| W | 100% | 100% | 100% | 100% | 100% | 100% | 100% |
| R | 34% | 50% | 57% | 55% | 55% | 41% | 48% |

| | Brain | Liver | Kidney | Heart |
|---|---|---|---|---|
| FW | 100% | 100% | 100% | 100% |
| LC | 54% | Not sign. | 107% | Not sign. |
| | | changed | | Changed |
| FC | 7% | Not sign. | 85% | Not sign. |
| | | changed | | Changed |

R mice are derived from crossing between R and W mice resulting in the production of W and R offspring. Measurements were performed in such R mice and their W littermates for comparison.
LC and FC mice are derived from crossing between F and LC mice resulting in the production of LW, LC, FW and FC offspring. Measurements were performed in such LC and FC mice and their FW littermates for comparison. FW mice are homozygous for the conditional allele but do not carry the Cre allele. Thus, they are expected to have the same level of WT BRD1 mRNA as the W mice.

TABLE 12
Sequence of targeting vector (pBrd1 FINAL Seq (UP257))

BASE COUNT 4401: a 4710: c 4935: g 5000: t 0 n
cggccgcatgttcccagcctgaactcagtgggtgggctgctctgcttggagagtttcttaaggttgagtgt
gcccagcgctggtggcgccagctgtgagcgcaggctttgacctccagtccatccagtcggcagcatctcag
ctggcagtggtcagtagccgtcactgtgtgtgtagacaggagcacaggggcaaagtggttaaagttttgtt
cacctgtgtctgctttagacgttgaacctggtgactcttgtggaggatgaaatctgtagttagttgaaggt
tatgaactgttttcagggacaggctcagggagagaactgcagtgtcctgtctagttttctaaatgcaaaca
cgtttaaatatccctttcgaagctaaactctcagttttttcatgttttagattaaataggattgcgaatca
ggtggccattcagcggaagaagcagtttgtggagcgagcccacagctactggttgctcaaaaggctgtcta
ggaatggtgctcccctgttgcggcggctccagtccagcctgcagtcccagagaaacacgcagcaggtatgt
gtgctcttctgcttttcagttacatgggctgccccccccccccccccccaggctggatgtgctgctgaccc
taagccccgggccttaaactctactaaactgcaggttattcgggtggctcctgtatcctcaaggtttgctg
tgactttggggttgagttgttctttactctgacaagtgtctgctctgtgcccagtcctctgtcagttccag
ggaaggaagggactgctcagagaacctggctcaacttcagctgcatgcatagtcaagacagagagggaggc
ctgatgaagtctatgcagttcctctacacattgcccaaaaactaggtgtctggtaatacctgctggttcca
ctgggaggagctagtcatttcatctgtaaaatagcaaccaactttaatggaagtttaagtctgtagaatcc
tgtgactccccatggctgtcacaggcatggctgtgaatgagcttagggttctcatcctgtatcctggctgt
cagatgagcagtggtactggagccctgttgtatggatcagacccttgtgtctgcaggttaccaagtattgc
tcttctgggagttaacaacttgctggactctgtctgggtctgatctgaatggaaggggcctccccagtgtt
agatcttctgttgccttctacaagccaacgttgtctattattcactgaggacacatacctccttggaggct
actggaatgtcctagttaggggtttccattgctgagaagagacacagtgaaggcaactcttacaagggaca
acatttaactgggctgacttcacaggttcagaggttcagtccattatcatcaggccggaagcatggcagtg
tccaggcaagagggtcttagagctattggtcatgaagtggggaagtgtttggtaaccctgggcactgggag
gaatgattgcctatgtgacggtaggtagcagtgttggaaagagaagtccgggagtgggtggctacttctga
gcttccccttctcagaagtctcttcctgggaagaattccagcattgatttctatgtagcaaagcagactgc
ttcggaatcgtaccgggacagcgggtttacagatgggatgatctgtgtagatttgtgtacagggtcctgtc
ttcgtgagcctatagcatggtggagtgcagacagtggctcaattacccatgaccttttaaagatgaaaacc
aggccaggagcaaaccacttgagttttgcctatccctaaatatacaagctcaggcctgttggaaacctatc
caaaatgctcttatgttactcagaagtctgtttctaaggagcaggaagctgtccagatgatgctaggatat
ttggttccttttttctttgtttatttggagatagggtcaacctgaatcttgctatatatgctggccttgaa
ctcgcagaactcagtctctgcctcctaagagttgaaattagaggtgcacatggccacagctggcaatgttt
gtgaactcccctttccatgtatttgctccctttgcctatatgtgatgagtgaggtacactgtgcattactg
tgggcgctaaagtgtgcatcaggacagaccatgccattcccatcctgtgctgccattttcataccatgaag
agtggctgtttatacagttgggttggtgacactttgctccgagaccctccatctttgaccgttgtgctggt
agcttgagttgcagtctctgctgtggtgtcactgggccatgagaggcaaagctgtccagagagaaggggct
cctgtgtgttctacagctgcaaggcagcactttgcttgtggctggcagatgtagatatttatttaggttac
tgtctagcagtagtgcagaaggacaaacttttgggtaggtcattttccatccctttataatagggacaggc
aggacatatggcttactgtgaggaggtaatcccatacattttccacagagtagagagtaggggatagcttt
ggataatgacttgtgttggatgagaaaccaagtcttggacaggttcactctggggaggcagaaagagaagt
atggggtggcaggaaaggagatctgggttgggggagcagagctctggggaacgtggttggataagatgcat
ggaattctgagaggatgaggcatgttgaatttcttggcaagtgactggaaaacctggtgctttgtagatag
ggctctggtcttgtttggtgttccttggttgctatcaagggatgtgtgctatccctgtggcagtaggtctt
gtccccgtacatttgtgaagtagtaagagtaccgtggttagccttgaggggcttactaggcttctggctgc
ttctcctgcttagaactctgagctgcttctcctgcttagaactctgagcagcagctcaaggatccacctcc
ctctggtgctgcagagctaggctgcttccctgctactgtctgtctcttggtgcttccactttgttggctag
gatagagaagtgctggtgcaggatgctgaccaagtgctatttggtgtactgcctgagaaggcagctgtgac
tggcaactacagtgcccacgcctagaactgaccgcggctcgagcctaggataacttcgtataatgtatgct
atacgaagttatggtaaccgaagttcctatactttctagagaataggaacttcggaataggaacttcttat
aatctagaactagtggatcgatccacgattcgagggcccctgcaggtcaattctaccgggtaggggaggcg
cttttcccaaggcagtctggagcatgcgctttagcagccccgctgggcacttggcgctacacaagtggcct
ctggcctcgcacacattccacatccaccggtaggcgccaaccggctccgttctttggtggccccttcgcgc
caccttctactcctcccctagtcaggaagttcccccccgccccgcagctcgcgtcgtgcaggacgtgacaa
atggaagtagcacgtctcactagtctcgtgcagatggacagcaccgctgagcaatggaagcgggtaggcct
ttggggcagcggccaatagcagctttgctccttcgctttctgggctcagaggctgggaaggggtgggtccg
ggggcgggctcaggggcgggctcaggggcggggcgggcgcccgaaggtcctccggaggcccggcattctgc
acgcttcaaaagcgcacgtctgccgcgctgttctcctcttcctcatctccgggcctttcgacctgcagcca
atatgggatcggccattgaacaagatggattgcacgcaggttctccggccgcttgggtggagaggctattc
ggctatgactgggcacaacagacaatcggctgctctgatgccgccgtgttccggctgtcagcgcaggggcg
cccggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggactgg
ctgctattgggcgaagtgccggggcaggatctcctgtcatctcaccttgctcctgccgagaaagtatccat
catggctgatgcaatgcggcggctgcatacgcttgatccggctacctgcccattcgaccaccaagcgaaac
atcgcatcgagcgagcacgtactcggatggaagccggtcttgtcgatcaggatgatctggacgaagagcat
caggggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgactgtg
gccggctgggtgtggcggaccgctatcaggacatagcgttggctacccgtgatattgctgaagagcttggc
ggcgaatgggctgaccgcttcctcgtgctttacggtatcgccgctcccgattcgcagcgcatcgccttcta
tcgccttcttgacgagttcttctgaggggatcgatccgctgtaagtctgcagaaattgatgatctattaaa
caataaagatgtccactaaaatggaagtttttcctgtcatactttgttaagaagggtgagaacagagtacc
tacattttgaatggaaggattggagctacgggggtgggggtggggtgggattagataaatgcctgctcttt
actgaaggctctttactattgctttatgataatgtttcatagttggatatcataatttaaacaagcaaaac
caaattaagggccagctcattcctcccactcatgatctatagatctatagatctctcgtgggatcattgtt
tttctcttgattcccactttgtggttctaagtactgtggtttccaaatgtgtcagtttcatagcctgaaga
acgagatcagcagcctctgttccacatacacttcattctcagtattgttttgccaagttctaattccatca
gaagctgactctagatcctgcaggaattaattcatatgaagttcctatactttctagagaataggaacttc
ggaataggaacttcaaaatgtcgcggcgcgccacctgcataatattccgccgccagtaagggtagcttagg
tttgtacctcttgtgtatctcctttctcgtactccctccattcctgcctcctggagtcaagccaagacccc
gttgtgtcgactagaccttcctgtcccattgtcacagcacatttatagggactgggtacatttatagagac
tagatcccaggtcctgctacccttttagtcttacctgttggatgagcttgttagatccctggcaggaagaa
ctttggggtgtgactgatggaaagtttcctctaattttctcagagagaaaatgatgaagagatgaaagctg
ccaaagagaagctaaagtactggcagcggctgcgacatgacctagagcgtgcacgcctgctaattgagctg
ctgcgcaagcgggagaaactcaagagagagcaggtgaggagggaggcccttgggttctgccaccctctggg
ctgtccctggatagacgtcttgctgccgtcatggagtgctctggagtggcccctgtgtacctgctgagtta
gtgctgtocccaccctgtagcatatcatatccctaccctatagttggtcctgtggtacctctgtgttgtcc
ttttcgattagccacctctggagtatacggggtcttaaaggagacccctgccgtggaagaagtacatgtcc
ttgcacagagaaggcagctttgtggtgggatggtagctggcacgtaggctgctctgtgctgctggttcaag
tggcgcttctgtgattgtgcagtacgtggaggtgcggtgatctccaggagaggtgtccctacactcctctg
gagacagtgtatgcagaggtgtccctgcatcttctagagacagtgtatgcatgctgttgttgccaggtgaa
ggtggagcagatggctatggagctccggctgacgccgctaactgtgctgctacgctcagtcctggagcagc
tacaggagaaggaccctgcaaagatctttgcccagcccgtgagtctcaaggaggtgcgtgtccctgcgact
gagctcttcggctgcttgcttaggaagcatgcaactggggagaggttacctgcattcttaattctcattag
ttagtagttaatgaatttttggtgaatagtattttaattataaaagattgtacctcgttgtaaagcactga
aagtgcataggtgaaaatttctacttagaacttaacaattggtgatgatagcccccctggtaccccatctg
tttgtacttttagttgaagtaggttgggagggtctctgcagtgattgggcttagtttgtattggcttagtg
ttgttatgtgaaattagtttcaggtgtggttgattttgtaaatgtttattttccctcctaaaattaggtac
cagattatttggatcacattaaacaccccatggactttgctacaatgaggaaacggctagaagctcaaggg
tataaaaacctccatgcctttgaggaggattttaatctcattgtagataactgcatgaagtacaatgccaa
ggacaccgtgttttatagagctgcagtgaggctgcgcgaccagggaggggttgtcctgaggcaggcccggc
gagaggtggagagcattggcctggaagaggcctcgggaatgcacctgcctgagcgacccatcgcagcccct
cggcggcccttctcctgggaagagggtaagaactgtatccaggaggacagcggatgctttttctctcagac
tgcactcactaagactccagcatgccggccgagtgagtgctcctgaggtgcatgcgccttgtatgggcacc
acgtgggcctcgccatgttttcacatacccactgcgagaaacacatatctaggtgctgaaggccccgaaga
cactatagttgaggatgcatccccaaagggtctgaccttgcttctgaggtcatgcattgagaaggcagcta
ttcattagttgtcatatttcagctgagaagcaaaagcaggagctggccggcctcgacataacttcgtataa
tgtatgctatacgaagttataagcttaaggaattcgctagcatgcatgttaacggatccttaattaaaatg
ttggctgtgcctctgatcctctctctgggatgcttgcaggtgtttattgagggcccagccttagcctgctt
ctaggacatggcctaacccttctaactctccagggcaagcttgtactctgggccccaccgtgcacatgctg
ttgtgctcttcattaatttcttccaagtaaggagctgtttttaaagataaggtctcagtgggtagtcttga
ctggcctggaactcaaaatgtggatcaggctggcttggacttgacagaagtccacctgcttctgcctcctg
agtgctgtggttaaagatgtgcattaccataccacatctggcctccaatcatttcttgtaagcttcttgcc
cctggattgtttattctgtaggtaaatgtctacagtaggtgaatggggtttggtggtcaaccttggaactt
ttattcacaaaacccaagatcctatgttcctgatttgacctaccttttctcctgctattgactgttcagga
aaatggtggaatcgtacggacttaggttttatccggtacgtttccttctcctggatgaccagctgcctggt
cactgtggcctgactcgtgaggtcagagcccttggagactcctcacttctggcttcctgtgtatctgaccc
agagaaactgtctgtctcaggcatctctagggcatacaggatagggttgaattctttttttctcaagatag
gatgtagtgccacactcaggaagctaagacaggaggttcaccacaaatttaaggtcagtctaaactatagt
gatttctaggctagtgagttacaccctgagaccctgcctaaaaaccaaaactgatcctaacagtataatta
gaaagaaaagcagccaggccagagtgtggcttagtagtgtttctttgcatgcacaacatttgggttcaatg
tcaacacagcataaactgggttgatacaaagattagaatttaaaggtcatattggctatagagtgaattaa
ggctagcctgggttacatgagaccttgctttgaaaaatagatatgcatgcacccacacaggtgacaagatt
tctgaaaccctagataggtccagcaggaactgagcctgatagccaccaggattacagagcgactctcagat
cttcacctgcatccatgttcttttctccagattgtgtgggaggcaagggtgggctccagcctcatctgttg
tggccgtgactgtgctttgggtggtatcggctgccctgagaagcagaggagcccagtgacatctgggagtc
tttgaccccacagcttctgattctcgtgctctgtagatgggcagggctcagaggcctcacagttgagattc
caggaaactggctttgtcattgctaaataaatttctgtgccagactttttgccaaaaaggaaagtaataat
gaaaagtacaaatttatttcttactcagtgattgcagtagaaagcatgacctgtggcagggtgagctctgg
gtactctgccgctgtcttgagcctgcagtaaggaagatacttgtcttagttagggtttttctgttgtgagc
agacatcatgaccaaggcaagtcttacaaggacaacatttagttggggctggcttacaggttctgaagttc
agtccattatcatcaaggtgaaaacatggcagcatccagacaggcatggtgcaggaggagctgagagttct
acatcttcatctgaaggctgctagcagaatattggctcccaagcagctaggagcccacacccacaaggcca
tacctcccaaaagtgccactccctgagctgaacatataatatacaaccattacattccaccccctggccct
cataggcttgtccaaacataagcctatgggagccatacctacacatagcataatgcaaaatacatttagtc
cgacttcaaaagcccccatagtctatggcagtctcaacaataatcgtccaataacttaactgtaatcccca
aagcaagacaggaagccagctgggctctgcatctccatgtctgatgtcttcagatcttctattcctttttc
atctttgttgactgcaacaaacttctttctcctgggctggttctactccctggtagcatagcagctttcct
tagcagatagtccaactaccactctggtatctccaaggcagcttcttgttttaatgtctgggcctcctctc
caaggtgacgtcacttccccagctctgccctcggtagctctaagctcaggttgatccctccactgccgctg
ctgctcttggtggccatcatctccaatacactgggggcttccgctgcaactagagcctctctaggctctct
tcatggtgccaagcctcaactcctttgcatggccccttcagtcctgggccatcatctgcaaccgaggctgc
actttgatcagtgatcttccgcctcagctgctcttcatggccccttcatgcctcaaggccagtgccacctg
ggggaccattgcagtcacccagcatagctgcagcatgaggtgcaaccttggctgtctctggaacacagctt
cttggtgctcagaaaacacttccagtgatgctggttgtcgtcatgatttatttattatatgagtacacagt
tctcttcagacacaccagaaagagggtattgggcccctgttacagatggtcgtgagccaccatgtggttgc
tgggaattgaactcaggacctctggaagagcagtcagtgctcttaaccacagagccatctctccagccctg
ccggtctcttaatcactgctaatgccttagctcccgctaaccagcatcagctgtcccaggagtctttctcc
tcgtgattataaagccagagacacatggccgaagctgcttgctggagctggaacatggcccctagttctat
tgcgtcatcactagcttccagctttcgcgctccttcaaggcctaagtttgtcacgtggggatcttgctcag
aactctgagatatgcaagcctgactcctgggattagaggtgtgtaccagcacgcccggaattaagcttttc
ttcacctacaacttgatctgtccttgaaagtagagatctgcctgcctttgcctccaggaattaaaaagctt
gttctgcccagtatagaccaaaacttaactgggtgggatcttgccccaaggtcactagtcccttaattcaa
actaatgtccttgaacacattcagctccattcacttccagtattcctttctaaccttgcaatgcttattca
catgctcttcctgagaacaaagtctacgatgggcctttctaaggcttcctttgtcattgtaattaacctga
gcctccttagcctcaggcagactcttcagccaagggcaaaaatagctacttcttcaccaaactacaaaaac
aaggctctagaccacataactgaaattcctcactgaaacctcttgtgctgggtctacacagttccgattac
tcacagcaacaaagtgttccatagtccagctaggatagaccatgaagccccacttgaaacattctgtggcc
ttccaaatcccaagttccccaacctacattcttataagcaaaaacacggtcaggcctattaccgcaatatc
tcagtccctggtgccacctgtcttagagtttttctgctgtgagcagacaccatgaccaaggcaagtcttct
aaggacaacatttaattggggctggcttacaggttccgaagttcagtccattatcaaggtggaaacatggc
agcatccagacaggcatggtacaggaggagctaagagttctacgtcttctgaaggctgctagcagagtact
gactcccaggcagctaggagcccacccatgaggccacacctactccaacagggctacacctcctagcattg
ccgctccctaagcagagcatatacaaaccgcaatactggccctgttgaaagagaagccaaccagcagagcc
tgcaggtctagcactcaggttgaggagggaggattacaagtttgaggccagcctggactcagcaagcacaa
aacagaagaaaggaggcttgagaagttgagtggtggtttttgttgcggtgactgtaagccagttggacagt
gtttgtcgtgtcccactgctaagttagtgctgtttagacagggcgctaatgagtctcctaggccagctacc
aggtctgggcagggctcatttatggtaggtgtctctgttggccctgctgttcctttggttttatcttcgca
tagattaaataattttttggctatttcactaatttaagtcctgcagtcaatgttcctagagtctggggaga
cctgcggactctgcagcctagtttccttttggtcatgatgtatgtgcaagaacttgagctaggatgatgtt
cacaatgtataaacagtccatgtgaacatatttacacacacgcagcgtctgtcagtagtccatcttgcgtc
tatgttggtgcactcagacatgtctggtggtctttgtgcctctcactttttacagagcaggactgagttgg
gtcttagtccaggaaaagccatgtgtgttacccacatctcctctgctacggccacactagtcctttgtgta
ctactgactgaaggagtgtcttgtctctttttttccctctttgtgacaacagccttgtcataggttcagaa
tcagggtagagaggagtatgtatggcaccaaatggtgaaattggaacacttgggaggcaggggcaggcaga
tctctgagttcaaggtcagcctgttacagaatgagttgcaggacagcctgggttacccagagaaacactgt
ctcaaaaacaaacaaataaaacaaaacaaacccaagaagctaaataaacaaacaaagattaaatgaatttg
aagcctgcgctttggccgtgggcaggcccaggcacatagttaagacagatgtgttgttatcagaggcggcc
atgaatccgaatcctgtggctaatgatacgtgtttttggttcagtggacaggttgctggacccagccaaca
gggcccacatgagcttggaggagcagctgagagaacttctggacaagttggacctgacctgctccatgaag
tccagcggctcacggagtaaacgggcaaagctgcttaaaaaagagattgctcttctccgaaacaagctgag
ccagcagcacagccaggctccgcccacaggggcaggcacgggaggctttgaagatgaggctgctccactgg
ccccggacacagcggaggaaggtaagcatggggtaggagggccatacctcacgggctcggggctctcttga
caggcttaaatgatgctctgtagtaatgatgagcttgtacattttgaaggtcacggaactcttggttactg
gatattcctgctaggctttttttgatgctctttgaaaggatgttttggtgtgttctgtctgctgtattttg
gcacttagtttacaagcttaaaggaacagaatgagattttcttttaactcgagcttgaaagacttagaagg
aatagtttagatccaatacagtgttgaaggtggcttctatggtgggaatggcaataacttagttgtatttt
gttaattgaggcagagtattatgtgagtagacaccctagaattgtttttaccttgtctacgtaggtcagag
gacagctagttggagttggttttcctggcatcttagcacgcttggggatcaagcgcaggtggttaggcctt
gtaagcacctctgcccttagctaagccctcctgcggctggagttaggaaaggaggactggctagagaacag
cccagccttgggctgggcatggtgggaggagtctgacgtgcacagacctgttcccagactctccctccacc
tcaggcctttcctgtggctcaccttcagtggacactgtcttattctggcagcgtgagtgacttctggggaa
agagctggatagctgagatgttagggtggagaggaaggaagggaggaagtacagaagaggctgtctgcccc
gtgcgatccacgagatgagcaggtcattgtgtggagggagggaggcttctgtgtgtggtgcatctaactgg
catgtttgatggtacaagcaccctttagtccacttgtcttgacatcaccacatttcaactccatgaaatgg
aaagaaaaataagacctacttcttctgccactgctattagcagcttgacttaggatctccctgtgcatttt
ttttttctgccccatccaaataagaaaaacattaacacaagaccattgtcaccatagtttgcatttttttg
atctgtatggctgcctgtcttagtagatgtgactttgccctattcctcagagtgacatggtttcagtatgt
ttatgccatgttaaatttagtcttataattttaacagttggtgacaatcttctaacccactttccccttct
ctggttgcttcttttatatggttatgctaggcaaccagcagaagctagggccaacaccagagttctcctgg
ccttacatccttctagtgtgttcacttgtaaactcacaaacacccttggccttgccattaggtaacgttta
aacagtaacgctagggataacagggtaatataatcgagctgcaggattcgagggccccggcaggtcaattc
taccgggtaggggaggcgcttttcccaaggcagtctggagcatgcgctttagcagccccgctgggcacttg
gcgctacacaagtggcctctggcctcgcacacattccacatccaccggtaggcgccaaccggctccgttct
ttggtggccccttcgcgccaccttctactcctcccctagtcaggaagttcccccccgccccgcagctcgcg
tcgtgcaggacgtgacaaatggaagtagcacgtctcactagtctcgtgcagatggacagcaccgctgagca
atggaagcgggtaggcctttggggcagcggcCaatagcagctttgctccttcgctttctgggctcagaggc
tgggaaggggtgggtccgggggcgggctcaggggcgggctcaggggcggggcgggcgcccgaaggtcctcc
ggaggcccggcattctgcacgcttcaaaagcgcacgtctgccgcgctgttctcctcttcctcatctccggg
cctttcgacctgcagccaatgcaccgtccttgccatcatggcctcgtaccccggccatcaacacgcgtctg
cgttcgaccaggctgcgcgttctcgcggccatagcaaccgacgtacggcgttgcgccctcgccggcagcaa
gaagccacggaagtccgcccggagcagaaaatgcccacgctactgcgggtttatatagacggtccccacgg
gatggggaaaaccaccaccacgcaactgctggtggccctgggttcgcgcgacgatatcgtctacgtacccg
agccgatgacttactggcgggtgctgggggcttccgagacaatcgcgaacatctacaccacacaacaccgc
ctcgaccagggtgagatatcggccggggacgcggcggtggtaatgacaagcgcccagataacaatgggcat
gccttatgccgtgaccgacgccgttctggctcctcatatcgggggggaggctgggagctcacatgccccgc
ccccggccctcaccctcatcttcgaccgccatcccatcgccgccctcctgtgctacccggccgcgcggtac
cttatgggcagcatgaccccccaggccgtgctggcgttcgtggccctcatcccgccgaccttgcccggcac
caacatcgtgcttggggcccttccggaggacagacacatcgaccgcctggccaaacgccagcgccccggcg
agcggctggacctggctatgctggctgcgattcgccgcgtttacgggctacttgccaatacggtgcggtat
ctgcagtgcggcgggtcgtggcgggaggactggggacagctttcggggacggccgtgccgccccagggtgc
cgagccccagagcaacgcgggcccacgaccccatatcggggacacgttatttaccctgtttcgggcccccg
agttgctggcccccaacggcgacctgtataacgtgtttgcctgggccttggacgtcttggccaaacgcctc
cgttccatgcacgtctttatcctggattacgaccaatcgcccgccggctgccgggacgccctgctgcaact
tacctccgggatggtccagacccacgtcaccacccccggctccataccgacgatatgcgacctggcgcgca
cgtttgcccgggagatgggggaggctaactgaggggatcgatccgtcctgtaagtctgcagaaattgatga
tctattaaacaataaagatgtccactaaaatggaagtttttcctgtcatactttgttaagaagggtgagaa
cagagtacctacattttgaatggaaggattggagctacgggggtgggggtggggtgggattagataaatgc
ctgctctttactgaaggctctttactattgctttatgataatgtttcatagttggatatcataatttaaac
aagcaaaaccaaattaagggccagctcattcctcccactcatgatctatagatctatagatctctcgtggg
atcattgtttttctcttgattcccactttgtggttctaagtactgtggtttccaaatgtgtcagtttcata
gcctgaagaacgagatcagcagcctctgttccacatacacttcattctcagtattgttttgccaagttcta
attccatcagaagctgactctaggccggacgcccgggcgaccggccgagctccaattcgccctatagtgag
tcgtattacaattcactggccgtcgttttacaacgtcgtgactgggaaaaccctggcgttacccaacttaa
tcgccttgcagcacatccccctttcgccagctggcgtaatagcgaagaggcccgcaccgatcgcccttccc
aacagttgcgcagcctgaatggcgaatgggacgcgccctgtagcggcgcattaagcgcggcgggtgtggtg
gttacgcgcagcgtgaccgctacacttgccagcgccctagcgcccgctcctttcgctttcttcccttcctt
tctcgccacgttcgccggctttccccgtcaagctctaaatcgggggctccctttagggttccgatttagtg
ctttacggcacctcgaccccaaaaaacttgattagggtgatggttcacgtagtgggccatcgccctgatag
acggtttttcgccctttgacgttggagtccacgttctttaatagtggactcttgttccaaactggaacaac
actcaaccctatctcggtctattcttttgatttataagggattttgccgatttcggcctattggttaaaaa
atgagctgatttaacaaaaatttaacgcgaattttaacaaaatattaacgcttacaatttaggtggcactt
ttcggggaaatgtgcgcggaacccctatttgtttatttttctaaatacattcaaatatgtatccgctcatg
agacaataaccctgataaatgcttcaataatattgaaaaaggaagagtatgagtattcaacatttccgtgt
cgcccttattcccttttttgcggcattttgccttcctgtttttgctcacccagaaacgctggtgaaagtaa
aagatgctgaagatcagttgggtgcacgagtgggttacatcgaactggatctcaacagcggtaagatcctt
gagagttttcgccccgaagaacgttttccaatgatgagcacttttaaagttctgctatgtggcgcggtatt
atcccgtattgacgccgggcaagagcaactcggtcgccgcatacactattctcagaatgacttggttgagt
actcaccagtcacagaaaagcatcttacggatggcatgacagtaagagaattatgcagtgctgccataacc
atgagtgataacactgcggccaacttacttctgacaacgatcggaggaccgaaggagctaaccgctttttt
gcacaacatgggggatcatgtaactcgccttgatcgttgggaaccggagctgaatgaagccataccaaacg
acgagcgtgacaccacgatgcctgtagcaatggcaacaacgttgcgcaaactattaactggcgaactactt
actctagcttcccggcaacaattaatagactggatggaggcggataaagttgcaggaccacttctgcgctc
ggcccttccggctggctggtttattgctgataaatctggagccggtgagcgtgggtctcgcggtatcattg
cagcactggggccagatggtaagccctcccgtatcgtagttatctacacgacggggagtcaggcaactatg
gatgaacgaaatagacagatcgctgagataggtgcctcactgattaagcattggtaactgtcagaccaagt
ttactcatatatactttagattgatttaaaacttcatttttaatttaaaaggatctaggtgaagatccttt
ttgataatctcatgaccaaaatcccttaacgtgagttttcgttccactgagcgtcagaccccgtagaaaag
atcaaaggatcttcttgagatcctttttttctgcgcgtaatctgctgcttgcaaacaaaaaaaccaccgct
accagcggtggtttgtttgccggatcaagagctaccaactctttttccgaaggtaactggcttcagcagag
cgcagataccaaatactgtccttctagtgtagccgtagttaggccaccacttcaagaactctgtagcaccg
cctacatacctcgctctgctaatcctgttaccagtggctgctgccagtggcgataagtcgtgtcttaccgg
gttggactcaagacgatagttaccggataaggcgcagcggtcgggctgaacggggggttcgtgcacacagc
ccagcttggagcgaacgacctacaccgaactgagatacctacagcgtgagctatgagaaagcgccacgctt
cccgaagggagaaaggcggacaggtatccggtaagcggcagggtcggaacaggagagcgcacgagggagct
tccagggggaaacgcctggtatctttatagtcctgtcgggtttcgccacctctgacttgagcgtcgatttt
tgtgatgctcgtcaggggggcggagcctatggaaaaacgccagcaacgcggcctttttacggttcctggcc
ttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcgg
aagagcgcccaatacgcaaaccgcctctccccgcgcgttggccgattcattaatgcagctggcacgacagg
tttcccgactggaaagcgggcagtgagcgcaacgcaattaatgtgagttagctcactcattaggcacccca
ggctttacactttatgcttccggctcgtatgttgtgtggaattgtgagcggataacaatttcacacaggaa
acagctatgaccatgattacgccaagcgcgcaattaaccctcactaaagggaacaaaagctgtcgagatct
agatatcgatggccatag
SEQ ID NO: 32

TABLE 13

| | Brd1 | Brd1 | Brd1 |
|---|---|---|---|
| ESC Clone | 2323 A-F7 | 2323 A-B8 | 2323 A-D1 |
| Karyotype 40 XY | n/a | n/a | n/a |
| Transferred blastocysts | 50 | 50 | 54 |
| Transfers | 3 | 3 | 3 |
| Litters | 3 | 3 | 3 |
| Pups born | 14 | 8 | 24 |
| Chimeric Pups | 12 | 4 | 15 |
| 50% chimeric male pups | 0 | 2 | 9 |

TABLE 14
Weaned Brd1 2323 A-F7

| ID | Sex | DOB | Chimerism [%] | Status |
|---|---|---|---|---|
| 120543 | f | 1 May 2008 | 25-50 | sacrificed |
| 120544 | f | 1 May 2008 | 25-50 | dead |
| 120545 | f | 1 May 2008 | 25-50 | sacrificed |

TABLE 15
Weaned Brd1 2323 A-B8

| ID | Sex | DOB | Chimerism [%] | Status |
|---|---|---|---|---|
| 119820 | m | 27 Mar. 2008 | 50-75 | sacrificed |
| 119821 | m | 27 Mar. 2008 | 50-75 | sacrificed |
| 119822 | m | 27 Mar. 2008 | 25-50 | sacrificed |

TABLE 16
Weaned Brd1 2323 A-D1

| ID | Sex | DOB | Chimerism [%] | States |
|---|---|---|---|---|
| 123153 | m | 2 Aug. 2008 | >75 | dead |
| 123154 | m | 2 Aug. 2008 | >75 | sacrificed |
| 123155 | m | 2 Aug. 2008 | 50-75 | sacrificed |
| 123156 | m | 2 Aug. 2008 | 50-75 | sacrificed |
| 123157 | m | 2 Aug. 2008 | 25-50 | sacrificed |
| 123158 | m | 2 Aug. 2008 | <25 | sacrificed |
| 123159 | m | 2 Aug. 2008 | 100 | dead |
| 123160 | m | 2 Aug. 2008 | >75 | sacrificed |
| 123161 | m | 2 Aug. 2008 | 50-75 | sacrificed |
| 123162 | m | 2 Aug. 2008 | 50-75 | sacrificed |
| 123163 | m | 2 Aug. 2008 | 25-50 | sacrificed |
| 123164 | m | 2 Aug. 2008 | 25-50 | sacrificed |
| 123165 | m | 2 Aug. 2008 | 25-50 | dead |
| 123166 | m | 2 Aug. 2008 | 50-75 | sacrificed |
| 123167 | m | 2 Aug. 2008 | 25-50 | sacrificed |
| 123168 | m | 2 Aug. 2008 | 25-50 | sacrificed |

TABLE 17
Chimera Breeding Clone Brd1 2323 A-B8
x Brd1 2323 A-B8 x FLP deleter

| ID | Breeding # | Setup | Stop | DOB pups | # born | # germline | # weaned | # typed |
|---|---|---|---|---|---|---|---|---|
| 119820 | 7566 | 15.05.2008 | 25.08.2008 | 06.06.2008 | 11 | 0 | | |
| | | | | 09.07.2008 | 8 | 0 | | |
| | | | | 14.07.2008 | 3 | 0 | | |
| | | | | 01.08.2008 | 14 | 0 | | |
| | | | | 28.08.2008 | 17 | 0 | | |
| 119821 | 7567 | 15.05.2008 | 25.08.2008 | 09.06.2008 | 4 | 0 | | |
| | | | | 09.07.2008 | 10 | 0 | | |
| | | | | 01.08.2008 | 11 | 0 | | |
| | | | | 27.08.2008 | 10 | 0 | | |
| 119822 | 7568 | 21.05.2008 | 06.08.2008 | | | | | |

TABLE 18
Chimera Breeding Clone Brd1 2323 A-D1
x Brd1 2323 A-D1 x FLP deleter

| ID | Breeding # | Setup | Stop | DOB pups | # born | # germline | # weaned | # typed |
|---|---|---|---|---|---|---|---|---|
| 123153 | 8177 | 02.10.2008 | 16.12.2008 | 01.12.2008 | 3 | 2 | 2 | 2 |
| | | | | 31.12.2008 | 1 | 0 | | |
| 123154 | 8178 | 02.10.2008 | 22.01.2009 | 16.12.2008 | 4 | 1 | 1 | 1 |
| | | | | 08.01.2009 | 7 | 7 | 7 | 7 |
| 123155 | 8179 | 02.10.2008 | 22.01.2009 | 26.10.2008 | 5 | 5 | 4 | 4 |
| | | | | 05.12.2008 | 6 | 6 | 6 | 6 |
| | | | | 04.01.2009 | 8 | 8 | 7 | 7 |
| | | | | 07.02.2009 | 8 | 8 | 8 | 8 |

TABLE 19
Genotyping results
Results for: Brd1 2323 A-D1 × FLP deleter
Line: A-D1

| ID | Sex | Loc, Mut 1 | Loc, Mut 2 | Loc, Mut 3 | Status |
|---|---|---|---|---|---|
| Breeding ID: 8178 | | | | | |
| Date: 8 Jan. 2009 | | | | | |
| 127104 | m | Brd1 W | Tg (ACTB-Flpe) W | — | sacrificed |
| 127105 | m | Brd1 cond/+ | Tg (ACTB-Flpe) tg/+ | — | sacrificed |
| 127106 | f | Brd1 W | Tg (ACTB-Flpe) tg/+ | — | sacrificed |
| 127107 | f | Brd1 cond/+ | Tg (ACTB-Flpe) tg/+ | — | Backup |
| 127108 | f | Brd1 W | Tg (ACTB-Flpe) tg/+ | — | sacrificed |
| 127109 | f | Brd1 cond/+ | Tg (ACTB-Flpe) tg/+ | — | Backup |
| 127110 | f | Brd1 W | Tg (ACTB-Flpe) tg/+ | — | sacrificed |
| Date: 16 Dec. 2008 | | | | | |
| 126461 | m | Brd1 cond/+ | Tg (ACTB-Flpe) tg/+ | — | sacrificed |
| Breeding ID: 8179 | | | | | |
| Date: 7 Feb. 2009 | | | | | |
| 127608 | m | Brd1 W | Tg (ACTB-Flpe) tg/+ | — | sacrificed |
| 127609 | m | Brd1 cond/+ | Tg (ACTB-Flpe) tg/+ | — | Backup |
| 127610 | m | Brd1 cond/+ | Tg (ACTB-Flpe) tg/+ | — | Backup |
| 127611 | m | Brd1 cond/+ | Tg (ACTB-Flpe) tg/+ | — | Backup |
| 127612 | f | Brd1 cond/+ | Tg (ACTB-Flpe) tg/+ | — | sacrificed |
| 127613 | f | Brd1 W | Tg (ACTB-Flpe) tg/+ | — | sacrificed |
| 127614 | f | Brd1 cond/+ | Tg (ACTB-Flpe) tg/+ | — | sacrifice |
| 127615 | f | Brd1 cond/+ | Tg (ACTB-Flpe) tg/+ | — | sacrifice |
| Date: 4 Jan. 2009 | | | | | |
| 126956 | m | Brd1 cond/+ | Tg (ACTB-Flpe) tg/+ | — | sacrificed |
| 126957 | m | Brd1 W | Tg (ACTB-Flpe) tg/+ | — | sacrificed |
| 126958 | m | Brd1 W | Tg (ACTB-Flpe) tg/+ | — | sacrificed |
| 126959 | m | Brd1 W | Tg (ACTB-Flpe) tg/+ | — | sacrificed |
| 126960 | f | Brd1 W | Tg (ACTB-Flpe) tg/+ | — | sacrificed |
| 126961 | f | Brd1 W | Tg (ACTB-Flpe) tg/+ | — | sacrificed |
| 126962 | f | Brd1 cond/+ | Tg (ACTB-Flpe) tg/+ | — | sacrificed |
| Date: 26 Oct. 2008 | | | | | |
| 125211 | m | Brd1 W | Tg (ACTB-Flpe) tg/+ | — | sacrificed |
| 125212 | m | Brd1 cond/+ | Tg (ACTB-Flpe) tg/+ | — | Shipped |
| 125213 | m | Brd1 W | Tg (ACTB-Flpe) tg/+ | — | sacrificed |
| 125214 | f | Brd1 W | Tg (ACTB-Flpe) tg/+ | — | sacrificed |
| Date: 5 Dec. 2008 | | | | | |
| 126164 | m | Brd1 cond/+ | Tg (ACTB-Flpe) tg/+ | — | sacrificed |
| 126165 | m | Brd1 cond/+ | Tg (ACTB-Flpe) tg/+ | — | sacrificed |
| 126166 | m | Brd1 W | Tg (ACTB-Flpe) | — | sacrificed |
| 126167 | f | Brd1 cond/+ | Tg (ACTB-Flpe) tg/+ | — | Shipped |
| 126168 | f | Brd1 cond/+ | Tg (ACTB-Flpe) tg/+ | — | Shipped |
| 126169 | f | Brd1 cond/+ | Tg (ACTB-Flpe) tg/+ | — | Shipped |
| Breeding ID: 8177 | | | | | |
| Date: 1 Dec. 2008 | | | | | |
| 126117 | m | Brd1 cond/+ | Tg (ACTB-Flpe) tg/+ | — | Shipped |
| 126118 | m | Brd1 W | Tg (ACTB-Flpe) tg/+ | — | sacrificed |

TABLE 20
Genotyping results summary

| Overview | Females | Males | Total |
|---|---|---|---|
| Brd1 cond/+; Tg(ACTB-Flpe) tg/+ | 9 | 10 | 19 |
| Brd1 w; Tg(ACTB-Flpe) | 0 | 1 | 1 |
| Brd1 w; Tg(ACTB-Flpe) W | 0 | 1 | 1 |
| Brd1 w; Tg(ACTB-Flpe) tg/+ | 7 | 7 | 14 |

| | | | |
|---|---|---|---|
| Line | DOB | ID | Status |

Brd1 cond/+; Tg(ACTB-Flpe) tg+
[Females = 9; Males = 10; Total = 19]

| | | | |
|---|---|---|---|
| females | | | |
| A-D1 | 5 Dec. 2008 | 126167 | Shipped |
| A-D1 | 5 Dec. 2008 | 126168 | Shipped |
| A-D1 | 5 Dec. 2008 | 126169 | Shipped |
| A-D1 | 4 Jan. 2009 | 126962 | sacrificed |
| A-D1 | 8 Jan. 2009 | 127107 | Backup |
| A-D1 | 8 Jan. 2009 | 127109 | Backup |
| A-D1 | 7 Feb. 2009 | 127612 | sacrificed |
| A-D1 | 7 Feb. 2009 | 127614 | sacrifice |
| A-D1 | 7 Feb. 2009 | 127615 | sacrifice |
| males | | | |
| A-D1 | 26 Oct. 2008 | 125212 | Shipped |
| A-D1 | 1 Dec. 2008 | 126117 | Shipped |
| A-D1 | 5 Dec. 2008 | 126164 | sacrificed |
| A-D1 | 5 Dec. 2008 | 126165 | sacrificed |
| A-D1 | 16 Dec. 2008 | 126461 | sacrificed |
| A-D1 | 4 Jan. 2009 | 126956 | sacrificed |
| A-D1 | 8 Jan. 2009 | 127105 | sacrificed |
| A-D1 | 7 Feb. 2009 | 127609 | Backup |
| A-D1 | 7 Feb. 2009 | 127610 | Backup |
| A-D1 | 7 Feb. 2009 | 127611 | Backup |

Brd1 w; Tg(ACTB-Flpe)
[Females = 0; Males = 1; Total = 1]

| | | | |
|---|---|---|---|
| males | | | |
| A-D1 | 5 Dec. 2008 | 126166 | sacrificed |

Brd1 w; Tg(ACTB-Flpe) W
[Females = 0; Males = 1; Total = 1]

| | | | |
|---|---|---|---|
| males | | | |
| A-D1 | 8 Jan. 2009 | 127104 | sacrificed |

Brd1 w; Tg(ACTB-Flpe) tg/+
[Females = 7; Males = 7; Total = 14]

| | | | |
|---|---|---|---|
| females | | | |
| A-D1 | 26 Oct. 2008 | 125214 | sacrificed |
| A-D1 | 4 Jan. 2009 | 126960 | sacrificed |
| A-D1 | 4 Jan. 2009 | 126961 | sacrificed |
| A-D1 | 8 Jan. 2009 | 127106 | sacrificed |
| A-D1 | 8 Jan. 2009 | 127108 | sacrificed |
| A-D1 | 8 Jan. 2009 | 127110 | sacrificed |
| A-D1 | 7 Feb. 2009 | 127613 | sacrificed |
| males | | | |
| A-D1 | 26 Oct. 2008 | 125211 | sacrificed |
| A-D1 | 26 Oct. 2008 | 125213 | sacrificed |
| A-D1 | 1 Dec. 2008 | 126118 | sacrificed |
| A-D1 | 4 Jan. 2009 | 126957 | sacrificed |
| A-D1 | 4 Jan. 2009 | 126958 | sacrificed |
| A-D1 | 4 Jan. 2009 | 126959 | sacrificed |
| A-D1 | 7 Feb. 2009 | 127608 | sacrificed |

1. A genetically modified non-human mammal comprising a genetic modification that inhibits and/or reduces BRD1 activity in one or more tissue or cell.

2. The genetically modified non-human mammal of claim 1 wherein the mammal exhibits one or more phenotype associated with a mental disorder.

3. The genetically modified non-human mammal of claim 1 or 2, wherein the genetic modification is a mutation in one or both genomic copy of the BRD1 gene.

4. The genetically modified non-human mammal of any preceding claim, wherein the genetic modification is a mutation in a coding or a non-coding region of the BRD1 gene.

5. The genetically modified non-human mammal of any preceding claim, wherein the genetic modification inhibits and/or reduces expression of one or both genomic copy of the BRD1 gene.

6. The genetically modified non-human mammal of any preceding claim, wherein the genetic modification inhibits and/or reduces the normal function of one or both genomic copy of the BRD1 gene.

7. The genetically modified non-human mammal of any preceding claim, wherein BRD1 activity is inhibited by approximately 100%, or is reduced by approximately 99% or less, for example, by approximately 90% or less; or approximately 80% or less; or approximately 70% or less; or approximately 60% or less; or approximately 50% or less; or approximately 40% or less; or approximately 30% or less; or approximately 20% or less; or approximately 10% or less; or approximately 5% or less.

8. The genetically modified non-human mammal of any preceding claim, wherein BRD1 activity is inhibited and/or reduced in all, or substantially all, tissues in the mammal.

9. The genetically modified non-human mammal of any one of claims 1-8, wherein BRD1 activity is inhibited and/or reduced in a selection of cells in the mammal, for example: cells of the CNS neurons; glia cells, forebrain, prefrontal cortex, hippocampus, amygdale, hypothalamus, gabaergic neurons, dopaminergic neurons, glutamitergic neurons and/or serotonergic neurons.

10. The genetically modified non-human mammal of claim 10, wherein BRD1 activity is reduced by approximately 50% in all, or substantially all, tissues in the mammal.

11. The genetically modified non-human mammal of any of claims 1-9, wherein BRD1 activity is reduced by approximately 100% in all, or substantially all, tissues in the mammal.

12. The genetically modified non-human mammal of any of claims 1-8 and 10, wherein BRD1 activity is reduced by approximately 50% in a selection of cells, for example, cells of the CNS neurons; glia cells, forebrain, prefrontal cortex, hippocampus, amygdale, hypothalamus, gabaergic neurons, dopaminergic neurons, glutamitergic neurons and/or serotonergic neurons.

13. The genetically modified non-human mammal of any of claims 1-8 and 10, wherein BRD1 activity is inhibited by approximately 100% in a selection of cells, for example, cells of the CNS neurons; glia cells, forebrain, prefrontal cortex, hippocampus, amygdale, hypothalamus, gabaergic neurons, dopaminergic neurons, glutamitergic neurons and/or serotonergic neurons.

14. The genetically modified non-human mammal of any one of the preceding claims, wherein the genetic modification comprises a mutation in exon 1B (amino acids 15 onwards), exon 2, exon 3, exon 4, exon 5, exon 6, exon 7, exon 7-long (where present), exon 8, exon 9, exon 10, exon 11 and/or exon 12 (amino acids 1-184), or any combination thereof.

15. The genetically modified non-human mammal of any one of the preceding claims, wherein the genetic modification comprises a mutation in exon 1A, the intron directly downstream of exon 1A, exon 1B (amino acids 1-14), the intron directly downstream of exon 1B, the intron directly downstream of exon 2, the intron directly downstream of exon 3, the intron directly downstream of exon 4, the intron directly downstream of exon 5, the intron directly downstream of exon 6, the intron directly downstream of exon 7A, the intron directly downstream of exon 7B, the intron directly downstream of exon 8, the intron directly downstream of exon 9, the intron directly downstream of exon 10, the intron directly downstream of exon 11 and/or the intron directly downstream of exon 12 (amino acids 1-184), or any combination thereof.

16. The genetically modified non-human mammal of any one of the preceding claims, wherein the genetic modification comprises:
(i) One or more mutation substituting, deleting or inserting nucleotides in the promoter or enhancer sequences of the BRD1 gene (resulting in reduced amounts of BRD1 mRNA);
(ii) One or more mutation introducing premature stop codons in exon 1B to 11 (resulting in nonsense-mediated RNA decay and, thereby, reduced amounts of BRD1 mRNA);
(iii) One or more mutation affecting splice donors, splice acceptors or intronic branch sites (interfering with proper splicing of the BRD1 mRNA, resulting in either the production of aberrant non-functional BRD1 protein or reduced amounts of BRD1 mRNA due to nonsense-mediated RNA decay); and/or
(iv) A reduction in copy number of the BRD1 gene e.g., complete deletion of one or both copies of the BRD1 gene (resulting in reduced amounts of BRD1 mRNA).
(v) One or more mutation introducing premature stop codons in exon 12 (resulting in the production of a truncated BRD1 protein and, thereby, in reduced activity either due to elimination of the BRD1 protein by protein quality control systems or reduced functional activity of the aberrant protein);
(vi) One or more mutation affecting splice donors, splice acceptors or intronic branch sites (interfering with proper splicing of the BRD1 mRNA and resulting in either the production of aberrant non-functional BRD1 protein or result in nonsense mediated RNA decay and, thereby, in reduced amounts of BRD1 mRNA);
(vii) One or more mutation substituting, deleting or inserting amino acid residues in the nuclear localization signals of BRD1 (resulting in faulty intracellular localization of BRD1 and, thereby, in reduced BRD1 activity);
(viii) One or more mutation substituting, deleting or inserting amino acid residues in the plant homeodomain finger, the bromodomain or the Pro-Trp-Trp-Pro domain (interfering with the three dimensional structure of the BRD1 protein and, thereby, in reduced activity either due to elimination of the BRD1 protein by protein quality control systems or reduced activity of the aberrant BRD1 protein); and/or
(ix) One or more mutation substituting, deleting or inserting amino acid residues in the nuclear receptor binding signals (interfering with the three dimensional structure of the BRD1 protein and, thereby, in reduced activity either due to elimination of the BRD1 protein by protein quality control systems or reduced activity of the aberrant protein).

17. A genetically modified non-human mammal according to any one of the preceding claims comprising a genomic mutation which is capable of reducing and/or inhibiting BRD1 activity in one or more tissue or cell.

18. The genetically modified non-human mammal of any of claims 1-17, wherein the mammal is selected from the group consisting of: cows, dogs, cats, goats, sheep, pigs, rabbits, mice and rats.

19. The genetically modified non-human mammal of any of claims 1-18, wherein the mammal is a rodent, and is preferably a mouse.

20. The genetically modified non-human mammal of claim 19, wherein the mammal is at least 15.5 days post coitus old, postpartum or adult (at least 21 days postpartum old).

21. A polynucleotide sequence comprising SEQ ID NO: 32.

22. A method of generating a genetically modified, non-human mammal as defined in claims 1-20.

23. The method according to claim 22 comprising the steps of:
A) Genetically modifying a host non-human mammal strain to be heterozygous for a inactivated BRD1 allele (constitutive or conditional inactivation);
B) Where the BRD1 allele in step (A) is conditionally inactivated, generating offspring heterozygous for a constitutively inactivated BRD1 allele.
C) Intercrossing of the heterozygously modified non-human mammal strain produced in step (A) or (B) to produce a non-human mammal strain homozygous for an inactivated BRD1 allele.

24. A cell isolated from a genetically modified non-human mammal as defined in any preceding claim, which comprises a genetic modification that inhibits and/or reduces BRD1 activity.

25. A method for identifying a compound for treating a mental disorder comprising the steps of:
(a) providing a test compound;
(b) administering the test compound to a genetically modified non-human mammal which comprises a genetic modification that inhibits and/or reduces BRD1 activity in one or more cell and exhibits one or more phenotype associated with a mental disorder;
(c) determining whether the test compound reduces and/or inhibits the one or more phenotype associated with a mental disorder exhibited by the genetically modified non-human mammal;
(d) identifying the test compound as a compound for treating a mental disorder if it reduces and/or inhibits the one or more phenotype associated with a mental disorder exhibited by the genetically modified non-human mammal.

26. The method according to claim 25, further comprising the step of formulating the compound identified in step (d) into a pharmaceutical composition.

27. Use of a genetically modified non-human mammal comprising a genetic modification which inhibits and/or reduces BRD1 activity in one or more cell, for identifying a compound for treating a mental disorder.

28. A method according to any of claims 26-27 or a use according to claim 27, wherein the genetically modified non-human mammal is as defined in any of claims 1-20, or is generated according to the method defined claim 22 or 23.

29. A compound identified or identifiable or obtained or obtainable by the method as defined in any of claim 25-26 or 28.

30. A pharmaceutical composition comprising a compound as defined in claim 29 and a pharmaceutical carrier or excipient.

31. A genetically modified non-human mammal substantially as described herein with reference to the accompanying description and drawings.

32. A polynucleotide or plasmid or isolated cell substantially as described herein with reference to the accompanying description and drawings.

33. A method or use substantially as described herein with reference to the accompanying description and drawings.


SEQUENCE LISTING (41 sequences)

SEQ ID NO: 1 - DNA, 47185 bp, Mus <mouse, genus>
  source 1..47185: /organism=“Mus <mouse, genus>” /note=“BRD1 gene” /mol_type=“unassigned DNA”
gctggggagcgagcagcgcctcggcaggcgtccgagcagctccgcgtccgcgtcctccgcccggccgggccccgagccggcctcagccggccgtgccggcgccgccgaccccgcccgagccgcggcgccctgcgggcccggagccgctggccgagcgcgccccggagcccggcggggcacggctgcgcggccgttggcggaggagccgcggcgccattagcgccgcctcggccgcgccggcctccgcgcccgcccgcccgccgggctcccgcggccgcggcgcccccgaaggtgagtgtctgacggtcgccgttcgccgcccgcctcgccggccggggcggaggtgcaggcgccatgtttggaggcggcagcggcggctccgcattgtccgcgggcggggaggccggagagtcggggcggcgaggccccgaggccgtgaggcctggcgggcgcgggagccggagggaccgagaaggccgggcggacgtgcgccgccgtgagccggcgcggccggggacgccggagatcggtgccggcggctcgcccaagaggccgggttcgggaggcgaggccgcggcgagatcgcggaggcggaggccgcagccgggtgggggcggagagggacacggaggccgcggcggggtcggggagacagaggagtagaaggaggccgccgcggcgcgggaggcgcggccaagagaatggagcgatcggcagggctcagtaggcggggaggccgccgggccgggcgggcgggctctgggcagctcggctgtctgggcggctggggcggccgaggggccgggcgtcggacagcggaggaggcggaaggcctggggtctcgtggcgtctgcccacgtcctcgcccgtagccttggcggtgcggagcgggtcgcattatgtaacagatcggtccgatctattttgccaagacaggaaactcccttgaagagggacgggctcggaagatttcctaagtggagcggggcctggtatctccggagcaagcccgcagctccgccacaactccgtggatgagtgcaggaaacgccgagaaacgagcgcgcgtgcgcggctttcttgggcctttaggagagaagcaactttcctgtgcgcttaatttgcagaaaacgcagctcctcatggtgccctgcagttgtgacacacttacacacacctaggaaacggccccccttcatggaggacattcacttcacccagctgcgactgttttagagtatctgtcatctggtaacaagtagttacagaatttccctattacttagttactgttttatcacttgttgggtcgcgtgcactgtcctgagtctgtgtttttctctccggatggtcaccttagagtaaggtgtgtctctttcctgtgtgcttttacggtgaggggtggaagctaggaagagtttaaatggcttgtccgcaaaccgggccggaaatgaacggagctgattttgagcatggagtctttcccctcgttttgccggcaaagctttttaggatgcgtttagcccagtgatttctggagaagcatgcttgttgcctttgctgattcctccgtggagagatgcttgttcctgcatagagccagaggggtaaagtgctgggtatatgaaaatgaggaagtagatgagattgttggtcactgtgccgggcagtactgttacatgtccgctttcccctggtcacaactaccttttcaaattacagagtagctgtggccattaagtattaggttcagttcttgtagaaaagtggtttaaagacagtccttcagtgctcactagaagaatgtgggatttgacaggctggctacagtactttactggagaggagaaaattacatgtttgtctttaatctgggagctgttgcttctgcccgtggttctttttgggaaggatatggtgctgacacctggatttgcacctatctcgacttagggatgccactagaggcctagggcaggctagggttgctttgacagtttcctgagaatccagtgttgagtaggcacctggaagtgcctcagaagcaggtgcattggggtctggctgactacagtgtcttcatattcttcttgttcatagagagatagtatagaatgtggctttctgcagcttgtaaagtctgtctttaaaaatgcattgtagagatttccttttgggacttaaaacatgaagtctgctctttgagggcttttcccaaagactagtaagataactatgagttgtgagttcaggctctggtgcgcgcgttctctctctctctctctctctctctctctctctctctctcgcccttcctctggactatcctgatatttcaacttggtatatttgggagtcagtctaactctacttcttgtcagtaaaataggtttgttgagctggaggggcgcgagcgagtgctcctggcacttgatgctccatgtgctcattctgcttgcccagtggttctgagtgggcttgtctgctcataaggtccatagataccacggcatgtcagagtccactacaaggaatgcgaatataggctcttggcgccctggttttgtccatcctggaaatgagcaaatctctgcattgaagttttcaggcgtgtgagccagagataaagggtggcggggaggccactgcaggctgtggtttgagggaacctgtcctttcttgggagcaagaactgagcattttcaggtgtgtcaggaagagagcagagatggcccttgattatcttgcccactgctaggtttgcttgaagagtatgtggcttagcatacccaggtcctggcctaatgagagggaaaggctggtggtgcccacggcagtttccaaggtggtcactgctgaggtgtcctgaaagctacactgtgctcttggggcaaaaatatcccacagatcagctcagcgttccctttagtcctgtgtaggatgtgtttgtggaaagaatggactactctatgctgttgacttatggaagcttctgggcccctgcaggaaagttcccaggagcgctctgctgggcagtagtgagaaagaaaggaggttgcttaggaattgctaagagtaggtggccacagcccagtaggcggctgctttgtggccacaggtctctgctgtgaagtctggcagaaaaacaatctatacttgtaggagagaggcctcgctcttaactctggagactgtgttgctgtttggggcttacttttggcttggtctaaagaggtgtcttgtgggtggaatgcacctgtgccctagctattcagcaggaaccctgagggctgcagcttcctgctgtctccggccttatctgtacctttacctgggtgtggtgagggagaggcttgctgaaatgtgagacattgtttggaagtcttcttcagagcctttaaactctgagctttgtttgcgggagatttgttagtgctacccaagcacattttgtagttctctgaaggcttctgtcatcctgcatagaggtaacttttcctttgactttattttaggtaatcattgccaaatgaggaggaaaggacgatgccatcgaggttctgcagcgaggcatccttcttccccgtgcagtattaaacactcccccactcgagaaacactgacctacgcacaagctcaaaggatggtggagatagaaatcgaagggcgcttgcatcggatcagtatttttgatcccttggagatcatactagaagatgacctcactgctcaggaaatgagtgaatgtaacagtaataaggagaacagcgagaggccgcctgtttgcttaagaactaagcgtcacaaaaacaacagagtcaaaaagaaaaatgaagtcctgcccagcacccacggcacaccggcgtcagccagtgcccttcccgagcccaaggtgcggattgtggagtacagtcctccctctgcacccaggaggccccctgtgtactacaagttcatcgagaagtcagccgaggagctggacaacgaggtagagtacgacatggatgaggaagactacgcctggctagagatcatcaatgagaagcggaagggtgactgcgtctctgccgtgtcacagaatatgtttgagttcctgatggaccgcttcgagaaggagtcttactgtgagaaccagaagcagggtgagcagcagtccttgatagatgaggacgctgtttgctgcatctgcatggacggggagtgccagaacagcaacgttatactcttctgtgacatgtgcaacctggctgtgcaccaggagtgctatggggtaccctacatccccgagggccagtggctttgccgccactgcctgcagtctcgggcccgccctgcggattgcgtgctgtgcccgaataagggcggtgccttcaaaaagacagacgatgaccgctggggccacgtggtatgtgccctgtggatcccagaggttggctttgccaacacggtattcattgagcccattgacggtgtgaggaacatccctcctgcccggtggaaactgacatgctacctctgtaagcagaaaggcgtgggtgcctgcattcagtgccacaaagcaaattgctacacagcattccatgtgacatgtgcccagaaggctggcctatacatgaagatggagcctgtgaaggagctgactggaggcagcgccacgttctctgtcagaaagactgcttactgtgatgtccacacgcctccaggctgtacccggaggccgttgaacatttatggagatgttgaaatgaaaaatggtgtgtgtcgaaaagaaagctcagtcaaaacggtcaggtctacgtccaaggtcaggaaaaaagcaaaaaaggctaagaaaacactggctgagccctgtgcggtcctgccgaccgtgtgcgctccgtatatcccccctcagaggtaagtgcatctgagcttccggctccgatgggcctgaagggaaagacttgatggtggacacaaatccgggccagcaggagttctgccacacctctgtcccacttcctgatagtcttcgtcctaagttgtagcctttaattgactggctactgtggagtggggtgtaaagtgtaaggcacggattgggatagtttacagttgtcacctgttggcctggaatataaggtaggtacactcacgggagccacagccacactagtattcattcaaccctgggtttctggacttcatagcatcctaagttttgtttctagctataatgccgttaaactcccttattaccagatttgaggaccttgtgtgaaagcatctggttgggaaagtgaactaccatcctcagtaaggtaacctttgaggtgaggttagaacaggagctgctgtcagcaggcagatggtggtctgtcttctactggccttgaactcacagggatcctctgcctgcctcccaagtgctcccaccatacttggcacattgtatgttcctggtgggaggacttgtcctctgcagtttagggactgcttcagcttcttcagtctgcattgggctgccctctctcctgtatcttctccactactctctggtttgctgtttttgttccattatttcaaaaaatgttccttttcacatcatagcctgaggatgccaaataaatccactctttttgtatctgtttgaacccttttttgagccttaaggaagtaattttctgtgaagggggtgtgggcttttagttgggtcaggtctgtaaagccccaaggagataaagttcatgtgaagcagacagcaacccacatgggttttactgtaaactgctccataaaaacgttcattctgtagcgaactggtagacagtagatttcagaggtttttttttggggggggggggagatctggtctctgtatctttggctgttttagaaagcctatagaccaggctgtcctggaactccatccgcctgcctctgcctcctgagtgtgctaggattgaagacatgagccaccagcattggctcagaacctgtctttaacatagtgaacattaggctttttgtgttactttcttatgaatgtctggtttgaagaaattaatctttttttgtttttgtttttgtttttttgagacagggtttctctgtatagccctgactgtcctggaactcactttgtagaccaggctggcctcgaactcagaaatccgcctgcctctgcctcccgagtgctgggattaaaggcgtgcgccaccaccaccaccgggggaaattaatcattcttgctagcatgcggtgattgattccactatggagttgggtagcaactgcctttgtattagagtttaaaacgggtaaataaatgcttttttttatagacctattcctactacttagagtcagtgagtcagaaacagagatctcgtaaccccttgttcagagaagagtcctggtagaaccagcatgcctgacttctgtgcctatagaggcggaaaggatagggttctatgagagctcaggaaagtttagctttaccgaaattgaagtaagtgaagcagcagtctgcttgctctcgctggagtgccaaatattccgtgttccaggtgatgggtgcgatcctgcaccccggcctgtggttcctgatgttcaggttttggaacatgaaagctgccaggtgggtgggacttgcaaggaggatctgcagtgagaacaaagaccatcgaagaagcttgaagctttaaaaaaatcttccagggtctgttgtagaattcagcagattctatttgtgcattgtggcccgtgtttccttccccagacaaggtcttatctgtagcccaagactgcctgaggcttatggaacacaagtcaggttggcctcagacttgtgagtctcttgcttcaacccgtcacatgctcactgtcctgtcctagcttgtcttactttgttttgtcatgttgtgttttgtgacagaatctcactctatatcccaggcagggttgaaactttttttaaagatttattttttatttatttttattgtatataagtacactgagctgtcttcagacactccagaagagggagtcagatctcattacggatggttgtgagccaccatgtggttgctgggatttgaacttcagaccttcggaagagcagtcggatgctcttacccactgagccatctcaccagcccgagccttggcctcttgaatgatggatttaaaagcataagccactgtgcatagctgcttgctactactgctgctgttgcttttttaattaattaattaattatatgtaagtacaatctagctgtcttcagacactccagaagaaggcatcagatttcattacggatggttgtgagccaccatgtagttgctgggatttgaactcaggacctttggaagagcagtcgtgttcttaaccgctgagccatctcaccagccccctgctgttgctttttacagatttattattcattttgtatgtgtgagtgttttgcctgtatgtatatatgtgcgccatgtgtatggctggttccctgcagtcagaagaggacttcagatgccctgggagtagagttgccgatgattttttgtgggtcagcagtggggtgcaatggaatacagttggacagctttaaccagtagacttcggacaggcagtgctggtcaacttggcttacacttttaatcccagccattgggaagcagaggcaggaggatttctgtttagagttcaaggccatcctggtctatgtggtgagctccaggacaaccagggctatggagagagactgtgtccaaaagaaaaaaaaagtttggggaaggttgaagaaggaaggtcaaaagagtacagattttgtgggtttttttgtttttgtttttgtttttgtttttttgttttttttgttttttttttccgagaagcctgttttgagccttaaggaagtaattttctgtgaagggggtgtgggcttttagttgggtcaggtctgtaaagccccaaggagataaagttcatgtgaagcagacagcaacccacatgggttttactgtaaactgctccataaaaacgttcattctgtagcgaactggtagatagtagatttcagaggtttttttttggggggggggagatctggtctctgtatctttggctgttttagaaagcctatagaccaggctgtcctggaactccatccgcctgcctctgcctcctgagtgtgctaggattgaagacatgagccaccagcattggctcagaacctgtctttaacatagtgaacattaggctttttgtgttactttcttatgaatgtctggtttgaagaaattaatctttttttgtttttgtttttgtttttttgagacagggtttctctgtatagccctgactgtcctggaactcactttgtagaccaggctggcctcgaactcagaaatccgcctgcctctgcctcccgagtgctgagattaaaggtgtgcgccaccactgcctggcttttttttttgggtttgtgtgtgtgtgtattttgtttttttgttttttgtggcagggttttcctgtatggtatggtcctggttgtccaggctggccttgaagttgacatctgcctgttcctgcctcccaaaggtgtgtaccaccaataccctacctattttttttttttcctaagaaaaatattttgatgcctgtttttctgtgctcttctgtgaccctgctcatccacccgattctgtgtagcaggaggaacgagcaagaccaggtaaagggcaacgcttcgtagttgtcccccccttacccccccccccaaacgaagtaccagtctcggtaacttccctgccctggccatatgaggccgtaatttatctccagaacagaagctgctggtgagtagctgtgcctgcccagatctggacttgactcactcagatcgcctctgtgccttggagaatgggtgtgcagtttattcagtgccgaggtgtacgttgtgacttggtgctgggtcagcagtgagactgaggcaccttctgtttgctgtttacactgccagtccttgatctggctttgggaaaagaccaggtggtgtgtgaacacccgatgcacttcatcaggtagactagggtttgcttttacatatactgttctggcttggattttgtgcacaccccctcctccatgcttctgctagttaacttgtcagcttctctctctctctctctctttctttctctctctgttaatggcatagctgtttgtttgtttgtttgtttgtctatttcgagttttagagaaacgtctttttctcttgtgtggtcctgactctaaatttttgagacagggtctcactgcgtgaccttggctgacctggagcttgctatgtagcctcagactcccccgtgcctctgcctcctgagctgggactaaaggcgtgtcaacagcatgcctgatttagttaccagttttgaaaacagtacatgtaaaatattgtatataatttgaattttgctctttctttgctagtggtatgtgtcacactctctggggatgcgacattgcactgctgtgagccacagcctcagtgagcggcacaaaggatggctgagcacttggtgggagctgtgctgtttaactgggctgttgggtagcatggctgctttgacttgtgtgaggtgatcagcgtgtagcctcctgtcaaagagcgtctgtatttgataaacatttccatctgccacggttggcgccatcccttccaagtggaagccctgccctgtatgtcctgggagcagtgtagggagggcttgctgctgtgccagggccttggaaagcaagcagatgcatctactgtagagatgctggggaagaagcatttgaacgaccgagaagtacaaaatgacacactgatgtggaaggcagagcccatctgacagccagtctgagatgagtgggtctacctgctcatctcgtgcccttaggaagctgggtcaatcataccgagctgaaatcactgtatactgactcttcccaccgtctggacaccttcccctagtggactgttgtccctgggcactcagcagagagggcatctccagtatgactgatttcctctttttgtttttaaagatttatttttattttatgtatatgagtatactgtgcttgtacagatggttgtgagccttcatgtggttgttggttgtttggaattgaatttaggacttccgcttgttccagtcaacccctcttgatccagtcaaccctgcttgctccggcccaaagatttattattataaataagtacactgtagctgtcttcagatgtaccagaagagggcattagatcttattatgcatggttgtgagccaccatgtggatgctgggatttgaactcaggatcttctgaagagcagtcagtgcttacccaccgagttactttggaataggtagaagtagatacttacttcattgctgggggcaggctgttctttggtctctctactgctgctgtgagtcagtccacttgaagctaacagtgggccttcgtgggaccctgaggtcagcaggactctcaagtttggtccacattagaaaaaaagattgcattacatggtcatgtgcccacggggcatgggttctaagttatcctttgcagtggggaggggcacttgcatgccctgtcctgtccatgcccaccttctagaggtaatcttggtgcctggttgttgctccataccgtgactccagctccatgcccctaacccagcctgcctcacacaatactcgggccctctgagtattaggaagaccattctgattattgctttgttctgaggggccagagcattgggcagatattaccaaatggaaggtcaggggccagagggccgggaggtgggcagacctgccactgccaggacatgggttgggtgttgtctctgctgacaccacgtgagccgctgctctgactgctcttcagctttcctggctttggatgctttgtctttgtctggtgtgtttccctctggttcactcaagttaaccgtccttatgttatggtgactgtcaaccataaattatttttgttaggaatcttggaggtttgacaaaggggtcacgacctacaggttgggaaccactggtctacagtattgctggtctttttacttgtttgaggcatgtctgtgttgaccaggctgacctagagctacctgcctttgcctctgaactgttgggattaaaggtgtgtgccgccattcctccatgtttctgagggtgatgtttcctggcagctagtttcacatctttgtcaagacttgaaaacaagtgcagattgagggttgtttggcctggccagtctttcctatgattattagcatcagtgatagtcctcggtccctgggcttttgtcctcccgagtttgtgctggtttgtcagttgcttgaagaggctgggaagttacccagtacataggacctgggcattgtgtggagaggaggcccggagtgtcaagagaggagccatttctcactacctcaggggagatgaatagtcaaccatatgatagcatttataatacagttggcctctgccacagttggcctgtcacctctgagatcttggccctgcttattttctgtggcaaatgccttcttataagcagccgaagaaggtgcgacttgccagctctcttttcgactaacttgtgttttttggcaattccaggtttcacatggccatctattgacttgggtgtatagtctgtgtctagaggtaaattgtagacttttgagtccttggaggcaaaaaaacctaggctttaaaaatgatgctttatttttttattttttattttcatgatgtaatgatgctatttgttgatactgtaaggttagagacacttgtcggcctgaccatgagctgtcctgaacatgagtggagttcattataaagatgtaggatgtgtaggaaatgttgcatcaagaaaggaggctggtttgtaaaattcactctccagaggtgactgtgtggagcatctggagagattgtgggtctatgcacatgtatgggtagaagtcatgttcttttcttacttttcatgatttttgtctagggaactctaaggaaaatcagagactaatgtaacctgagttatcaagtatagcagcaagccacagttaccgtggaggcctgcaatctctgggttcattctcctgcttagaacagcattcatagccggcagtggtggcgcacgcctttaatcccagcacttgggaggcagagacaggcggattcctgagttcgaggccagcctggtctacaaagtgagttccaggacagccagggctacatagagaaaccttgtctcaaaacaacaacaacaaaaaaccaacaacaacaaaaaaccaacaacaacaaaaaaagagaacagcattcaggtgactctgggacttgcgtgcacttgacatccttgggcacggcttgttttctcatttctagtgatagctgtgattgacaaaggggaaaataagctttaagaagtacagagaaaacctattggttcagcaacttaacttcaaaagttcctgtgactggttcatcctccttgcctgtacctccctctgtcccagccatgtgactccatgactgcagctgtaaagaaccttctcagagctgtagattgatgctaatgaagtgagtgctggtcggtccttttctgtgaaaagtgtccccagaggtcagggaggctttggggttctggaattgtttgttgcggatggtatgtgggagcctaagagcctgttcctccatactgctgtggttcctgctgtgtagaccttcctgctggctcccagccccacagtttctcacctcctgtgttcttggtctggttccacagtattgctgagcataggggtagctcatagcactacgggctttttactgactgtcccatgactgcatggttgtccccatgacatcagtgttctgtgggaattctggtagggacgaccttgccactcacataggtttatttatttatttattttttcttttctttacttgagatagggtttcttcataggttcggctggaaattactatgtaaaccagactgccttgcctctgcctccctaggactgggattaaggtttctaccaccacacctgctaatatgagagttaactggtgaggccctgtctcaacaatagccacaactccacccaccccactttccaaaatgtccctcccccgattaaattagccgtttgtgactttgttaggatacaggatttttgtttttatatattatatataaaatttataaattttaggctatctttgtagacagtatctgtgcaaatggcagttttgttgggcttttctgcttttttagctttttactcaaagtcagacgaggcctgccttttgagctgcccagaacgggattgactctgatgcatgcatgtactgtatgtatgttcctacatagtgtatgtagggatttgtttgttttgttttttaagcagtcttactgtgaagccctagctgccctgaaacttatgtgtagaccacagagatccatctgcctctgtctgagtgctggggagaattcttaacaatcagtactatttaattcataatggagtcactggttttgttaaaagccggttactgggctggtgagatggctcagtgggtaagagcacccgactgctcttctgaaggtccagagttcaaatcccagcaaccacatggtggctcacaaccatccttaatgagatctgactccctcttctggagtggctgaagacagctacagtgtacttacatataataaataaataaatctttttaaaaaaaaaaaagccggttactacttccctgggggaggggtgttagtgttgggcggaggtggagctggccctgtttctcttgccgttcttttactttttttttttttttcaagacagggtttctctgtgtagccctagctgtcctggaactcactttgtagaccagctggcctagaactcagaaatccgcctgtctctgcctcccaagtgctgggattaaaggcgtgccaccacgcccgactacttttactttttaagacagtctctctgtgcagcttaggtggcctccgcgagactctgaagtgctgggtgacaggtatgccgccatgcccagctttcttcctggtttgttcttactgtcggaggagttcgaaatcctggccatgtgtataagtagactataaaagtggcttgttgtggtactgtatgtgcaaagctacaagttggctgtaagcagtgcattccactttagacctagggtctttctcactaaaagtggatacaacctaggccgagaaagcttagaaggacccgacagtgtgagtcacttgccaccttcactttgtaaacataacttcacacttttcagcaaaatggtccagttaatttttctcttgtatttttgtttttattagctgtatttaggaaggcttgagaaacttgtgagtgtattcttgctaacatttaaaatttttaaatagactataatattaagaaattcatagctgggcgtggtggcgcacgcctttaatcccagcacttgggaagcagaggcaggcggatttctgagttcgaggccagcctggtctacaaagtgagttccaggacagccagggccatacagagaaaccctttctcgagaaacaaaaaaaaaaaaaaaacaacaacaacaaaaaaagaaattcataagacagatgtgtggttattaagttacaatggaacaattgtagcgattgtttgtgtcgtggagcccttcttgttgcatggctagggctgaaagtggtttggctcctgtagggcttgcttcatgggcttttcctctctgtaatcttggtttatttgtgcttttgacataacactcatcagattttagttgcaataactatgcagataagattggggagtttataaaggatttttttttttagctcacaatttgaggagctgagagcacaagattggacatcacatcaacttattctgtggctgaatcagtggggcagtggcatcgtggcgagagcgtgtgtgggacgtagaaatgctgcagtgagataggactccagagcacagggagtggccagcctggtcttcctgctgggtacctatctccagggatctgggacagagtatccagactagagtagcgcctctgtttccttctagagatccattttggtcatgtctacttccaggttcctgtgtgcgtgggtctcaggtctgtctgtgttggttgtctgtcagtggtagtttggcctgttcttcctgtggtttctgagttggtagttggcctgatcattgatgagtgtgggatgaacttgttggacatgcttgcttttggctggtctgcttctgaggacctacagtattagtgcctgttgtctacctttctccacagtgtgcaattgctcaccaggggggagtcagactctgcttatgtagtgtttggatacatacctgtagaggacatattttaaatttgtttgtttgtttttgttgttgttgttgtttgagacagggtttctctgtgtagccttggctgtcctgggacttactctgtagaccaggctgccctcgaactcagaaatccgcctgcctctgcctcccaggtgctgggattaaaggcatatgccaccactgcctggcattaaatgtattttctataaatcttgtttacaacttgcaagctatttacagtttcccaagttcttgcactggggaaggtgtgggtctagtatgaagttggaagctttattaaagcaaattgctaattattactattttttttgacttttaaattgttaacaaatcttgtatctggctgggagcggtggtgcacactgttaatcttagcactgggaggtagaggcagaactctgagctcatgactataaagctagttctgggacagccatggctccattacacagagaaactgtcttgaaaaaaacaacaagaagcaagcaaaagtcttctatctctgcgctgcttctgaaggttaaagtaaccatcagtgtagtgttgaacatctgtttgctgtacagatgttacacctcagtcagaagtgaaaacacaagctgttaccaacactgcagctgtggcgtggccgggcctcctgcccgctccatggagactttggtccatcctcaggtgtcgtggttgccttctggtgcagcctggtgtcctgcctcttgatgggtttgtcattggagataatgcttcgtggtcttggtgtttgacccaccacattgagcatgcagagccgcagagggcactgcatcatccagcggagctcagccaggaggctcgaccacctcgaggtttgaagcattctcaagagcaagcaaaccttggcagagccgggccttggcagagccgggccttccagctgatgctggtgttcttgattgcgttctttaaaaaaaaagtgaacttagaaaattttaaagcctgttgtgtaattttgatgtgtggtacagtgaaggaacaccttcttgtagccttttgtagtgggatttgctggagtttgtctttcagtgtctttgtgaggcggcataccaagccccatcttcttcagagggagggaagcaggctgtggtataagcagccgcgcagaagctctctggccggcattcacagcactcacacacagcctgagggctttgagcctcccttctgcagaggtttttacagcttggcacgaggatggttgtcatttactaggagcagaccatgttcccagcctgaactcagtgggtgggctgctctgcttggagagtttcttaaggttgagtgtgcccagcgctggtggcgccagctgtgagcgcaggctttgacctccagtccatccagtcggcagcatctcagctggcagtggtcagtagccgtcactgtgtgtgtagacaggagcacaggggcaaagtggttaaagttttgttcacctgtgtctgctttagacgttgaacctggtgactcttgtggaggatgaaatctgtagttagttgaaggttatgaactgttttcagggacaggctcagggagagaactgcagtgtcctgtctagttttctaaatgcaaacacgtttaaatatccctttcgaagctaaactctcagttttttcatgttttagattaaataggattgcgaatcaggtggccattcagcggaagaagcagtttgtggagcgagcccacagctactggttgctcaaaaggctgtctaggaatggtgctcccctgttgcggcggctccagtccagcctgcagtcccagagaaacacgcagcaggtatgtgtgctcttctgcttttcagttacatgggctgccccccccccccccccccaggctggatgtgctgctgaccctaagccccgggccttaaactctactaaactgcaggttattcgggtggctcctgtatcctcaaggtttgctgtgactttggggttgagttgttctttactctgacaagtgtctgctctgtgcccagtcctctgtcagttccagggaaggaagggactgctcagagaacctggctcaacttcagctgcatgcatagtcaagacagagagggaggcctgatgaagtctatgcagttcctctacacattgcccaaaaactaggtgtctggtaatacctgctggttccactgggaggagctagtcatttcatctgtaaaatagcaaccaactttaatggaagtttaagtctgtagaatcctgtgactccccatggctgtcacaggcatggctgtgaatgagcttagggttctcatcctgtatcctggctgtcagatgagcagtggtactggagccctgttgtatggatcagacccttgtgtctgcaggttaccaagtattgctcttctgggagttaacaacttgctggactctgtctgggtctgatctgaatggaaggggcctccccagtgttagatcttctgttgccttctacaagccaacgttgtctattattcactgaggacacatacctccttggaggctactggaatgtcctagttaggggtttccattgctgagaagagacacagtgaaggcaactcttacaagggacaacatttaactgggctgacttcacaggttcagaggttcagtccattatcatcaggccggaagcatggcagtgtccaggcaagagggtcttagagctattggtcatgaagtggggaagtgtttggtaaccctgggcactgggaggaatgattgcctatgtgacggtaggtagcagtgttggaaagagaagtccgggagtgggtggctacttctgagcttccccttctcagaagtctcttcctgggaagaattccagcattgatttctatgtagcaaagcagactgcttcggaatcgtaccgggacagcgggtttacagatgggatgatctgtgtagatttgtgtacagggtcctgtcttcgtgagcctatagcatggtggagtgcagacagtggctcaattacccatgaccttttaaagatgaaaaccaggccaggagcaaaccacttgagttttgcctatccctaaatatacaagctcaggcctgttggaaacctatccaaaatgctcttatgttactcagaagtctgtttctaaggagcaggaagctgtccagatgatgctaggatatttggttccttttttctttgtttatttggagatagggtcaacctgaatcttgctatatatgctggccttgaactcgcagaactcagtctctgcctcctaagagttgaaattagaggtgcacatggccacagctggcaatgtttgtgaactcccctttccatgtatttgctccctttgcctatatgtgatgagtgaggtacactgtgcattactgtgggcgctaaagtgtgcatcaggacagaccatgccattcccatcctgtgctgccattttcataccatgaagagtggctgtttatacagttgggttggtgacactttgctccgagaccctccatctttgaccgttgtgctggtagcttgagttgcagtctctgctgtggtgtcactgggccatgagaggcaaagctgtccagagagaaggggctcctgtgtgttctacagctgcaaggcagcactttgcttgtggctggcagatgtagatatttatttaggttactgtctagcagtagtgcagaaggacaaacttttgggtaggtcattttccatccctttataatagggacaggcaggacatatggcttactgtgaggaggtaatcccatacattttccacagagtagagagtaggggatagctttggataatgacttgtgttggatgagaaaccaagtcttggacaggttcactctggggaggcagaaagagaagtatggggtggcaggaaaggagatctgggttgggggagcagagctctggggaacgtggttggataagatgcatggaattctgagaggatgaggcatgttgaatttcttggcaagtgactggaaaacctggtgctttgtagatagggctctggtcttgtttggtgttccttggttgctatcaagggatgtgtgctatccctgtggcagtaggtcttgtccccgtacatttgtgaagtagtaagagtaccgtggttagccttgaggggcttactaggcttctggctgcttctcctgcttagaactctgagctgcttctcctgcttagaactctgagcagcagctcaaggatccacctccctctggtgctgcagagctaggctgcttccctgctactgtctgtctcttggtgcttccactttgttggctaggatagagaagtgctggtgcaggatgctgaccaagtgctatttggtgtactgcctgagaaggcagctgtgactggcaactacagtgcccacgcctagaactgaacctgcataatattccgccgccagtaagggtagcttaggtttgtacctcttgtgtatctcctttctcgtactccctccattcctgcctcctggagtcaagccaagaccccgttgtgtcgactagaccttcctgtcccattgtcacagcacatttatagggactgggtacatttatagagactagatcccaggtcctgctacccttttagtcttacctgttggatgagcttgttagatccctggcaggaagaactttggggtgtgactgatggaaagtttcctctaattttctcagagagaaaatgatgaagagatgaaagctgccaaagagaagctaaagtactggcagcggctgcgacatgacctagagcgtgcacgcctgctaattgagctgctgcgcaagcgggagaaactcaagagagagcaggtgaggagggaggcccttgggttctgccaccctctgggctgtccctggatagacgtcttgctgccgtcatggagtgctctggagtggcccctgtgtacctgctgagttagtgctgtccccaccctgtagcatatcatatccctaccctatagttggtcctgtggtacctctgtgttgtccttttcgattagccacctctggagtatacggggtcttaaaggagacccctgccgtggaagaagtacatgtccttgcacagagaaggcagctttgtggtgggatggtagctggcacgtaggctgctctgtgctgctggttcaagtggcgcttctgtgattgtgcagtacgtggaggtgcggtgatctccaggagaggtgtccctacactcctctggagacagtgtatgcagaggtgtccctgcatcttctagagacagtgtatgcatgctgttgttgccaggtgaaggtggagcagatggctatggagctccggctgacgccgctaactgtgctgctacgctcagtcctggagcagctacaggagaaggaccctgcaaagatctttgcccagcccgtgagtctcaaggaggtgcgtgtccctgcgactgagctcttcggctgcttgcttaggaagcatgcaactggggagaggttacctgcattcttaattctcattagttagtagttaatgaatttttggtgaatagtattttaattataaaagattgtacctcgttgtaaagcactgaaagtgcataggtgaaaatttctacttagaacttaacaattggtgatgatagcccccctggtaccccatctgtttgtacttttagttgaagtaggttgggagggtctctgcagtgattgggcttagtttgtattggcttagtgttgttatgtgaaattagtttcaggtgtggttgattttgtaaatgtttattttccctcctaaaattaggtaccagattatttggatcacattaaacaccccatggactttgctacaatgaggaaacggctagaagctcaagggtataaaaacctccatgcctttgaggaggattttaatctcattgtagataactgcatgaagtacaatgccaaggacaccgtgttttatagagctgcagtgaggctgcgcgaccagggaggggttgtcctgaggcaggcccggcgagaggtggagagcattggcctggaagaggcctcgggaatgcacctgcctgagcgacccatcgcagcccctcggcggcccttctcctgggaagagggtaagaactgtatccaggaggacagcggatgctttttctctcagactgcactcactaagactccagcatgccggccgagtgagtgctcctgaggtgcatgcgccttgtatgggcaccacgtgggcctcgccatgttttcacatacccactgcgagaaacacatatctaggtgctgaaggccccgaagacactatagttgaggatgcatccccaaagggtctgaccttgcttctgaggtcatgcattgagaaggcagctattcattagttgtcatatttcagctgagaagcaaaagcaggagctaatgttggctgtgcctctgatcctctctctgggatgcttgcaggtgtttattgagggcccagccttagcctgcttctaggacatggcctaacccttctaactctccagggcaagcttgtactctgggccccaccgtgcacatgctgttgtgctcttcattaatttcttccaagtaaggagctgtttttaaagataaggtctcagtgggtagtcttgactggcctggaactcaaaatgtggatcaggctggcttggacttgacagaagtccacctgcttctgcctcctgagtgctgtggttaaagatgtgcattaccataccacatctggcctccaatcatttcttgtaagcttcttgcccctggattgtttattctgtaggtaaatgtctacagtaggtgaatggggtttggtggtcaaccttggaacttttattcacaaaacccaagatcctatgttcctgatttgacctaccttttctcctgctattgactgttcaggaaaatggtggaatcgtacggacttaggttttatccggtacgtttccttctcctggatgaccagctgcctggtcactgtggcctgactcgtgaggtcagagcccttggagactcctcacttctggcttcctgtgtatctgacccagagaaactgtctgtctcaggcatctctagggcatacaggatagggttgaattctttttttctcaagataggatgtagtgccacactcaggaagctaagacaggaggttcaccacaaatttaaggtcagtctaaactatagtgatttctaggctagtgagttacaccctgagaccctgcctaaaaaccaaaactgatcctaacagtataattagaaagaaaagcagccaggccagagtgtggcttagtagtgtttctttgcatgcacaacatttgggttcaatgtcaacacagcataaactgggttgatacaaagattagaatttaaaggtcatattggctatagagtgaattaaggctagcctgggttacatgagaccttgctttgaaaaatagatatgcatgcacccacacaggtgacaagatttctgaaaccctagataggtccagcaggaactgagcctgatagccaccaggattacagagcgactctcagatcttcacctgcatccatgttcttttctccagattgtgtgggaggcaagggtgggctccagcctcatctgttgtggccgtgactgtgctttgggtggtatcggctgccctgagaagcagaggagcccagtgacatctgggagtctttgaccccacagcttctgattctcgtgctctgtagatgggcagggctcagaggcctcacagttgagattccaggaaactggctttgtcattgctaaataaatttctgtgccagactttttgccaaaaaggaaagtaataatgaaaagtacaaatttatttcttactcagtgattgcagtagaaagcatgacctgtggcagggtgagctctgggtactctgccgctgtcttgagcctgcagtaaggaagatacttgtcttagttagggtttttctgttgtgagcagacatcatgaccaaggcaagtcttacaaggacaacatttagttggggctggcttacaggttctgaagttcagtccattatcatcaaggtgaaaacatggcagcatccagacaggcatggtgcaggaggagctgagagttctacatcttcatctgaaggctgctagcagaatattggctcccaagcagctaggagcccacacccacaaggccatacctcccaaaagtgccactccctgagctgaacatataatatacaaccattacattccaccccctggccctcataggcttgtccaaacataagcctatgggagccatacctacacatagcataatgcaaaatacatttagtccgacttcaaaagcccccatagtctatggcagtctcaacaataatcgtccaataacttaactgtaatccccaaagcaagacaggaagccagctgggctctgcatctccatgtctgatgtcttcagatcttctattcctttttcatctttgttgactgcaacaaacttctttctcctgggctggttctactccctggtagcatagcagctttccttagcagatagtccaactaccactctggtatctccaaggcagcttcttgttttaatgtctgggcctcctctccaaggtgacgtcacttccccagctctgccctcggtagctctaagctcaggttgatccctccactgccgctgctgctcttggtggccatcatctccaatacactgggggcttccgctgcaactagagcctctctaggctctcttcatggtgccaagcctcaactcctttgcatggccccttcagtcctgggccatcatctgcaaccgaggctgcactttgatcagtgatcttccgcctcagctgctcttcatggccccttcatgcctcaaggccagtgccacctgggggaccattgcagtcacccagcatagctgcagcatgaggtgcaaccttggctgtctctggaacacagcttcttggtgctcagaaaacacttccagtgatgctggttgtcgtcatgatttatttattatatgagtacacagttctcttcagacacaccagaaagagggtattgggcccctgttacagatggtcgtgagccaccatgtggttgctgggaattgaactcaggacctctggaagagcagtcagtgctcttaaccacagagccatctctccagccctgccggtctcttaatcactgctaatgccttagctcccgctaaccagcatcagctgtcccaggagtctttctcctcgtgattataaagccagagacacatggccgaagctgcttgctggagctggaacatggcccctagttctattgcgtcatcactagcttccagctttcgcgctccttcaaggcctaagtttgtcacgtggggatcttgctcagaactctgagatatgcaagcctgactcctgggattagaggtgtgtaccagcacgcccggaattaagcttttcttcacctacaacttgatctgtccttgaaagtagagatctgcctgcctttgcctccaggaattaaaaagcttgttctgcccagtatagaccaaaacttaactgggtgggatcttgccccaaggtcactagtcccttaattcaaactaatgtccttgaacacattcagctccattcacttccagtattcctttctaaccttgcaatgcttattcacatgctcttcctgagaacaaagtctacgatgggcctttctaaggcttcctttgtcattgtaattaacctgagcctccttagcctcaggcagactcttcagccaagggcaaaaatagctacttcttcaccaaactacaaaaacaaggctctagaccacataactgaaattcctcactgaaacctcttgtgctgggtctacacagttccgattactcacagcaacaaagtgttccatagtccagctaggatagaccatgaagccccacttgaaacattctgtggccttccaaatcccaagttccccaacctacattcttataagcaaaaacacggtcaggcctattaccgcaatatctcagtccctggtgccacctgtcttagagtttttctgctgtgagcagacaccatgaccaaggcaagtcttctaaggacaacatttaattggggctggcttacaggttccgaagttcagtccattatcaaggtggaaacatggcagcatccagacaggcatggtacaggaggagctaagagttctacgtcttctgaaggctgctagcagagtactgactcccaggcagctaggagcccacccatgaggccacacctactccaacagggctacacctcctagcattgccgctccctaagcagagcatatacaaaccgcaatactggccctgttgaaagagaagccaaccagcagagcctgcaggtctagcactcaggttgaggagggaggattacaagtttgaggccagcctggactcagcaagcacaaaacagaagaaaggaggcttgagaagttgagtggtggtttttgttgcggtgactgtaagccagttggacagtgtttgtcgtgtcccactgctaagttagtgctgtttagacagggcgctaatgagtctcctaggccagctaccaggtctgggcagggctcatttatggtaggtgtctctgttggccctgctgttcctttggttttatcttcgcatagattaaataattttttggctatttcactaatttaagtcctgcagtcaatgttcctagagtctggggagacctgcggactctgcagcctagtttccttttggtcatgatgtatgtgcaagaacttgagctaggatgatgttcacaatgtataaacagtccatgtgaacatatttacacacacgcagcgtctgtcagtagtccatcttgcgtctatgttggtgcactcagacatgtctggtggtctttgtgcctctcactttttacagagcaggactgagttgggtcttagtccaggaaaagccatgtgtgttacccacatctcctctgctacggccacactagtcctttgtgtactactgactgaaggagtgtcttgtctctttttttccctctttgtgacaacagccttgtcataggttcagaatcagggtagagaggagtatgtatggcaccaaatggtgaaattggaacacttgggaggcaggggcaggcagatctctgagttcaaggtcagcctgttacagaatgagttgcaggacagcctgggttacccagagaaacactgtctcaaaaacaaacaaataaaacaaaacaaacccaagaagctaaataaacaaacaaagattaaatgaatttgaagcctgcgctttggccgtgggcaggcccaggcacatagttaagacagatgtgttgttatcagaggcggccatgaatccgaatcctgtggctaatgatacgtgtttttggttcagtggacaggttgctggacccagccaacagggcccacatgagcttggaggagcagctgagagaacttctggacaagttggacctgacctgctccatgaagtccagcggctcacggagtaaacgggcaaagctgcttaaaaaagagattgctcttctccgaaacaagctgagccagcagcacagccaggctccgcccacaggggcaggcacgggaggctttgaagatgaggctgctccactggccccggacacagcggaggaaggtaagcatggggtaggagggccatacctcacgggctcggggctctcttgacaggcttaaatgatgctctgtagtaatgatgagcttgtacattttgaaggtcacggaactcttggttactggatattcctgctaggctttttttgatgctctttgaaaggatgttttggtgtgttctgtctgctgtattttggcacttagtttacaagcttaaaggaacagaatgagattttcttttaactcgagcttgaaagacttagaaggaatagtttagatccaatacagtgttgaaggtggcttctatggtgggaatggcaataacttagttgtattttgttaattgaggcagagtattatgtgagtagacaccctagaattgtttttaccttgtctacgtaggtcagaggacagctagttggagttggttttcctggcatcttagcacgcttggggatcaagcgcaggtggttaggccttgtaagcacctctgcccttagctaagccctcctgcggctggagttaggaaaggaggactggctagagaacagcccagccttgggctgggcatggtgggaggagtctgacgtgcacagacctgttcccagactctccctccacctcaggcctttcctgtggctcaccttcagtggacactgtcttattctggcagcgtgagtgacttctggggaaagagctggatagctgagatgttagggtggagaggaaggaagggaggaagtacagaagaggctgtctgccccgtgcgatccacgagatgagcaggtcattgtgtggagggagggaggcttctgtgtgtggtgcatctaactggcatgtttgatggtacaagcaccctttagtccacttgtcttgacatcaccacatttcaactccatgaaatggaaagaaaaataagacctacttcttctgccactgctattagcagcttgacttaggatctccctgtgcattttttttttctgccccatccaaataagaaaaacattaacacaagaccattgtcaccatagtttgcatttttttgatctgtatggctgcctgtcttagtagatgtgactttgccctattcctcagagtgacatggtttcagtatgtttatgccatgttaaatttagtcttataattttaacagttggtgacaatcttctaacccactttccccttctctggttgcttcttttatatggttatgctaggcaaccagcagaagctagggccaacaccagagttctcctggccttacatccttctagtgtgttcacttgtaaactcacaaacacccttggccttgccattaggtaacaagtttgattggtccacacagtaaaggttttattcctcagtgtgtgacacgttttctcctcattttctaaaagcctaatgacctgcacatggcaattttctgcctctgttggggcctctatgctttctttaaggaacattgctcatgggacctttgacaaacagatgcatccaggatacagttattgtttgcattctgtggtgaggcccatatagtgccattgcctggttctcatggcagcccttctcaggctcctcttgtcactgcttgaactggcccagtaggacccttggtccagccacttagtgagtgacctgtacactttgtcctaaagagtcagctggggagaagggttaggcaggaccgctcactgacattgcagtagctttacaggattgagggtctgtccaccttttgtatctaaattggaggaagagcagtgctattggaagactggatctggtgcgttgcactgctgcggccacttcacaggaagcacattggtgctacccgagacccgggcctaacattgctgctggccagtgtttaagatgcaggaaaggggcactttgcttttagctgagaagaaagatgagtggagaaggaaagagcctgacagtttgttctgaggcagagctgtgagggtggaatttagggcctcttaaagagactgagttccagacaggcaacaggggagcacttcagttctggtgaacagcagacacagaactgtgaaattgctatatgcatgttgggacagaaccctgaactcaagacattacgtagtaattcagcatattcttcccaaagaggatgttttggttggatgcagtcatacatcctagaggcagaggcaggtagagctctgagttcagaggccagcctgatgtatagagtaagttctagaccagctagggccctgagacatcatgaccaattaaaaaaaaaaaaatctgtgattattattttttttttgagaaggctcatagatttttcacacctaggaaggcatatcttaatataaaataagcaatttcacttaaattgtaattaaacaacatttttgtgtattataccatgtagggtgtttgcattagaggaaacatcccctgaaggctaacatctgaggaacaaaacaggccctagctgtcctggacagtggacatgcctggcttgcttgtacaaagggcaagctgtttgtcaggaggcctcccatgctgaccttagggttgaagagttcagtcagttgaagtctgagggacacatggaatggggccatgataaacctggggacaagcttgagctcttagacgtttttactcattcatttctaactaggagctttggtgagctcagagtctatgtgctggtgattactcttggccagatcagcacttccagggggacatcactgttgctgcagcagccatgtgctgtccctactgtatgtaccccatattgaatacagtacacactgttctttcagggctggcagaagggagcaggatagatcgctggtgtggatggtgcagtctctagttatggaaagtctctgcacactttgctgtgggatccagagttatctgtggctttggtggaagcattcggttggcttggtggccttgtgtatagagaatcatggtcaaagggactagctggtcctgagtagatgtctgtcgaatccggatgtgatagttgctagcagacagtgaggtttttaaaaggacaatgtttaacgtttgtatattaactgccagtaaggtttttcttcctgcctgagggacctgatgggagtgttagctatggcactggtgctgccctgtgttctggcgtgagagttcactcatcaaggagcctgacgccttgggtgttgctaaatctatctcagtgtgagttttagtgctttgtgtagcctagccctatggctgctggagatggtgcttcacttgggcctgggcaacgccttttgtatccagtgtgattgtttttgtaacacccaggagtatgccagtgaactatagggcagtagttgggaacctgggctcctccacctcattggttgtcacagagcagggagaatgcaggactggagtgtagaggggaccatagatgggtgtgactagctatgcagtccctgtgggcaagcagcttttgatagacagtggttgggggggatgaaatgtggtggagaccttgtgggaagggacagcatgttcacttgttgtcttagcagcagtgaccgaatctgaaaagttaagcaggaggcagaaaataggtctttggtacctcttagccatggagagaacggatggaagatctacagtgcctggagccctgggcaggaggcctctggtaccattctctggtagtcttgtatgtagggattggattggacatcctggaagcctcaggataagctgcctggagtgagggaagaggtacagagctgtggaggaggaggaggaggaggaggaggaggaggaggaggaggaggaggaggaggcggcggcggaggcaggctacaggcggcaaactgaagtacgcagtgtgagcccaatggtgggcatccaacacagccatgtgctgttctaccagcctaacacaccatccagcgacttctgttttcatatttgtgtgcaccaagagtgccatgcacggctctgcactgttcctaatcccatcttctcagtgtccctgtatgctttgcaaataatactgtcctaaagctgttactgaacctaacccgagtgggcaggaagaatgtcgtcatatccaacatgtcagtgattcaagatttggcatttccacttcattggaagtttaacctcccaagtacagaggctttggttctgtgacatgagtttgtgcactccttatcttgtgggtaaagcttttcaaccaaaggcctcttaaaaggctgtttgaggctgtttccactgcttggtggtggagttgtctccaacaggcttctcatacacacaattagcccaagccacctgacttgccagagttgctgtttccagtctggtgttgctgagtcccgaggagatcttggcgcagagcctttgttctgagttaatcaagtgacgcagggacatcctcaaggcttttaagttgtgtagccttttactttcagagtgacttttaaatgtaaagtattgtgacatagtgtaaatgttttgtgggaaacttgtagtttgaataaaatagaaaccatacctagggataacatatgcatgctcatgctactttggaatgttgaaactgatgctgttgaattttctgtcatatgtcctgtagtgagagtctcagaatcttgcccagaaagaacctttagcagttgagcccagcgggctgagccttccacgatgagctctgctgctgtttgttgcttgagctttgtgtgtggagacaggaaggtgcttctccctggtcaggtccatattggttttgtttttattttattttattgtatgactgtgtgccaagtgcacaggccttgtgccacagaggccaggtgagggcgttggatttcttggagctggagttctagagggttgttggctgcccacgtggatgctgggaaccgaacctatgccccttacaggagcagcaagtgctcttagccactgagccagctcttcagctcccatgttggtaatttgtaaataccttactaaagaggttgaaataattttgggggtcttttttatttttagagatatgaaggtagagtgggggaactaagggagtattgtctgtgtatcctaagggagtggaagaccatgctgcctcgttagagtctcccagccactcacctccccaaagttgggctttggcttagaaagtcctggctagccgggcgtggtggcgcacgcctttaatcccagcacttgggaggcagaggcagaggcaggtggatttctgagttccaggacagccagggctacacagagaaaccctgtctcgaaaaaacaaaaacaaaaacaacaacaaaaaagaaagtcccggttagacagttgagtatttgtttcttctctttatcactctgtggcgcagcataaactgctgtgggacctttaaggctccctgagttctgtcactgtctcaccctcctcaaggtaagtaaatactgatgcagagattgtcctgaactcagatgaggtttttaatattgctgtgtgttaaatgctctttcacagttttttccagaaagtaacttgtgcacctgggcttaggacaccaggcccgaaatcttttgttagggaaacacacagtgttacctgacggccggggctacacactgctccgcagaccaatgtgtgtgatgctgtttccttaattgaattagtgttttggtggacttcacatttatataagttttataatagattttataatcttcagttttcaaaatcactttatttataattttttcaggagctaactctccccctaaacttgaaccatcagatgcattacctcttccttcaaactcggagactaactcagaaccaccaaccctcaacccagtagaactccaccccgagcagagtaaactattcaaaagagtcacatttgataatgaatcacatagcacttgcactcagagcgcactggtaagcggacaccctccagagcccaccctcgccagtagtggcgatgtgccggcggcggcggcctccgcagtggcggagccatcaagcgatgtaaacagacgcacttctgttctcttctgcaaatcgaaaagtgtaagccccccaaagtctgccaagaacactgaaacccagccaacttctcctcagctagggaccaaaacctttttgtctgtagtccttccgaggttggagactctactgcagccaaggaaaaggtcgaggagcacatgtggagactccgaagtggaggaggagtccccgggaaagcgcctggacacaggtaaatggcaggggcagctctcccccagggctcataatagaaaaccatggtgctcagcttttgtttttgcggcatcctctcactcatgtcaacatgtcagtgattcaagatttggcatttccacttcattggaagtttaacctcccaagtacagaggctttggttctgtgacatgagtttgtgcactccttatcttgtgggtaaagcttttcaaccaaaagacctcttaaaaggctgtttgaggctgtttccactgctaaagctgtaagctgtcctctggcagtggccattcagcctcttggcagcccagcagctggctatgcagtgggcatggctggctccgcccctcccttgttcctttcttgttgacttgtatgtagtttgattgcataccttgactattgtgtgcatgtatatgtgtaaactgggacctgggaatggccacatctggcactaggtgcccggggggtggggtcttctaagagcagttcccacagctcagaaccatcaatttaaacctgaaccttccttactgagggctgcttcttccctgagctctttgaaaatatgctgtcatctcattacttgtaacacttcatacttggcttaaggagtaccgtgatgttccctcagtggttttgtatgttgttttagtacatgtgcgcctgactaggagggaagcacatatcagggtgcacatactacacatgcctggtaaaatcccactcaagcctttctcctttactgccaggcttttttctttctaatggaggtagtctgtctgtctgtctgtctgtctgtctgtctgtctctctttcttttttttaaatgctaaacttgctgagtagtcatggctcaccccttagagcaagttccaggaaagccagggctacacagagaaaccccgttttttaaaagacaaacaaaaaaaagctaagtgagtttggttgagtgctaaagtgtggtgtgggttggggaataaatcttgagagaaccggaagttgattgtctcctttcactctggggttgaactcaggtcactgaattcccactgagctatcacctagtcctattgttaacctgagatgtgttattttctaagatgttgcctgtgtgcctatacctttagcatcctgtgctgtccttgacttgacggctcttacttggcttcctcaaattccctgtgttgtgcttgacagcactggcttggcttcctcacagcctcctcctgtctaatccctagattaaacactgcggaagcgggtgcttgtgttcttaaacgatgattgccagcatcagatgtgatgttcaacgtctgctgtctgtgtaaggcaggtttgcactttgcttttggtcctaggtgcctaggattagtgtcttagttatttttctattgctgtgaagagataccacgagacaaaggcaacttataagagaaatgatggaatttgtggctcatggttcttgaggtttagtccatccccatcatggcagggagcatggcaggcattctggcaggcatggtgctagaaagttagccgagcgcctacatctgatccataagcttgttgggatggtagagagaggagagagattgtgcaagcaggagagaacgctaactggaaatggcttgtgttttgaaaccttaaaactggtgacacaccttttccaataaggtcatatctcctaatctttcccaaacagttgcactaactggggataaacattcagatatatgatcctgtggggacagtttcattgaaagcaccacattccgtttcttgggcccctgtaggcttgtggctatatcacaatgcaaagtgtatctagtccaacttcagaagtccccattgtttcataatttcactggtttgaaagttcagagtcttctgagactcctaattttaacctcttgtaaaagcaaaataaaaaatcacatacttccaacataaaatacattagcattccaatagttaggaaataccagaccatagcaggcctttaacccagccaggcaaactctgaatcctctggctctgtgtccagtgtcaggactgacagagatggctctccccttccagctttgctgactgcagaacatctctgaggaactgcttccatgttgtgtttgtagctctccttggtagacatctcatgactttggcaacttcaacatcgggacatctagcacaattcaggcagcttcacacagcagcctttccgacttccccatgcagggattgacctgccaggagcctggtttcagtggctttccctaacaggaggaagagtccacaactccttattcctgtatccttccagactgtgaagtcagagccaccaggctggagagctgtgttaggcgtcagcttgccctgcttgagtgacgttggcattggctttggtttgttatttattgctttttaggaacagatcattccttagccccgttcttcttgcctgagtagtctcgtcgtaaggacgccactcccgttacttcatttctcctgacctctgtcagcacaagccttgtctttttttttttttttaagatttatttattattatatataagtacactgtagctgtcttcagacactccagaagagggagtcagatcttacggatggttgtaagccaccatgtggttgctgggatttgaactcaggaccttcagaagagcagttgggtgcccttacctactgagccatctcaccagccccaagctttgtcttgaacattgatgggggccattctcagaccaccacagtaaggaaagttgcattctcatccaggcctctggtgttggctggtcttgtctacatgtcagcagtgagcaacacataggtcttggctgatgagggaattctgtttcctggaaccctgtcaggtggtccagtcagagggttgggagggcaaggctggcctggtagttagagtggactattgacacctcttcatctttgtttctccatctgtttgagttccctggcctgaagcaccacaccttgaagagcacttcttgccaaagtcaatacattgtggtttcttccccccatctctctctctcgtgtgtgtgtgtgtgtgtgtgtgtgtgtgtgtgtgtgtgtgtgtgtgtgtgcgcgtgtgtgtgcgcgtgtgtgactggcaccttgtggcaagttgctgtgtactatggagtgccttttaatgtgtgtgtgtgttgggggtggggttggggttaaaaaattgtctctcccagcgtgcattagacatgtacagcaagtacctttacactgacacatcttggtgaacctttcaaaaaatgttgagacaagttggtcttgaaccttttctataataaaggcctttagctcattagcctcttacctcagcctctgaagtagctgaaatcacagacctgtactagtaggcccagttaatttaatttttattttgcagtcagtaattttatagaaatttctctcaatgccctcttgttttgatgagttgataatggcactttaaaatccaataacccttggttttaatgaagcaattttaatatgccacaggaagtagattgaaactgaagttatctcagttcttgttggaagtttctgaaactatatagctttagcttttcttagccacatttagtgaaagactctggtgtctaaatcctttgtcactgaactgatcatggcatgctgtgctctgctgctaggagattgctcagtgtctcaataccaggtgccttgcacgcacaggatggctttctgcctcctcagggttcatatctaccagcagcagaaagtttgttaaccctgtaagacactgtagaaaaagctttcaccatgtagctgctgtccagagctcctgctttgtacctggcagcttctgccaggaagcctagttagccatgtgggctctgctgggccatttgtcgccgggggctactaatctcgaggtcatgaaatgttatgtgtttgcacatctcagttcttttctgggttctcagtgacaacggtgagtcccaaggagtcttaactttaggatgtgtgatccccagtgcctttgatcctgactgaaatggagatttctgctttcttattccagaatggcagtagctttcagtggatgcatgatgaattcctaatcgcactcctgagcagccgggagccttgttagcactaagatctgaccctcaggaacaggaggcgtccactgctgcatctgcctggcccatggtgggccaggcctgggctgaacgggcccatcctaccatcggtgctggctgtgcctccacttgaaccttgtggtgctctctgcgcacctggatttcttgtttcaagttgcagttcttcgctgtttgaggacttggagtattcagaaccttctggtcttttccaggttcatcgggcactgaacttgtagggaattctctggtgctctccagtgcactgcaagattccaagttagattaagcgtggacttacctattttaaaactgcccacccacaggcctcagcttgctcatgcctgcaggacaggcaggccatgtgggcagtgccgagcatggtgtgactgctcttatggttttcatattttttggagctggctctgtttcgtaggttttttttactctgcctgtttatttccatcaatggaccgtcaggccaggacctgtgtcacctcttactcgtactctgtggtgtggagattctcaatgaaatgtgtggtgtggtagtggtgagcaggtgaaatgtcttccctggccatggactgttgagggaggagatgatccctgcccttgcagtcagactaaatggcttctcactgttttccaggttttcagttaaccactaatgtgcctgggtagctcactctttggatcctaatccttttctcttaacctcgacttggactggagttctgctaaatggcctcttggaatgcaaagccttcgctgcctccttaccttctcctagttcttgaggaccacattggaatcacctgctgagccatcctctaaacagacacctacctaaccttgaaggagatctgtcctgggccagcgtacccctctctcagcccagcagttgagaggagcctggtgcctgagcagatgtctctcggtgcctccgtcttgctggtgctatagcagagcctgctgtagcttggacaacacaatccaacagtttgagcctcatcctgagcacactcagaactgacctgggatggccgggggctccggttaggccgtcgtggtctaggtgtctgaagggactgactgcacatacactgcccgaatgggcctaaatagagctccttacttggttgttagcatcttttatttctttgttctgttctccctcctttcttctctctcaatgtttcttggattcgtaggcattgctaatctagttggaacctgtgcagatatctgcagagccagctgagaagtcctcctgcagtgcccttgagttgggagggcctttggcatggcctctggcttgtgttggccatgtgcagctgtcttcatagactgtacttatgaaaagcagagtggtgggtggggtggtcttgctcactgtgctttattgaaaggtggagcgatgcacccaacataaagttcttttggagacaacaaagtcaagtgtgatcagaggacagtaatagatgcttttgctgccacccttagatggttgcattcctagcctagggcacggtccagcctgggacacaagcttgtcgatgtgcactaggtgggaacaagctggagctttgggcagcatgattctgtgctgtctcagaggaacctgtgctccggaggcgtctgtggtggcaggtgtatcagcaaggatgcactcagtgactacagtctcaggcacagctcatggcttctgtagagggtgggaagcctgtcagtgccctgatgctctcacgtgctgcagcctgtgagctcttggcactgtgctggttgggagcccagggtagaagggatcacgtccctacttccccactgtgtgcttctgagctttccaggtagaccacctgggacctctccctggcctgtgggtgagagctggagacctgagcacctcagaggaccctttattggccactccgtgtaacccagttgtgctgtagaagacctgggctaagggggagcttttagggaagatgtggtagttaaaaggctggggctgggcctgtcttgggacacagttgcagtggtattttggtgggttcctctgggcctgagtaaggtatttgaacactctctttggaggcctcaggaccctctgtgactgacatgtgcttggggtccttggtcttgtgtaggagacatctcacctttgttttggttgtcaagagcagtaggcatgtgtgtgatcttctacagtaagctttggggggggaggtgagggggtgggaggagcatcatttggctctccttcctgcagctaattggagagtcattgtaggaagtggatcacaaagagcgaagcagagacacaaagctctttacttggctctgtgtcaaagccatgtcatcttcagacagtgtctgcaagagcttttcaagcttgtgtctccggcccctgttgcttctcagggcatgtccagggccttggaagccgacaagaatctggctgggccatgcaagcaccgtagttgtgtttgggctgtagcaccctatgaaaagcaggcctggagatggctctgctcagcgggccccagaggtctttcctgaaagctgctcaggtttcatagtggctctgccagcttctcaaggtgttaggtatttttatgtaatgtgtgaaaactttctgtaaacttaggagcccagatgcagtgtgccctaataattagacacttgggaaaatgagagagagatttaatcatattttttcctttctcaaagttataaatgttctcttagtttttctaaaccccttcccccacaagggactgtttaggccctgacaaagtaccttgctatgggtaaagctgttgccatctttgttgggaacaccaagtgtgtgtgtgtgaactgtacttctgggttcttctaggtttctttctagcacagagatgccagtgttggggacctgcttgtccaagacctttagactctgcagacctgcctgcaggtgcctgctctgcctagccatggcagagattctgggcttgtagtcttctaagatcttgagtcctggagcaagggctttgcctccgtccgtccatccaggtgacagggccatctctgtgttgactttgctaacctaagtcagcaggtgtccatgtccgagttgtgtgttgcaccggccaaggcagcaccacgctctgttgcctcctaagagttgctgggccttgaggccctttaggagaggggtgtggcttcttccctgtcttgtgtcttctgctttgccagtgagcaagcaaacgagaacttcttgaagcgtttttgaccttttttagcacaagcaggtcctttcccaggcgtgatatgggagaacgcaacgaagaccttgtaatctagacagtcacctcatacttttaagaaaatgtttttcaaaaataagtttacatgttttactttggaaaatagtttaaaaaatttttttaaggttatatggggaagatgggtatatgtgaacaaaaagagtgttgtctgtttgctgttcccgtccccttctctttcttaagctggtgtagccagcaggagccatgcaagcgcacagcctggggacaggatccttctgattgagggaggtctgcgaggaccatgggtggccgggcctttcctgctttaccgacatagagtcagggtcagacttgcctgccaggaatgtggtgtggccttgactcagattggtctttattaaagcacttcacaaatctccagatgctgtgcttgcctttgtgcagacattgtacctcagggagaccgtggccgcatggctcagcctcctgccaactttacattcttcctgcttggggacctgacatcgctcggatgacttgggcccacacttgcaggtttaaagtgttttatccatatttttaaaaagttcttgttaacgttgatttttttttaaaaaaatttaatttaattttactttggctggtggtcagacctagggccttgcatgcctgctaggcacgtgctctcccactgggatagactgccctggttttccttctttttccttcctttctttctctcttttcttactccctccctttttgtttgtttttgtttttaaagcctaattttctatactagtccttaaagttctacatggtcagtgtgttttgtggaatcttgtagtgttcctccactctagggcactggagtgtttgtgtctcgacaccgcttattccttgtctagtggacagtctagttttgatgtaagctcccagcccttactccatgtgatctgttaacttgggaagtggtaacagtttctttttgctttgctcccctgggactagttgagcatgtgcagagctgctctgactttctgtggtctgttgtgttcattttactcagcagtgcctctgcatttgtccacggaggtcacaggagacatgagatactggcttttgtgtggacagtgttctttgtggggccagtgatgtagctcaggtggtagagtgcttaactagcatgcacaaaactctgggcttgaaccccagcaccacacagaccaggtctggaggcaagatcagaaattcaagagcaacctgaactctatgagaccctgtcttacaaaagcaaagttatttggaaaacactgtagtttttaaggaagagagagagacgtaggactaagttgggctaaagctactgctctggtgtgttgtgactgagcatccgtctgcttcttgccttccaggtctcaccaatggctttgggggtgctagaagcgaacaggagccaggagggggcccagggaggaaagctgcgccccggcggcgctgtgcatctgaatccagtatttgttccagcaacagcccactctgcgactcaaggtaggcccggtcctctggagatggagatggactgccctggagctagttcatgggtgtgctttgccatcaggaacagcttctcgggataaattgatttgtttagtcggatttaactgaagtcagaagttgatttaagttagtttataattaaactaacactttatacctcccacgccccaaatcttttctctgattaagattgtgatgtgcagtgccctgcctatgtgtactgtagtggccactgtcgagtgggtaagggtgacccaagtggcctcctggggacaggcttacttttcttgggtctccacaccacattgtccgttggcagcctggcacctggactaggataaagacacaggcgggggacgctaaactgtgctctcagtttgattcatctctgctttcctccgaagctttagcacacccaagtgtgggcgagggaaacctgcccttgtgcgaaggcacacgctagaagaccgaagcgagctgatatcttgtattgaaaatggaaactacgctaaggcggccaggattgcagctggtaagttgggatacagataatggatggaaaggcagtgtctggtcttggtggcttggggctgtgagtcaggcacactcccccccaccccctgcgcgcgcgcgcgcgcgcgcgcgcacacacacacacacacacacacacacacttgaggaaaggagtaaactcatgcttacttaactcactgaggtgagaactgctgcctgcctggggtttcaggtctgtcctgggctgcgtggcacttggctctaggagttcctcatcgttaggtctattcaggaggagatctgctttctgactgaatgtgtcccagcagggtggtcatccctagcttcaggccacagtgtaagggagtgtgtgtgtctgccgggctgatgctgtttgtttagatttccctgtcagtggaccgggccaactccagggatagaaatcctcgctgtttgaggcttgtggggcagagacctgggattgagaagggctggagactgcagggaatctctgtggctctagaggctgcagtgcattcagtgtgataggagtactggaggccctgagttacagcgccactaatagattgtgctgctgtgaggtgggacacaccatttaccaacagtagtcagtgagggcctgtacacacacagtacatacacagtggactccttttttttttttttttttggttttttgagacagggtttctctgtgtagccctgaatgtcctggaactcactctgtagaccaggctggcctcaaacttagaaatccgcctgtctctacctcccaagctgggctcaaaggtgtgtgccaccactgcccggccacagtggactcttgaggtgtgtcctgggctgctggacgtgctcagcgaggctcagaagagcgatgtcgtggtagtttggagcaagccaggacttgtatttggctgtttggttgtgtgataggcatctggtacatgcttaaggatcccatctttagaatggaggttcaagtatggtgaggtacaggggacacgaagtcataggccttagaactgggggtggtgggaagcagggaggccttggacaggcttctaggcctcccttcccctggaggaacagtgaggtagaactgtcctgctccagcagctgggaagcggggcctgacaggagagtggggctttttctagccccaggctaggagactgtgctgagtgtgttagcggttctcctgcttgctctgactctgctggaccttttctcctagaggtgggccagagcaacatgtggatttccactgacgctgctgcctccgtcctggagcccctgaaggtggtatgggccaagtgcagcggctacccctcctacccagcactggtgagtctgcaggcagggaggagggtgttgtggtgggacctggggaggggccccagtgcatgctctgtaccttgcagttcctctgctgccaaaggtgtatgattgttgtcgctctggaggcaggtgtgtggatggctgtgagcttagaaggctctgagtttgagagtactgtgagtcactggagcatgtttttggcagattattgaccccaagatgccacgagtgcctggccaccacaatggcgtcaccatccctgcgccgccgctggatgtgctgaagatcggtgaacacatgcagaccaagtccgaggagaagctcttccttgttctgtttttcgacaataagaggagctggtgagtgtgctgtctgcagcaggcagagctggggttctatccgacctggggcttagcttgacccatgctgagtaagggtgtctccaagtagttttttttcctggtccttgtctgctgctgcctaatgacacctggggattgtgaggtgctggtctcttctgacagctcctatcacttcgtcgatccctgagtggctcagactgtcttcagttcttgatccaggctcacttgcagtgggctttctgaacccactgctctgcccctttccatcctgtccatcttcctgtcctctctccacccagagctattggaactcctctcatggttgagatcctaatactccctgagggtgatatctgctgacatcttgaccatatttagttgaatccagccctttcctatgcagaccattgtaactgggtcctttcagctggccatgcttaggactgaaaggtgcttcaataccatggaggggcccctcttggggtgctaccaggttccctgtggtcctctttttctctctgctgactctggctcctggcagttcatcattgaggcttacactggctttgcccacttgatggttctgtttgtagtcttttcacccaccctaggatgcctcttcactctgctccagggttcactgacctatattatgtgcacacacacattctagtttgtcccttgtgtgcatatgtgtatgtacctgggctcacctagtgggtcactcacttgcgttatcttgggcctgttctgtgtgcatagctgtatgttccaggtttatctctgtagccacttggcataagcttgagaaagagagttgtattgtggttttcgtgccttagctgagtccagagaggactgagtaggtgggtcccctacgcacccaatccatcctgcatgaggcccagcgtgtgggagcttgggttgggtaccaccaggttccttcctgtgtgcatgggctgatggctggtgagccacaccgagtatgagctggtggttcttatgctctgacttctctttaaaaggcagtggcttcccaagtccaagatggtccctcttggtgtcgacgagaccatcgacaagttgaaaatgatggaagggaggaactctagcatccggaaggctgtgcggattgcatttgatcgagccatgaatcatctgagccgggtccatggggagccagccagtgacctcagtgacattgactgaggtggtttccagcaaaggcggtggccaaagcctcagccagccgggagctctgtccatagtgttgataagctgtacatgtttgtatattgttcagaacttaacttattctggttttctaggcgtagttctttaattctttttcccctggggaggggaggtttcacttccaagttttctatgaaaccatctggtcttggctttgcaagtgaggagggtctgttgcgagcagtgtggtgttggggtcccactgcaggtgccgagggccgaggcctcacttattctaatctgtagggtttttttttttttttaaagacttttgaatgtttaataattttgtagatcatgctctttacacagagtaccgcttatttaataagacgggatgtaaatttacaatgacaaatgtgtattttaagaaagaaaatgacattattttgaatggtactttgtgcaaagagggaataaatttatgctgtgtgcatcacttgcaaatcaccaaaaaatgtcccgccagctgctgccggacagggcccgttctcctcgttgatctgactgccctgagtctcctgctctgccctggctcctgcaggcgtgcctcccagcgggttatttattgtagaaagtgtactcatttgctttataatgaaaaataaatttgcaaaggtatattgatatgcatttttatacaggcacataaaaattcaacttggtgtgggagcagaatgtgttgcgaggttatatacacgactggcctgtgtgtactttgattttgtaacttgtaatcttttgtttacaatgaggagctttctgtaacttgttttcatttagaacactttggtagcaatagactttggatacattttgtatggtacatgtgatgtatatagaattagtcctttatttttatttctaagaggtaaagcattatgttaggggaaaggcagggtgggtttccaaatttgcatttttatattaaaaataaagtgaagatttggacagtgtggccctctcattcctgcatcactaggaggctgggtgagctgtagcctgagggacgtgagggactcggagcaccgggcctggagtgggtggtgtgacacacttgatctaacagctgactcgggatggcattatttattattttgcctaatcatatttttattttaaagctaaatagttactaaaaattttaaatgttcttttaaatctacatgtttgtaatatctccatagaaacttgaaaataaaaagtcttcctttggt

SEQ ID NO: 2 - DNA, 291 bp, Mus <mouse, genus>
  source 1..291: /organism=“Mus <mouse, genus>” /note=“BRD1 Exon 1A / Promotor” /mol_type=“unassigned DNA”
gctggggagcgagcagcgcctcggcaggcgtccgagcagctccgcgtccgcgtcctccgcccggccgggccccgagccggcctcagccggccgtgccggcgccgccgaccccgcccgagccgcggcgccctgcgggcccggagccgctggccgagcgcgccccggagcccggcggggcacggctgcgcggccgttggcggaggagccgcggcgccattagcgccgcctcggccgcgccggcctccgcgcccgcccgcccgccgggctcccgcggccgcggcgcccccgaag

SEQ ID NO: 3 - DNA, 1381 bp, Mus <mouse, genus>
  source 1..1381: /organism=“Mus <mouse, genus>” /note=“BRD1 Exon 1B” /mol_type=“unassigned DNA”
gtaatcattgccaaatgaggaggaaaggacgatgccatcgaggttctgcagcgaggcatccttcttccccgtgcagtattaaacactcccccactcgagaaacactgacctacgcacaagctcaaaggatggtggagatagaaatcgaagggcgcttgcatcggatcagtatttttgatcccttggagatcatactagaagatgacctcactgctcaggaaatgagtgaatgtaacagtaataaggagaacagcgagaggccgcctgtttgcttaagaactaagcgtcacaaaaacaacagagtcaaaaagaaaaatgaagtcctgcccagcacccacggcacaccggcgtcagccagtgcccttcccgagcccaaggtgcggattgtggagtacagtcctccctctgcacccaggaggccccctgtgtactacaagttcatcgagaagtcagccgaggagctggacaacgaggtagagtacgacatggatgaggaagactacgcctggctagagatcatcaatgagaagcggaagggtgactgcgtctctgccgtgtcacagaatatgtttgagttcctgatggaccgcttcgagaaggagtcttactgtgagaaccagaagcagggtgagcagcagtccttgatagatgaggacgctgtttgctgcatctgcatggacggggagtgccagaacagcaacgttatactcttctgtgacatgtgcaacctggctgtgcaccaggagtgctatggggtaccctacatccccgagggccagtggctttgccgccactgcctgcagtctcgggcccgccctgcggattgcgtgctgtgcccgaataagggcggtgccttcaaaaagacagacgatgaccgctggggccacgtggtatgtgccctgtggatcccagaggttggctttgccaacacggtattcattgagcccattgacggtgtgaggaacatccctcctgcccggtggaaactgacatgctacctctgtaagcagaaaggcgtgggtgcctgcattcagtgccacaaagcaaattgctacacagcattccatgtgacatgtgcccagaaggctggcctatacatgaagatggagcctgtgaaggagctgactggaggcagcgccacgttctctgtcagaaagactgcttactgtgatgtccacacgcctccaggctgtacccggaggccgttgaacatttatggagatgttgaaatgaaaaatggtgtgtgtcgaaaagaaagctcagtcaaaacggtcaggtctacgtccaaggtcaggaaaaaagcaaaaaaggctaagaaaacactggctgagccctgtgcggtcctgccgaccgtgtgcgctccgtatatcccccctcagag

SEQ ID NO: 4 - DNA, 157 bp, Mus <mouse, genus>
  source 1..157: /organism=“Mus <mouse, genus>” /note=“BRD1 Exon 2” /mol_type=“unassigned DNA”
attaaataggattgcgaatcaggtggccattcagcggaagaagcagtttgtggagcgagcccacagctactggttgctcaaaaggctgtctaggaatggtgctcccctgttgcggcggctccagtccagcctgcagtcccagagaaacacgcagcag

SEQ ID NO: 5 - DNA, 132 bp, Mus <mouse, genus>
  source 1..132: /organism=“Mus <mouse, genus>” /note=“BRD1 Exon 3” /mol_type=“unassigned DNA”
agagaaaatgatgaagagatgaaagctgccaaagagaagctaaagtactggcagcggctgcgacatgacctagagcgtgcacgcctgctaattgagctgctgcgcaagcgggagaaactcaagagagagcag

SEQ ID NO: 6 - DNA, 129 bp, Mus <mouse, genus>
  source 1..129: /organism=“Mus <mouse, genus>” /note=“BRD1 Exon 4” /mol_type=“unassigned DNA”
gtgaaggtggagcagatggctatggagctccggctgacgccgctaactgtgctgctacgctcagtcctggagcagctacaggagaaggaccctgcaaagatctttgcccagcccgtgagtctcaaggag

SEQ ID NO: 7 - DNA, 313 bp, Mus <mouse, genus>
  source 1..313: /organism=“Mus <mouse, genus>” /note=“BRD1 Exon 5” /mol_type=“unassigned DNA”
gtaccagattatttggatcacattaaacaccccatggactttgctacaatgaggaaacggctagaagctcaagggtataaaaacctccatgcctttgaggaggattttaatctcattgtagataactgcatgaagtacaatgccaaggacaccgtgttttatagagctgcagtgaggctgcgcgaccagggaggggttgtcctgaggcaggcccggcgagaggtggagagcattggcctggaagaggcctcgggaatgcacctgcctgagcgacccatcgcagcccctcggcggcccttctcctgggaagagg

SEQ ID NO: 8 - DNA, 261 bp, Mus <mouse, genus>
  source 1..261: /organism=“Mus <mouse, genus>” /note=“BRD1 Exon 6” /mol_type=“unassigned DNA”
tggacaggttgctggacccagccaacagggcccacatgagcttggaggagcagctgagagaacttctggacaagttggacctgacctgctccatgaagtccagcggctcacggagtaaacgggcaaagctgcttaaaaaagagattgctcttctccgaaacaagctgagccagcagcacagccaggctccgcccacaggggcaggcacgggaggctttgaagatgaggctgctccactggccccggacacagcggaggaag

SEQ ID NO: 9 - DNA, 498 bp, Mus <mouse, genus>
  source 1..498: /organism=“Mus <mouse, genus>” /note=“BRD1 Exon 7A” /mol_type=“unassigned DNA”
gagctaactctccccctaaacttgaaccatcagatgcattacctcttccttcaaactcggagactaactcagaaccaccaaccctcaacccagtagaactccaccccgagcagagtaaactattcaaaagagtcacatttgataatgaatcacatagcacttgcactcagagcgcactggtaagcggacaccctccagagcccaccctcgccagtagtggcgatgtgccggcggcggcggcctccgcagtggcggagccatcaagcgatgtaaacagacgcacttctgttctcttctgcaaatcgaaaagtgtaagccccccaaagtctgccaagaacactgaaacccagccaacttctcctcagctagggaccaaaacctttttgtctgtagtccttccgaggttggagactctactgcagccaaggaaaaggtcgaggagcacatgtggagactccgaagtggaggaggagtccccgggaaagcgcctggacacag

SEQ ID NO: 10 - DNA, 105 bp, Mus <mouse, genus>
  source 1..105: /organism=“Mus <mouse, genus>” /note=“BRD1 Exon 7B” /mol_type=“unassigned DNA”
tccttccgaggttggagactctactgcagccaaggaaaaggtcgaggagcacatgtggagactccgaagtggaggaggagtccccgggaaagcgcctggacacag

SEQ ID NO: 11 - DNA, 136 bp, Mus <mouse, genus>
  source 1..136: /organism=“Mus <mouse, genus>” /note=“BRD1 Exon 8” /mol_type=“unassigned DNA”
gtctcaccaatggctttgggggtgctagaagcgaacaggagccaggagggggcccagggaggaaagctgcgccccggcggcgctgtgcatctgaatccagtatttgttccagcaacagcccactctgcgactcaag

SEQ ID NO: 12 - DNA, 128 bp, Mus <mouse, genus>
  source 1..128: /organism=“Mus <mouse, genus>” /note=“BRD1 Exon 9” /mol_type=“unassigned DNA”
ctttagcacacccaagtgtgggcgagggaaacctgcccttgtgcgaaggcacacgctagaagaccgaagcgagctgatatcttgtattgaaaatggaaactacgctaaggcggccaggattgcagctg

SEQ ID NO: 13 - DNA, 110 bp, Mus <mouse, genus>
  source 1..110: /organism=“Mus <mouse, genus>” /note=“BRD1 Exon 10” /mol_type=“unassigned DNA”
aggtgggccagagcaacatgtggatttccactgacgctgctgcctccgtcctggagcccctgaaggtggtatgggccaagtgcagcggctacccctcctacccagcactg

SEQ ID NO: 14 - DNA, 155 bp, Mus <mouse, genus>
  source 1..155: /organism=“Mus <mouse, genus>” /mol_type=“unassigned DNA”
attattgaccccaagatgccacgagtgcctggccaccacaatggcgtcaccatccctgcgccgccgctggatgtgctgaagatcggtgaacacatgcagaccaagtccgaggagaagctcttccttgttctgtttttcgacaataagaggagctg

SEQ ID NO: 15 - DNA, 1446 bp, Mus <mouse, genus>
  source 1..1446: /organism=“Mus <mouse, genus>” /note=“BRD1 Exon 12 / Terminator region” /mol_type=“unassigned DNA”
gcagtggcttcccaagtccaagatggtccctcttggtgtcgacgagaccatcgacaagttgaaaatgatggaagggaggaactctagcatccggaaggctgtgcggattgcatttgatcgagccatgaatcatctgagccgggtccatggggagccagccagtgacctcagtgacattgactgaggtggtttccagcaaaggcggtggccaaagcctcagccagccgggagctctgtccatagtgttgataagctgtacatgtttgtatattgttcagaacttaacttattctggttttctaggcgtagttctttaattctttttcccctggggaggggaggtttcacttccaagttttctatgaaaccatctggtcttggctttgcaagtgaggagggtctgttgcgagcagtgtggtgttggggtcccactgcaggtgccgagggccgaggcctcacttattctaatctgtagggtttttttttttttttaaagacttttgaatgtttaataattttgtagatcatgctctttacacagagtaccgcttatttaataagacgggatgtaaatttacaatgacaaatgtgtattttaagaaagaaaatgacattattttgaatggtactttgtgcaaagagggaataaatttatgctgtgtgcatcacttgcaaatcaccaaaaaatgtcccgccagctgctgccggacagggcccgttctcctcgttgatctgactgccctgagtctcctgctctgccctggctcctgcaggcgtgcctcccagcgggttatttattgtagaaagtgtactcatttgctttataatgaaaaataaatttgcaaaggtatattgatatgcatttttatacaggcacataaaaattcaacttggtgtgggagcagaatgtgttgcgaggttatatacacgactggcctgtgtgtactttgattttgtaacttgtaatcttttgtttacaatgaggagctttctgtaacttgttttcatttagaacactttggtagcaatagactttggatacattttgtatggtacatgtgatgtatatagaattagtcctttatttttatttctaagaggtaaagcattatgttaggggaaaggcagggtgggtttccaaatttgcatttttatattaaaaataaagtgaagatttggacagtgtggccctctcattcctgcatcactaggaggctgggtgagctgtagcctgagggacgtgagggactcggagcaccgggcctggagtgggtggtgtgacacacttgatctaacagctgactcgggatggcattatttattattttgcctaatcatatttttattttaaagctaaatagttactaaaaattttaaatgttcttttaaatctacatgtttgtaatatctccatagaaacttgaaaataaaaagtcttcctttggt

SEQ ID NO: 16 - protein, 1189 aa, Mus <mouse, genus>
MRRKGRCHRGSAARHPSSPCSIKHSPTRETLTYAQAQRMVEIEIEGRLHRISIFDPLEIILEDDLTAQEMSECNSNKENSERPPVCLRTKRHKNNRVKKKNEVLPSTHGTPASASALPEPKVRIVEYSPPSAPRRPPVYYKFIEKSAEELDNEVEYDMDEEDYAWLEIINEKRKGDCVSAVSQNMFEFLMDRFEKESYCENQKQGEQQSLIDEDAVCCICMDGECQNSNVILFCDMCNLAVHQECYGVPYIPEGQWLCRHCLQSRARPADCVLCPNKGGAFKKTDDDRWGHVVCALWIPEVGFANTVFIEPIDGVRNIPPARWKLTCYLCKQKGVGACIQCHKANCYTAFHVTCAQKAGLYMKMEPVKELTGGSATFSVRKTAYCDVHTPPGCTRRPLNIYGDVEMKNGVCRKESSVKTVRSTSKVRKKAKKAKKTLAEPCAVLPTVCAPYIPPQRLNRIANQVAIQRKKQFVERAHSYWLLKRLSRNGAPLLRRLQSSLQSQRNTQQRENDEEMKAAKEKLKYWQRLRHDLERARLLIELLRKREKLKREQVKVEQMAMELRLTPLTVLLRSVLEQLQEKDPAKIFAQPVSLKEVPDYLDHIKHPMDFATMRKRLEAQGYKNLHAFEEDFNLIVDNCMKYNAKDTVFYRAAVRLRDQGGVVLRQARREVESIGLEEASGMHLPERPIAAPRRPFSWEEVDRLLDPANRAHMSLEEQLRELLDKLDLTCSMKSSGSRSKRAKLLKKEIALLRNKLSQQHSQAPPTGAGTGGFEDEAAPLAPDTAEEGANSPPKLEPSDALPLPSNSETNSEPPTLNPVELHPEQSKLFKRVTFDNESHSTCTQSALVSGHPPEPTLASSGDVPAAAASAVAEPSSDVNRRTSVLFCKSKSVSPPKSAKNTETQPTSPQLGTKTFLSVVLPRLETLLQPRKRSRSTCGDSEVEEESPGKRLDTGLTNGFGGARSEQEPGGGPGRKAAPRRRCASESSICSSNSPLCDSSFSTPKCGRGKPALVRRHTLEDRSELISCIENGNYAKAARIAAEVGQSNMWISTDAAASVLEPLKVVWAKCSGYPSYPALIIDPKMPRVPGHHNGVTIPAPPLDVLKIGEHMQTKSEEKLFLVLFFDNKRSWQWLPKSKMVPLGVDETIDKLKMMEGRNSSIRKAVRIAFDRAMNHLSRVHGEPASDLSDID

SEQ ID NO: 17 - protein, 1058 aa, Mus <mouse, genus>
  BRD1 (short) amino acid
MRRKGRCHRGSAARHPSSPCSIKHSPTRETLTYAQAQRMVEIEIEGRLHRISIFDPLEIILEDDLTAQEMSECNSNKENSERPPVCLRTKRHKNNRVKKKNEVLPSTHGTPASASALPEPKVRIVEYSPPSAPRRPPVYYKFIEKSAEELDNEVEYDMDEEDYAWLEIINEKRKGDCVSAVSQNMFEFLMDRFEKESYCENQKQGEQQSLIDEDAVCCICMDGECQNSNVILFCDMCNLAVHQECYGVPYIPEGQWLCRHCLQSRARPADCVLCPNKGGAFKKTDDDRWGHVVCALWIPEVGFANTVFIEPIDGVRNIPPARWKLTCYLCKQKGVGACIQCHKANCYTAFHVTCAQKAGLYMKMEPVKELTGGSATFSVRKTAYCDVHTPPGCTRRPLNIYGDVEMKNGVCRKESSVKTVRSTSKVRKKAKKAKKTLAEPCAVLPTVCAPYIPPQRLNRIANQVAIQRKKQFVERAHSYWLLKRLSRNGAPLLRRLQSSLQSQRNTQQRENDEEMKAAKEKLKYWQRLRHDLERARLLIELLRKREKLKREQVKVEQMAMELRLTPLTVLLRSVLEQLQEKDPAKIFAQPVSLKEVPDYLDHIKHPMDFATMRKRLEAQGYKNLHAFEEDFNLIVDNCMKYNAKDTVFYRAAVRLRDQGGVVLRQARREVESIGLEEASGMHLPERPIAAPRRPFSWEEVDRLLDPANRAHMSLEEQLRELLDKLDLTCSMKSSGSRSKRAKLLKKEIALLRNKLSQQHSQAPPTGAGTGGFEDEAAPLAPDTAEEVLPRLETLLQPRKRSRSTCGDSEVEEESPGKRLDTGLTNGFGGARSEQEPGGGPGRKAAPRRRCASESSICSSNSPLCDSSFSTPKCGRGKPALVRRHTLEDRSELISCIENGNYAKAARIAAEVGQSNMWISTDAAASVLEPLKVVWAKCSGYPSYPALIIDPKMPRVPGHHNGVTIPAPPLDVLKIGEHMQTKSEEKLFLVLFFDNKRSWQWLPKSKMVPLGVDETIDKLKMMEGRNSSIRKAVRIAFDRAMNHLSRVHGEPASDLSDID

SEQ ID NO: 18 - DNA, 47511 bp, Rattus
  source 1..47511: /organism=“Rattus” /note=“BRD1 gene” /mol_type=“unassigned DNA”
cattgtttgcttcgctggggagcgagcagcgcctcggcaggcgtccgagcagctccgcgttcgcgtcctccgcccggccgggccccgagccggccttagccggctgtgccggcgccgccgaccccgcccgagccgtggcgcctgcgggtccggagccgctggccgagcgcgccccggagcccggcggggcacggctgcgcggccgttggcggaggagccgcggcgccattagcgccgctcggccgcgccatctatatccgccgctcgcgccacacactcgccctcccgctccatccacacccccgacccccgcaccgccccacgccctccctcacagcagcggcccccgccgcgattccgccccacctatccccggttcgcccacacctataaccttctccccccctcctgagcacatcagccggtcccccccccccccaagattctaggtacacttacgccaagcgccgccactccccatcttgcacaaaaaacaaaagaagaggatcacacgccttctgccatacatccccgccccgactgccacggcctccgaatccgcccgcccgccgggctcccgcggccgcggcgccccgaaggtgagtgtctgacggtcgccgttcgccgcccgcctcgccggccggggcggaggtgcaggcgccatgtttagaggcggcagcggcggctccgcattgtccgcgggcggggaggccggagagtcggggcggcgaggcccggaggccgtgaggcctggtgggcgcgggagccggaggaactgagaaggccgagcgggcgagtgccgccgtgagccggcgcggccggggacgccgagatgggtgccggcggcttgcccgagaggccgggtctgggaggcgaggccgcggcgaaatcgcggaggcggaggccgcagccgggtgggggcggagagggacacggaggccgcggcggggtcggggagacagaggagtagaaggaggccgccgcggcgcgggaggggcggccaagagaatggagcgggcggcaggtttcaggaggcggggaagccgccgggccgggcgggctctgggcggcccggctgtctgtgcagctggggcaactgcggggacgggcgtcggacagcggaggaggcggaaggcctggggtctcgtggcgtctgcccacgtcctcgcctgtagccttggcggtgcggagccggtcgcattatgtaacagataggtccgatctattttgccaagacaggaaactcccttgaagagggacgggctcggaagatttcctaagtcgagcggggcctggtatctccggagtaagcccgcagctccgccaaactccgtggatgtgtgcaggaaacgccgagaaacgaacgcgcgtgcgcggctttcttgggcctttaggagagaagcaactttcctatgcttaatttgcagaaaacactgctcctcatcgtgcactgcagttgtgacacacttacacacacctaggaaaccgcccccttaatggaggacattcacttcacccagccgcgactgttttagagtatctgtcatctggtaacacatagttacagaattttgatattatttagttactgttttatcacttgttggatctagcactgttctgagtctgtgtttactcctcagattgtcactttagagtaagtgtctttcctgtgtgctttcacagtgaggggtagaagctggaagagtttaaatggcttgtctacaaaccaggcaggaaatgaactgagctgattttgagcagagtctttccctctttctgctaacaaagctttttaggatgcgtttagcacagttatttctggagaaccatgcttattgcctttgctgattctttcatggaaatgctcattcctgcatagagccagagggtcaaagtgctgggtgtatgaaaatgaggaagcagatgagattgttggtcactgctgggcagtgcctctaaatgccctctttcccccggtcacaattacattttcaaattacagagtagctgtggccattaagtattaggttcagttcttgtagaaaagtggtttaaagaccttcagtgctcactaggagaatgtggggtttgacaggctggttacagtactttactgtagaggagaaaattacatgtttgtttttaatctgggagctgttgcttctgcctgcctcagtagtaaattgtgaagcatccgaggtgaactgtggttctttctgtgcagaatatggtgctgacacctggatttgcacctatctcatctcagggatgttgctagaggcctagggctggctagggctgctttgatgacagctcccttagaatcctttgctgagcaggcacctggaagctcctcagatgcaggtgcattggggtctgctgttcttgttcatagagcgatagtatctacagaatgtgggtttctgcaatctgcaaggtctgtctttaaaaatgcgtataagatttgcagagatttccttttgggatttaaaacatgaagtctgctcttggagggcttttctcagagactagtaagataagtatgagctgagaattcggggttcctggagagccctgcttgtgggctattctgacatttcaacttggtatattttgggagtcagtctttatctacttgtcagttgagtgggcttgttcagtgggaggcatgagtnnnnnnnnnnnnnnnnnnnnnnnnnnnnnnnnnnnnnnnnnnnnnnnnnncctcagctaggcgtacttggagagtatgtggcccagcccacagccaggtcctggcctgagtgaaagggaaaggctggtgctgccacggcagcttccagggtggtcactgctgaggcgtcctaaaagctccattgggctcttggggcaaagatctcccacagaccagcacagcgttcccttcaagtcctgtatgggatgtttgtggaaagaatggactactttatgctgttgagttatggatgcttctggcccccagcacaaagttcccaggagcactctgctgggcagtagtgagaaagaaagacctaagggattgctaagagtaggtggccacaggccatagtctctgttgtgaagtctgtcagtaaaacagtctgacttgtgggcaagaggccaagctctcagctctggagacctatgttgctgtttgtaggtaacttttggcttggtctaaaaaggtgacttgtgggtggaatgcacctgtgccctagctattcagcaggaaccccgagggctgcagcttcctgctgtcttccctgactgtcagtacctttacctgggtgtggtgagggaggttactgttggaggcttgttgtaatgtgtttggaagtcttcaactctgagctttgtggggtgatttgttagtgctgcccaagcatattttgtagttttctgaagtcttctgtcaccctgcatggagttaacttttctttgactttattctaggtaatcattgccaaatgaggaggaaaggacgatgtcatcgaggttctgcagcgaggcatccttcttccccgtgcagtattaaacactcccccactcgtgaaacattgacatacgcacaagctcaaaggatggtggagatagaaatcgaagggcgtttgcatcggatcagtattttcgatcccttggagatcattctagaagatgacctcactgctcaagaaatgagtgaatgcaacagtaataaagaaaacagtgagaggccacctgtttgcttaagaactaagcgtcacaaaaacaacagagtcaaaaagaaaaatgaagtcttgcccagcacccatggcacaccggcttcagccagtgcccttcctgagcccaaggtgcggattgtggagtatagtcctccatctgcacccaggaggccccctgtgtactacaagttcatcgagaagtcagccgaggagctggacaacgaggtagagtacgacatggatgaggaagattacgcctggttagagatcatcaatgagaagcggaagggcgactgtgtctctgccgtgtcacagaacatgtttgagttcctgatggaccgctttgagaaggagtcctactgtgagaaccagaagcagggtgaacaccagtccttgatagacgaggacgctgtgtgctgcatctgcatggatggcgaatgccagaacagcaacgttatactcttctgtgacatgtgcaacctggctgtgcaccaggagtgctacggggtgccctacatccctgagggccagtggctttgccgccactgcctgcagtctcgggcccgccctgcggattgcgtgctgtgcccgaataagggtggtgccttcaaaaagacagacgatgaccgctggggccatgtggtatgtgcactgtggatcccagaggttggctttgccaacacggtattcattgagcccatcgatggtgtgaggaacatacctcctgcccggtggaaactgacgtgctacctctgtaagcagaaaggcgtgggtgcctgcattcagtgccacaaagcaaattgctacacagcattccatgtgacgtgtgcccagaaggctggtctgtacatgaagatggagcctgtgaaggagctgactggaggcagcaccaccttctctgtcagaaagactgcttactgtgatgtccacacacctccaggctgtacccggaggcctctgaacatttatggagatgttgaaatgaaaaatggtgtgtgtcgaaaagaaagctcagtcaaaacggtcaggtctacatccaaggtcaggaaaaaagcaaaaaaggctaagaaagcactggctgagccctgcgcggtcctgccgaccgtgtgtgctccatatatcccccctcagaggtaagtgcatctgagcttcagctcagatgggcctggagggaaggacttgatgcaggacacaagtcagggcctgcaggagtcctggcacatctccaccgcacctcctgatagtctgtgtcctaagctgtagccattcattcactactgcccagtggggcgtaagtgcaagagaaattacagattgggataatgtatggttcttagtcacctgttgaccgtgaatataaggtaggtatgctcaatgggagccacagccacaccagtgttcaaccctgggcttctggatctcagcatcctgagttttgtttctatctacaatgccattaaactgccttcttaccagattttaggaccttgtagaaaagcatctggaaaagtgaaccaccatcctcagtaaggtgaccattgagatgaggttagaaccagggctgctgtcagcagggagatggtgttctgtcttcctgcctgccttgaactcccagggatcctctccctgtctccctggtgctgggattcaggatgctccaaccatacttgacttcttctactacttacgtctgcagtagtgcacatgtcgcttgatctgcaggagggctgctgtgccaagccctgcatctgtgttccctgagggaggacttgtctccttgtgtcttctggacttgctctgtggcatgctgtttttgttccattatttcaagaaatgtactgtatatcacatcatagcctgtgaatgccaagtgaatccactccttttgcatccatttgaatccattttagagccttgaggaagtgatttttttgtgaagggggcgtggacttttagtctggtcaggtttgtagagccccaagatgacgaagttcatgtgaggcaactgccctaaagcaacatacatggtggacagtagatttcaggggtgtgtgtgtgtgtgtgtgtgtgtgtttgtttgtgttttgagatcaagtctcttgtgttcttggctgtcctggaactcccatccactgcctgcccctgccttatgagtgctatgatcaaaggcgtgcaccaccaccactgctcagaagctgtctttaacacagcgagcgaacattagttttgtgtgtgtgtgttattatgaatgtctggtttgaagaagttaatcattcttactagcatgctgtggttataccgtggagttgggcattgtgccacagtgggcttactgtttgagctaggagcacaaatggaaaaagagctagcactgcctttatgctagagtttgaaatgggtaaatgctgtttgtttttgtagacctatacttctagtcagtgaatcaaacacagaggtctcataaccaaccccttgttcagagaagagtcccattaggaccagcatgcctgaaaagtttttcagcctgacttagaagatggtctctctgggatttctgtgtctgtagaggcagaaagcatagggttatgtgaagagcctctgctcagggaggttctgctttaccaaaagtgaaatagctgagccatcagtcgtcttgttgctttctctggccagtgccagatgctctgttgcaggtggtgtgatcctgcaccctgtcctgtggttcctgatgttcaggttttgggacatgaaagctgccaggtgggcgggactgttgcaaggaggatctgcaggtgacaacaaagactctgtccttcagagccattgaggaaagaactcgaagctttaaacttaaatcttccagggtctgttgtggaattcagcagataagaggtgcattgtggctcgtgtttttctcctccagacaaggtcttatttataggccaagactacctggggcttatggagcacacgcccggtcggcctcagaactgtgagtctcttgcttcagcttgtcaagtgctcactgtcctgtcctggcttgccttcctttgttttgtcctgttttgttttgtgacaggatttcactatataacccaggcaggtttcagactgccagcctcagcctcgtgaataatggaattacaggtgtaagtcatcatgcatagctgcttgctgctgctgctgctgctttttaaagatttattcattttgtatgtgtgttttgcctgtacctatatatgcgtaccatgtgtatgactggttccctctactcagatcccctggaactagagttggggtgggttttgagtcaccagtgaggtggaattgaatatggttgaacagctccttggcaccagctgtaactagtagactgagggcaggcagtgctggggaccatactgtactgtaccgttccgtgcccagcccttgggaagcagaggcaggaggatttctgtacagagttcaaggccaccctagtctatgtggtgagctccaggagagccagggttacatagagagactgaggaaggtctaaagaagacaatttttgtgtgggtttttgtccttttgtgtgtgtgtgtgtgtgtgtgtgtgtgtgtgtgtgtatgttttctgaggcagggttttcctgtatggtcctggctgtcctggaactcaccctgtagaccaggctggccttgaacttgacatctccctgtccctgcgtcccaaaggcgtgcaccaccactactctgccaatttcttttaagtatgagtacatactatagtatgaattttctgggaattgaactcaggacctctggaagagcagtcagtgctcttaaccgctgagccatctctccagaccttgccaatttttttttttttttttaaagaaaaacattttgatgtgtttttctatgctcttctgatagaatttgctgttaccctgctcatccatctgattctgtctcacagaggaggaaagagaaggaacaggtaaagggcaaagcttatttgttccctcccctgaagtatgaagcctggttcttccctgccttggccatatggggccatattctttatccctggaacaggctgctggtgagcagcagctgtgtgtgcccagatctgggacttgactcagatagcctctgtcgccagatgcttggcgtgcagtttactcagatgcccaagtagtggttggtgctgggctagtgagactgctctcctcctctttgctgttcacactgccagtcctgatgtctctgggaaaagaccaagtgacgtgtgagcacttgatgcattcagtaggtagaccgggtcttgccttttcgtgtggtctctggtttgagttctgtacactcccctccccatgcttctggtggcttactgtatggccagtccctgacttaagatttgacttaccagcttttttctctttttgttaatggcacagctgtttgtttgtttatttagcatttgacagaaagggtttttcctcttgcatggtactgactgtacgagaactcgctctgtaggtcctgctggccaccaactcagagatctgtctgtttttgcttcccaagtgttgtgattaaagacgtgtaccaacacacctgacttttttctctctaccctctcttccctcctcccctagtcagtcagtcagtctgtctgtctacgtaagtttttgaggtaagatccgactccctccatgtgcctctgccccctgaaggctggggttgaaggcgtgtccacagcatgcctggcttagttaccagttttgaaaaacggtacatgcagaaatattatgcattcagtaaaaatataatgtgaattttgctctttgtgggctggtatgtatcatactccttggggatgctggacattgcgctggtgtgagccagggcttcagtgagctgcacgaagagggggccacgtcgtttaactgggctgttgggtagcatcgttgtaagaatgaacctttgacttgttatgtgaggttgtccgcatgtagcctcctcctggcaaagagcatctgcatttgataaacatttccgtctgccacggttggctccttccctgccaggtggaagccatgccctgcatctcctgagagcagtgtaaggagggcttgctgctgtgcagggccttggaaagcaggcagatgcagctacagaagtgatgttggggaagcatttaaacaaccaacagaaaagtacaaatgacacacactgtcatgtgtggaaggcagagcccacctgagagctggccactggaaagagaggaactctctgtctgagagatgagtgggcccgcctactgagctgcagcagcgtgcccctagaaagctggatcactgtcgagctgaaatcactgtgtaccgactctgtccccaacctgtacaccttcccttagtggatctgggtctcctgtgcatctccagtgtggctgatgtcctcttaattcttacatagttactttggcataggtagaagtggacttcctttagacgtttattgggggtagggttgttcctttgcctcactactgctgctgtgagtccacttgaagctgacattgggatggacttagtgccagagctgcccagagtctcttcttgtgggatggagcaggacttccaagtctggctgttagaatttttttagtgataacatttcaaaacataaaaataaagggaacaaaaaggactgcataagaaagaaggactgcattacatcacccagttatttgctgctagtgttctgctgtgaattctgacaatccttatgtatgggttgtaggagaggccttacatggtcatgtgctctcagggcgcaggtttaagttaccctttgcagtggggaaggacacgcaggtgtcctgtcctgtacatggtcaccttctagaggtgatcttgatacctggtcgttgctccatatctgtgcctccagctccatgccccaacccagcctgcctcacacaacactcaggccctgtgagtcataggaagaccattctgatacctgctttgttctaaggtgctagagcattgggtcaaatggaaggtaaggggccagagggcctagaggtaggcaggctctacctagaggcaggggacaccattttggctgtttagtcttttcagctttcctgggtttggatggtttgtctttgtcttctgtgatacctggaagaatgttcttccttttgttcttattttataactgtaattttgctactgttacaattaaatatttttggagatagagttttgccaaaagggtcacagcctacagattgagaaccactggtctacagtattgatggtcttttttgtttgtttgagacaggtctatgttgaccaggctggatttgaactcaagagatccacctgcctctgccttccaactgctgagaataaaggtgtgtgccaccgtgcctcagtgtttcagagtgtgttgtttcttagatctagtttgacatctttggtgggacttcagaacaagtccagactgagggtggtttctgcctgaccagtcttcctatgatgattagaataggtgttcatcctcagggcctggggtcttccttctctgtggggcttttgtcttcatacttttgtgctggtttgtcagttacctgagtaggctgggaagttacttaccacataggcatgggcatggtgagagtgtctgagaggagccttccttttcttatttcatgggaggagaacagtcaaccatatgatagcacttataatacacttggcccctgcctagaggtaaattgtagacttttgggtcctgggaggcagaaaacctaggctttaaaatgatgcttgggttttttttttttttttttttttatttccatgacgtaatgattctattcattgatactaaggttagagactcctgccagcttgaccatggagctgtcctgaatatgaatggagttcattataaagatgtaggatgtggggttggggatttagctcagtggtagagtgcttgcctagcaaacgctaggccctgggttcagtccccagctccgaaaaaaagaaaaagaaaaaaaaaaagatgtaggatgtataggaaatgttgcattaagaaaggaggctggggtggagagatggttcagtgttaagagcactgctcttccagaggtcctgagttcaattcctggcaaccacatggtggctcacaactatctgtaatattagagatctggtgccctcttctggtgtgtctgaagacattgatggtgtactcaaatacataaaataagtaaataaatctttaaaaaagtgaaaaagaaaggaggctggtttctaaaattcactgtccagaagtgactggggcatcttgaggttgtggatttgtctgtgcacacggatgggcagaagttcttttcctacttttcatgatttttgcctaggacaaaccagagactaacataatctgaatcatcaagtgtcacagagagcctcagttccctttggaggcctgcagtcctggatccattctcgttcttagggcggcattcctgtgcattcccgtgcattcctgtgcattcccgtgcatctggttggtttgtaccagcttctttctgcaaggcttgccttctactttctagtgattgctggaatttataaaggaaaaaaaaagctgtaagaagtacagagaggggttggggatttagctcagtggtagcaagcgcaaggccctgagttcggtccccagctccgaaaaaaagaaaaaagaaaaaagaaaaggaaaaaaaaaaaagaagtacagagaaccatttgttgagcaacttaagctgtgactgcttagtcctcccttgcctgtacctccctttgccttgtttagaagccagtcccagccatgtgactgtgtccatgactctacagtatataaagatccttctcagaactgaagattgatgctgactgatgaagtgagtgttgatccgtccttttctgtgtagagtaagcccagtggtcaggagcctttggggtcctagaattgtttgttgaggatggtgtgagggagcctaagtgttccagccccacagttacttacctcatgtgttcttagtctggttccacagcattgctgagcctggaggtagatcataacacctggggctttttactgactgtcccatgactgcatgactgtccccgtgacatcagtgctctacaggaatactgactggtagggactaccctgccactcacatagggttttttttttttctctccccttctttttttagagatagggtctcttcataggtctggctgtcctggaaatcactatgtaatccagactgccttgcttctgcctctctagtactgggattagggtttataccaccacacctggctaacgtgaaaggattacctggtgaggccgtctcaaaacaataaccacaactctacccaccttcttttccaaaatacccccaaaattacccttttgtgactttgctaggttttttgtttttatatcaatatataatacatcttaggttatttttgtagacagcatctgtgaaaatggcagtttaggtgggcttttttggtctgtcagcttttttactcaacctcagacgaggccctgcctattgagctgcccagaaggagattgactctgctatgcaatgtactgtgctgtgttttttgtttttttggttttttttgttgtttgtttgttttttaaagcagtcttactgtgtagctccagctggcctgaaacttgtatgtagaccaggctggcctccaactcacagagatccatctgcctctgtgtattgagtgctggggagaattcttaataataagtaatatttaaatcacaaccgagtcacttgtttttaaaaaagactagttaggatttccatggatggatactagtattagaaggggacgaggtagcccaatgtttcttgctgttctttcctttccttttgagatggtctctctgtgcagctcaagtggcctcaacctcaccatctttctgcctcagactcggaagtgctgtgtgtgcggcctgccccgctttcttcctggtttgttcttactgacggagctgctggaaatcctgccctatgtagacggtataaaagtgactttgtgtggcagtgtatgtatcaagttaccaagttagctgtaagcagtgacttccaccttagacctaggcctgtaaagactggaatggcgggcagtgtacgtcacttgccaccctcactctgtacgcatctcttcacacctctcagcataacggtccagttagttttcctcctgtgtttttgttttgtttagtagctgtgtttaggaatgcttgaggtttttgagtgtactctcgccagcatttaaaatttttaaatagactatgatattaaaagattcacaagacagacgtgtggttaataaggtacaatggagtgattgtaattagtctgtgtccagtgagcccttgcttgtggcagcccctgcacagtcccttcctatgagtgttctggcttgtgttaggtttgcttcatggacttttcctctagtaatcttggtgtatttgtgcctttgatataatgcctgtaccagattttagttataaaagctaaatagacaaggttggatagtttataaagaagtttttttgagctcacaatttggggagcagagagcacaagattgggcttcacatcaacttactgtgtggctgaatcctatccgggcagtggcatgcatggtggtgggagcatgtgtgggaagtggaaatgatgtggtgaaataggaagccagagcacagggagctgccggcttttggtcttcctgctggcacctgtcactccagggatctggggcaggctagggtaagtgcctgcttccttctagaggttcattttggtcttgtccacttccaggttcctgtatgctgtgggtgggtctgccttgtgttggttgcctgtcagtggtatggtttggcctgttttctctgagttagttggtagttggcttgagtgttgatgagtgtggggtgcactggttggacatgcttgctcttgactggtctgtttttggggagctacagtattgtagtgcctgttgtccacctttctccgaggtgtgaggttgctcacggggtgagtcagggtctgcatgtaacgtttgggtacatcctgtagaatacatggaaattatttttatataagtcttgtttacaacttgcaagctattcacaacttcccaagttcttgcactggagaagggggtggggctaatatgaaattggtatcttaattaaagcaaattgctaaccattatttctttggatttttaaattgttaaaaaattcttgtatctggctgggtatggtggtgcacatggtttatctcagcacctgggaggcagaggcagaactctgagctcatggctagcctggcctataaagctagttccgggacagccatggctccattattacacagagaaccctgtcttgaaaatcaaccaaaaccagccaaaattcttgtatctctgtactgcttcctgcaaacattaaaataaccacgagtgtagtgttaaacatttgttcgcatgttgttgggatgtgtgtagtttactgcccagatgtcacacctctgaaaacacaagcagtactagtgaagtagccaggcctcctgacctgctgatgtagcttcctgtgggtcttgatcactgtctgctgtagcctggtttcctgtctccatctgggtgtcctttggtgggtttgtcattagagatgatgcttcgtggacttggtgtctgacccacccacactgaacaggcagagccacctagagcagtgcaccatttagtggagctcagccaggaggcttgacagcctcactgtgtgaagcattctcacgggcaagccggccttggcagagctgggccttctacctgtgctggtgtgttttgattgttctgtggaatttaggtttgcattcttcttcttcttcttttttttttttttaaagcaagaaaaaacgaaaaaactgaacttcgaaaattttagagcctgttctgaaattttgatgtgtggtacaatgaaggaacaccttcttgtagccttttggagtttcattcttttgaaattgtggggtttggtggagtttgtctttcagtatctttgtgaggcacactgagctctttttctgcggctgtggtgtaaagcagcccagaatttctcagaggtttttacagcttggtgctgctagtccacaaaggacgaagtttctcagatggttgtcatttactaagagcagactgttcccaacctaagtgagtgggtgagccactctgtttctggagtttcttcaaggttcagtgtgaccagggctggtggtgccacctggtgagagcaggctgtgacctcagagtccagccatcagcatctcagctgacagtgatcaatagtggttgctgtgtgtgtagataggacgtcacacaggagcagtttgttaagctgtttcttttagatgtttgacctgatgactgttttggtggatgaaatctttagttagttgaaggttatgaactgtttctatagtaccagggacaggctcaggagagaactgcagtgttattgaaggtaacattgtcctgtctagttttctaaatgcaaacactttttaatgtgcttttcaaagctaaactctcagtttttccatgttttagattaaataggattgcgaatcaggtggccattcagcggaagaagcagtttgtggagcgagcccacagctactggttactcaaaaggctgtctaggaatggtgctcccctgctgcggcggctccagtccagcctgcagtcccagagaaacacgcagcaggtatgtgtacatgctcatctgcctttcggtgtcacgtgcctccaaacacaggctgccccttcaggctggatgtgctgaccccgacccctgacccctgagccttgaactactaagctgcagattattcaggtggctcctatttgcccaaggtttgctgtggctccagggttgagttgtgcctccttcagcccagggaaaagggagtgcgaagagaacctagttcagcttgagccacatgcatagtcaagacaggagacctgatgaggcctatgcagttctctacacattgcccgagaaacacatctggtggtttctgctggttccagtgggaggagctgatcatttcatttgtaacatatcaaccaaggttatcggaagtttacaactgtagaatcctgttgcgtccctgtggctgtcacaggtatggctgtcacaggcacagttgtgggatgagctcagggctctcatcctgtatcctgtctgtcagatgtgtagtggttctggagccctcttgtgtggattagacacttatatctggaagttaccaagtattgctcaatggacgaggttcagtccattatcatcctggcgggaagcatggcagcatccaggcagacagagtgctgttgaaggaaccaagagttctacgtcttgatccaaaggcagttaggagaagactgctgtcttctaggcagctaagaggaggatctcaaagctcacccccacagtgacatactccttctaaagaaaggacacacctcctaacagtgccactccctgggccaagcatgtacaaaccaccagatagggtatctgaagcagcccatctgaggagtgtcatggtataaccaggctttgaagtgcctagaacagtagaggctgttttagttgttgagtggatcaaccatgttctttacgtcaacatactgtgagaggctgctgttctcaataccgcccttaactcttggcgcccagccctaccatgagatggcttgtcctgggaaagagggactctcctttgtaccagaggagaaaaaggttttagggcaagagagtcttagggatattggtcattaagtgggggaggtgtttggtaaccctgggcactggtaggaacgatgcttatgtgatggtggcagtgttgggaagagaagatgtggagagaaaagtggagaatccagaccacaggtattgtcttgggctagaggagagtcctagtgaaccgctgaggaagagtgttttggtagattatcagaatggaatgccgttagtatagtggtggggccttcagctgttggcttcttctcctctgcatacataggagcatagaacaaagggcatgggatgccagtgtccctgtgggtagcttccctagagaggtggatctgggaggaagaagaggctacaggaaggaagataggaaggaggtgataagggaaggaaacaaggctggccccctgatggtcatccctgtcaaagggcaggcctttgcacaggacattgaggccgtcagagagagggagcaccgaattgtgaggatgcaagctctgtggtgttctgggctgggtcattctagaattaccagaagggaagtagaaggctttgtccatggcagagaggtctgctctgctgtgttggaccaggcaggacatgaaattggaagttgtaaactatacccacattgtcttagtaaggcttgatgtaatggccctgagccttgcctttgacatcgtgtatgctccgtctcagcctggatctatagactagaaatactgaatgttagaatttgacttacctttgactttagctgtctctgctcagcccagctttggaaaaaggctgtgcgatttctctactgtgactaaccttgtggaggatggatgaggcatagggatggtgggacaggatgagctgctatgagaggacatcactgacgttggtgttgtggggagtctttcactgtggtggctagaagcttcccagctgtgcggtgactccgtaggccctactctggtaggaaagcaggcatgttgcttgtgccctctgctgaggtatagtaggaggtgggttggtgtggctccttagatttggtccaacagtttgtcaggtgcaagcccccattcatcctgttttggtttttttttttttttttttttaatttcctgttttcctttccctctagctctgggcctctacttgtacccatttattcatagaattctggaagtcttgggtctgacatggctgagcctagctgcccttagggtcatggttgaagtgtatgggagccactgctgccgatctgctgtgtgcttcacagatacgctgaccagttctcccagtacaggggcccttggcctcactgctggactggtccttgtcacagggctgggtttctgccgtcctcctttatcccagcactagatcgtgacctgtgttaggagtggaaacactgaatgcttgtgctcttcttgggcgtgagcttcctcttctcagaagtctctcctggaagactcccagcattggttgctatgtaccaaagtagactgcttcaggatcgtactgggaaagctggttcatagatgggatggttggtgtagattggtgtacagggtcctgtcttcatgagcctgaggcatgttggagtacagacagtggcccagttaccccatgaccttataaagattaaaaccaggccacgagcaaaccaccgagttttgcctatccctaaatactcaagctcagatctattggcaatcgggagatttcttttgcttcatgggggttctctgtgagtaaccaagtctgtttctaagtagcagataggaagttgtccagatgttagggtattagtttcttttttctttgtttatttttgagaccgggttgacctggagcttgctatatatgctggccttgaactcatagaactcagtctctgcctcctaagagttgaaattaaaggtgtacatggacacacctggtggtggttggtttctgaacctccctttccttgtatttacttacttggcctatatgagatgatactgtcatcaaccccaactaaatgcttaagaattgtcggtaatatcaggtacagcgtacattactgtgggtgctgaagtatgtgcattgagacagatcatgccatacccattctgtgctgtcattttcaaccatgaagagtggctgtcgacagagtttttggtcggtgacacttttccctgagatcctccatccttgaccagtgtgctggtagcttgggttgcagaatctctgctgtggtgtcattgggctgtgagaggcaaaactgtccagagagagaagggtctcatgtctgtgttctacagctggctgtcagcactttgctcgtggttgacagatgtggctattactgtccagtagtgcagaaacttttgggtaggctattctccatccctttaccatagggacaggacactgtgttactgcaaggaggtcatcccatgtctttaacacagaatagagagtggggatatagctttggatgatgactattgtgttggatgaggacccgggtcttggacaggctcactatggggtggcaggaaagagtgatatctgggttgggagagcagagctctggggaacttggtttaaataagatgcatggattactgagaggatgtggcatgttgaatttcttaggaagtggctggaaaacctggtcctttgtagatagggctctggtcttgtttggtgtccttggttgctatcgagggacatgtgctatccctgtggcattggctcttgtcccctgtacatttgtgaggtagtaagagtaccctttggacatttcagccttgagtggctccatcaggagtctgtcgtcgtcttcttttttttttaaatttattcatttattatatataagtacactgtagctgtcttcagatacaccagaagagggcattggatctcttttttttttttttttttttttggttctttttttcggagctggggaccgaacccagggccttgcgcttcctaggcaagcgctctaccactgagctaaatccccaaccccggatctctttacagatgttgcaagccaccatgtggggttgctgggaattgaacttaggacctttggaagagcagtcgttgctcttagccgctgagccatctctccagcccggggtttctgtcttcttaatcctgcttagaactctgagcttctcaaggattcacatcccatgtgaccaggcagagccccactgcttttctgctactgtctgtgtcgcttgacttcccagtgctgtactttttgcacattttgatggttagggtagagaggggctggtgcaagatgctgaccaagttaggagaggtgctatctggtgtactgctctgtcacctgagaaggcagctgtgactggcaactacagtgcccatgctagtctatggggttagttagaagtgatccctacacttacctgccgagccccgaactgagcctgtgtaatattccgctgccagtaaggattgcttaggtttgtaccttttgtacatctcctttctaatactccctccattcctacctcctggagtcaaaccaagaccccttgtgccgtggtcccattagaccttcctgttccttgtcactgggtcccaggtcctgttacccttttagtctcacttgttgtatgagcttgttagacccctggcaggaacttctggctttgactgatggaaagtttcatttaattttctcagagagaaaatgatgaagagatgaaagctgccaaagagaagctgaagtactggcagcggctacggcatgacctagagcgtgcccgcctgctgatcgagctgctgcgcaagcgggagaaactcaaacgggagcaggtgagtgtgtggggccctcgggagctgccaccttcagggctggctctctctagatggacatcttgctgctggcccctgtgtacctgctgattctgtgtgctgtcccctccctacagcatatccctaccttatagttggtcctgtggtacctctgtgttctttttgggtagccactgcctcaatgtcttaaaggagaatacttgtccttgcagagagaaggctgccttgtggtagggtggtagcgttcacgtaggctgctctgtgctgatggttggagtgtcgcttctgtgattgtgcagtatgtggaggtgcacgatctgtctctaagagagctgtccctacactcctctagagatagtctatgctgttgttgccaggtgaaggtggagcagatggctatggagctccggttgacacctctgactgtgctgctacgctcagtcctggagcagctacaggagaaggaccctgcaaagatctttgcccagcccgtgagtctcaaggaggtgcgtgctgctgtgactctgttctttttcatgtggttggatccatactgctgcttggttaggaagcacgggactagggagagcaggttacctgcttccttaattctcattattatttaatatttaatgaattttagtggatagtagtttaattataaaagattgtgcctctttgtaaggcactgagaatttctactcaaaaattagctattggtaaagagaaccctgctggttccccatctgttgtacttttagttcaaggaagtaggttgggagggtccctgcagtgactgggcttagtttgtattgcctagagttgatgggagggcggggcggagttgtatgtctcaggtgtgattgactatagaaagcatgaaataagttttgattttttttctttggtttgtaaatgtttattttccttcctaaaattaggtaccagattatttggatcacattaaacatcccatggactttgctacaatgaggaaacggctagaagctcaagggtataaaaacctccatgcgtttgaggaggattttaatctcattgtagataactgcatgaagtacaatgccaaggacaccgtgttttatagagctgcagtgaggctgcgagatcagggaggtgttgtcttgaggcaggcccggcgtgaggtggatagcatcggcctggaagaggcctcgggaatgcacctgcctgagcgacccatcgcagcccctcggcggcccttctcctgggaagagggtaagaacccggcactgcatccaggaggacagcggatgctttttctctcagactgtacttattaagactccagcatgcaggcagcatgcgtgctcctgaggtgcatgtgcaccgtatatgcagcacatctcacatgggccttgccacattttcacacacttactgcaagaagcaggggtctaggtggtgaaggccgtgaagacaccatagttgagcattcatccccaaagggactagccttgcttctgaggaggtcttgcagtgagaaggcagccattagtcatcatatttcagctgagaaataaaagcaggaactaaaattggctgtgcctctgatcctctctctgggatgctttcaggtcctcagagggcccagccttagcctgtttttaggacatggcctaaccctcctagcccttcagggtgagcttgtactctggaccccaccaggcacatgctgttgtgctgttcattaatttcctccaagtacggtgctgattttggagataaggtcttgatgggcagccttggctgcacgtgggtcaggctggcttgggattgacagaagtcacctgctcctgcctcctgagcgctgtggttacatgtgcaccgccatgtctggccttcaagcagttcttgtgaaggttttgccccttaatctttattttgtaggtgcatgaagtttgcttgtatttacctaagatcctgtgttcctgttttgactgcccaggacatggtggaactgtactgacttaggtttatccagtgcttttccttctcctggatggtcagccagctctgactctgcctttgctttcccattggttatatttgtgacttagtgacgtcagggccctcaaaggctccctcactccccagagaaactgtctctttagtactcgcgcttctgcagggcatacaggatagatagaattctttttttcaagataggatgtagtgccacactcaggaagctaaaacaggacatttgccgcaaattcaaggccagtctagactgtagtgatttccaggctactgtgagttacactccgagaccctgcctaaaaaccaaaattgatccaaaaagtataattagaaagaaaacacaagcaggccagagtgtggtttagtagtttcttttcatgcacaaagggttcagtgtcagcacagcataaactgggtatgttgatacaaagattaggatttaaaggtcatattggctacatagtgaattaaggctagcctgtgttacatgagaccttgtttggaaaaatagatgcattgcacacagacaggtgagagacaggtgagagatttgtgcaaccctagatacaggtccagtgcaactggttagtgggagccatcttgtgctgagatgtcccccgagcaggagacgagcctgattgcgcccaggattagagtgactctcagtccttcatgtacatcctgttctttcttcagcctgtgtgggaggcaagggtagtgctccagtcttagctgatgtggctatgactgctctgaatggtattgggtgccttagaagcagaggagtaatggcgtctgggagtctccgaccccatagcttctgattctcatgctctgtggatggacagggcctggaggcctcaaagttgatacttccaggaaactagctttgccaaaggggaaagtagtaatgaaaagcacaaactgatttctccctcagtgattgagtaggatgagctctgggtacctctgccactgttttgagcctgctctaatgaagatgcttgtcttagggtttactgctgtgaacagaaacccctccagaaaccccctatcccatcccccttcatcctcctacttctatgagggtgctccctcacccaccgacccactccttcccacctccccctgacattcccctacaagcaacatttaattggggctggcttacaggttcaaggttcagtccattatcatcaagaagggaacatggcagtatccagacaggcacacggtgcagaagaagctaagaattctacatcttcctctgtaggctgctagtagaatactggctcccaggcagctaggagccacgcctactccaacaagaccacacctcctagcagtgccactcctgagccttgcctatacaaaccatcacattccactccctggcccccagaggcttgttcagacaagtctgtgagaggccatacctaaacataacataatgcaaattacatttagtccaatttcaaagtcgtggtctcaacaatgttctaagttcaaagtctcttcagagattcattcagttgtttagctaatctccaaagcaggacaggaaccagcggggcaaagtttgcatctccatgtctgtcaaagtgatcttcagatcacccaccccctttgccatccttgttgactgcagcaacgtctttcttctgggctggccccattccctgttagcagctttccccagcagagtctccaaggccacctctgttttatagcttcttgatttagcttctgggatccacttacgatcctctgggctccttcaaagggctggtgtcatgtctccagctctgccctctgtagccctctgaactcagaggacctgccactactgtacttggtgatcatcccatggtactggcatcttcaatacactggggacttctgctgcagctaggccttaccaataacctctcacaggctctcttcatggtgccaagcctcctttgcatgaccttttcagtcctgggccatcaactacacctgaggctgtaccttcaccatggccacagtgcccagcctcagctgcttttcatgacccttcctaccttcaaaaccagtgccacccgggtgactcttacacattaataagtatggaatacagcttctttgtgttctcagaaaaaactcccagaagatttcatctcagtgatggtctaatttttttaatgagtacagtatagctctcttcagacacacaagaacagagtattggtccctgttatagatggttgcgagccaccacgtggtttccggaattgaactcaggacctctgacccctgagccaactcttcagccctgctggtctcttcttaatcaccactaattttttagctccagttaactagcatcaattgtcccagtagtctgttttctcttgaccaaaaagccagagacacatgactaaagctgccaaattctgctgcttgcaggagctggaatatggtccccttctataacactgtcaccagcttcctgttttccaccctagctcggctgtctcggttcttgctcagtagattgaccttgaactcagagatcggcatgcctggctcctgggattaaaggtgtgtaacaccaggcctggatttacgcttttcttcacctacaacttgctcctaggctggccttgaatttagagatctgcttgcctttgcctggggaggggggtcaaaggcttgttctaccttgtctggacctaaatttagctgagtgggatcttgccccaaggttctgccactcccttaattcaatttattatctttgaatataggttttagctcacttcctgatttcctttctaaccttggtatgcttattcaaaacactcttgaattttaaccggagaagaaagtctgtgatgggtgtttccgagacgtcctttgtaaatgcaattattctgagtctcttcaccttagcctcaggcagactcttcaggcaagggcaaaaagcagccatattcttcaccaaactacaaaaccagtctctaggccacaactgaaattcttctccactgaaacctcttgggccaggtctacacagttcaaatcactcacagcaacaaagtcttccatattcctactagaatatcccttaagccctacttaaaacattatggctttccaaattcaaagtcccccaaatgtacattcttccacatgaaaacatggtcactcctgtcacagcagtgccccagtccctggaccaatgtcttacggttcactgctgtgaacagacaccatgaccaagaccgctcttatataattggggctggcttacgggtttcgaggttcagtccattatcatcaaggtgggagcatggcaacaggcagacatggtgcaagaggagctgagggttctccatcttcctctggaggctgctgacggaatactggctcccaggcagcgagcctacactcacaaggccacacctactccgacaaggctgtacctcccaacagggccactccctaagccaagcacatacaaatccaaaagaagcggacaagcagggtgtgcaggcctagcactcagtggttgaggaaggagagtcactagtgaggccagcctgtgagatcctatctcagcaagcgaagaacagagcaaaaggaaaccagcattggaaagttttgaggggaggggtgttaagattattttttattttcggtacttcagattaaaggaattttgtttaccggaactcatttgaggtgttaacttttagattttgttagaaatagtgtgactatgagccctgaggtagccagccgggcagggtttgctcgtgtctagtgctggtcagtgctgttcttcagacagggcagttcgggttctcactggtcagctgccaggtctgggcaggtctcctttatgctgtgtatgtctctctgttgcccctgctggtctttggttttatctttgcaagattaaagaatttctttggctgttttactaagttctgtagtcagtgttcttagaatttggggaaacccgcggactgggcgcctgctgttgatgtgggcgtagtaccctgcagctcctgttggctgtctcacacatttctggtggtcttcgcgcccctcacgttttacacagcaggactgtgtgggagcctcttccaggagaggccacacacgctttctgcatgtcctctgctgtggccacgttagtcctttgtgtcacactaactgaaggagtgccttttttctagcgccagccttgtcatgtgttcagaatcagggtagaggggactatatatggcatcaaatggtgaaatgaaacaaaacaaaaaccaaccaaccaaacaaaaaagaaatggtgaagcttgtgctatggccatgggcaggctttaaagaatacttgggatcagtgtgttattcttagaggagcccgagagtcgggtggctgatgatgtctgttctttggttcagtggacaggttgctggacccagccaacagggcccacatgagcttggaggagcagctgagagaactactggacaagttggacctgacctgctccatgaagtccagcggctcacggagtaaacgggcaaagctgctcaaaaaagagattgctcttctccgaaacaagctgagccagcagcacagccagaccccatccataggggcaggcacaggaggctttgaagacgatgctgctccactggcgccagacacaggggaggaaggtaagcatgatggggtgggagggccgtacctcatggacatgggtgtctcctgacaggcttagatgatgctctgtagtaatcaatcgtgaacttgtaagttttgaaggtcacagaactcttggtcactggatagtcctcctaggttttctttttaacttgagcctgaaagactttacaagggatagtttatagagctgatgctggattgaaggtggcttctatggagggaataagaaattcttagttgtattttctaaattgaggcagagtattagatggttagatcccctgaaattgtttttactttgtgtgtgtaggtcagaggactagttggagttggttttcctggcatcttacaacatctgggtatcaagccaaggcgatgaggcctcgtgagcacctctacccctttgcccctgctgccttatgccagctttttttttaaagatttatttatttattatatataagtacactgtagctgtcttcagatacaccagaagagggcatcggatctctttacagatggttgtgagccaccatgtggttgctgggaattgaactcatgacctctggaagagcagtcgggtgctcttaaccactgagccatctctccagccctatgccagctttttgaaaggaatgattcttgctagagtggagcctggccctggctggaggggactgacgtatgccagctttttgaaaggaatgattcttgctagagtggagcctggccctggctggaggggactgacgtgcttctgagcacaggcctctcccgactctccgcttcaggcccttcctgtgggtcaccacagcagtggacatggtcttactctggcagcagcaagtggcatctgggaagagctggatagctgagatgttagggtggagaggaagggaggagtacagaagaggctgtctgcccagtgggctctacacctgataagcaggtcattgtgtggtggcacgtttagagaagcatagcaccctataatccacttgccttaccgtcaccacattccagttccatgaaatggaaaggaaaataaaactgcttctgccactgctgttagcagtttgacttagtatcttcctgggtattttttctgccccatccaaataagaatatgaaaacattagcacaaggcagatgtagctgtggtttgcatttggtctgtatgctgactgttagtagatatcctcagaatgacatggtctcagtcatgcttgtgccatgttaaatttagtcttattttaatagctggtgacaatcttctagcccacttcatccttctctggttgcttctttcatgtggttatgctaggcaaccagcagaagctagggctaacactactgagttctccgggccttacacccttccagtgtgtccacttgtaaatccacaaacaccctttgccttgccattagggaacaggtttgtgtggtccacacagtagaggttttattcttcagtgtgtgacacattttcccctcattttctagaagccaaatgatgtgcacatggctattttctgcctctgttgggggctctatgctttctttaaggaacttttattgatgggacctttgacaaacatgcatccagggtactgttattgtttgcattctgtggtgattccctgtagtgccattgcctgcttcccatggagcccttgcaggctcctcttcccactgctagagtcggacccttggtccagccacccagtgagtgagtctgtgcgctgtttcttgtgaagagtcagctggggagaaggtttaggcaggacagctcatggatattgcagtttgatattattgctcttgatagagaaacctcttttctcacagctgtgtgcaggtgtgcagaatcccctccccacctccccaaccccccaagttccctcaccagtctggttttacagggctgaggaagagcagtgctattggaagaccagatctggtgttgtgtactgctgtggcccctttaaaggaagcaataggtgtttcctgaagcagaattgctattggccagtgtttaaaatgcaggaaaggagcattttccttttagctgagaggaaagataaatggagaaggaaatagcctgatggtttgttctgaggcagagctgtggggtggaatttagggcctcttaaagagattgaattccagacaggcagtgggggagaacttaaattctgctgtaaacaacagaagcagaactgtgaaaattgctatatgcatgttgggacagaaccccgaactcaagacattacgtaattcagcatattcttccccaagagggtgttttggttgggtgcagtcatacatctcagaggcagaggcagaggcagaggcaggggcaggggcagaggcagaggcagaggcagaggcagaggcagaggcagaggcaggtagagctctatgaacttacaggccagcctgatctatagagcaagtgccagaccagctagggccctgagaccttatgaccaattaaaaataattgttttttgtttgtttgtttgtttgttttgttttgttttgtttttgagaatgatcatagatttttttttcacactaggaaggcttatcaatataaaataagcaatttcactaaaaactgtaattaaataacatttttgtattgtaacatttagggtgtttgcattagaaggaacatcccaaaggctaatgtctgaggaacaaaataggtcttattctcttggacagtggacatgccctggctttcttgtgcaacgggaaggctgttaggaggccttccatgctgaacttaaggttgaagaattcagtcagttgaagtctaagggacacatgaaatagggccatgataaacctgtgggacaaacttgagctcttagacctttttattcattcatttttaactaggagctttggggagcccagagtctatgtaggttgtggggtgtggaagactctgggccagatccgcactgccagttactgttcctgctctgtgcaccccatgttaaactccactgaatgagtggcgactgctccttcagggctggctggagagggaagcaggagtagattgctggcaggggttggtgccgttcctagctgttaaatgtgtctacacattctgctgtggtatccagagttgtcagtggctttggtggaagcattcaattggctttgtgtagagcgtcatggtcaaacagcatagctggtctgagtgaagtctgtggtccgcatgtgaaagaggatgggcagctttccctctctgctctgggttgtaagttgagttggggagttttgagtactgctattcttactcattttcaagttgtgtggcactggttctggaagttaacagaacacggtttacaagtaatgttcagttgttagcagacagtgaggttttgaaaatcaaaatgtttttttttctattctttttttcccggagctggggactgaacccagggccttgcgcttgctaggcaagcgctctaccactgagccaaatccccaacccctgaaaataaaaatgttttacgtttgtattttaactgccagtaagagttctttctgcctgagggagggacctgatggagtgttagctgcagccctggcactgcccagtgtgctagagtgagagttcactcataaggagcctgactgcctcaggggttgctagggctcactgtggtgaggagactaaggaacacccccagttgtggtccatgtaaccataaggttactggaggcgatgcttcacttgacctgtatagccttatgtatccagtgtgcttgtttctgtaacacctaggagtatgacagtgaactctggtggtggttgagacccagggcttctcctcaggttgctacaaagcagggaggatacatggcttgagtgtagaggggaccatagatgagtggcctggctatgcagtccctcgtggataagcagctttggattagacagtgggtgcagggaatggagtgtggtggaggccttgtggggagggacaggcatgttcacttgtcttagcagtagtgactgaatctggaagttaagcaggaggcacaaaatgggtctttggtacctctaggctgtggaaagatgggagagctacagtgtctggagccctgggtaggaggcttctggtgctgttctctggtggtcttgtactgcttggggctgcccattaattagccttggccttgaagaggccagagggactggattggacattttggaagcctcagtcaggataagctgcgtggactcagttaaaaggtacagacccatgaaggaagaggaggtaggaggcagactggagacttcagtgtaagtgagccagaaagtggccactcacccaccccagcttatctaccagcctgacacagcagccagtggcttctgttttcatgtttatgtaccaagaatgccatgcatggctcagcactgctcctaatcccatcttctcagtgtccctgtgtgccttgcaaatcatactgtccttctgaagctgttaatgaacctaacccaagcgggcaggaagagtttcatattgaacatgtaagtgattcaagattgagcatttccacttcattggaagtttaatcttcaagtacagagttttggttcctgtagcaggagtttgtgcagtccttaactcttgggtaaagcttttcaaccacagccctcttaaacaggctgtttgttgaggctgtgtcaccactgtggtggggttgtttcttacaggctccataggcacacagttagccccgagccactgacgtgctggagtggctgtctccagtctggtgtccctcagctttgtgttgctggtagggggaggacaaggagaccagtcttggcatagagcctttgttgtgagttaatcaagtgaccctgagtagccttttattttcacagtgacttttgaatgtaaagtattgtgacacagtgtaaatgttttgtgggagatttgtactttgaataaagtagaaactatacctagtggtaacacgtgcatgctactttggaatgttgaaatggatctcttaagtttcctaccacatgtcctgtagtgagaatttctgaaagaatccttagcagtttaaccggggggcctaaccttacacagtgggtttcactgctcttctgttgtgagccctttgtgtgtggagacaggaagatatttctccctgggcttgcgtttagtgagtaagatgtcaggtcatattggttttatttttatttttattttattttactgtatgagtgttttgcctctgcaagtgtgcccagtgcacatgccttgtgccacagagaccagaagagggtgttggattggttagagctggagttagagagagttgttgactgctacgtgggtgctgggaaccgaacctctgtcccttgcaggagcagcgcgtgctcttaaccactgagccagctcttcagctcccgtgttggtgatttgtaaatacctaaacttcctgaagaggttgaaataagtttgggggtcttttttatttttaaagatatgagggtagagtgggcaactcgctggtctgtgtattctaagggagcgaagatgagcctacctccgttagagtcctctccagccacttaccacccccagactttggctttgactttggcttagaagccctggttcggcagttcagtgtttgttttctttctcttcgtcactttgtgctgcagcataagctactgtggaacctttatggctccctgagttctgtgactgtttcctcaaggtaagtacatactgatgcagagattgtcctgaacttagataagagttttaatattgctgtgtgttaaatgctctttcacagttttttccagaaagtaacttgtgcacctgggcgtaggacaccaggcccgaaatctcttgttagggaaacacacagtgttacctgaggccccgggctgcacacgagagcagaccattgtgtgtgatgctgtttccttaattgaattagtgttttggtggacttcacatttatataagttttataatagattttataatcttcagttttcaaaatcactttatttataattttttcaggagataaatctccccctaaacttgaaccatcagatgcattacctcttccttcagacccggagactaattcagaaccaccaaccctcaaaccagtagaactcaaccccgagcagagtaagctattcaaaagagtcacatttgataatgaatcacatagcacttgcactcagagcgcactggtaagcggacaccctccagagcccaccctcgccagtagtggcgatgtgccggcggcggcggcctccgcagtggcggagccatcaagcgatgtaaacagacgcacttctgttctcttctgcaaatcgaaaagtgtaagccccccaaagtctgccaagaacactgaaacccagccaacttctcctcagctagggaccaaaacctttttgtctgtagtccttccgaggttggagactctactgcagccaaggaaaaggtcgaggagcacatgtggagactccgaagtggaggaggagtccccgggaaagcgcctggatacaggtaaatgtcaggggcagccctccggggaactcttaatgtaaaactgtggtgctgagcatcctctcagtcctaaagctgcagaattgtttcaaccagcggccattcagcctcttggcaacccagcagctggccatacagcagtggcatgtctggccccgccctcctttgttcctcctctttctctgtggcttttcacctattgactttgaatgtgatttgcgtaccttgactattgtgtgcatgtgtgtgtaaactggtacctgtgaatggccacacctggcactaggtgtcctggggtggtgggtgtcgcctaagagcagtgcccacaaactcagccatagatttgaacctgaacctctctttactgaagactgcatcttccctgagctttctgaaaatattctgtcatctcattacttgtaacacttcataattggcttaaagaaaattgtgatgttccctcgatgtgttttgtatcttgttttagtacacgtgcacttgactggtaagtacatgtcaaggtacatgtactactaatgcttggtaaaatcatactcaaaacgtttctccttttttggtaagcttttttctttttttttcccttaaaggtttgaaggcnnnnnnnnnnnnnnnnnnnnnnnnnnnnnnnnnnnnnnnnnnnnnnnnnnagacttttatgagtttgaggccagcatggtctgtagagtgagttccaggatagccagggctacacagagaaaccctgtctcaaaaaaacaaaaacaaacaaaaaaagctaaattagtttggttggatgccacagtgtacatacatatggggtggggttgggaataaatcatcagagaactgggaagttcattatctctgggattgaactggggccatcagcttggtggcacatgaattcccactgaaccatcaccaaggcccacggttaacctgagaagtgtttttattttctaagacgttgccatgtgtgcctgtacctttagaacctccatggaaattccctgtgctgtgcttggacagtactggcctggcttcctcacagcctccttcctgtctaatcccagataagacacagtggggaatgggtgtttgtgctctgaggcagtggttgccagcatcagatgtgatgttcaatgtctgctctgttggaggcaggcttacttacacctttgttttttacctggattagcgtcttagttacttctctgttgctataaagagacacatgacaaaggcgacttccttggaagagaaatgaggttcatgtttcttgagggttaagagtctgtccccatcatggcagggagatgtcaggcatcctggcaggcatggcattagagagaggagagatagtgatcacagaagcaggagagatcgctgactggaaatggcttgggttttgaaaccttaaaactggcgacacacctcttccaataaggtcattcctccctaatctttcccaaacagttacactaactggggacaaatattcaaatacgtgatgctgtagggatcattcaaaacatcacattccatttcttggacccctgtaggcttgtggctatatcacagtgaaaagtgtatttatttagtccaacttcaaaagtccccatagtctcacagtttcaacagtttaaaagttcaaagtcttctgagactcctgattttaaccccttgtaaaatcaaaattaaaaaaaaaaacaaatcacatacttgcagcatacaatggcacagaaaatacatgaacattccaaaaggcaggagagggagcacagtgaggaaatactagaccacagcaaggccttaatccagcagggcaaactcccagtcctttagctctgtgtccaatgtcaaagactgaggtggctttccttccagctctgcggattgcaaaccatctccctgatgaactggttccatgctgtttgtagctctccttggtagacgtcccgtaacgttgggagctttaacatcttggcatctccaacacagttcagccacactcagtagcctttcggacttccccatgcagagactgaccttcaacgagtctggtttcagtgactttccttaagggaggaggaagattccatacctccttcattcctgtattcttcaacaagactctgaagtcagaacgactgggctgaagagctgtattaggctgccagctgggatggaacttggcctgacttgaattacattggcataagccttgacttgttgctttttaggaacagatcattctttagccctgttcttctcacaagggtcagctgagtagaatctcatcctaaggacaccactccttttattccatttctcctcctctctgttagaacaagcctgggctccattattaaatttggttctatttcttttctccttaaactctgtattttgtgctttcttttttccacacttgttctttttcattgtagataacacataagagtgattactaacaactatacaacagagtttattagattaaatccccctcccccattctaatttagtttcgggctgattgttgttgttgttgtttttccaagacagaaagcctctctttgtaatcctggctgccctgggtctgtagaccaggctgacctgaagcctggagatctgcctgcttctgctcccataggctgggataaaaggcacacaccaccacctcctgctatctccggctgattcacattgtttgcaaaaacatatcacaagaatggtctttagcccagtcgctaatgttgtttccctcttaaagctcttgaacgggcccttcctagtctacgttgctttcaggatggtcttccaggcttcctattactatggctcattaaccccacttacagtgttcaaccagtccaaagtcccaaggttttctaaaaagtaccatggtcaggccggtcaccctaactccctggtaccagcttctgtcttagttacctttctgttgctgtgaagagtaacttgtaagagcaggcatttgatttatggctcatggttccatagggttagagtctgtcagtccgacaccattttggtgctgagagctcttatctgatctacaagcatgactcagagaaagggaatgctagcccaacggcctgggctttggaacctggaaacccctccccagcaacacagttcctacaaggccacacctctaaatgttactaaacacttcaccaactggggaccaggcattcaaatgtgatcagatggggggccattctcactcagaccaccacactaaggaaattgcatttccttccaggcactctagtgttggctgttcttgtctacacgtcagcaatgagcaatacataagttgctgatgagtgaaatctgtttcctggaaccttgccaggtggtccaggtcagagatttggaagggcaaggctggcttggtagtgacagtggactgttggcacctctccatctctctccatctctcttgagnnnnnnnnnnnnnnnnnnnnnnnnnnnnnnnnnnnnnnnnnnnnnnnnnngntttatatctgtctgtctatcttggaggttgactctggtgcttcatggcaagttgtgtactatggaacccctttcaactgtgtgtgtgtgtgtgtgtgtgtgtgtgtgtgtgtgtgtgtgtgtgtgtgtgtgtgtgtatgccacagtgtgtgggggatcaaaagattgtctctaccatgtgggtcccagcatacattagacttgtgtagcaagtacctttacctttgcactgacccatcttggtgaccctttcaaaaaagtttgaagcaagctggttttggaccttttctataacaaaggccttaactcattagcctgttacctcagcctctgaagtagctgaaatcacaggcctgtactagtaggcccagctaatttcatttttattttgcacttagtaattttatagaaactgttctcaatgccctcttatttaaatccagtaacacttggttttgatgaagcagttctaacatgccagaggaagcagcttgaaacggaagttgtttcacttctgggtgtttctgaagccatgtaactttagcttttcttagccgcatttagtgcaagactggtgtcttttgagctctctgaaagtcctttgtcactgaactggccgtactatgctgtgttctactgctaggagattactcagtgtctcagtaccaagtgacttgcgtgcacaggatggctttctacctcctcagggttcatatctttcagcaacagaaagtttattgaccctgcaagacactgtagaaaaagctttcaccgtggagctgctgttttgagcccctgctctgtacctggcagcttctgccaagtactgtggctaactaggccatgtgggctctgcgagactgtttgtcacctctgggttactaatcaggagttcatgaaatgttatgtgtgtgcacatctcagttcttttctgggttcttagtaataacgatgagtcccacggaatcttaataatagacctttagtttgtgtgattccccagtgcctttgatcctgactgaaatggagatttctgctttcttattccagaatggcagtagatttcagtggatgcatgatgaattcctaatcgcactcctgagcagccgggagccttgttagcactaagatctgaccctcaggaacaggaggcgtctactgctgcatctgcttgcccgtggtgggccaggcatgggctgaatgggcccatcctaccatcggtgctggctgtgcctccacttgaaccttctggtgctttctgcgcacctggatttcttgtttcaagttgcagttcttcgctgtttgaggacttggaatattcagaaccttctgatcttttccaggttcatctggcactgaacttttaggggaattctctggtgctctccagtgcactgcaagattccaagttagattaagtatggacttacttatttttaaactgcccatccacaggcctccgcttgctcatgcctgcaggacaggcggggatgtgggcagtgccgagcatggtgtgactgctgttatggttatcataatttttggagctggctctgtttcgtagatttttttactctgcctgttttatttccgtcaatggaccatcaggccaggacccgtgtcactccttactcatactgtggtgtggagattctccatgaaatgtgtggtgtggtgataagcaagtgaatgtcttccatggccacaggctgttgagggaggagacattcctgcccttgcagtcagactaaatggcttctcactgttttccaggttctcagttaaccactaatgtgcctgggtagctcactctttggatcctaatccttttctcttaacctcgacttggattggagttctgctaaatggcctcttggattgcaaagccttcgctgccttcttaccttgctcctagttcttgaggatcacattggagtcatctgctgagccgtcctctaaacagacactcagacacacctaccccggaggagatctgtcccgggccagcagttggaggagcctggtgcctgagctgatgtctcggtgcctcaggtcttcctggtgctatagcagaacctgctgtagcttggacaacaaatccagcagttttgcctcatcctgagcacatccaaaactgacctgtgatgactgggggctctggttagggcgtcttggtctagatgtctgaagggactgactgtacacacatgttgccctaatggccctaaatagagctccttacttggttgttagcatcttttgttctctgtctggtgtccctttcctctccttccatgtgtcttggcttactaagcactgctcgtctagttattagctgtgcagatatctgctgagccaaccgggaagtcctgcatggccctcaagagggcatttcggcttggcttctggcctgtcgtggccatatgcagccgtgtcttcactcatgaaaagcagagtggtgggtggggtggtattgtttgccgtgcttttatcagaaggtgggaacatgcacccactgtgacattcttttggtgccatcggaagaccatagatgcctctgctgccaccctcaagtggtcgtgttccgagcctaggacgcaggcttaacaagctggagctttgggcacacgatcctgtgctgtctgaaatgagtccgtggactctggagagctgtctcttggtagtgggtgttaccagcaaggatgcactcagtgactataatatctcataggccttgtgtgttctgtaggtgagagcctggtcagtgtagcttactaggccccaacattctcacttgctgcagcccgtgagctcttgcattgtgcaggttaggagcccatggtagaaaggatcatgtccctactatcccaccttgtgcctctcagctttgcaaatagacaacctgggacttctccctggcctgtgggtgagagctgaagacctgagcacctcagggtaccctttattggccactctgtgtaccccagttgtgctgtagatacctgggccggggggagcttttagggatgctgtggtagttacaaggctggggctggcccacctagggacatgttgcagtggtactttgtggcctcctttgggtctgagtaaggcaggcgttcgaactctgccttagtctttggagacatcaggaccctctgcaccatatgcatggggccattggctttgtgtagaagccatctcatcttttgctctggttgtcaggagtagttggcatgtatgtgatcttctacagtaaacttcagtgctaggagggcactttcctttgctctcctttccgcagctaatgggagaatcattgtaggaagtggatcacaaagagggaggcagagactgctcattacctggctttgggtcacagccatgcattcttcagacagtggctgcaagagcttttcaagctcgtgtctctggcctgtgttgcctcttggggcatgtccagggccttggaataagagtctgattgggccatgcaagcactgtagtagtttgggctgtagcaccctctgaaaagcaggcccagagaactgcttgtctctgcaggccccaggggtctctcctggaagcttctcaggtttcacagtggctctgccagcttttcaaggtgttacgtgtctttatgaaacgtgtgaaaactttctgtaaacttaggagcccagatgcagtgtaccctggtaattaaacacttgggaaaatggcagagacatttaatcatattttttcccttctcaaagttataaactttctcttagtttttccaacctcctccagactccccaaggggctgtttaggccctgacaaggccccttgttacaggtaaaagctattgccatccttgtcgggaataccaagtgtttttgggaactgtacttctgggttctttcctggggtgtcttctagcacagagaggcttgacctgccagttctgcctagccatggcagatgatttggggcttgtagttttctaagatcttgggtcctggagcaagggctctgcgtttctctgtccatccaggtaacagggctgtctctgtgttgactttgctgacctaagtcagcaggtgtccattctacgttgtgtgttgcacctacccaaggcagcaccatgttctcctacctcctaagaattcttgggccttgaggcttttaggagaggaatgcggcttcttccctgtcttgtgtcttctgctttgccagtgagcaaacaagaagcttctcagaagtctttttagcacaagcaggtccttttcacaggtgggagaatgcaatgaagaccttagtcacctcatacgtccaagaaaatgttctttaaaaataagtttacatgctttactttggaaaatagagcttacatttttaaggttatatggggaagatgggcatatgtgaacaaaaagtgttgtctgtttgctgttcccgtccccttccctttcccaaactggtgcagccagaagaagccagacaagcacacagcctggggacatgatccttctgattcagggaggtctgcaaggaccatgggtggatgtgcctttttctacttactgacttaaattgagggtcacgcttgcttgcaaggaatatggtggttgccttgactcagatttgcctttattaaaatactttacaaatatccagatgctgtggttgcgtttgtgcagacattatacctgatgtatatcttggctcagcttcctgccaagttccacattttttggtgcttggggacctggcattgctcaggtgaattgggcccacacttgctagtttaaaatgttttatctatatgtttaaaaagtccttgttaaaacattgatgtttctattttttttttttttttttttttgctggtggtcaacccagggcctgcatgcatgctaggcaggtgctctgttactggactgtattgccctgatctctctttnnnnnnnnnnnnnnnnnnnnnnnnnnnnnnnnnnnnnnnnnnnnnnnnnntcccctcccttcctacagttgtgcatgaccagtgtattttgtggaggactgcagtgttccacctccctcgggtactgcagggctctcatctagacactgctaccttgtctagtgaacagtctagttttgttgtaagctcacagaccttacagcatgggatacgttaacttgggaagtggtgacagtttctttttgctttgcactcatgggactcattgagcaagtgcagaactgctctgactttttccttacacagcagtgcctctgcgtctgtccactgaggtcatgggagtgagagatgaggtgctggcttttttgtgtatagaatgttctttgtgggaccagtgatgtagctcaactagctggactagcagccgggaactctgggcttgagccccagcacctcaggaaccagggatggaggcaagaagatcagagattcaggagcaacctggacctgaaaccctgcctcacagaagcacagttatttggaaaactgattgtaatttttgaggaggagaaaggtattaagttgagctagtgctctggtgtattgtgaccatgtatctgtctgcttcttgtgttccaggtctcaccaatggctttgggggtactagaagcgagcaggagccaggcagcggcccagggaggaaagctgcgccccgacggcgctgtgcatctgagtccagcatttgttccagcaacagcccactgtgtgactcaaggtagccctctgccttctgcagcaggggccttggccgccttctgaatgaagatctagaaagattgccctggagttttatgtgtgtgctctgccattcagggacagtttctcatgggataaattgaacctgctcagtagaatttaacagaagtcagaagttactctaaattatactgtataattaaatcagcataattaaactctgcacctcccactccccaaaccttttctcttactaagattgtggtgtgcactgagtgctctgcctgccggtactgtagtgaccactctgtcaagtggcctcatggggacaggcttactttccttgggtctccacaccacattgtcctttggcagcctggcacctggtataggatgaagccccaggaagggcactataaaccgtagtctgagtttgactcatctctgctttcctcccgaagctttagtacacccaagtgtggccgagggaagcctgcgcttgtacgaaggcacacactggaagaccgcagtgagctgatatcttgtattgaaaatggaaactacgccaaggcggccaggattgcagctggtgagttgggatacagacttggatggaaaggcaaggtctggtcttggtggcttggggctgtgagtcaagccccccacattagaggaaaggaataagcatatgcttaactcactgatgctggggtttcaggcctgtccggagctgcattgtatttggcttgggtagttcctgatgatggaatttgtccaggacagggtctgttgaggatatctgccttgtaactgaaggtgtcacagcaggctgatcattcctagcttcaggctccgtgtaagggaggagtgcctgctaggctgatgctgtttagattttcctgttagtggaccaagcccactccggggatagaagtacttggctgttgtttgaggcttgtcagggcaaagacctgaaattgagaagggctggagactgcagggaatgggctgtggccatagaggctgcagtgcagtgcgacagcagtgctggaggtcctgggttacggagaagctaggacatggtgctgctgagagtggcatcacttaccaacagtagtcggtgaggcctgtacacaggcgtgtcctgggcggctggatagactcagcgaggctcacaagagccaggtggcagtagtttggaataagccagaacttgtatttggctgtttggttgtgtaataggcgtctggtacatgctaaggatcctgtctttagaatcgaggtttaagtatggcgaggtacaggggacctgaagtcctaggccttagaactgggggtggtgggaagcagggagccctttggcaggcttctaggcctcacttccacgggaggaacagtgaggttcctgcttctcctggctgaggtgttgtagaactatctgctctagcagctaggggagctggggcctgagaggagtgggactttttctagccccaggcttagtggctgtgttgggtattagtggttctcctgcttggtctgactcaggtgttccttttttcctagaggtgggacagaacagcatgtggatttccaccgatgccgctgcctccgtcctggagcccctgaaggtggtgtgggccaagtgtagcggctatccctcctacccagcactggtgagtctggaggcagtgagtagggtgttctggtgggacctgggttggggcgccagtgcatgctctgcaccttgcagtctgtgctgccaaaggtgtgattgtcagagcttggggcaggtgtatgaatggctgtgggtttaggcatgtggctctgagtcactggaacatgtccttggcagattattgaccccaagatgccacgagtgcctggccaccacaatggtgtcaccatccccgccccgcccctggatgtgctgaagatcggtgaacacatgcagaccaagtctgaggagaagctgttccttgttttgttctttgacaataagagaagctggtgagtgtggtgtttagaactctacagcaggcggagctggggttctgttacacctggggcttcgcttacccatgctgagtcagggtgcctccgagcagtctttcctggtccttgtctgctgccgcctgggcacggtggagtgctggtctctcctgacagctcctctcacttcctagatccctgagtggctcaggctgtcttcagttcttgctctagactctcttgcaatgggctttttgatcctacagctctgccaggacacaagctccacccctttccaccctctctatcttcctgtcctctctctacccagagctaccggaactcccttatgccacggatcagaccctaatattccctgagggtattatctagtgacaccttgaccatatttagttgaatccagccctttcctgtgtaactaggtcctttcagctggccttgcttaggactaagaggtgcgtcagtagttagtgtcctgttgagtggccactcttggggagatagcaggtcccctgtggtcctcttctctctgcctggctctggctcctggcagttggtgttcattgaggcttagactggccttgcccactcgatggttctgtgtatagtcttctcacccatcctaggaggcctccttactctgctccagagttcactggcctatgttatgtgtatacatatcctgcttggtcccttacatacacgtgtacctgggctcacctagtgggccacacacttgtgttatcttgggcctgttctgtgtgcatagctgtatgttccagatttatctccaaggccactttcatgtgtatggaaaagaggattgtattatggtttacatgccttacctgagtccagacagggctggctaggtggatgccctatgcagctgaacttcctgcatgaggtgtccagcatgtggggagcttgggttgggtaccgccaggttccttgttgtgcacatgggctgatggttagtgggccataccaagtatgggctggcggttcttatgctctgtcttctctttaaaaggcagtggcttcccaagtccaagatggttcctcttggtgtggatgagaccattgacaaactgaaaatgatggaagggaggaactctagcatccggaaggctgtgcggattgcatttgatcgagccatgaatcatctgagccgagtccatggggagccagccagtgacctcagtgacattgactgaggtggcttccagcaaaaggcagtggctaaagccacagccaaccaggagccctgtcaatagtgttgataagctgtacatgtttgtatattgttcagaacttaacttattctgattttctaggtgtagttctttaattctttttcccccccccgggaggggaggtttcacttccaagttttctatgaaaccatctggtcttggctttgcaggtgaggagggtctgttccgagcagtgtggtgtggggtcccactgcaggtgccgagtgccgaggcctcacttacttctaatctgtagggtttttttttttaaagacttttgaatgtttaataattttgtagatcatgctctttacacagagtaccgcttatttaataagacggggtgtaaatttacaatgacaaatgtgtattttaagaaagaaaatgacattattttgaatggtactttgtgcaaagagggaataaatttatgctgtgtgcatcacttgcaaatcaccaaaaaatgtcccgccagctgctgccggccagggcccgttctcaccgttctgactgccctgagtctcctgttctgccctggctcctgcaggcgtgcctcccagcgggttatttattgtagaaagtgtactcatttgctttataatgaaaaaataaatttgcaaaggtatattgatatgcatttttatacaggcacataaaaactcaacttggtgtgggagcagaatgtgttgcgaggttatatacatgatgggcctgtgtgtactttgatttttgtaacttgtaatcttttgtttacaatgaggagctttctgtaacttgttttaatttagaacactttggtagcaatagacctttggatacatttttgtatggtacatgtgatgtatatagaattagtactttatttttatttctaagaggtaaagcattatgttaggggaaaaggcagggtgggtttccaaatttgcatttttatattaaaaataaagtgaagatttggacagtgtggccctctcattcctgcatcactaggaggctgggtgagctgtagcctgaggtacatgtgggagcactgaggccttgagtgggtggtgtgaccaggtgtgacacacttgatctaacagctgacctggggtggcattatttattattttgcctaatcatatttttattttaaagctaaatagttactaaaaattttaaatgttcttttaagtctacatgtttgtaatatctccatagaaacttgaaaaataaaaagtcttcctttggt

SEQ ID NO: 19 - DNA, 39 bp, Rattus
  source 1..39: /organism=“Rattus” /note=“BRD1 Exon 1A / Promoter” /mol_type=“unassigned DNA”
cattgtttgcttcgctggggagcgagcagcgcctcggca

SEQ ID NO: 20 - DNA, 1381 bp, Rattus
  source 1..1381: /organism=“Rattus” /note=“BRD1 Exon 1B” /mol_type=“unassigned DNA”
gtaatcattgccaaatgaggaggaaaggacgatgtcatcgaggttctgcagcgaggcatccttcttccccgtgcagtattaaacactcccccactcgtgaaacattgacatacgcacaagctcaaaggatggtggagatagaaatcgaagggcgtttgcatcggatcagtattttcgatcccttggagatcattctagaagatgacctcactgctcaagaaatgagtgaatgcaacagtaataaagaaaacagtgagaggccacctgtttgcttaagaactaagcgtcacaaaaacaacagagtcaaaaagaaaaatgaagtcttgcccagcacccatggcacaccggcttcagccagtgcccttcctgagcccaaggtgcggattgtggagtatagtcctccatctgcacccaggaggccccctgtgtactacaagttcatcgagaagtcagccgaggagctggacaacgaggtagagtacgacatggatgaggaagattacgcctggttagagatcatcaatgagaagcggaagggcgactgtgtctctgccgtgtcacagaacatgtttgagttcctgatggaccgctttgagaaggagtcctactgtgagaaccagaagcagggtgaacaccagtccttgatagacgaggacgctgtgtgctgcatctgcatggatggcgaatgccagaacagcaacgttatactcttctgtgacatgtgcaacctggctgtgcaccaggagtgctacggggtgccctacatccctgagggccagtggctttgccgccactgcctgcagtctcgggcccgccctgcggattgcgtgctgtgcccgaataagggtggtgccttcaaaaagacagacgatgaccgctggggccatgtggtatgtgcactgtggatcccagaggttggctttgccaacacggtattcattgagcccatcgatggtgtgaggaacatacctcctgcccggtggaaactgacgtgctacctctgtaagcagaaaggcgtgggtgcctgcattcagtgccacaaagcaaattgctacacagcattccatgtgacgtgtgcccagaaggctggtctgtacatgaagatggagcctgtgaaggagctgactggaggcagcaccaccttctctgtcagaaagactgcttactgtgatgtccacacacctccaggctgtacccggaggcctctgaacatttatggagatgttgaaatgaaaaatggtgtgtgtcgaaaagaaagctcagtcaaaacggtcaggtctacatccaaggtcaggaaaaaagcaaaaaaggctaagaaagcactggctgagccctgcgcggtcctgccgaccgtgtgtgctccatatatcccccctcagag

SEQ ID NO: 21 - DNA, 132 bp, Rattus
  source 1..132: /organism=“Rattus” /note=“BRD1 Exon 3” /mol_type=“unassigned DNA”
agagaaaatgatgaagagatgaaagctgccaaagagaagctgaagtactggcagcggctacggcatgacctagagcgtgcccgcctgctgatcgagctgctgcgcaagcgggagaaactcaaacgggagcag

SEQ ID NO: 22 - DNA, 129 bp, Rattus
  source 1..129: /organism=“Rattus” /note=“BRD1 Exon 4” /mol_type=“unassigned DNA”
gtgaaggtggagcagatggctatggagctccggttgacacctctgactgtgctgctacgctcagtcctggagcagctacaggagaaggaccctgcaaagatctttgcccagcccgtgagtctcaaggag

SEQ ID NO: 23 - DNA, 313 bp, Rattus
  source 1..313: /organism=“Rattus” /note=“BRD1 Exon 5” /mol_type=“unassigned DNA”
gtaccagattatttggatcacattaaacatcccatggactttgctacaatgaggaaacggctagaagctcaagggtataaaaacctccatgcgtttgaggaggattttaatctcattgtagataactgcatgaagtacaatgccaaggacaccgtgttttatagagctgcagtgaggctgcgagatcagggaggtgttgtcttgaggcaggcccggcgtgaggtggatagcatcggcctggaagaggcctcgggaatgcacctgcctgagcgacccatcgcagcccctcggcggcccttctcctgggaagagg

SEQ ID NO: 24 - DNA, 261 bp, Rattus
  source 1..261: /organism=“Rattus” /note=“BRD1 Exon 6” /mol_type=“unassigned DNA”
tggacaggttgctggacccagccaacagggcccacatgagcttggaggagcagctgagagaactactggacaagttggacctgacctgctccatgaagtccagcggctcacggagtaaacgggcaaagctgctcaaaaaagagattgctcttctccgaaacaagctgagccagcagcacagccagaccccatccataggggcaggcacaggaggctttgaagacgatgctgctccactggcgccagacacaggggaggaag

SEQ ID NO: 25 - DNA, 105 bp, Rattus
  source 1..105: /organism=“Rattus” /note=“BRD1 Exon 7” /mol_type=“unassigned DNA”
tccttccgaggttggagactctactgcagccaaggaaaaggtcgaggagcacatgtggagactccgaagtggaggaggagtccccgggaaagcgcctggatacag

SEQ ID NO: 26 - DNA, 136 bp, Rattus
  source 1..136: /organism=“Rattus” /note=“BRD1 Exon 8” /mol_type=“unassigned DNA”
gtctcaccaatggctttgggggtactagaagcgagcaggagccaggcagcggcccagggaggaaagctgcgccccgacggcgctgtgcatctgagtccagcatttgttccagcaacagcccactgtgtgactcaag

SEQ ID NO: 27 - DNA, 128 bp, Rattus
  source 1..128: /organism=“Rattus” /note=“BRD1 Exon 9” /mol_type=“unassigned DNA”
ctttagtacacccaagtgtggccgagggaagcctgcgcttgtacgaaggcacacactggaagaccgcagtgagctgatatcttgtattgaaaatggaaactacgccaaggcggccaggattgcagctg

SEQ ID NO: 28 - DNA, 110 bp, Rattus
  source 1..110: /organism=“Rattus” /note=“BRD1 Exon 10” /mol_type=“unassigned DNA”
aggtgggacagaacagcatgtggatttccaccgatgccgctgcctccgtcctggagcccctgaaggtggtgtgggccaagtgtagcggctatccctcctacccagcactg

SEQ ID NO: 29 - DNA, 155 bp, Rattus
  source 1..155: /organism=“Rattus” /note=“BRD1 Exon 11” /mol_type=“unassigned DNA”
attattgaccccaagatgccacgagtgcctggccaccacaatggtgtcaccatccccgccccgcccctggatgtgctgaagatcggtgaacacatgcagaccaagtctgaggagaagctgttccttgttttgttctttgacaataagagaagctg

SEQ ID NO: 30 - DNA, 1454 bp, Rattus
  source 1..1454: /organism=“Rattus” /note=“BRD1 Exon 12” /mol_type=“unassigned DNA”
gcagtggcttcccaagtccaagatggttcctcttggtgtggatgagaccattgacaaactgaaaatgatggaagggaggaactctagcatccggaaggctgtgcggattgcatttgatcgagccatgaatcatctgagccgagtccatggggagccagccagtgacctcagtgacattgactgaggtggcttccagcaaaaggcagtggctaaagccacagccaaccaggagccctgtcaatagtgttgataagctgtacatgtttgtatattgttcagaacttaacttattctgattttctaggtgtagttctttaattctttttcccccccccgggaggggaggtttcacttccaagttttctatgaaaccatctggtcttggctttgcaggtgaggagggtctgttccgagcagtgtggtgtggggtcccactgcaggtgccgagtgccgaggcctcacttacttctaatctgtagggtttttttttttaaagacttttgaatgtttaataattttgtagatcatgctctttacacagagtaccgcttatttaataagacggggtgtaaatttacaatgacaaatgtgtattttaagaaagaaaatgacattattttgaatggtactttgtgcaaagagggaataaatttatgctgtgtgcatcacttgcaaatcaccaaaaaatgtcccgccagctgctgccggccagggcccgttctcaccgttctgactgccctgagtctcctgttctgccctggctcctgcaggcgtgcctcccagcgggttatttattgtagaaagtgtactcatttgctttataatgaaaaaataaatttgcaaaggtatattgatatgcatttttatacaggcacataaaaactcaacttggtgtgggagcagaatgtgttgcgaggttatatacatgatgggcctgtgtgtactttgatttttgtaacttgtaatcttttgtttacaatgaggagctttctgtaacttgttttaatttagaacactttggtagcaatagacctttggatacatttttgtatggtacatgtgatgtatatagaattagtactttatttttatttctaagaggtaaagcattatgttaggggaaaaggcagggtgggtttccaaatttgcatttttatattaaaaataaagtgaagatttggacagtgtggccctctcattcctgcatcactaggaggctgggtgagctgtagcctgaggtacatgtgggagcactgaggccttgagtgggtggtgtgaccaggtgtgacacacttgatctaacagctgacctggggtggcattatttattattttgcctaatcatatttttattttaaagctaaatagttactaaaaattttaaatgttcttttaagtctacatgtttgtaatatctccatagaaacttgaaaaataaaaagtcttcctttggt

SEQ ID NO: 31 - protein, 1058 aa, Rattus
  BRD1 amino acid
MRRKGRCHRGSAARHPSSPCSIKHSPTRETLTYAQAQRMVEIEIEGRLHRISIFDPLEIILEDDLTAQEMSECNSNKENSERPPVCLRTKRHKNNRVKKKNEVLPSTHGTPASASALPEPKVRIVEYSPPSAPRRPPVYYKFIEKSAEELDNEVEYDMDEEDYAWLEIINEKRKGDCVSAVSQNMFEFLMDRFEKESYCENQKQGEHQSLIDEDAVCCICMDGECQNSNVILFCDMCNLAVHQECYGVPYIPEGQWLCRHCLQSRARPADCVLCPNKGGAFKKTDDDRWGHVVCALWIPEVGFANTVFIEPIDGVRNIPPARWKLTCYLCKQKGVGACIQCHKANCYTAFHVTCAQKAGLYMKMEPVKELTGGSTTFSVRKTAYCDVHTPPGCTRRPLNIYGDVEMKNGVCRKESSVKTVRSTSKVRKKAKKAKKALAEPCAVLPTVCAPYIPPQRLNRIANQVAIQRKKQFVERAHSYWLLKRLSRNGAPLLRRLQSSLQSQRNTQQRENDEEMKAAKEKLKYWQRLRHDLERARLLIELLRKREKLKREQVKVEQMAMELRLTPLTVLLRSVLEQLQEKDPAKIFAQPVSLKEVPDYLDHIKHPMDFATMRKRLEAQGYKNLHAFEEDFNLIVDNCMKYNAKDTVFYRAAVRLRDQGGVVLRQARREVDSIGLEEASGMHLPERPIAAPRRPFSWEEVDRLLDPANRAHMSLEEQLRELLDKLDLTCSMKSSGSRSKRAKLLKKEIALLRNKLSQQHSQTPSIGAGTGGFEDDAAPLAPDTGEEVLPRLETLLQPRKRSRSTCGDSEVEEESPGKRLDTGLTNGFGGTRSEQEPGSGPGRKAAPRRRCASESSICSSNSPLCDSSFSTPKCGRGKPALVRRHTLEDRSELISCIENGNYAKAARIAAEVGQNSMWISTDAAASVLEPLKVVWAKCSGYPSYPALIIDPKMPRVPGHHNGVTIPAPPLDVLKIGEHMQTKSEEKLFLVLFFDNKRSWQWLPKSKMVPLGVDETIDKLKMMEGRNSSIRKAVRIAFDRAMNHLSRVHGEPASDLSDID

SEQ ID NO: 32 - DNA, 19046 bp, Artificial Sequence
  source 1..19046: /organism=“Artificial Sequence” /note=“pBRD1” /mol_type=“unassigned DNA”
cggccgcatgttcccagcctgaactcagtgggtgggctgctctgcttggagagtttcttaaggttgagtgtgcccagcgctggtggcgccagctgtgagcgcaggctttgacctccagtccatccagtcggcagcatctcagctggcagtggtcagtagccgtcactgtgtgtgtagacaggagcacaggggcaaagtggttaaagttttgttcacctgtgtctgctttagacgttgaacctggtgactcttgtggaggatgaaatctgtagttagttgaaggttatgaactgttttcagggacaggctcagggagagaactgcagtgtcctgtctagttttctaaatgcaaacacgtttaaatatccctttcgaagctaaactctcagttttttcatgttttagattaaataggattgcgaatcaggtggccattcagcggaagaagcagtttgtggagcgagcccacagctactggttgctcaaaaggctgtctaggaatggtgctcccctgttgcggcggctccagtccagcctgcagtcccagagaaacacgcagcaggtatgtgtgctcttctgcttttcagttacatgggctgccccccccccccccccccaggctggatgtgctgctgaccctaagccccgggccttaaactctactaaactgcaggttattcgggtggctcctgtatcctcaaggtttgctgtgactttggggttgagttgttctttactctgacaagtgtctgctctgtgcccagtcctctgtcagttccagggaaggaagggactgctcagagaacctggctcaacttcagctgcatgcatagtcaagacagagagggaggcctgatgaagtctatgcagttcctctacacattgcccaaaaactaggtgtctggtaatacctgctggttccactgggaggagctagtcatttcatctgtaaaatagcaaccaactttaatggaagtttaagtctgtagaatcctgtgactccccatggctgtcacaggcatggctgtgaatgagcttagggttctcatcctgtatcctggctgtcagatgagcagtggtactggagccctgttgtatggatcagacccttgtgtctgcaggttaccaagtattgctcttctgggagttaacaacttgctggactctgtctgggtctgatctgaatggaaggggcctccccagtgttagatcttctgttgccttctacaagccaacgttgtctattattcactgaggacacatacctccttggaggctactggaatgtcctagttaggggtttccattgctgagaagagacacagtgaaggcaactcttacaagggacaacatttaactgggctgacttcacaggttcagaggttcagtccattatcatcaggccggaagcatggcagtgtccaggcaagagggtcttagagctattggtcatgaagtggggaagtgtttggtaaccctgggcactgggaggaatgattgcctatgtgacggtaggtagcagtgttggaaagagaagtccgggagtgggtggctacttctgagcttccccttctcagaagtctcttcctgggaagaattccagcattgatttctatgtagcaaagcagactgcttcggaatcgtaccgggacagcgggtttacagatgggatgatctgtgtagatttgtgtacagggtcctgtcttcgtgagcctatagcatggtggagtgcagacagtggctcaattacccatgaccttttaaagatgaaaaccaggccaggagcaaaccacttgagttttgcctatccctaaatatacaagctcaggcctgttggaaacctatccaaaatgctcttatgttactcagaagtctgtttctaaggagcaggaagctgtccagatgatgctaggatatttggttccttttttctttgtttatttggagatagggtcaacctgaatcttgctatatatgctggccttgaactcgcagaactcagtctctgcctcctaagagttgaaattagaggtgcacatggccacagctggcaatgtttgtgaactcccctttccatgtatttgctccctttgcctatatgtgatgagtgaggtacactgtgcattactgtgggcgctaaagtgtgcatcaggacagaccatgccattcccatcctgtgctgccattttcataccatgaagagtggctgtttatacagttgggttggtgacactttgctccgagaccctccatctttgaccgttgtgctggtagcttgagttgcagtctctgctgtggtgtcactgggccatgagaggcaaagctgtccagagagaaggggctcctgtgtgttctacagctgcaaggcagcactttgcttgtggctggcagatgtagatatttatttaggttactgtctagcagtagtgcagaaggacaaacttttgggtaggtcattttccatccctttataatagggacaggcaggacatatggcttactgtgaggaggtaatcccatacattttccacagagtagagagtaggggatagctttggataatgacttgtgttggatgagaaaccaagtcttggacaggttcactctggggaggcagaaagagaagtatggggtggcaggaaaggagatctgggttgggggagcagagctctggggaacgtggttggataagatgcatggaattctgagaggatgaggcatgttgaatttcttggcaagtgactggaaaacctggtgctttgtagatagggctctggtcttgtttggtgttccttggttgctatcaagggatgtgtgctatccctgtggcagtaggtcttgtccccgtacatttgtgaagtagtaagagtaccgtggttagccttgaggggcttactaggcttctggctgcttctcctgcttagaactctgagctgcttctcctgcttagaactctgagcagcagctcaaggatccacctccctctggtgctgcagagctaggctgcttccctgctactgtctgtctcttggtgcttccactttgttggctaggatagagaagtgctggtgcaggatgctgaccaagtgctatttggtgtactgcctgagaaggcagctgtgactggcaactacagtgcccacgcctagaactgaccgcggctcgagcctaggataacttcgtataatgtatgctatacgaagttatggtaaccgaagttcctatactttctagagaataggaacttcggaataggaacttcttataatctagaactagtggatcgatccacgattcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattaattcatatgaagttcctatactttctagagaataggaacttcggaataggaacttcaaaatgtcgcggcgcgccacctgcataatattccgccgccagtaagggtagcttaggtttgtacctcttgtgtatctcctttctcgtactccctccattcctgcctcctggagtcaagccaagaccccgttgtgtcgactagaccttcctgtcccattgtcacagcacatttatagggactgggtacatttatagagactagatcccaggtcctgctacccttttagtcttacctgttggatgagcttgttagatccctggcaggaagaactttggggtgtgactgatggaaagtttcctctaattttctcagagagaaaatgatgaagagatgaaagctgccaaagagaagctaaagtactggcagcggctgcgacatgacctagagcgtgcacgcctgctaattgagctgctgcgcaagcgggagaaactcaagagagagcaggtgaggagggaggcccttgggttctgccaccctctgggctgtccctggatagacgtcttgctgccgtcatggagtgctctggagtggcccctgtgtacctgctgagttagtgctgtccccaccctgtagcatatcatatccctaccctatagttggtcctgtggtacctctgtgttgtccttttcgattagccacctctggagtatacggggtcttaaaggagacccctgccgtggaagaagtacatgtccttgcacagagaaggcagctttgtggtgggatggtagctggcacgtaggctgctctgtgctgctggttcaagtggcgcttctgtgattgtgcagtacgtggaggtgcggtgatctccaggagaggtgtccctacactcctctggagacagtgtatgcagaggtgtccctgcatcttctagagacagtgtatgcatgctgttgttgccaggtgaaggtggagcagatggctatggagctccggctgacgccgctaactgtgctgctacgctcagtcctggagcagctacaggagaaggaccctgcaaagatctttgcccagcccgtgagtctcaaggaggtgcgtgtccctgcgactgagctcttcggctgcttgcttaggaagcatgcaactggggagaggttacctgcattcttaattctcattagttagtagttaatgaatttttggtgaatagtattttaattataaaagattgtacctcgttgtaaagcactgaaagtgcataggtgaaaatttctacttagaacttaacaattggtgatgatagcccccctggtaccccatctgtttgtacttttagttgaagtaggttgggagggtctctgcagtgattgggcttagtttgtattggcttagtgttgttatgtgaaattagtttcaggtgtggttgattttgtaaatgtttattttccctcctaaaattaggtaccagattatttggatcacattaaacaccccatggactttgctacaatgaggaaacggctagaagctcaagggtataaaaacctccatgcctttgaggaggattttaatctcattgtagataactgcatgaagtacaatgccaaggacaccgtgttttatagagctgcagtgaggctgcgcgaccagggaggggttgtcctgaggcaggcccggcgagaggtggagagcattggcctggaagaggcctcgggaatgcacctgcctgagcgacccatcgcagcccctcggcggcccttctcctgggaagagggtaagaactgtatccaggaggacagcggatgctttttctctcagactgcactcactaagactccagcatgccggccgagtgagtgctcctgaggtgcatgcgccttgtatgggcaccacgtgggcctcgccatgttttcacatacccactgcgagaaacacatatctaggtgctgaaggccccgaagacactatagttgaggatgcatccccaaagggtctgaccttgcttctgaggtcatgcattgagaaggcagctattcattagttgtcatatttcagctgagaagcaaaagcaggagctggccggcctcgacataacttcgtataatgtatgctatacgaagttataagcttaaggaattcgctagcatgcatgttaacggatccttaattaaaatgttggctgtgcctctgatcctctctctgggatgcttgcaggtgtttattgagggcccagccttagcctgcttctaggacatggcctaacccttctaactctccagggcaagcttgtactctgggccccaccgtgcacatgctgttgtgctcttcattaatttcttccaagtaaggagctgtttttaaagataaggtctcagtgggtagtcttgactggcctggaactcaaaatgtggatcaggctggcttggacttgacagaagtccacctgcttctgcctcctgagtgctgtggttaaagatgtgcattaccataccacatctggcctccaatcatttcttgtaagcttcttgcccctggattgtttattctgtaggtaaatgtctacagtaggtgaatggggtttggtggtcaaccttggaacttttattcacaaaacccaagatcctatgttcctgatttgacctaccttttctcctgctattgactgttcaggaaaatggtggaatcgtacggacttaggttttatccggtacgtttccttctcctggatgaccagctgcctggtcactgtggcctgactcgtgaggtcagagcccttggagactcctcacttctggcttcctgtgtatctgacccagagaaactgtctgtctcaggcatctctagggcatacaggatagggttgaattctttttttctcaagataggatgtagtgccacactcaggaagctaagacaggaggttcaccacaaatttaaggtcagtctaaactatagtgatttctaggctagtgagttacaccctgagaccctgcctaaaaaccaaaactgatcctaacagtataattagaaagaaaagcagccaggccagagtgtggcttagtagtgtttctttgcatgcacaacatttgggttcaatgtcaacacagcataaactgggttgatacaaagattagaatttaaaggtcatattggctatagagtgaattaaggctagcctgggttacatgagaccttgctttgaaaaatagatatgcatgcacccacacaggtgacaagatttctgaaaccctagataggtccagcaggaactgagcctgatagccaccaggattacagagcgactctcagatcttcacctgcatccatgttcttttctccagattgtgtgggaggcaagggtgggctccagcctcatctgttgtggccgtgactgtgctttgggtggtatcggctgccctgagaagcagaggagcccagtgacatctgggagtctttgaccccacagcttctgattctcgtgctctgtagatgggcagggctcagaggcctcacagttgagattccaggaaactggctttgtcattgctaaataaatttctgtgccagactttttgccaaaaaggaaagtaataatgaaaagtacaaatttatttcttactcagtgattgcagtagaaagcatgacctgtggcagggtgagctctgggtactctgccgctgtcttgagcctgcagtaaggaagatacttgtcttagttagggtttttctgttgtgagcagacatcatgaccaaggcaagtcttacaaggacaacatttagttggggctggcttacaggttctgaagttcagtccattatcatcaaggtgaaaacatggcagcatccagacaggcatggtgcaggaggagctgagagttctacatcttcatctgaaggctgctagcagaatattggctcccaagcagctaggagcccacacccacaaggccatacctcccaaaagtgccactccctgagctgaacatataatatacaaccattacattccaccccctggccctcataggcttgtccaaacataagcctatgggagccatacctacacatagcataatgcaaaatacatttagtccgacttcaaaagcccccatagtctatggcagtctcaacaataatcgtccaataacttaactgtaatccccaaagcaagacaggaagccagctgggctctgcatctccatgtctgatgtcttcagatcttctattcctttttcatctttgttgactgcaacaaacttctttctcctgggctggttctactccctggtagcatagcagctttccttagcagatagtccaactaccactctggtatctccaaggcagcttcttgttttaatgtctgggcctcctctccaaggtgacgtcacttccccagctctgccctcggtagctctaagctcaggttgatccctccactgccgctgctgctcttggtggccatcatctccaatacactgggggcttccgctgcaactagagcctctctaggctctcttcatggtgccaagcctcaactcctttgcatggccccttcagtcctgggccatcatctgcaaccgaggctgcactttgatcagtgatcttccgcctcagctgctcttcatggccccttcatgcctcaaggccagtgccacctgggggaccattgcagtcacccagcatagctgcagcatgaggtgcaaccttggctgtctctggaacacagcttcttggtgctcagaaaacacttccagtgatgctggttgtcgtcatgatttatttattatatgagtacacagttctcttcagacacaccagaaagagggtattgggcccctgttacagatggtcgtgagccaccatgtggttgctgggaattgaactcaggacctctggaagagcagtcagtgctcttaaccacagagccatctctccagccctgccggtctcttaatcactgctaatgccttagctcccgctaaccagcatcagctgtcccaggagtctttctcctcgtgattataaagccagagacacatggccgaagctgcttgctggagctggaacatggcccctagttctattgcgtcatcactagcttccagctttcgcgctccttcaaggcctaagtttgtcacgtggggatcttgctcagaactctgagatatgcaagcctgactcctgggattagaggtgtgtaccagcacgcccggaattaagcttttcttcacctacaacttgatctgtccttgaaagtagagatctgcctgcctttgcctccaggaattaaaaagcttgttctgcccagtatagaccaaaacttaactgggtgggatcttgccccaaggtcactagtcccttaattcaaactaatgtccttgaacacattcagctccattcacttccagtattcctttctaaccttgcaatgcttattcacatgctcttcctgagaacaaagtctacgatgggcctttctaaggcttcctttgtcattgtaattaacctgagcctccttagcctcaggcagactcttcagccaagggcaaaaatagctacttcttcaccaaactacaaaaacaaggctctagaccacataactgaaattcctcactgaaacctcttgtgctgggtctacacagttccgattactcacagcaacaaagtgttccatagtccagctaggatagaccatgaagccccacttgaaacattctgtggccttccaaatcccaagttccccaacctacattcttataagcaaaaacacggtcaggcctattaccgcaatatctcagtccctggtgccacctgtcttagagtttttctgctgtgagcagacaccatgaccaaggcaagtcttctaaggacaacatttaattggggctggcttacaggttccgaagttcagtccattatcaaggtggaaacatggcagcatccagacaggcatggtacaggaggagctaagagttctacgtcttctgaaggctgctagcagagtactgactcccaggcagctaggagcccacccatgaggccacacctactccaacagggctacacctcctagcattgccgctccctaagcagagcatatacaaaccgcaatactggccctgttgaaagagaagccaaccagcagagcctgcaggtctagcactcaggttgaggagggaggattacaagtttgaggccagcctggactcagcaagcacaaaacagaagaaaggaggcttgagaagttgagtggtggtttttgttgcggtgactgtaagccagttggacagtgtttgtcgtgtcccactgctaagttagtgctgtttagacagggcgctaatgagtctcctaggccagctaccaggtctgggcagggctcatttatggtaggtgtctctgttggccctgctgttcctttggttttatcttcgcatagattaaataattttttggctatttcactaatttaagtcctgcagtcaatgttcctagagtctggggagacctgcggactctgcagcctagtttccttttggtcatgatgtatgtgcaagaacttgagctaggatgatgttcacaatgtataaacagtccatgtgaacatatttacacacacgcagcgtctgtcagtagtccatcttgcgtctatgttggtgcactcagacatgtctggtggtctttgtgcctctcactttttacagagcaggactgagttgggtcttagtccaggaaaagccatgtgtgttacccacatctcctctgctacggccacactagtcctttgtgtactactgactgaaggagtgtcttgtctctttttttccctctttgtgacaacagccttgtcataggttcagaatcagggtagagaggagtatgtatggcaccaaatggtgaaattggaacacttgggaggcaggggcaggcagatctctgagttcaaggtcagcctgttacagaatgagttgcaggacagcctgggttacccagagaaacactgtctcaaaaacaaacaaataaaacaaaacaaacccaagaagctaaataaacaaacaaagattaaatgaatttgaagcctgcgctttggccgtgggcaggcccaggcacatagttaagacagatgtgttgttatcagaggcggccatgaatccgaatcctgtggctaatgatacgtgtttttggttcagtggacaggttgctggacccagccaacagggcccacatgagcttggaggagcagctgagagaacttctggacaagttggacctgacctgctccatgaagtccagcggctcacggagtaaacgggcaaagctgcttaaaaaagagattgctcttctccgaaacaagctgagccagcagcacagccaggctccgcccacaggggcaggcacgggaggctttgaagatgaggctgctccactggccccggacacagcggaggaaggtaagcatggggtaggagggccatacctcacgggctcggggctctcttgacaggcttaaatgatgctctgtagtaatgatgagcttgtacattttgaaggtcacggaactcttggttactggatattcctgctaggctttttttgatgctctttgaaaggatgttttggtgtgttctgtctgctgtattttggcacttagtttacaagcttaaaggaacagaatgagattttcttttaactcgagcttgaaagacttagaaggaatagtttagatccaatacagtgttgaaggtggcttctatggtgggaatggcaataacttagttgtattttgttaattgaggcagagtattatgtgagtagacaccctagaattgtttttaccttgtctacgtaggtcagaggacagctagttggagttggttttcctggcatcttagcacgcttggggatcaagcgcaggtggttaggccttgtaagcacctctgcccttagctaagccctcctgcggctggagttaggaaaggaggactggctagagaacagcccagccttgggctgggcatggtgggaggagtctgacgtgcacagacctgttcccagactctccctccacctcaggcctttcctgtggctcaccttcagtggacactgtcttattctggcagcgtgagtgacttctggggaaagagctggatagctgagatgttagggtggagaggaaggaagggaggaagtacagaagaggctgtctgccccgtgcgatccacgagatgagcaggtcattgtgtggagggagggaggcttctgtgtgtggtgcatctaactggcatgtttgatggtacaagcaccctttagtccacttgtcttgacatcaccacatttcaactccatgaaatggaaagaaaaataagacctacttcttctgccactgctattagcagcttgacttaggatctccctgtgcattttttttttctgccccatccaaataagaaaaacattaacacaagaccattgtcaccatagtttgcatttttttgatctgtatggctgcctgtcttagtagatgtgactttgccctattcctcagagtgacatggtttcagtatgtttatgccatgttaaatttagtcttataattttaacagttggtgacaatcttctaacccactttccccttctctggttgcttcttttatatggttatgctaggcaaccagcagaagctagggccaacaccagagttctcctggccttacatccttctagtgtgttcacttgtaaactcacaaacacccttggccttgccattaggtaacgtttaaacagtaacgctagggataacagggtaatataatcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccggacgcccgggcgaccggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatag

SEQ ID NO: 33 - DNA, 20 bp, Artificial Sequence
  source 1..20: /organism=“Artificial Sequence” /note=“Primer 1643_27” /mol_type=“unassigned DNA”
gtaagagtaccgtggttagc

SEQ ID NO: 34 - DNA, 21 bp, Artificial Sequence
  source 1..21: /organism=“Artificial Sequence” /note=“Primer 1643_28” /mol_type=“unassigned DNA”
gaggtacaaacctaagctacc

SEQ ID NO: 35 - DNA, 19 bp, Artificial Sequence
  source 1..19: /organism=“Artificial Sequence” /note=“Primer 1307_1” /mol_type=“unassigned DNA”
ggcagaagcacgcttatcg

SEQ ID NO: 36 - DNA, 21 bp, Artificial Sequence
  source 1..21: /organism=“Artificial Sequence” /note=“Primer 1307_2” /mol_type=“unassigned DNA”
gacaagcgttagtaggcacat

SEQ ID NO: 37 - DNA, 19 bp, Artificial Sequence
  source 1..19: /organism=“Artificial Sequence” /note=“Primer 1307_1” /mol_type=“unassigned DNA”
ggcagaagcacgcttatcg

SEQ ID NO: 38 - DNA, 21 bp, Artificial Sequence
  source 1..21: /organism=“Artificial Sequence” /note=“Primer 1307_2” /mol_type=“unassigned DNA”
gacaagcgttagtaggcacat

SEQ ID NO: 39 - DNA, 20 bp, Artificial Sequence
  source 1..20: /organism=“Artificial Sequence” /note=“Primer 1260_1” /mol_type=“unassigned DNA”
gagactctggctactcatcc

SEQ ID NO: 40 - DNA, 21 bp, Artificial Sequence
  source 1..21: /organism=“Artificial Sequence” /note=“Primer 1260_2” /mol_type=“unassigned DNA”
ccttcagcaagagctggggac

SEQ ID NO: 41 - DNA, 157 bp, Rattus
  source 1..157: /organism=“Rattus” /note=“BRD1 Exon 2” /mol_type=“unassigned DNA”
attaaataggattgcgaatcaggtggccattcagcggaagaagcagtttgtggagcgagcccacagctactggttactcaaaaggctgtctaggaatggtgctcccctgctgcggcggctccagtccagcctgcagtcccagagaaacacgcagcag